(19) United States
(12) Patent Application Publication
(10) Pub. No.: US 2021/0379195 A1
(43) Pub. Date: Dec. 9, 2021

(54) METHODS FOR ALLOGENEIC HEMATOPOIETIC STEM CELL TRANSPLANTATION

(71) Applicant: Magenta Therapeutics, Inc., Cambridge, MA (US)

(72) Inventors: Rahul Palchaudhuri, Somerville, MA (US); Jennifer Lynn Proctor, Medford, MA (US); Geoffrey O. Gillard, Harvard, MA (US); Anthony Boitano, Newton, MA (US); Sharon Hyzy, Malden, MA (US); Michael Cooke, Boston, MA (US); Adam Hartigan, Brookline, MA (US)

(21) Appl. No.: 17/244,864

(22) Filed: Apr. 29, 2021

Related U.S. Application Data
(63) Continuation of application No. PCT/US2019/058973, filed on Oct. 30, 2019.
(60) Provisional application No. 62/882,362, filed on Aug. 2, 2019.
(60) Provisional application No. 62/773,873, filed on Nov. 30, 2018.
(60) Provisional application No. 62/752,828, filed on Oct. 30, 2018.

(51) Int. Cl. A61K 47/68; A61K 31/675; A61K 35/28; A61P 37/06

(52) U.S. Cl. CPC A61K 47/6849; A61K 31/675; A61N 5/10; A61K 47/6817; A61P 37/06; A61K 35/28

(57) ABSTRACT

Described herein are compositions and methods useful for the depletion of CD117+ or CD45+ cells and for the treatment of various hematopoietic diseases, metabolic disorders, cancers, and autoimmune diseases, among others. The compositions and methods described herein can be used to treat a disorder, for instance, by depleting a population of CD117+ or CD45+ cancer cells or autoimmune cells. The compositions and methods described herein can also be used to prepare a patient for allogeneic hematopoietic stem cell transplant therapy and to improve the engraftment of allogeneic hematopoietic stem cell transplants by selectively depleting endogenous hematopoietic stem cells prior to the transplant procedure.

### RELATED APPLICATIONS

This application claims priority to International Application No. PCT/US2019/058973, filed Oct. 30, 2019, which claims priority to U.S. Provisional Application No. 62/752,828, filed on Oct. 30, 2018; U.S. Provisional Application No. 62/773,873, filed on Nov. 30, 2018; and U.S. Provisional Application No. 62/882,362, filed on Aug. 2, 2019. The entire content of each of the foregoing priority applications is incorporated by reference herein.

### SEQUENCE LISTING

The instant application contains a Sequence Listing which has been submitted electronically in ASCII format and is hereby incorporated by reference in its entirety. Said ASCII copy, created on Apr. 29, 2021, is named M103034_1480US_C1_Sequence_Listing.txt and is 365 kilobytes in size.

### FIELD

The present disclosure relates to the treatment of patients suffering from various pathologies, such as blood diseases, metabolic disorders, cancers, and autoimmune diseases, among others, by administration of an antibody drug conjugate capable of binding CD117 or CD45 expressed by a hematopoietic cell, such as a hematopoietic stem cell.

### BACKGROUND

Allogeneic cell therapy includes the transplantation of cells to a patient, where the transplanted cells are derived from a donor other than the patient. Common types of allogeneic donors used for allogeneic cell therapy include HLA-matched siblings, matched unrelated donors, partially matched family member donors, related umbilical cord blood donors, and unrelated umbilical cord blood donors. An ultimate goal in cell therapy is to identify allogeneic cell therapies that can form the basis of “off the shelf” products (Brandenberger, et al. (2011). BioProcess International. 9 (suppl. I): 30-37), which will expand the use of allogeneic cell therapy.

Despite its promise, the therapeutic use of allogeneic cells presently can have complications making this therapy challenging. In immune-competent hosts, transplanted allogeneic cells are rapidly rejected, a process termed host versus graft rejection (HvG). HvG can substantially reduce the efficacy of the transferred cells, as well as create adverse events in recipients, making the use of allogeneic cells limiting. There is currently a need for compositions and methods for promoting the engraftment of allogeneic hematopoietic stem cell grafts such that the multi-potency and hematopoietic functionality of these cells is preserved following transplantation.

### SUMMARY

Provided herein are antibodies or ADCs useful in conditioning procedures, in which a patient is prepared for receipt of a transplant including allogeneic hematopoietic stem cells. According to the methods described herein, a patient may be conditioned for an allogeneic hematopoietic stem cell transplant therapy by administration to the patient of an ADC, antibody or antigen-binding fragment thereof capable of binding an antigen expressed by hematopoietic cells (e.g., hematopoietic stem cells), such as CD117 (e.g., GNNK+CD117) or CD45 in combination with an immunosuppressant. As described herein, the antibody may be covalently conjugated to a cytotoxin so as to form an antibody drug conjugate (ADC).

In one aspect, provided herein is a method of depleting a population of CD117+ cells in a human patient in need of a hematopoietic stem cell transplant, the method comprising administering to the patient an effective amount of an anti-CD117 antibody drug conjugate and an immunosuppressant prior to the patient receiving a transplant comprising allogeneic hematopoietic stem cells.

In another aspect, provided herein is a method comprising administering to a human patient an anti-CD117 antibody drug conjugate and an immunosuppressant in an amount sufficient to deplete a population of CD117+ cells in the patient; and subsequently administering to the patient a transplant comprising allogeneic hematopoietic stem cells.

In another aspect, provided herein is a method comprising administering to a human patient a transplant comprising allogeneic hematopoietic stem cells, wherein the patient has been previously administered an anti-CD117 antibody and an immunosuppressant drug conjugate in an amount sufficient to deplete a population of hematopoietic stem cells in the patient.

In some embodiments, the CD117 is GNNK+CD117.

In another aspect, provided herein is a method of depleting a population of CD45+ cells in a human patient in need of a hematopoietic stem cell transplant, the method comprising administering to the patient an effective amount of the conjugate of an anti-CD45 antibody drug conjugate and an immunosuppressant prior to the patient receiving a transplant comprising allogeneic hematopoietic stem cells.

In another aspect, provided herein is a method comprising administering to a human patient an anti-CD45 antibody drug conjugate and an immunosuppressant in an amount sufficient to deplete a population of CD45+ cells in the patient; and subsequently administering to the patient a transplant comprising allogeneic hematopoietic stem cells.

In another aspect, provided herein is a method comprising administering to a human patient a transplant comprising allogeneic hematopoietic stem cells, wherein the patient has been previously administered an anti-CD45 antibody drug conjugate and an immunosuppressant in an amount sufficient to deplete a population of hematopoietic stem cells in the patient.

In another aspect, provided herein is a method comprising administering to a human patient a transplant comprising allogeneic hematopoietic stem cells, wherein the patient has been previously administered an anti-CD45 antibody drug conjugate in an amount sufficient to deplete a population of hematopoietic stem cells in the patient.

In some embodiments, the method further comprises administering the immunosuppressant to the patient after the patient has received the transplant.

In another aspect, provided herein is a method of depleting a population of CD117+ cells in a human patient in need of a hematopoietic stem cell transplant, the method comprising (a) administering to the human patient an anti-CD117 antibody drug conjugate in an amount sufficient to deplete a population of CD117+ cells in the patient; (b) administering to the human patient a transplant comprising allogeneic hematopoietic stem cells; and (c) subsequently administering an immunosuppressant to the patient.

In a further aspect, provided herein is a method of depleting a population of CD45+ cells in a human patient in need of a hematopoietic stem cell transplant, the method comprising (a) administering to the human patient an anti-CD45 antibody drug conjugate in an amount sufficient to deplete a population of CD45+ cells in the patient; (b) administering to the human patient a transplant comprising allogeneic hematopoietic stem cells; and (c) subsequently administering an immunosuppressant to the patient.

In some embodiments, the transplant comprises MHC-matched (e.g., HLA-matched) allogeneic hematopoietic stem cells. Accordingly, in some embodiments, the transplant comprises allogeneic hematopoietic stem cells in which all of the HLA antigens match the HLA antigens in the human patient.

In certain embodiments, the transplant comprises allogeneic hematopoietic stem cells that comprise at least one HLA-mismatch relative to the HLA antigens in the human patient. In certain embodiments, the allogeneic hematopoietic stem cells comprise at least two HLA-mismatches relative to the HLA antigens in the human patient. In certain embodiments, the allogeneic hematopoietic stem cells comprise at least three HLA-mismatches relative to the HLA antigens in the human patient. In certain embodiments, the allogeneic hematopoietic stem cells comprise at least four HLA-mismatches relative to the HLA antigens in the human patient. In certain embodiments, the allogeneic hematopoietic stem cells comprise at least five HLA-mismatches relative to the HLA antigens in the human patient. In certain embodiments, the allogeneic hematopoietic stem cells comprise at least six HLA-mismatches relative to the HLA antigens in the human patient. In certain embodiments, the allogeneic hematopoietic stem cells comprise at least seven HLA-mismatches relative to the HLA antigens in the human patient. In certain embodiments, the allogeneic hematopoietic stem cells comprise at least eight HLA-mismatches relative to the HLA antigens in the human patient. In certain embodiments, the allogeneic hematopoietic stem cells comprise at least nine HLA-mismatches relative to the HLA antigens in the human patient. In certain embodiments, the allogeneic hematopoietic stem cells comprise a full HLA-mismatch relative to the HLA antigens in the human patient. In certain embodiments, the transplant comprises allogeneic hematopoietic stem cells that comprise between one and four HLA-mismatches, between one and three HLA-mismatches, between one and two HLA-mismatches, between two and four HLA-mismatches, between two and three HLA-mismatches, or between three and four HLA-mismatches relative to the HLA antigens in the human patient.

In some embodiments, the transplant comprises allogeneic hematopoietic stem cells that comprise at least one minor histocompatibility antigen (miHA)-mismatch relative to the minor histocompatibility antigens in the human patient.

In some embodiments, the transplant comprises HLA-mismatched allogeneic hematopoietic stem cells.

In some embodiments, the method is effective to establish at least 80% donor chimerism. In some embodiments, the method is effective to establish at least 85% donor chimerism. In some embodiments, the method is effective to establish at least 90% donor chimerism. In some embodiments, the method is effective to establish at least 95% donor chimerism. In some embodiments, the donor chimerism is assessed at least 6 weeks, 7 weeks, 8 weeks, 9 weeks, or 10 weeks post-transplantation. In some embodiments, the donor chimerism is peripheral myeloid chimerism. In some embodiments, the donor chimerism is T-cell chimerism.

In some embodiments, the immunosuppressant is cyclophosphamide. In some embodiments, the immunosuppressant is 30F11. In some embodiments, the immunosuppressant is cyclophosphamide (Cytoxan, e.g., low-dose Cytoxan). In some embodiments, the immunosuppressant is 30F11 and cyclophosphamide. In some embodiments, the immunosuppressant is total body irradiation (TBI, e.g., low-dose TBI). In some embodiments, the immunosuppressant (e.g., Cytoxan) is administered post-transplant. In some embodiments, the immunosuppressant (e.g., 30F11) is administered pre-transplant. In some embodiments, the immunosuppressant is administered at substantially the same time as the patient receives the transplant.

In some embodiments, the conjugate is internalized by a cancer cell, autoimmune cell, or hematopoietic stem cell following administration to the patient.

In some embodiments, the transplant comprising hematopoietic stem cells is administered to the patient after the concentration of the conjugate has substantially cleared from the blood of the patient.

In some embodiments, the hematopoietic stem cells or progeny thereof maintain hematopoietic stem cell functional potential after two or more days following transplantation of the hematopoietic stem cells into the patient.

In some embodiments, the hematopoietic stem cells or progeny thereof are capable of localizing to hematopoietic tissue and/or reestablishing hematopoiesis following transplantation of the hematopoietic stem cells into the patient.

In some embodiments, upon transplantation into the patient, the hematopoietic stem cells give rise to recovery of a population of cells selected from the group consisting of megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

In some embodiments, the patient is suffering from a stem cell disorder.

In some embodiments, the patient is suffering from a hemoglobinopathy disorder, an autoimmune disorder, myelodysplastic disorder, immunodeficiency disorder, or a metabolic disorder.

In some embodiments, the patient is suffering from cancer.

In some embodiments, the ADC comprises an anti-CD117 antibody comprising a heavy chain/light chain (HC/LC) CDR set (CDR1, CDR2, or CDR3) or a HC/LC variable region set as described in Table 3.

In some embodiments, the antibody of the conjugate has a dissociation rate (KOFF) of 1×10⁻² to 1×10⁻³, 1×10⁻³ to 1×10⁻⁴, 1×10⁻⁵ to 1×10⁻⁸, 1×10⁻⁶ to 1×10⁷ or 1×10⁻⁷ to 1×10⁻⁸ as measured by bio-layer interferometry (BLI).

In some embodiments, the antibody of the conjugate binds CD117 with a KD of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

In some embodiments, the antibody of the conjugate is a human antibody.

In some embodiments, the antibody of the conjugate is an intact antibody.

In some embodiments, the antibody of the conjugate is an IgG. In some embodiments, the IgG is an IgG 1 isotype, a IgG2 isotype, a IgG3 isotype, or a IgG4 isotype.

In some embodiments, the antibody is conjugated to a cytotoxin via a linker. In some embodiments, the cytotoxin is an RNA polymerase inhibitor. In some embodiments, the RNA polymerase inhibitor is an amatoxin.

In some embodiments, the RNA polymerase inhibitor is an amanitin. In some embodiments, the amanitin is selected from the group consisting of α-amanitin, β-amanitin, γ-amanitin, ε-amanitin, amanin, amaninamide, amanullin, amanullinic acid, and proamanullin. In some embodiments, the cytotoxin selected from the group consisting of an pseudomonas exotoxin A, deBouganin, diphtheria toxin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, and indolinobenzodiazepine pseudo dimer. In some embodiments, the auristatin is MMAE or MMAF.

In some embodiments, the antibody is conjugated to the toxin by way of a cysteine residue in the Fc domain of the antibody. In some embodiments, the cysteine residue is introduced by way of an amino acid substitution in the Fc domain of the antibody. In some embodiments, the amino acid substitution is D265C.

### BRIEF DESCRIPTION OF THE FIGURES

FIGS. 1A-1E graphically depict the design and results of an in vivo study of ADC conditioning with an anti-CD45 ADC (104-saporin; “CD45-SAP”) or anti-CD117 ADC (2B8-saporin; “CD117-SAP”) combined with 30F11 and post-transplant Cytoxan prior to a murine minor mismatch transplant of Balb/c donor cells into DBA/2 recipients. FIGS. 1A and 1B depict a schematic of the in vivo mouse model (FIG. 1A) and dosing schedule for the various experimental groups (FIG. 1B). CD45-SAP, CD117-SAP, or control treatments (e.g., 2 Gy TBI or No TBI) were administered to the transplant recipients in combination with an immunosuppressant (30F11) pre-transplant and Cytoxan post-transplant. FIG. 1C graphically depicts the degree of bone marrow depletion (as measured by the number of long term-HSC (LT-HSC) per femur (y-axis)) as a function of treatment condition seven days post administration in C57BL/6 mice. FIG. 1D graphically depicts the percent of overall blood donor chimerism (CD45.1+) in the peripheral blood 12-weeks post-transplantation. FIG. 1E graphically depicts the percent of myeloid chimerism, B cell chimerism, and T cell chimerism 12 weeks post-transplantation.

FIGS. 2A-2C graphically depict the results of an in vivo study of ADC conditioning with an anti-CD45 ADC (104-saporin; “CD45-SAP”) combined with post-transplant Cytoxan prior to a murine minor mismatch transplant of Balb/cByJ donor cells into DBA/2 recipients. FIGS. 2A-2C graphically depict the percent of total donor chimerism (y-axis), the percent of peripheral donor myeloid chimerism (FIG. 2A), and the percent of donor T cell chimerism (FIG. 2B) in transplant recipients as a function of treatment mode in DBA/2 mice transplanted with CD45.1+ cells at 8 weeks post-transplantation. FIG. 2D graphically depicts the number of donor-derived long term-HSC (LT-HSC) per femur (y-axis) in transplant recipients 12-weeks post-transplant.

FIGS. 3A-3B graphically depict the results of an in vivo depletion assay showing that CD45-ADC effectively depletes murine HSCs and lymphocytes in C57 mice. FIG. 3A is a schematic of an in vivo study to assess murine HSC depletion by an anti-CD45-ADC (CD45-saporin or “CD45-SAP”). FIG. 3B depicts the flow cytometry gating strategy and results showing depletion of long-term HSCs by CD45-SAP in bone marrow collected on Day 7. FIG. 3C graphically depicts the level of long-term HSCs in bone marrow seven days post dosing of PBS, isotype-SAP, or CD45-SAP. FIG. 3D graphically depicts the level of peripheral lymphocytes seven days post-dosing of PBS, isotype-SAP, or CD45-SAP. The asterisk (*) indicates p<0.05 when comparing against any control group.

FIGS. 4A-4C graphically depict the results of an in vivm study of a murine model of a full mismatch bone marrow transplant. C57Bl/6 (H-2b, CD45.2+) mice were conditioned with an anti-CD45-ADC (anti-CD45-PDB or “CD45-PBD”) alone or with an anti-CD4 and anti-CD8 antibody and transplanted with Balb/c (H-2d, CD45.1+) bone marrow. FIG. 4A graphically depicts the percentage of donor chimerism in transplant recipients as detected at 3- and 8-weeks post-transplant in blood using the CD45.1+ antigen. FIG. 4B graphically depicts the percent of peripheral donor myeloid chimerism, the percent of B cell chimerism, and the percent of T cell chimerism as a function of treatment mode in transplant recipients at 8 weeks post-transplantation. FIGS. 4C and 4D graphically depicts the total cell number (CD45+) in the peripheral blood (FIG. 4C) and spleen (FIG. 4D) two days post ADC administration.

FIGS. 5A-5G graphically depict the results of an in vivo study of a murine model of a full mismatch bone marrow transplant. C57Bl/6 (H-2b, CD45.2+) mice were conditioned with an anti-CD45-ADC (“104-PBD”) alone or with lose-dose TBI and transplanted with Balb/c (H-2d, CD45.1+) bone marrow. FIG. 5A graphically depicts the number of long term-HSC (LT-HSC) per femur (y-axis) as a function of treatment condition at different levels of irradiation in transplant recipients two days post ADC administration. FIGS. 5B-6E graphically depict the degree of bone marrow depletion (cells per femur (y-axis)) of total CD45+ cells (FIG. 5B), myeloid cells (FIG. 5C), B cells (FIG. 5D), or T cells (FIG. 5E) as a function of treatment condition at different levels of irradiation in transplant recipients two days post ADC administration. FIG. 5F graphically depicts the percent of donor chimerism in the peripheral blood of transplant recipients four weeks post-transplant. FIG. 5G graphically depicts the percent of myeloid chimerism, B cell chimerism, and T cell chimerism in transplant recipients four weeks post-transplant.

### DETAILED DESCRIPTION

Provided herein are antibodies or ADCs useful in conditioning procedures, in which a patient is prepared for receipt of a transplant including allogeneic hematopoietic stem cells. Such procedures promote the engraftment of an allogeneic hematopoietic stem cell transplant. According to the methods described herein, a patient may be conditioned for an allogeneic hematopoietic stem cell transplant therapy by administration to the patient of an ADC, antibody or antigen-binding fragment thereof capable of binding an antigen expressed by hematopoietic cells (e.g., hematopoietic stem cells), such as CD117 (e.g., GNNK+CD117) or CD45 in combination with an immunosuppressant. As described herein, the antibody may be covalently conjugated to a cytotoxin so as to form an antibody drug conjugate (ADC). Administration of an ADC, antibody, antigen-binding fragment thereof, or drug-antibody conjugate capable of binding one or more of the foregoing antigens in combination with an immunosuppressant to a patient in need of hematopoietic stem cell transplant therapy can promote the engraftment of an allogeneic hematopoietic stem cell graft, for example, by selectively depleting endogenous hematopoietic stem cells, thereby creating a vacancy filled by an exogenous hematopoietic stem cell transplant.

### Definitions

As used herein, the term “about” refers to a value that is within 5% above or below the value being described.

As used herein, the term “allogeneic”, when used in the context of transplantation, is used to define cells (or tissue or an organ) that are transplanted from a genetically dissimilar donor to a recipient of the same species.

As used herein, the term “autologous” refers to cells or a graft where the donor and recipient are the same subject.

As used herein, the term “xenogeneic” refers to cells where the donor and recipient species are different.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered antibodies, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab)₂, Fab, Fv, rigG, and scFv fragments, so long as they exhibit the desired antigen-binding activity.

The antibodies of the present disclosure are generally isolated or recombinant. “Isolated,” when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody.” refers to an antibody which is substantially free of other antibodies having different antigenic specificities. For instance, an isolated antibody that specifically binds to CD117 is substantially free of antibodies that specifically bind antigens other than CD117. Similarly, an isolated antibody that specifically binds to CD45 is substantially free of antibodies that specifically bind antigens other than CD45.

The term “monoclonal antibody” as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof. Unless otherwise indicated, the term “monoclonal antibody” (mAb) is meant to include both intact molecules, as well as antibody fragments (including, for example. Fab and F(ab′)₂ fragments) that are capable of specifically binding to a target protein. As used herein, the Fab and F(ab′)₂ fragments refer to antibody fragments that lack the Fc fragment of an intact antibody. In one embodiment, an antibody fragment comprises an Fc region.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)₂ fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-CD117 antibody” or “an antibody that binds to CD117” refers to an antibody that is capable of binding CD117 with sufficient affinity such that the antibody is useful as a diagnostic and/or therapeutic agent in targeting CD117.

As used herein, the term “anti-CD45 antibody” or “an antibody that binds to CD45” refers to an antibody that is capable of binding CD45 with sufficient affinity such that the antibody is useful as a diagnostic and/or therapeutic agent in targeting CD45.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the VH and VL domains of neighboring peptide chains spatially proximal to one another (see, for example, Holliger et al., Proc. Natl. Acad. Sci. USA 90:6444-48, 1993).

As used herein, the term “bispecific antibody” refers to, for example, a monoclonal, e.g., a human or humanized antibody, that is capable of binding at least two different antigens or two different epitopes. For instance, one of the binding specificities can be directed towards an epitope on a hematopoietic stem cell surface antigen, such as CD117 (e.g., GNNK+CD117) or CD45, and the other can specifically bind an epitope on a different hematopoietic stem cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody). An “intact” or “full length” antibody, as used herein, refers to an antibody having two heavy (H) chain polypeptides and two light (L) chain polypeptides interconnected by disulfide bonds. Each heavy chain is comprised of a heavy chain variable region (abbreviated herein as HCVR or VH) and a heavy chain constant region. The heavy chain constant region is comprised of three domains, CH1. CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

The term “specifically binds”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., an antigen expressed by hematopoietic stem cells, such as CD117 (e.g., GNNK+CD117), or CD45, or an antigen expressed by mature immune cells (e.g., T-cells), such as CD4 or CD8, if the antibody has a K_{D} for the target of at least about 10⁻⁴ M, about 10⁻⁵ M, about 10⁻⁶ M, about 10⁻⁷ M, about 10⁻⁸ M, about 10⁻⁹ M, about 10⁻¹⁰ about M, 10⁻¹¹ about M, about 10⁻¹² M, or less (less meaning a number that is less than about 10⁻¹², e.g. 10⁻¹³). In one embodiment, the term “specifically binds” refers to the ability of an antibody to bind to an antigen with an Kd of at least about 1×10⁻⁶ M, 1×10⁻⁷ M, about 1×10⁻⁸ M, about 1×10⁻⁹ M, about 1×10⁻¹⁰ M, about 1×10⁻¹¹ M, about 1×10⁻¹² M, or more and/or bind to an antigen with an affinity that is at least two-fold greater than its affinity for a nonspecific antigen. In one embodiment, K_{D} is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of an antigen, e.g., CD117 (e.g., GNNK+CD117) or CD45.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397. The terms “Fc”, “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the second constant domain CH2 (e.g., residues at EU positions 231-340 of human IgG1) and the third constant domain CH3 (e.g., residues at EU positions 341-447 of human IgG1). As used herein, the Fc domain includes the “lower hinge region” (e.g., residues at EU positions 233-239 of human IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or Clq as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

The variant Fc domains described herein are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdeIG”. As yet another example, the 1253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fc gamma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIIIb-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-80, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, L234A, L235A, and/or H435A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC, an antibody or an antigen-binding fragment thereof capable of binding an antigen expressed by hematopoietic stem cells, such as CD117 (e.g., GNNK+CD117) or CD45. As described herein, the antibody may be covalently conjugated to a cytotoxin so as to form an ADC. Administration of an ADC, an antibody, or an antigen-binding fragment thereof capable of binding one or more of the foregoing antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “effective amount” or “therapeutically effective amount” refers to an amount that is sufficient to achieve the desired result or to have an effect on an autoimmune disease or cancer.

As used herein, the term “half-life” refers to the time it takes for the plasma concentration of the antibody drug in the body to be reduced by one half or 50%. This 50% reduction in serum concentration reflects the amount of drug circulating.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo).

However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU): incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B cells and T cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119. CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential).

Any of these HSCs can be used in the methods described herein. ST-HSCs are particularly useful because they are highly proliferative and thus, can more quickly give rise to differentiated progeny.

As used herein, the term “anti-hematopoietic cell antibody” or “anti-HC antibody” refers to an antibody that specifically binds an antigen expressed by hematopoietic stem cells, such as CD117 (e.g., GNNK+CD117), or CD45, or an antigen expressed by mature immune cells (e.g., T-cells) such as CD45.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, T cells and B cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “donor chimerism” refers to the percentage of donor cells in the lymphohematopoietic system of a recipient (i.e., host) of an allogeneic hematopoietic stem cell transplant. For example, 85% donor chimerism refers to a lymphohematopoietic system comprising 85% donor cells following an allogeneic hematopoietic stem cell transplant. In some embodiments, the methods herein are effective to establish at least 80% donor chimerism, at least 85% donor chimerism, or at least 90% chimerism in vivo. Engraftment and the degree of chimerism (e.g., percentage of donor stem cells in the host) can be detected by any number of standard methods. The presence of donor markers, such as sex chromosome-specific markers, in the host can be determined, for example, using standard cytogenetic analysis, polymerase chain reaction (PCR) with appropriate primers, variable number of tandem repeats-PCR (VNTR-PCR), microsatelite markers or other finger-printing techniques, or fluorescence in situ hybridization (FISH). Host-donor chimerism can also be determined by determining the percentage of donor-type cells in host blood using, for example, standard complement-dependent microcytotoxicity tests.

As used herein, the term “mismatch” (e.g., “MHC-mismatch”, “HLA-mismatch”, or “miHA-mismatch”), in the context of hematopoietic stem cell transplants, refers to the presence of at least one dissimilar (e.g., non-identical) cell surface antigen on an allogeneic cell (or tissue or an organ) (e.g., a donor cell) relative to a variant of the antigen expressed by the recipient. An allogeneic transplant can, in some embodiments, contain “minor mismatches” with respect to the transplant recipient. Such “minor mismatches” include individual differences in cell surface antigens other than MHC antigens or HLA antigens. Minor mismatches include differences in minor histocompatibility antigens. In some embodiments, an allogeneic transplant can contain “major mismatches” with respect to the transplant recipient. Such “major mismatches” refer to differences in the MHC haplotype or HLA haplotype between the transplant and the recipient. In an exemplary embodiment, an allogeneic transplant can share the same MHC or HLA haplotype as the transplant recipient, but can contain one or more minor mismatches (also referred to herein as a “minor mismatch allogeneic transplant”). In another exemplary embodiment, an allogeneic transplant can contain one or more major mismatches, alone or in addition to one or more minor mismatches. A “full mismatch” allogeneic transplant refers to an allogeneic transplant that contains one or more major mismatches and one or more minor mismatches. The presence of major and/or minor mismatches can be determined by standard assays used in the art, such as serological, genomic, or molecular analysis. In some embodiments, at least one major histocompatibility complex antigen is mismatched relative to an allele expressed by the recipient. Alternatively or additionally, at least one minor histocompatibility antigen is mismatched relative to an allele expressed by the recipient.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain, scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

As used herein, the phrase “substantially cleared from the blood” refers to a point in time following administration of a therapeutic agent (such as an anti-CD117 antibody, an anti-CD45 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments, and protein ligands, such as ELISA-based detection assays known in the art or described herein. Additional assays that can be used to detect antibodies, or antibody fragments, include immunoprecipitation techniques and immunoblot assays, among others known in the art.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein “to treat” or“treatment”, refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease, any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting the engraftment of exogenous hematopoietic cells in a patient following antibody conditioning therapy as described herein and subsequent hematopoietic stem cell transplant therapy Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+ T-cell that expresses a T-cell receptor that cross-reacts with a self antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome. Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

The term “immunosuppressive agent” or “immunosuppressant” as used herein refers to substances that act to suppress or mask the immune system of the recipient of the hematopoietic transplant. This would include substances that suppress cytokine production, downregulate or suppress self-antigen expression, or mask the MHC antigens. Examples of such agents include calcineurin/MTOR inhibitors (e.g. tacrolimus, sirolimus, rapamycin, ciclosporin, everolimus), co-stimulatory blockade molecules (e.g. CTLA4-lg, anti-CD40L), NK depletion agents, Anti-thymocyte globulin (ATG), alkylating agents (e.g., nitrogen mustards, e.g., cyclophosphamide; nitrosoureas (e.g., carmustine); platinum compounds), methotrexate, anti-TCR agents (e.g., muromonab-CD3), anti-CD20 antibodies (e.g., rituximab, ocrelizumab, ofatumumab, and veltuzumab), fludarabine, Campath (alemtuzumab), 2-amino-8-aryl-5-substituted pyrimidines (see U.S. Pat. No. 4,665,077, supra, the disclosure of which is incorporated herein by reference), azathioprine (or cyclophosphamide, if there is an adverse reaction to azathioprine); bromocryptine; glutaraldehyde (which masks the MHC antigens, as described in U.S. Pat. No. 4,120,649, supra); antiidiotypic antibodies for MHC antigens; cyclosporin A; one or more steroids, e.g., corticosteroids, e.g., glucocorticosteroids such as prednisone, methylprednisolone, hydrocortisone, and dexamethasone; anti-interferon-γ antibodies; anti-tumor necrosis factor-α antibodies; anti-tumor necrosis factor-β antibodies; anti-interleukin-2 antibodies; anti-cytokine receptor antibodies such as anti-IL-2 receptor antibodies; heterologous anti-lymphocyte globulin; pan-T antibodies, e.g., OKT-3 monoclonal antibodies; antibodies to CD4; antibodies to CD8, antibodies to CD45 (e.g., 30-F11, YTH24.5, and/or YTH54.12 (e.g., a combination of YTH24.5 and YTH54.12)); streptokinase; streptodomase; or RNA or DNA from the host.

Additional immunosuppressants include, but are not limited to, total body irradiation (TBI), low-dose TBI, and/or Cytoxan.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease.” ASH Education Book, 1:319-338 (2000), the disclosure of which is incorporated herein by reference in its entirety as it pertains to pathologies that may be treated by administration of hematopoietic stem cell transplant therapy.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of antibodies and antibody fragments of the present disclosure include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “conjugate” or “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the term “microtubule-binding agent” refers to a compound which acts by disrupting the microtubular network that is essential for mitotic and interphase cellular function in a cell. Examples of microtubule-binding agents include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof.

As used herein, the term “amatoxin” refers to a member of the amatoxin family of peptides produced by Amanita phalloides mushrooms, or a variant or derivative thereof, such as a variant or derivative thereof capable of inhibiting RNA polymerase II activity. Amatoxins useful in conjunction with the compositions and methods described herein include compounds such, as but not limited to, compounds of Formulas (III), (IIIA), (IIIB), and (IIIC), each as described herein below (e.g., an α-amanitin, β-amanitin, γ-amanitin, ε-amanitin, amanin, amaninamide, amanullin, amanullinic acid, or proamanullin) As described herein, amatoxins may be conjugated to an antibody, or antigen-binding fragment thereof, for instance, by way of a linker moiety (L) (thus forming an ADC). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

The term “acyl” as used herein refers to —C(═O)R, wherein R is hydrogen (“aldehyde”), alkyl, alkenyl, alkynyl, carbocyclyl, aryl, heteroaryl, or heterocyclyl, as defined herein, as defined herein. Non-limiting examples include formyl, acetyl, propanoyl, benzoyl, and acryloyl.

As used herein, the term “alkyl” refers to a straight- or branched-chain alkyl group having, for example, from 1 to 20 carbon atoms in the chain. Examples of alkyl groups include methyl, ethyl, n-propyl, isopropyl, butyl, isobutyl, sec-butyl, tert-butyl, pentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. Examples of alkenyl groups include vinyl, propenyl, isopropenyl, butenyl, tert-butylenyl, hexenyl, and the like.

As used herein, the term “alkenylene” refers to a straight- or branched-chain divalent alkenyl group.

The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain. Examples of alkynyl groups include propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocyloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like. The divalent positions may be one or more heteroatoms.

As used herein, the term “arylene” refers to a divalent aryl group. The divalent positions may be on the same or different atoms.

“Heteroaralkyl” as used herein refers to an acyclic alkyl radical in which one of the hydrogen atoms bonded to a carbon atom, typically a terminal or sp3 carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

As used herein, the term “heterocycloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group in which one or more ring atoms is a heteroatom, e.g., nitrogen, oxygen, or sulfur. Heteroaryl groups include pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroarylene” refers to a divalent heteroaryl group. The divalent positions may be on the same or different atoms. The divalent positions may be one or more heteroatoms.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl. 5-pyridyl, 8-pyridyl, 3-pyridazinyl, 4-pyridazinyl, 5-pyridazinyl, 6-pyridazinyl, 2-pyrimidinyl, 4-pyrimidinyl. 5-pyrimidinyl, 6-pyrimidinyl, 2-pyrazinyl, 3-pyrazinyl, 5-pyrazinyl, 8-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine. 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted with, for example, from 1 to 5 substituents selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO—, —SOH, —S(═O)₂R, —OS(═O)₂OR, —S(═O)₂NH₂. —S(═O)₂N(R)₂, —S(═O)R, —OP(═O)(OH)₂. —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from alkyl, aryl, heterocycloalkyl or heteroaryl, protecting group and prodrug moiety. Wherever a group is described as “optionally substituted,” that group can be substituted with one or more of the above substituents, independently for each occasion. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂— and the like. Other radical naming conventions clearly indicate that the radical is a di-radical such as “alkylene,” “alkenylene,” “arylene,” “heterocycloalkylene,” and the like.

As used herein, the term “coupling reaction” refers to a chemical reaction in which two or more substituents suitable for reaction with one another react so as to form a chemical moiety that joins (e.g., covalently) the molecular fragments bound to each substituent. Coupling reactions include those in which a reactive substituent bound to a fragment that is a cytotoxin, such as a cytotoxin known in the art or described herein, reacts with a suitably reactive substituent bound to a fragment that is an antibody, or antigen-binding fragment thereof, such as an antibody, or antigen-binding fragment thereof, specific for CD117 (such as GNNK+CD117) known in the art or described herein. Examples of suitably reactive substituents include a nucleophile/electrophile pair (e.g., a thiol/haloalkyl pair, an amine/carbonyl pair, or a thiol/α,β-unsaturated carbonyl pair, among others), a diene/dienophile pair (e.g., an azide/alkyne pair, among others), and the like. Coupling reactions include, without limitation, thiol alkylation, hydroxyl alkylation, amine alkylation, amine condensation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein.

As used herein, “CRU (competitive repopulating unit)” refers to a unit of measure of long-term engrafting stem cells, which can be detected after in-vivo transplantation.

As used herein, “drug-to-antibody ratio” or “DAR” refers to the number of cytotoxins, e.g., amatoxin, attached to the antibody of an ADC. The DAR of an ADC can range from 1 to 8, although higher loads are also possible depending on the number of linkage sites on an antibody. Thus, in certain embodiments, an ADC described herein has a DAR of 1, 2, 3, 4, 5, 6, 7, or 8.

Wherever a substituent is depicted as a di-radical (i.e., has two points of attachment to the rest of the molecule), it is to be understood that the substituent can be attached in any directional configuration unless otherwise indicated.

### Method of Treatment

Disclosed herein are methods of depleting a population of CD117+ cells and/or a population of CD45+ cells in a patient in need of an allogeneic transplant, e.g., an allogeneic hematopoietic stem cell (HSC) transplant. Also provided herein are methods of increasing the level of engraftment of allogeneic cells in a recipient subject. The methods provided herein can be used for treating a variety of disorders relating to allogeneic transplantation, such as diseases of a cell type in the hematopoietic lineage, cancers, autoimmune diseases, metabolic disorders, graft versus host disease, host versus graft rejection, and stem cell disorders, among others. The compositions and methods described herein can (i) directly deplete a population of cells that give rise to a pathology, such as a population of cancer cells (e.g., leukemia cells) and autoimmune cells (e.g., autoreactive T-cells), and/or (ii) can deplete a population of endogenous hematopoietic stem cells so as to promote the engraftment of transplanted hematopoietic stem cells by providing a niche to which the transplanted cells may home. Depletion of endogenous hematopoietic cells in a subject in need of a transplant, e.g., a HSC transplant can be achieved by administration of an ADC, antibody, or antigen-binding fragment thereof, capable of binding an antigen expressed by an endogenous hematopoietic stem cell. In the case of preparing a patient for transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. ADCs, antibodies, or antigen-binding fragments thereof, capable of binding an antigen expressed by hematopoietic stem cells (e.g., CD117+(e.g., GNNK+CD117) or CD45+ cells) or an antigen expressed by immune cells (e.g., mature immune cells), such as T-cells (e.g., CD45) can be administered to a patient to effect cell depletion. Thus, ADCs, antibodies, or antigen-binding fragments thereof, that bind an antigen expressed by hematopoietic stem cells (e.g., CD117 (e.g., GNNK+CD117) or CD45) or an antigen expressed by immune cells (e.g., mature immune cells), such as T-cells (e.g., CD45) can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted cells, e.g., hematopoietic stem cells.

Transplant patients can receive a transplant that is autologous, in which the transplant comprises the subject's own cells. In other embodiments, transplant patients can receive a transplant that is allogeneic, in which the transplant comprises cells obtained or derived from another individual. In the case of allogeneic transplantation, engraftment of transplanted cells is complicated by the potential for an immune response against the transplant mediated by immune cells of the host (host vs graft disease), or by the potential for an immune response against cells of the host mediated by immune cells present in the transplant (graft vs host disease). The likelihood of the foregoing complications increases with the degree of dissimilarity in the antigenic makeup of the transplant, in relation to the transplant recipient patient. Accordingly, allogeneic transplants are typically performed between patients having the highest degree of similarity possible between HLA antigens and minor histocompatibility antigens. Due to the need for a very high degree of antigenic similarity between an autologous transplant donor and recipient, there are patients in need of a transplant who are unable to receive this therapy because a suitably matched donor is not available.

The methods provided herein are based, at least in part, on the discovery that conditioning a patient in need of an allogeneic transplant with both (i) an ADC capable of binding CD117 or CD45, and (ii) an immunosuppressive agent, significantly increases the engraftment of allogeneic donor cells, including in situations where the allogeneic cells contain a high degree of antigenic mismatch with respect to the transplant recipient. Without wishing to be bound by theory, it is believed that the immunosuppressive agent inhibits the activity of residual immune cells, e.g., residual T cells, present in the patient following administration of the ADC, which can limit engraftment of autologous cells. When the ADC is administered in conjunction with an immunosuppressive agent, engraftment of autologous donor cells is increased, leading to an increase in donor chimerism. Accordingly, the methods described herein can be used, in some embodiments, to increase engraftment of autologous hematopoietic stem cells, and increase donor chimerism in the bone marrow and the peripheral blood (including myeloid chimerism, B cell chimerism, and T cell chimerism).

As described herein, hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells. B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population.

The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additionally or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The antibodies, or antigen-binding fragments thereof, and conjugates described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In addition, the compositions and methods described herein can be used to treat cancers directly, such as cancers characterized by cells that are CD117+(e.g., GNNK+CD117) or CD45+. For instance, the compositions and methods described herein can be used to treat leukemia, such as in patients that exhibit CD117+ leukemic cells. By depleting CD117+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

In addition, the compositions and methods described herein can be used to treat autoimmune disorders. For instance, an antibody, or antigen-binding fragment thereof, can be administered to a subject, such as a human patient suffering from an autoimmune disorder, so as to kill a CD45+ immune cell. For example, a CD45+ immune cell may be an autoreactive lymphocyte, such as a T-cell that expresses a T-cell receptor that specifically binds, and mounts an immune response against, a self antigen. By depleting self-reactive, CD45+ cells, the compositions and methods described herein can be used to treat autoimmune pathologies, such as those described below. Additionally or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

The antibody or antibody-drug conjugate can be administered to the human patient in need prior to transplantation of cells or a solid organ to the patient. In one embodiment, an anti-CD45 ADC or anti-CD117 ADC is administered to the human patient in need thereof prior to (e.g., about 3 days before, about 2 days before, about 12 hours before; about 12 hours to 3 days before, about 1 to 3 days before, about 1 to 2 days before, or about 12 hours to 2 days before) transplantation of cells or a solid organ. In one embodiment, the transplant is administered to the patient after the ADC has cleared or substantially cleared the blood of the patient.

By administering an immunosuppressant, the methods described herein are also useful for preventing host versus graft (HvG) reactions. Graft failure or graft rejection, including failure after allogeneic hematopoietic stem cell transplantation, may be manifested generally as either lack of initial engraftment of donor cells, or loss of donor cells after initial engraftment (for review see Mattsson et al. (2008) Biol Blood Marrow Transplant. 14(Suppl 1): 165-170).

A variety of immunosuppressants can be used in combination with an anti-CD117 antibody, anti-CD45 antibody, or antibody-drug conjugate thereof, to prevent host versus graft (HvG) reactions thereby preventing or reducing the risk of allogeneic graft failure. Use of an immunosuppressant in a patient at risk for a HvG reaction enables engraftment of donor cells with a greater degree of MHC-mismatch (e.g, HLA-mismatch) or minor histocompatibility antigen (miHA)-mismatch.

In some embodiments, the anti-CD117 antibody, anti-CD45 antibody, or antibody-drug conjugate thereof, is administered in combination with one or more immunosuppressants (e.g., one, two, or three immunosuppressants). In some embodiments, the anti-CD117 antibody, anti-CD45 antibody, or antibody-drug conjugate thereof, is administered in combination with two or more immunosuppressants, such as those described herein.

In one embodiment, the anti-CD117 antibody, anti-CD45 antibody, or antibody-drug conjugate thereof, is administered in combination with an immune depleting agent that enables B-cell and/or T-cell depletion.

In some embodiments, the immune depleting agent is an anti-CD4 antibody, an anti-CD8 antibody, or both an anti-CD4 antibody and an anti-CD8 antibody. Examples of anti-CD4 antibodies are known in the art including, for example, ibalizumab (also known as Trogarzo, TMB-355, TNX-355, or Hu5A8; see, e.g., U.S. Pat. Nos. 9,790,276 and 9,587,022B2, which are hereby incorporated by reference), zanolimumab (also known as HuMax-CD4 or 6G5.2; see, e.g., WO1997013852, which is hereby incorporated by reference), tregalizumab (also known as BT-061; see, e.g., U.S. Pat. No. 7,452,981, which is hereby incorporated by reference), priliximab (also known as Centara, cM-T412, CEN 000029, MT 412,), MTRX1011A (see, e.g., WO2008134046, which is hereby incorporated by reference), cedelizumab (also known as OKT-4A), clenoliximab (also known as IDEC-151, BB-217969), keliximab (also known as IDEC CE9.1, SB210396), M-T413, and TRX1 (see, e.g., WO2002102853, which is hereby incorporated by reference). Examples of anti-CD8 antibodies are similarly known in the art including, for example, the anti-CD8 antibodies described in WO2019033043, WO2017134306, WO2019032661, WO2019023148, WO2014025828, U.S. Ser. No. 10/414,820, and U.S. Ser. No. 10/377,826, which are hereby incorporated by reference. In certain embodiments, the immunosuppressant is a lymphodepleting antibody. For example, the lymphodepleting antibody can be an anti-CD45 antibody, such as clone 30-F11, a naked antibody that mimics ATG by relying on effector function to enable potent peripheral B- and T-cell depletion.

In other embodiments, the anti-CD117 antibody, anti-CD45 antibody, or antibody-drug conjugate thereof, is administered in combination with cyclophosphamide (Cytoxan, e.g., low-dose Cytoxan).

In yet further embodiments, the anti-CD117 antibody, anti-CD45 antibody, or antibody-drug conjugate thereof, is administered in combination with total body irradiation (TBI, e.g., low-dose TBI). Traditional conditioning protocols can use high doses of TBI prior to receipt of an allogeneic transplant. In some embodiments of the methods provided herein, when TBI is used in combination with an anti-CD117 antibody, anti-CD45 antibody, or antibody-drug conjugate thereof, a reduced dose of TBI can be used to effectively condition a patient for allogeneic transplant therapy. Accordingly, in some embodiments, the invention provides a method of reducing the level of TBI used to condition a patient for allogeneic transplant therapy, comprising administering to the patient an anti-CD117 ADC and/or an anti-CD45 ADC as described herein, in combination with low dose TBI. In one embodiment, the level of TBI is 5 Gy or less, e.g., 4.5 Gy or less, 4 Gy or less, 3.5 Gy or less, 3 Gy or less, 2.5 Gy or less, 2 Gy or less, 1.5 Gy or less, 1 Gy or less, or 0.5 Gy or less. In some embodiments, the level of TBI is about 5 Gy, about 4.5 Gy, about 4 Gy, about 3.5 Gy, about 3 Gy, about 2.5 Gy, about 2 Gy, about 1.5 Gy, about 1 Gy, or about 0.5 Gy.

In other embodiments, the anti-CD117 antibody, anti-CD45 antibody, or antibody-drug conjugate thereof, is administered in combination with an unconjugated anti-CD45 antibody capable of depleting CD45+ cells through effector function (i.e., complement-dependent cytotoxicity (CDC) or antibody-dependent cellular cytotoxicity (ADCC).

In other embodiments, an anti-CD117 ADC and/or an anti-CD45 ADC can be used in accordance with the methods provided herein in combination with one or more of the following immunosuppressants: calcineurin/MTOR inhibitors (e.g tacrolimus, sirolimus, rapamycin, ciclosporin, everolimus), co-stimulatory blockade molecules (e.g. CTLA4-Ig, anti-CD40L), NK depletion agents, Anti-thymocyte globulin (ATG), alkylating agents (e.g., nitrogen mustards, e.g., cyclophosphamide; nitrosoureas (e.g., carmustine); platinum compounds), methotrexate, anti-TCR agents (e.g., muromonab-CD3), anti-CD20 antibodies (e.g., rituximab, ocrelizumab, ofatumumab, and veltuzumab), fludarabine, Campath (alemtuzumab), 2-amino-6-aryl-5-substituted pyrimidines (see U.S. Pat. No. 4,665,077, supra, the disclosure of which is incorporated herein by reference), azathioprine (or cyclophosphamide, if there is an adverse reaction to azathioprine); bromocryptine; glutaraldehyde (which masks the MHC antigens, as described in U.S. Pat. No. 4,120,649, supra); antiidiotypic antibodies for MHC antigens; cyclosporin A; one or more steroids, e.g., corticosteroids, e.g., glucocorticosteroids such as prednisone, methylprednisolone, hydrocortisone, and dexamethasone; anti-interferon-γ antibodies; anti-tumor necrosis factor-α antibodies; anti-tumor necrosis factor-0 antibodies; anti-interleukin-2 antibodies; anti-cytokine receptor antibodies such as anti-IL-2 receptor antibodies; heterologous anti-lymphocyte globulin; pan-T antibodies, e.g., OKT-3 monoclonal antibodies; antibodies to CD4; antibodies to CD8, antibodies to CD45 (e.g., 30-F11, YTH24.5, and/or YTHS4.12 (e.g., a combination of YTH24.5 and YTH54.12)); streptokinase; streptodornase; or RNA or DNA from the host.

In one exemplary embodiment, the patient is conditioned with an anti-CD117-PBD ADC in combination with TBI, Cytoxan, an anti-CD4 antibody, an anti-CD8 antibody, or a combination thereof.

In another exemplary embodiment, the patient is conditioned with an anti-CD45-PBD ADC in combination with TBI, Cytoxan, an anti-CD4 antibody, an anti-CD8 antibody, or a combination thereof.

The foregoing immunosuppressants (including but not limited to an anti-CD4 antibody, an anti-CD8 antibody, Cytoxan, and/or TBI) can be administered to the patient prior to receipt of a transplant comprising allogeneic cells, e.g., allogeneic HSCs. In some embodiments, the immunosuppressant is administered to the subject post-transplant. In some embodiments, the immunosuppressant is administered to the subject both pre- and post-transplant.

In certain embodiments, the antibodies or ADCs described herein are used to treat a subject receiving a mismatched allogeneic transplant. In some embodiments, the donor is a mismatched donor. Mismatched donor cells, organs, or tissues comprise at least one dissimilar (e.g., non-identical) major histocompatibility complex (MHC) antigen (i.e., human leukocyte antigen (HLA) in humans), e.g., class I, class II, or class III MHC antigen or minor histocompatibility antigen (miHA), relative to a variant expressed by the recipient, as typically determined by standard assays used in the art, such as serological, genomic, or molecular analysis of a defined number of MHC or miHA antigens. In an exemplary embodiment, the allogeneic transplant shares the same MHC or HLA haplotype as the transplant recipient, but can contain one or more minor mismatches (e.g., a minor mismatch allogeneic transplant). In another exemplary embodiment, the allogeneic transplant contains one or more major mismatches, alone or in addition to one or more minor mismatches. In another exemplary embodiment, the allogeneic transplant is a “full mismatch” allogeneic transplant, that contains one or more major mismatches and one or more minor mismatches.

MHC proteins are important for signaling between lymphocytes and antigen presenting cells or diseased cells in immune reactions, where the MHC proteins bind peptides and present them for recognition by T cell receptors. The proteins encoded by the MHC genes are expressed on the surface of cells, and display both self antigens (peptide fragments from the cell itself) and non-self antigens (e.g., fragments of invading microorganisms) to a T cell.

The MHC region is divided into three subgroups, class I, class II, and class III. MHC class I proteins contain an α-chain and β-microglobulin (i.e., B2M) and present antigen fragments to cytotoxic T cells. On most immune system cells, specifically on antigen-presenting cells, MHC class II proteins contain α- and β-chains and present antigen fragments to T-helper cells. The MHC class III region encodes for other immune components, such as complement components and some that encode cytokines. The MHC is both polygenic (there are several MHC class I and MHC class II genes) and polymorphic (there are multiple alleles of each gene).

In humans, the major histocompatibility complex is alternatively referred to as the human leukocyte antigen (HLA) complex. Each class of MHC is represented by several loci in humans: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. MHCs exhibit extreme polymorphism: within the human population there are, at each genetic locus, a great number of haplotypes comprising distinct alleles. Different polymorphic MHC alleles, of both class I and class II, have different peptide specificities: each allele encodes proteins that bind peptides exhibiting particular sequence patterns. The HLA genomic loci and methods of testing for HLA alleles or proteins in humans have been described in the art (see, e.g., Choo et al. (2007). Yonsei medical journal. 48.1: 11-23; Shiina et al. (2009). Journal of human genetics. 54.1: 15: Petersdorf. (2013). Blood. 122.11: 1863-1872; and Bertaina and Andreani. (2018). International journal of molecular sciences. 19.2. 621, which are hereby incorporated by reference in their entirety).

In some embodiments, at least one major histocompatibility complex antigen (e.g., an HLA antigen) is mismatched in the subject receiving a transplant in accordance with the methods provided herein relative to the donor. In certain embodiments, the MHC antigen is a MHC class I molecule or a MHC class molecule. In particular embodiments, MHC antigen is any one of, or any combination of, a B2M, HLA-A, HLA-B, HLA-C. HLA-DRA, HLA-DRB1, HLA-DRB3, HLA-DRB4, HLA-DRB5, HLA-DPA1, HLA-DPA2, HLA-DQA1, and/or HLA-DQB1. In some embodiments, transplant comprises allogeneic hematopoietic stem cells that comprise at least one HLA-mismatch relative to the HLA antigens in the human patient. For example, in certain instances, the allogeneic hematopoietic stem cells comprise at least one, at least two, at least three, at least four, at least five, at least six, at least seven, at least eight, at least nine, or more than nine HLA-mismatches relative to the HLA antigens in the human patient. In some embodiments, the allogeneic hematopoietic stem cells comprise a full HLA-mismatch relative to the HLA antigens in the human patient.

Alternatively or additionally, at least one minor histocompatibility antigen is mismatched in the subject receiving a transplant in accordance with the methods provided herein relative to the donor. In some embodiments, transplant comprises allogeneic hematopoietic stem cells that comprise at least one miHA-mismatch relative to the miHA antigens in the human patient. For example, in certain instances, the allogeneic hematopoietic stem cells comprise at least one, at least two, at least three, at least four, at least five, at least six, at least seven, at least eight, at least nine, or more than nine miHA-mismatches relative to the miHA antigens in the human patient. In certain embodiments, the minor histocompatibility antigen is a HA-1, HA-2, HA-8. HA-3, HB-1, HY-A1, HY-A2, HY-B7, HY-B8, HY-B60, or HY-DQ5 protein. Examples of other minor histocompatibility antigens are known in the art (e.g., Perreault et al. (1990). Blood. 76.7: 1269-1280; Martin et al. (2017). Blood. 129.6: 791-798; and U.S. patent Ser. No. 10/414,813B2, which are hereby incorporated by reference in their entirety).

The methods described herein can increase the level of allogeneic donor cell chimerism in the transplant recipient, relative to a patient that receives either an anti-CD117 ADC, an anti-CD45 ADC, or an immunosuppressant alone. In some embodiments, the methods are effective to establish at least 80% donor chimerism in the transplant recipient (e.g., at least 80%, at least 85%, at least 90%, at least 95%, or at least 99% donor chimerism). The level of donor chimerism following allogeneic HSC transplant can be, for example, total chimerism, bone marrow chimerism, peripheral myeloid chimerism, B-cell chimerism, or T-cell chimerism.

Routes of Administration and Dosing Antibodies, antigen-binding fragments thereof, or ADCs described herein can be administered to a patient (e.g., a human patient suffering from cancer, an autoimmune disease, or in need of hematopoietic stem cell transplant therapy) in a variety of dosage forms. For instance, antibodies, antigen-binding fragments thereof, or ADCs described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

Pharmaceutical formulations comprising an anti-HC antibody (e.g., an anti-CD117 antibody or an anti-CD45 antibody), or conjugates thereof (e.g., ADCs as described herein) are prepared by mixing such antibody or ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol): low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The antibodies, antigen-binding fragments, or ADCs described herein may be administered by a variety of routes, such as orally, transdermally, subcutaneously, intranasally, intravenously, intramuscularly, intraocularly, or parenterally. The most suitable route for administration in any given case will depend on the particular antibody, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an antibody, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of about 0.0001- about 5000 μg/mL) of the antibody, or antigen-binding fragment thereof. The dose may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant.

In one embodiment, the dose of an anti-HC ADC (e.g, an anti-CD117 antibody or anti-CD45 antibody conjugated via a linker to a cytotoxin) administered to the human patient is about 0.1 mg/kg to about 0.3 mg/kg.

In one embodiment, the dose of an anti-HC ADC (e.g, an anti-CD117 antibody or anti-CD45 antibody conjugated via a linker to a cytotoxin) administered to the human patient is about 0.15 mg/kg to about 0.3 mg/kg.

In one embodiment, the dose of an anti-HC ADC (e.g, an anti-CD117 antibody or anti-CD45 antibody conjugated via a linker to a cytotoxin) administered to the human patient is about 0.15 mg/kg to about 0.25 mg/kg.

In one embodiment, the dose of an anti-HC ADC (e.g, an anti-CD117 antibody or anti-CD45 antibody conjugated via a linker to a cytotoxin) administered to the human patient is about 0.2 mg/kg to about 0.3 mg/kg.

In one embodiment, the dose of an anti-HC ADC (e.g, an anti-CD117 antibody or anti-CD45 antibody conjugated via a linker to a cytotoxin) administered to the human patient is about 0.25 mg/kg to about 0.3 mg/kg.

In one embodiment, the dose of an anti-HC ADC (e.g, an anti-CD117 antibody or anti-CD45 antibody conjugated via a linker to a cytotoxin) administered to the human patient is about 0.1 mg/kg.

In one embodiment, the dose of an anti-HC ADC (e.g, an anti-CD117 antibody or anti-CD45 antibody conjugated via a linker to a cytotoxin) administered to the human patient is about 0.2 mg/kg.

In one embodiment, the dose of an anti-HC ADC (e.g, an anti-CD117 antibody or anti-CD45 antibody conjugated via a linker to a cytotoxin) administered to the human patient is about 0.3 mg/kg.

In one embodiment, the dose of an anti-HC ADC described herein administered to the human patient is about 0.001 mg/kg to 10 mg/kg, about 0.01 mg/kg to 9.5 mg/kg, about 0.1 mg/kg to 9 mg/kg, about 0.1 mg/kg to 8.5 mg/kg, about 0.1 mg/kg to 8 mg/kg, about 0.1 mg/kg to 7.5 mg/kg, about 0.1 mg/kg to 7 mg/kg, about 0.1 mg/kg to 6.5 mg/kg, about 0.1 mg/kg to 6 mg/kg, about 0.1 mg/kg to 5.5 mg/kg, about 0.1 mg/kg to 5 mg/kg, about 0.1 mg/kg to 4.5 mg/kg, about 0.1 mg/kg to 4 mg/kg, about 0.5 mg/kg to 3.5 mg/kg, about 0.5 mg/kg to 3 mg/kg, about 1 mg/kg to 10 mg/kg, about 1 mg/kg to 9 mg/kg, about 1 mg/kg to 8 mg/kg, about 1 mg/kg to 7 mg/kg, about 1 mg/kg to 6 mg/kg, about 1 mg/kg to 5 mg/kg, about 1 mg/kg to 4 mg/kg, or about 1 mg/kg to 3 mg/kg.

In one embodiment, anti-HC ADC described herein that is administered to a human patient for treatment or conditioning has a half-life of equal to or less than 24 hours, equal to or less than 22 hours, equal to or less than 20 hours, equal to or less than 18 hours, equal to or less than 16 hours, equal to or less than 14 hours, equal to or less than 13 hours, equal to or less than 12 hours, equal to or less than 11 hours, equal to or less than 10 hours, equal to or less than 9 hours, equal to or less than 8 hours, equal to or less than 7 hours, equal to or less than 6 hours, or equal to or less than 5 hours. In one embodiment, the half-life of the anti-HC ADC is 5 hours to 7 hours; is 5 hours to 9 hours; is 15 hours to 11 hours; is 5 hours to 13 hours; is 5 hours to 15 hours; is 5 hours to 20 hours; is 5 hours to 24 hours; is 7 hours to 24 hours; is 9 hours to 24 hours; is 11 hours to 24 hours; 12 hours to 22 hours; 10 hours to 20 hours; 8 hours to 18 hours; or 14 hours to 24 hours.

In one embodiment, the methods disclosed herein minimize liver toxicity in the patient receiving the ADC for conditioning. For example, in certain embodiments, the methods disclosed herein result in a liver marker level remaining below a known toxic level in the patient for more than 24 hours, 48 hours, 72 hours, or 96 hours. In other embodiments, the methods disclosed herein result in a liver marker level remaining within a reference range in the patient for more than 24 hours, 48 hours, 72 hours, or 96 hours. In certain embodiments, the methods disclosed herein result in a liver marker level rising not more than 1.5-fold above a reference range, not more than 3-fold above a reference range, not more than 5-fold above a reference range, or not more than 10-fold above a reference range for more than 24 hours, 48 hours. 72 hours, or 96 hours. Examples of liver markers that can be used to test for toxicity include alanine aminotransaminase (ALT), lactate dehydrogenase (LDH), and aspartate aminotransaminase (AST). In certain embodiments, administration of an ADC as described herein, i.e., where two doses are administered instead of a single dose, results in a transient increase in a liver marker, e.g., AST, LDH, and/or ALT. In some instances, an elevated level of a liver marker indicating toxicity may be reached, but within a certain time period, e.g., about 12 hours, about 18 hours, about 24 hours, about 36 hours, about 48 hours, about 72 hours, above 3 days, about 3.5 days, about 4 days, about 4.5 days, about 5 days, about 5.5 days, about 6 days, about 6.5 days, about 7 days, about 7.5 days, or less than a week, the liver marker level returns to a normal level not associated with liver toxicity. For example, in a human (average adult male), a normal, non-toxic level of ALT is 7 to 55 units per liter (U/L); and a normal, non-toxic level of AST is 8 to 48 U/L. In certain embodiments, at least one of the patient's blood AST, ALT, or LDH levels does not reach a toxic level between administration of a first dose of the ADC and 14 days after administration of the first dose to the patient. For example, the patient may be administered a first dose and subsequently a second dose, a third dose, a fourth dose, or more doses within, e.g., 5, 10, or 14 days of being administered the first dose, yet at least one of the patient's blood AST, ALT, or LDH levels does not reach a toxic level between administration of a first dose of the ADC and 14 days after administration of the first dose to the patient.

In certain embodiments, at least one of the patient's blood AST, ALT, or LDH levels does not rise above normal levels, does not rise more than 1.5-fold above normal levels, does not rise more than 3-fold above normal levels, does not rise more than 5-fold above normal levels, or does not rise more than 10-fold above normal levels.

In the case of a conditioning procedure prior to hematopoietic stem cell transplantation, the antibody, or antigen-binding fragment thereof can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days) or more prior to administration of the exogenous hematopoietic stem cell transplant. Ranges including the numbers recited herein are also included in the contemplated methods.

Dosing ranges described above may be combined with anti-HC ADCs having half-lives recited herein.

Using the methods disclosed herein, a physician of skill in the art can administer to a human patient in need of hematopoietic stem cell transplant therapy an ADC, an antibody or an antigen-binding fragment thereof capable of binding an antigen expressed by hematopoietic stem cells (e.g., CD117 (e.g., GNNK+CD117) or CD45) or an antigen expressed by mature immune cells, such as T-cells (e.g., CD45). In this fashion, a population of endogenous hematopoietic stem cells can be depleted prior to administration of an exogenous hematopoietic stem cell graft so as to promote engraftment of the hematopoietic stem cell graft. The antibody may be covalently conjugated to a toxin, such as a cytotoxic molecule described herein or known in the art. For instance, an anti-CD117 antibody or antigen-binding fragment thereof (such as an anti-HC antibody (e.g., anti-CD117 antibody or anti-CD45 antibody) or antigen-binding fragment thereof) can be covalently conjugated to a cytotoxin, such as pseudomonas exotoxin A, deBouganin, diphtheria toxin, an amatoxin, such as γ-amanitin, α-amanitin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof. This conjugation can be performed using covalent bond-forming techniques described herein or known in the art. The antibody, antigen-binding fragment thereof, or drug-antibody conjugate can subsequently be administered to the patient, for example, by intravenous administration, prior to transplantation of exogenous hematopoietic stem cells (such as autologous, syngeneic, or allogeneic hematopoietic stem cells) to the patient.

The anti-HC antibody (e.g., anti-CD117 antibody or anti-CD45 antibody) antigen-binding fragment thereof, or drug-antibody conjugate can be administered in an amount sufficient to reduce the quantity of endogenous hematopoietic stem cells, for example, by about 10%, about 20%, about 30%, about 40%, about 50%, about 60%, about 70%, about 80%, about 90%, about 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of hematopoietic stem cells has reached a minimum value in response to conditioning therapy with an anti-HC antibody (e.g., an anti-CD117 antibody or an anti-CD45 antibody) antigen-binding fragment thereof, or drug-antibody conjugate, the physician may conclude the conditioning therapy, and may begin preparing the patient for hematopoietic stem cell transplant therapy.

The anti-HC antibody (e.g., anti-CD117 antibody or anti-CD45 antibody) antigen-binding fragment thereof, or drug-antibody conjugate can be administered to the patient in an aqueous solution containing one or more pharmaceutically acceptable excipients, such as a viscosity-modifying agent. The aqueous solution may be sterilized using techniques described herein or known in the art. The antibody, antigen-binding fragment thereof, or drug-antibody conjugate can be administered to the patient at a dosage of, for example, from about 0.001 mg/kg to about 100 mg/kg, from about 0.001 mg/kg to about 10 mg/kg, about 0.01 mg/kg to 9.5 mg/kg, about 0.1 mg/kg to 9 mg/kg, about 0.1 mg/kg to 8.5 mg/kg, about 0.1 mg/kg to 8 mg/kg, about 0.1 mg/kg to 7.5 mg/kg, about 0.1 mg/kg to 7 mg/kg, about 0.1 mg/kg to 6.5 mg/kg, about 0.1 mg/kg to 6 mg/kg, about 0.1 mg/kg to 5.5 mg/kg, about 0.1 mg/kg to 5 mg/kg, about 0.1 mg/kg to 4.5 mg/kg, about 0.1 mg/kg to 4 mg/kg, about 0.5 mg/kg to 3.5 mg/kg, about 0.5 mg/kg to 3 mg/kg, about 1 mg/kg to 10 mg/kg, about 1 mg/kg to 9 mg/kg, about 1 mg/kg to 8 mg/kg, about 1 mg/kg to 7 mg/kg, about 1 mg/kg to 6 mg/kg, about 1 mg/kg to 5 mg/kg, about 1 mg/kg to 4 mg/kg, or about 1 mg/kg to 3 mg/kg, prior to administration of a hematopoietic stem cell graft to the patient. The antibody, antigen-binding fragment thereof, or drug-antibody conjugate can be administered to the patient at a time that optimally promotes engraftment of the exogenous hematopoietic stem cells, for instance, from about 1 hour to about 1 week (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, or about 7 days) or more prior to administration of the exogenous hematopoietic stem cell transplant.

Immunosuppression therapy typically involves the administration of an effective amount of an immunosuppressive agent. The immunosuppressant compositions will be formulated and dosed in a fashion consistent with good medical practice. Factors for consideration in this context include the clinical condition of the individual patient, the cause of the transplant, the site of delivery of the agent, the method of administration, the scheduling of administration, and other factors known to practitioners. The effective amount in this context, which is determined by such considerations, is the minimum amount necessary to prevent an immune response that would result in rejection of the graft by the host. Such amount is preferably below the amount that is toxic to the host or renders the host significantly more susceptible to infections. The amount of immunosuppressant required for the disclosure herein may be lower than that normally required for transplanted grafts that have not been pre-treated, and depends on the individual circumstances surrounding the transplant and the type of immunosuppressant employed.

As noted above, however, these suggested amounts of immunosuppressant are subject to a great deal of therapeutic discretion. The key factor in selecting an appropriate dose and scheduling is the result obtained, i.e., graft survival. For example, relatively higher doses may be needed initially for the treatment of hyperacute graft rejection, which can be attributed to antibody-mediated graft destruction, or at a later stage for the treatment of acute rejection, which is characterized by a sudden decline in graft function.

The immunosuppressant is administered by any suitable means, including parenteral, and, if desired for local immunosuppressive treatment, intralesional, administration. Parenteral infusions include intramuscular, intravenous, intraarterial, intraperitoneal, or subcutaneous administration. In addition, the immunosuppressant is suitably administered by pulse infusion, particularly with declining doses of the immunosuppressive agent, or by continuous infusion.

In some embodiments, the immunosuppressant is administered prior to a stem cell transplant (i.e., pre-transplant). In some embodiments, the immunosuppressant is administered following a stem cell transplant (i.e., post-transplant). In some embodiments, the immunosuppressant is administered at substantially the same time as the patient receives the transplant.

Following the conclusion of conditioning therapy, the patient may then receive an infusion (e.g., an intravenous infusion) of exogenous hematopoietic stem cells, such as from the same physician that performed the conditioning therapy or from a different physician. The physician may administer the patient an infusion of autologous, syngeneic, or allogeneic hematopoietic stem cells, for instance, at a dosage of from 1×10³ to 1×10⁹ hematopoietic stem cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from 1 hour to 6 months, or more, following hematopoietic stem cell transplant therapy (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or more). A finding that the concentration of hematopoietic stem cells or cells of the hematopoietic lineage has increased (e.g., by about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, about 100%, about 200%, about 500%, or more) following the transplant therapy relative to the concentration of the corresponding cell type prior to transplant therapy provides one indication that treatment with the anti-HC antibody (e.g., anti-CD117 antibody or anti-CD45 antibody) antigen-binding fragment thereof, or drug-antibody conjugate has successfully promoted engraftment of the transplanted hematopoietic stem cell graft.

Engraftment of hematopoietic stem cell transplants due to the administration of an anti-HC antibody (e.g., an anti-CD117 antibody or an anti-CD45 antibody), antigen-binding fragments thereof, or ADCs, can manifest in a variety of empirical measurements. For instance, engraftment of transplanted hematopoietic stem cells can be evaluated by assessing the quantity of competitive repopulating units (CRU) present within the bone marrow of a patient following administration of an antibody or antigen-binding fragment thereof capable of binding capable of binding an antigen expressed by hematopoietic stem cells (e.g., CD117 (e.g., GNNK+CD117), or CD45) and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

### Anti-HC Antibodies

The present disclosure is based in part on the discovery that antibodies, or antigen-binding fragments thereof, capable of binding an antigen expressed by hematopoietic cells, such as CD117 (e.g., GNNK+CD117), or CD45 can be used as therapeutic agents alone or as antibody drug conjugates (ADCs) to (i) treat cancers and autoimmune diseases characterized by CD117+(e.g., GNNK+CD117) or CD45+ hematopoietic cells; and (ii) promote the engraftment of transplanted hematopoietic stem cells in a patient in need of transplant therapy. These therapeutic activities can be caused, for instance, by the binding of an anti-hematopoietic cell (HC)-antibody (e.g., anti-CD117 antibody or anti-CD45 antibody) or antigen-binding fragment thereof, that binds to an antigen (e.g., CD117 (e.g., GNNK+CD117) or CD45) expressed by a hematopoietic cell (e.g., hematopoietic stem cell), leukocyte, or immune cell, e.g., mature immune cell (e.g., T cell)), such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

The anti-HC antibodies (e.g., anti-CD117 antibody or anti-45 antibody) described herein can be in the form of full-length antibodies, bispecific antibodies, dual variable domain antibodies, multiple chain or single chain antibodies, and/or binding fragments that specifically bind human CD117 or CD45, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), lgD, IgE, IgG (e.g. IgG1, IgG2, IgG3 or IgG4), or IgM. In some embodiments, the anti-HC antibody (e.g., anti-CD117 antibody or anti-CD45 antibody) is an IgG (e.g. IgG1, IgG2, IgG3 or IgG4).

Antibodies for use in conjunction with the methods described herein include variants of those antibodies described above, such as antibody fragments that contain or lack an Fc domain, as well as humanized variants of non-human antibodies described herein and antibody-like protein scaffolds (e.g., ¹⁰Fn3 domains) containing one or more, or all, of the CDRs or equivalent regions thereof of an antibody, or antibody fragment, described herein. Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

In certain embodiments, an anti-CD117 antibody, or antigen binding fragment thereof, has a certain dissociation rate which is particularly advantageous when used as a part of a conjugate. For example, an anti-CD117 antibody has, in certain embodiments, an off rate constant (Koff) for human CD117 and/or rhesus CD117 of 1×10⁻² to 1×10⁻³, 1×10⁻³ to 1×10⁻⁴, 1×10⁴ to 1×10⁴, 1×10⁴ to 1×10⁻⁷ or 1×10⁻⁷ to 1×10⁻⁸, as measured by bio-layer interferometry (BLI). In some embodiments, the antibody or antigen-binding fragment thereof binds CD117 (e.g., human CD117 and/or rhesus CD117) with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay.

In one embodiment, anti-HC antibody (e.g., anti-CD117 antibodies or anti-CD45 antibodies) comprising one or more radiolabeled amino acids are provided. A radiolabeled anti-CD117 antibody may be used for both diagnostic and therapeutic purposes (conjugation to radiolabeled molecules is another possible feature).

Nonlimiting examples of labels for polypeptides include, but are not limited to 3H, 14C, 15N, 35S, 90Y, 99Tc, and 125I, 131I, and 186Re. Methods for preparing radiolabeled amino acids and related peptide derivatives are known in the art (see for instance Junghans et al., in Cancer Chemotherapy and Biotherapy 655-686 (2d edition, Chafner and Longo, eds., Lippincott Raven (1996)) and U.S. Pat. Nos. 4,681,581, 4,735,210, 5,101,827, U.S. Pat. No. 5,102,990 (U.S. RE35,500), U.S. Pat. Nos. 5,648,471 and 5,697,902. For example, a radioisotope may be conjugated by a chloramine T method.

The anti-HC antibodies (e.g., anti-CD117 or anti-CD45 antibodies), binding fragments, or conjugates thereof, described herein may also include modifications and/or mutations that alter the properties of the antibodies and/or fragments, such as those that increase half-life, increase or decrease ADCC, etc., as is known in the art.

In one embodiment, the anti-HC antibody (e.g., anti-CD117 antibody, or anti-CD45 antibody) or binding fragment thereof, comprises a modified Fc region, wherein said modified Fc region comprises at least one amino acid modification relative to a wild-type Fc region, such that said molecule has an altered affinity for or binding to an FcgammaR (FcγR). Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). In some embodiments, the antibodies described herein may comprise variant Fc regions comprising modification of at least one residue that makes a direct contact with an FcγR based on structural and crystallographic analysis. In one embodiment, the Fc region of the anti-HC antibody (e.g., anti-CD117 antibody or anti-CD45 antibody) (or fragment thereof) comprises an amino acid substitution at amino acid 265 according to the EU index as in Kabat et al., Sequences of Proteins of Immunological Interest, 5th Ed. Public Health Service, NH₁, MD (1991), expressly incorporated herein by reference. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat. In one embodiment, the Fc region comprises a L234A mutation. In some embodiments, the Fc region of the anti-HC antibody (e.g., anti-CD117 antibody or anti-CD45 antibody) (or fragment thereof) comprises an amino acid substitution at amino acid 235 according to the EU index as in Kabat. In one embodiment, the Fc region comprises a L235A mutation.

In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A. S239C, L234A, and L235A mutation (also referred to herein as “D265A.S239C.L234A.L235A). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and deIG236 (deletion of 236) mutation (also referred to herein as “E233P.L234V.L235A.deIG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, deIG236 (deletion of 236) and H435A mutation (also referred to herein as “E233P.L234V.L235A.deIG236.H435A” or as “EPLVLAdeIG.H435A”). In another embodiment, the Fc region comprises a E233P, L234V, L235A, deIG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR1. In some embodiments, the FcγR is FcγR2A. In some embodiments, the FcγR is FcγR2B. In other embodiments, the FcγR is FcγR2C. In some embodiments, the FcγR is FcγR3A. In some embodiments, the FcγR is FcγR3B. In other embodiments, the decrease in binding is at least a 70% decrease, at least an 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In other embodiments, the decrease in binding is at least a 70% to a 100% decrease, at least an 80% to a 100% decrease, at least a 90% to a 100% decrease, at least a 95% to a 100% decrease, or at least a 98% to a 100% decrease, in antibody binding to a FcγR relative to binding of the identical antibody comprising an unmodified Fc region to the FcγR In some embodiments, the antibody has a modified Fc region such that, the antibody decreases cytokine release in an in vitro cytokine release assay with a decrease in cytokine release of at least 50% relative to cytokine release of an identical antibody comprising an unmodified Fc region. In some embodiments, the decrease in cytokine release is at least a 70% decrease, at least an 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In some embodiments, the decrease in cytokine release is at least a 70% to a 100% decrease, at least an 80% to a 100% decrease, at least a 90% to a 100% decrease, at least a 95% to a 100% decrease in cytokine release relative to cytokine release of the identical antibody comprising an unmodified Fc region. In certain embodiments, cytokine release is by immune cells.

In some embodiments, the antibody has a modified Fc region such that, the antibody decreases mast cell degranulation in an in vitro mast cell degranulation assay with a decrease in mast cell degranulation of at least 50% relative to mast cell degranulation of an identical antibody comprising an unmodified Fc region. In some embodiments, the decrease in mast cell degranulation is at least a 70% decrease, at least an 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In some embodiments, the decrease in mast cell degranulation is at least a 70% to a 100% decrease, at least an 80% to a 100% decrease, at least a 90% to a 100% decrease, or at least a 95% to a 100% decrease, in mast cell degranulation relative to mast cell degranulation of the identical antibody comprising an unmodified Fc region.

In some embodiments, the antibody has a modified Fc region such that, the antibody decreases or prevents antibody dependent cell phagocytosis (ADCP) in an in vitro antibody dependent cell phagocytosis assay, with a decrease in ADCP of at least 50% relative to ADCP of an identical antibody comprising an unmodified Fc region. In some embodiments, the decrease in ADCP is at least a 70% decrease, at least an 80% decrease, at least a 90% decrease, at least a 95% decrease, at least a 98% decrease, at least a 99% decrease, or a 100% decrease in cytokine release relative to cytokine release of the identical antibody comprising an unmodified Fc region.

In some embodiments, the anti-HC antibody (e.g., anti-CD117 antibody, or anti-CD45 antibody) described herein comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdeIG *), D265C (EPLVLAdeIG)/H435A, D265C/N297Q/H435A, D265C/N297Q, EPLVLAdelG/H435A, EPLVLAdeIG/D265C, EPLVLAdeIG/D265A, N297A, N297G, or N297Q.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry. Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least an 80% decrease, at least a 90% decrease, at least a 95% decrease, at least a 98% decrease, at least a 99% decrease, or a 100% decrease in binding to a Fc gamma receptor relative to binding of the identical antibody comprising an unmodified Fc region to the Fc gamma receptor (e.g., as assessed by biolayer interferometry (BLI)).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in vitro or for their ability to trigger cytokine release, e.g. by human peripheral blood mononuclear cells.

The antibodies of the present disclosure may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380. 428, 434 and 435. Exemplary mutations that may be made singularly or in combination are T250Q. M252Y, 1253A, S254T. T256E, P2571, T307A, D376V, E380A, M428L, H₄₃₃K, N434S, N434A. N434H, N434F, H435A and H435R mutations.

Thus, in one embodiment, the Fc region comprises a mutation resulting in a decrease in half-life (e.g., relative to an antibody having an unmodified Fc region). An antibody having a short half-life may be advantageous in certain instances where the antibody is expected to function as a short-lived therapeutic, e.g., the conditioning step described herein where the antibody is administered followed by HSCs. Ideally, the antibody would be substantially cleared prior to delivery of the HSCs, which also generally express a target antigen (e.g., CD117 (e.g., GNNK+CD117) or CD45) but are not the target of the anti-HC antibody (e.g., anti-CD117 antibody or anti-CD45 antibody) unlike the endogenous stem cells. In one embodiment, the Fc region comprises a mutation at position 435 (EU index according to Kabat). In one embodiment, the mutation is an H435A mutation.

In one embodiment, the anti-HC antibody (e.g., anti-CD117 antibody or anti-CD45 antibody) described herein has a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-HC antibody (e.g., anti-CD117 antibody or anti-CD45 antibody) described herein has a half-life (e.g., in humans) of about 1-5 hours, about 5-10 hours, about 10-15 hours, about 15-20 hours, or about 20 to 25 hours. In one embodiment, the half-life of the anti-HC antibody is about 5-7 hours; about 5-9 hours; about 5-11 hours; about 5-13 hours; about 5-15 hours; about 5-20 hours; about 5-24 hours; about 7-24 hours; about 9-24 hours; about 11-24 hours; about 12-22 hours; about 10-20 hours; about 8-18 hours; or about 14-24 hours.

In some aspects, the Fc region comprises two or more mutations that confer reduced half-life and reduce an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof.

In some embodiments of these aspects, the cysteine residue is naturally occurring in the Fc domain of the antibody or antigen-binding fragment thereof. For instance, the Fc domain may be an IgG Fc domain, such as a human IgG1 Fc domain, and the cysteine residue may be selected from the group consisting of Cys261, Csy321, Cys367, and Cys425.

In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-HC antibody (e.g., anti-CD117 antibody or anti-CD45 antibody) (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-HC antibody (e.g., anti-CD117 antibody or anti-CD45 antibody), or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

The variant Fc domains described herein are defined according to the amino acid modifications that compose them. For all amino acid substitutions discussed herein in regard to the Fc region, numbering is always according to the EU index. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as LALA. It is noted that the order in which substitutions are provided is arbitrary. Notably, Fc amino acid positions are in reference to the EU numbering index unless otherwise indicated.

In some embodiments, the anti-CD117 antibody or anti-CD45 antibody herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2), D265C (IgG2)/H435A, D265C/N297Q/H435A, D265C/N297Q, EPLVLAdeIG/H435A, N297A, N297G, or N297Q.

The antibodies, and binding fragments thereof, disclosed herein can be used in conjugates, as described in more detail below.

Antibodies may be produced using recombinant methods and compositions, e.g., as described in U.S. Pat. No. 4,816,567. In one embodiment, isolated nucleic acid encoding an anti-CD117 antibody or anti-CD45 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 antibody or anti-CD45 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060582); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD117 antibody, or antigen binding fragment thereof, or anti-CD45 antibody, or antigen binding fragment thereof, comprises variable regions having an amino acid sequence that is at least 95%, 96%, 97% or 99% identical to the SEQ ID Nos disclosed herein (Table 3). Alternatively, the anti-CD117 antibody, or antigen binding fragment thereof, or anti-CD45 antibody, or antigen binding fragment thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein (Table 3).

In one embodiment, the anti-CD117 antibody, or antigen binding fragment thereof, comprises a heavy chain variable region and a heavy chain constant region having an amino acid sequence that is disclosed herein. In another embodiment, the anti-CD117 antibody, or antigen binding fragment thereof, comprises a light chain variable region and a light chain constant region having an amino acid sequence that is disclosed herein.

In yet another embodiment, the anti-CD117 antibody, or antigen binding fragment thereof, comprises a heavy chain variable region, a light chain variable region, a heavy chain constant region and a light chain constant region having an amino acid sequence that is disclosed herein.

In one embodiment, the anti-CD45 antibody, or antigen binding fragment thereof, comprises a heavy chain variable region and a heavy chain constant region having an amino acid sequence that is disclosed herein. In another embodiment, the anti-CD45 antibody, or antigen binding fragment thereof, comprises a light chain variable region and a light chain constant region having an amino acid sequence that is disclosed herein.

In yet another embodiment, the anti-CD45 antibody, or antigen binding fragment thereof, comprises a heavy chain variable region, a light chain variable region, a heavy chain constant region and a light chain constant region having an amino acid sequence that is disclosed herein.

Examples of anti-CD117 antibodies and anti-CD45 examples are described further herein.

### Anti-CD117 Antibodies

Antibodies and antigen-binding fragments capable of binding human CD117 (also referred to as c-Kit, mRNA NCBI Reference Sequence: NM_000222.2, Protein NCBI Reference Sequence: NP_000213.1), including those capable of binding GNNK+CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms. GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins. The amino acid sequences of human CD117 isoforms 1 and 2 are described in SEQ ID Nos: 145 and 148, respectively. In certain embodiments, anti-human CD117 (hCD117) antibodies disclosed herein are able to bind to both isoform 1 and isoform 2 of human CD117.

Examples of anti-CD117 antibodies are described in US 2019/0153114 A1 and US 2019/0144558 A1, the content of both applications are hereby expressly incorporated by reference in their entirety.

For example, the amino acid sequences for the various binding regions of anti-CD117 antibodies Ab54, Ab55, Ab56, Ab57, Ab58, Ab61, Ab66. Ab67. Ab68, and Ab69 are described in Table 3. Included in the present disclosure are human anti-CD117 antibodies comprising the CDRs as set forth in Table 3, as well as human anti-CD117 antibodies comprising the variable regions set forth in Table 3.

In one embodiment, the present disclosure provides an anti-CD117 antibody, or antigen-binding fragment thereof, comprising binding regions, e.g., CDRs, variable regions, corresponding to those of Antibody 55. The heavy chain variable region (VH) amino acid sequence of Antibody 55 (i.e., Ab55) is set forth in SEQ ID NO: 19 (see Table 3). The VH CDR domain amino acid sequences of Antibody 55 are set forth in SEQ ID NO: 21 (VH CDR1); SEQ ID NO: 22 (VH CDR2), and SEQ ID NO: 23 (VH CDR3). The light chain variable region (VL) amino acid sequence of Antibody 55 is described in SEQ ID NO: 20 (see Table 3). The VL CDR domain amino acid sequences of Antibody 55 are set forth in SEQ ID NO: 24 (VL CDR1); SEQ ID NO: 25 (VL CDR2), and SEQ ID NO: 26 (VL CDR3). The heavy chain constant region of Antibody 55 is set forth in SEQ ID NO: 122. The light chain constant region of Antibody 55 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 21, 22, and 23, and a light chain variable region CDR set as set forth in SEQ ID Nos: 24, 25, and 26. In other embodiments, an anti-CD117 antibody, or antigen-binding portion thereof, comprises a variable light chain comprising the amino acid residues set forth in SEQ ID NO: 20, and a heavy chain variable region as set forth in SEQ ID NO: 19.

In one embodiment, the present disclosure provides an anti-CD117 antibody, or antigen-binding fragment thereof, comprising binding regions, e.g., CDRs, variable regions, corresponding to those of Antibody 54. The heavy chain variable region (VH) amino acid sequence of Antibody 54 (i.e., Ab54) is set forth in SEQ ID NO: 29 (see Table 3). The VH CDR domain amino acid sequences of Antibody 54 are set forth in SEQ ID NO: 31 (VH CDR1); SEQ ID NO: 32 (VH CDR2), and SEQ ID NO: 33 (VH CDR3). The light chain variable region (VL) amino acid sequence of Antibody 54 is described in SEQ ID NO: 30 (see Table 3). The VL CDR domain amino acid sequences of Antibody 54 are set forth in SEQ ID NO: 34 (VL CDR1); SEQ ID NO: 35 (VL CDR2), and SEQ ID NO: 36 (VL CDR3). The heavy chain constant region of Antibody 54 is set forth in SEQ ID NO: 122. The light chain constant region of Antibody 54 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 31, 32, and 33, and a light chain variable region CDR set as set forth in SEQ ID Nos: 34, 35, and 36. In other embodiments, an anti-CD117 antibody, or antigen-binding portion thereof, comprises a variable light chain comprising the amino acid residues set forth in SEQ ID NO: 30, and a heavy chain variable region as set forth in SEQ ID NO: 29.

In one embodiment, the present disclosure provides an anti-CD117 antibody, or antigen-binding fragment thereof, comprising binding regions, e.g., CDRs, variable regions, corresponding to those of Antibody 56. The heavy chain variable region (VH) amino acid sequence of Antibody 56 (i.e., Ab56) is set forth in SEQ ID NO: 39 (see Table 3). The VH CDR domain amino acid sequences of Antibody 56 are set forth in SEQ ID NO: 41 (VH CDR1); SEQ ID NO: 42 (VH CDR2), and SEQ ID NO: 43 (VH CDR3). The light chain variable region (VL) amino acid sequence of Antibody 56 is described in SEQ ID NO: 40 (see Table 3). The VL CDR domain amino acid sequences of Antibody 56 are set forth in SEQ ID NO: 44 (VL CDR1); SEQ ID NO: 45 (VL CDR2), and SEQ ID NO: 46 (VL CDR3). The heavy chain constant region of Antibody 56 is set forth in SEQ ID NO: 122. The light chain constant region of Antibody 56 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 41, 42, and 43, and a light chain variable region CDR set as set forth in SEQ ID Nos: 44, 45, and 46. In other embodiments, an anti-CD117 antibody, or antigen-binding portion thereof, comprises a variable light chain comprising the amino acid residues set forth in SEQ ID NO: 40, and a heavy chain variable region as set forth in SEQ ID NO: 39.

In one embodiment, the present disclosure provides an anti-CD117 antibody, or antigen-binding fragment thereof, comprising binding regions, e.g., CDRs, variable regions, corresponding to those of Antibody 57. The heavy chain variable region (VH) amino acid sequence of Antibody 57 (i.e., Ab57) is set forth in SEQ ID NO: 49 (see Table 3). The VH CDR domain amino acid sequences of Antibody 57 are set forth in SEQ ID NO: 51 (VH CDR1); SEQ ID NO: 52 (VH CDR2), and SEQ ID NO: 53 (VH CDR3). The light chain variable region (VL) amino acid sequence of Antibody 57 is described in SEQ ID NO: 50 (see Table 3). The VL CDR domain amino acid sequences of Antibody 57 are set forth in SEQ ID NO: 54 (VL CDR1); SEQ ID NO: 55 (VL CDR2), and SEQ ID NO: 56 (VL CDR3). The heavy chain constant region of Antibody 57 is set forth in SEQ ID NO: 122. The light chain constant region of Antibody 57 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 51, 52, and 53, and a light chain variable region CDR set as set forth in SEQ ID Nos: 54, 55, and 56. In other embodiments, an anti-CD117 antibody, or antigen-binding portion thereof, comprises a variable light chain comprising the amino acid residues set forth in SEQ ID NO: 50, and a heavy chain variable region as set forth in SEQ ID NO: 49.

In one embodiment, the present disclosure provides an anti-CD117 antibody, or antigen-binding fragment thereof, comprising binding regions, e.g., CDRs, variable regions, corresponding to those of Antibody 58. The heavy chain variable region (VH) amino acid sequence of Antibody 58 (i.e., Ab58) is set forth in SEQ ID NO: 59 (see Table 3). The VH CDR domain amino acid sequences of Antibody 58 are set forth in SEQ ID NO: 61 (VH CDR1); SEQ ID NO: 62 (VH CDR2), and SEQ ID NO: 63 (VH CDR3). The light chain variable region (VL) amino acid sequence of Antibody 58 is described in SEQ ID NO: 60 (see Table 3). The VL CDR domain amino acid sequences of Antibody 58 are set forth in SEQ ID NO: 64 (VL CDR1); SEQ ID NO: 65 (VL CDR2), and SEQ ID NO: 66 (VL CDR3). The heavy chain constant region of Antibody 58 is set forth in SEQ ID NO: 122. The light chain constant region of Antibody 58 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 61, 62, and 63, and a light chain variable region CDR set as set forth in SEQ ID Nos: 64, 65, and 66. In other embodiments, an anti-CD117 antibody, or antigen-binding portion thereof, comprises a variable light chain comprising the amino acid residues set forth in SEQ ID NO: 60, and a heavy chain variable region as set forth in SEQ ID NO: 59.

In one embodiment, the present disclosure provides an anti-CD117 antibody, or antigen-binding fragment thereof, comprising binding regions, e.g., CDRs, variable regions, corresponding to those of Antibody 61. The heavy chain variable region (VH) amino acid sequence of Antibody 61 (i.e., Ab61) is set forth in SEQ ID NO: 69 (see Table 3). The VH CDR domain amino acid sequences of Antibody 61 are set forth in SEQ ID NO: 71 (VH CDR1); SEQ ID NO: 72 (VH CDR2), and SEQ ID NO: 73 (VH CDR3). The light chain variable region (VL) amino acid sequence of Antibody 61 is described in SEQ ID NO: 70 (see Table 3). The VL CDR domain amino acid sequences of Antibody 61 are set forth in SEQ ID NO: 74 (VL CDR1); SEQ ID NO: 75 (VL CDR2), and SEQ ID NO: 76 (VL CDR3). The heavy chain constant region of Antibody 61 is set forth in SEQ ID NO: 122. The light chain constant region of Antibody 61 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 71, 72, and 73, and a light chain variable region CDR set as set forth in SEQ ID Nos: 74, 75, and 76. In other embodiments, an anti-CD117 antibody, or antigen-binding portion thereof, comprises a variable light chain comprising the amino acid residues set forth in SEQ ID NO: 70, and a heavy chain variable region as set forth in SEQ ID NO: 69.

In one embodiment, the present disclosure provides an anti-CD117 antibody, or antigen-binding fragment thereof, comprising binding regions, e.g., CDRs, variable regions, corresponding to those of Antibody 66. The heavy chain variable region (VH) amino acid sequence of Antibody 66 (i.e., Ab66) is set forth in SEQ ID NO: 79 (see Table 3). The VH CDR domain amino acid sequences of Antibody 66 are set forth in SEQ ID NO: 81 (VH CDR1); SEQ ID NO: 82 (VH CDR2), and SEQ ID NO: 83 (VH CDR3). The light chain variable region (VL) amino acid sequence of Antibody 66 is described in SEQ ID NO: 80 (see Table 3). The VL CDR domain amino acid sequences of Antibody 66 are set forth in SEQ ID NO: 84 (VL CDR1); SEQ ID NO: 85 (VL CDR2), and SEQ ID NO: 86 (VL CDR3). The heavy chain constant region of Antibody 66 is set forth in SEQ ID NO: 122. The light chain constant region of Antibody 66 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 81, 82, and 83, and a light chain variable region CDR set as set forth in SEQ ID Nos: 84, 85, and 86. In other embodiments, an anti-CD117 antibody, or antigen-binding portion thereof, comprises a variable light chain comprising the amino acid residues set forth in SEQ ID NO: 80, and a heavy chain variable region as set forth in SEQ ID NO: 79.

In one embodiment, the present disclosure provides an anti-CD117 antibody, or antigen-binding fragment thereof, comprising binding regions, e.g., CDRs, variable regions, corresponding to those of Antibody 67. The heavy chain variable region (VH) amino acid sequence of Antibody 67 is set forth in SEQ ID NO: 9 (see Table 3). The VH CDR domain amino acid sequences of Antibody 67 are set forth in SEQ ID NO 11 (VH CDR1); SEQ ID NO: 12 (VH CDR2), and SEQ ID NO: 13 (VH CDR3). The light chain variable region (VL) amino acid sequence of Antibody 67 is described in SEQ ID NO: 10 (see Table 3). The VL CDR domain amino acid sequences of Antibody 67 are set forth in SEQ ID NO 14 (VL CDR1); SEQ ID NO: 15 (VL CDR2), and SEQ ID NO: 16 (VL CDR3). The full length heavy chain (HC) of Antibody 67 is set forth in SEQ ID NO: 110, and the full length heavy chain constant region of Antibody 67 is set forth in SEQ ID NO: 122. The light chain (LC) of Antibody 67 is set forth in SEQ ID NO: 109. The light chain constant region of Antibody 67 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 11, 12, and 13, and a light chain variable region CDR set as set forth in SEQ ID Nos: 14, 15, and 16. In other embodiments, an anti-CD117 antibody, or antigen-binding portion thereof, comprises a variable heavy chain comprising the amino acid residues set forth in SEQ ID NO: 9, and a heavy chain variable region as set forth in SEQ ID NO: 10. In further embodiments, an anti-CD117 antibody comprises a heavy chain comprising SEQ ID NO: 110 and a light chain comprising SEQ ID NO: 109.

In one embodiment, the present disclosure provides an anti-CD117 antibody, or antigen-binding fragment thereof, comprising binding regions, e.g., CDRs, variable regions, corresponding to those of Antibody 68. The heavy chain variable region (VH) amino acid sequence of Antibody 68 (i.e., Ab68) is set forth in SEQ ID NO: 89 (see Table 3). The VH CDR domain amino acid sequences of Antibody 68 are set forth in SEQ ID NO: 91 (VH CDR1); SEQ ID NO: 92 (VH CDR2), and SEQ ID NO: 93 (VH CDR3). The light chain variable region (VL) amino acid sequence of Antibody 68 is described in SEQ ID NO: 90 (see Table 3). The VL CDR domain amino acid sequences of Antibody 68 are set forth in SEQ ID NO: 94 (VL CDR1); SEQ ID NO: 95 (VL CDR2), and SEQ ID NO: 96 (VL CDR3). The heavy chain constant region of Antibody 68 is set forth in SEQ ID NO: 122. The light chain constant region of Antibody 68 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 91, 92, and 93, and a light chain variable region CDR set as set forth in SEQ ID Nos: 94, 95, and 96. In other embodiments, an anti-CD117 antibody, or antigen-binding portion thereof, comprises a variable light chain comprising the amino acid residues set forth in SEQ ID NO: 90, and a heavy chain variable region as set forth in SEQ ID NO: 89.

In one embodiment, the present disclosure provides an anti-CD117 antibody, or antigen-binding fragment thereof, comprising binding regions, e.g., CDRs, variable regions, corresponding to those of Antibody 69. The heavy chain variable region (VH) amino acid sequence of Antibody 69 (i.e., Ab69) is set forth in SEQ ID NO: 99 (see Table 3). The VH CDR domain amino acid sequences of Antibody 69 are set forth in SEQ ID NO: 101 (VH CDR1); SEQ ID NO: 102 (VH CDR2), and SEQ ID NO: 103 (VH CDR3). The light chain variable region (VL) amino acid sequence of Antibody 69 is described in SEQ ID NO: 100 (see Table 3). The VL CDR domain amino acid sequences of Antibody 69 are set forth in SEQ ID NO: 104 (VL CDR1); SEQ ID NO: 105 (VL CDR2), and SEQ ID NO: 106 (VL CDR3). The heavy chain constant region of Antibody 69 is set forth in SEQ ID NO: 122. The light chain constant region of Antibody 69 is set forth in SEQ ID NO: 121. Thus, in certain embodiments, an anti-CD117 antibody, or antigen-binding portion thereof, comprises a variable heavy chain CDR set (CDR1, CDR2, and CDR3) as set forth in SEQ ID Nos: 101, 102, and 103, and a light chain variable region CDR set as set forth in SEQ ID Nos: 104, 105, and 106. In other embodiments, an anti-CD117 antibody, or antigen-binding portion thereof, comprises a variable light chain comprising the amino acid residues set forth in SEQ ID NO: 100, and a heavy chain variable region as set forth in SEQ ID NO: 99.

Certain of the anti-CD117 antibodies described herein are neutral antibodies, in that the antibodies do not substantially inhibit CD117 activity on a CD117 expressing cell. Neutral antibodies can be identified using, for example, an in in vitro stem cell factor (SCF)-dependent cell proliferation assay. In an SCF dependent cell proliferation assay, a neutral CD117 antibody will not kill CD34+ cells that are dependent on SCF to divide, as a neutral antibody will not block SCF from binding to CD117 such as to inhibit CD117 activity.

Neutral antibodies can be used for diagnostic purposes, given their ability to specifically bind to human CD117, but are also effective for killing CD117 expressing cells when conjugated to a cytotoxin, such as those described herein. Typically, antibodies used in conjugates have agonistic or antagonistic activity that is unique to the antibody. Described herein, however, is a unique approach to conjugates, especially in the context wherein the conjugate is being used as a conditioning agent prior to a stem cell transplantation. While antagonistic antibodies alone or in combination with a cytotoxin as a conjugate can be effective given the killing ability of the antibody alone in addition to the cytotoxin, conditioning with a conjugate comprising a neutral anti-CD117 antibody presents an alternative strategy where the activity of the antibody is secondary to the effect of the cytotoxin, but the internalizing and affinity characteristics, e.g., dissociation rate, of the antibody are important for effective delivery of the cytotoxin.

Examples of neutral anti-CD117 antibodies include Ab58, Ab61, Ab66, Ab67, Ab68, and Ab69. A comparison of the amino acid sequences of the CDRs of neutral, anti-CD117 antibody CDRs reveals consensus sequences among two groups of neutral antibodies identified. Ab58 and Ab61 share the same light chain CDRs and HC CDR3, with slight variations in the HC CDR1 and HC CDR2. Consensus sequences for the HC CDR1 and CDR2 are described in SEQ ID Nos: 133 and 134. Ab66, Ab67, Ab68, and Ab69 are also neutral antibodies. While Ab66, Ab67, Ab68, and Ab69 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 139 and 140, respectively.

For example, in one embodiment, the Fc region of Antibody 67 is modified to comprise a D265C mutation (e.g., SEQ ID NO: 111). In another embodiment, the Fc region of Antibody 67 is modified to comprise a D265C, L234A, and L235A mutation (e.g., SEQ ID NO: 112). In yet another embodiment, the Fc region of Antibody 67 is modified to comprise a D265C and H435A mutation (e.g., SEQ ID NO: 113). In a further embodiment, the Fc region of Antibody 67 is modified to comprise a D265C, L234A, L235A, and H435A mutation (e.g., SEQ ID NO: 114).

In regard to Antibody 55, in one embodiment, the Fc region of Antibody 55 is modified to comprise a D265C mutation (e.g., SEQ ID NO: 117). In another embodiment, the Fc region of Antibody 55 is modified to comprise a D265C, L234A, and L235A mutation (e.g., SEQ ID NO: 118). In yet another embodiment, the Fc region of Antibody 55 is modified to comprise a D265C and H435A mutation (e.g., SEQ ID NO: 119). In a further embodiment, the Fc region of Antibody 55 is modified to comprise a D265C, L234A, L235A, and H435A mutation (e.g., SEQ ID NO: 120).

The Fc regions of any one of Antibody 54, Antibody 55. Antibody 56, Antibody 57, Antibody 58, Antibody 61. Antibody 66, Antibody 67, Antibody 68, or Antibody 69 can be modified to comprise a D265C mutation (e.g., as in SEQ ID NO: 123); a D265C, L234A, and L235A mutation (e.g., as in SEQ ID NO: 124); a D265C and H435A mutation (e.g., as in SEQ ID NO: 125); or a D265C, L234A, L235A, and H435A mutation (e.g., as in SEQ ID NO: 126).

Antagonist antibodies are also provided herein, including Ab54, Ab55, Ab56, and Ab57. While Ab54, Ab55, Ab56, and Ab57 share the same light chain CDRs and the same HC CDR3, these antibodies have variability within their HC CDR1 and HC CDR2 regions. Consensus sequences for these antibodies in the HC CDR1 and HC CDR2 regions are provided in SEQ ID Nos: 127 and 128, respectively.

In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 148. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 149. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 150. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 151. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 152. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 153. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 154. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 155. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 156. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 157. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 158. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 159. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 160. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 161. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 162. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 163. In one embodiment, the anti-CD117 antibody, or antigen binding portion thereof, comprises a heavy chain variable region as set forth in the amino acid sequence of SEQ ID NO: 164, and a light chain variable region as set forth in the amino acid sequence of SEQ ID NO: 165. In one embodiment, the anti-CD117 antibody, or antigen binding portion thereof, comprises a heavy chain variable region as set forth in the amino acid sequence of SEQ ID NO: 166, and a light chain variable region as set forth in the amino acid sequence of SEQ ID NO: 167. In one embodiment, the anti-CD117 antibody, or antigen binding portion thereof, comprises a heavy chain variable region as set forth in the amino acid sequence of SEQ ID NO: 168, and a light chain variable region as set forth in the amino acid sequence of SEQ ID NO: 169. In one embodiment, the anti-CD117 antibody, or antigen binding portion thereof, comprises a heavy chain variable region as set forth in the amino acid sequence of SEQ ID NO: 170, and a light chain variable region as set forth in the amino acid sequence of SEQ ID NO: 171. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 173. In one embodiment, the anti-CD117 antibody, or antigen binding portion thereof, comprises a heavy chain variable region as set forth in the amino acid sequence of SEQ ID NO: 174, and a light chain variable region as set forth in the amino acid sequence of SEQ ID NO: 175. In one embodiment, the anti-CD117 antibody, or antigen binding portion thereof, comprises a heavy chain variable region as set forth in the amino acid sequence of SEQ ID NO: 176, and a light chain variable region as set forth in the amino acid sequence of SEQ ID NO: 177. In one embodiment, the anti-CD117 antibody, or antigen binding portion thereof, comprises a heavy chain variable region as set forth in the amino acid sequence of SEQ ID NO: 178, and a light chain variable region as set forth in the amino acid sequence of SEQ ID NO: 179. In one embodiment, the anti-CD117 antibody, or antigen binding portion thereof, comprises a heavy chain variable region as set forth in the amino acid sequence of SEQ ID NO: 180, and a light chain variable region as set forth in the amino acid sequence of SEQ ID NO: 181. In one embodiment, the anti-CD117 antibody, or antigen binding portion thereof, comprises a heavy chain variable region as set forth in the amino acid sequence of SEQ ID NO: 172, and a light chain variable region as set forth in the amino acid sequence of SEQ ID NO: 182. In one embodiment, the anti-CD117 antibody, or antigen binding portion thereof, comprises a heavy chain variable region as set forth in the amino acid sequence of SEQ ID NO: 183, and a light chain variable region as set forth in the amino acid sequence of SEQ ID NO: 184. In one embodiment, the anti-CD117 antibody, or antigen binding portion thereof, comprises a heavy chain variable region as set forth in the amino acid sequence of SEQ ID NO: 185, and a light chain variable region as set forth in the amino acid sequence of SEQ ID NO: 186. In one embodiment, the anti-CD117 antibody, or antigen binding portion thereof, comprises a heavy chain variable region as set forth in the amino acid sequence of SEQ ID NO: 187, and a light chain variable region as set forth in the amino acid sequence of SEQ ID NO: 188. In one embodiment, the anti-CD117 antibody, or antigen binding portion thereof, comprises a heavy chain variable region as set forth in the amino acid sequence of SEQ ID NO: 189, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 191, and a light chain variable region as set forth in the amino acid sequence of SEQ ID NO: 192. In one embodiment, the anti-CD117 antibody, or antigen binding portion thereof, comprises a heavy chain variable region as set forth in the amino acid sequence of SEQ ID NO: 193, and a light chain variable region as set forth in the amino acid sequence of SEQ ID NO: 194. In one embodiment, the anti-CD117 antibody, or antigen binding portion thereof, comprises a heavy chain variable region as set forth in the amino acid sequence of SEQ ID NO: 195, and a light chain variable region as set forth in the amino acid sequence of SEQ ID NO: 196. In one embodiment, the anti-CD117 antibody, or antigen binding portion thereof, comprises a heavy chain variable region as set forth in the amino acid sequence of SEQ ID NO: 197, and a light chain variable region as set forth in the amino acid sequence of SEQ ID NO: 198. In one embodiment, the anti-CD117 antibody, or antigen binding portion thereof, comprises a heavy chain variable region as set forth in the amino acid sequence of SEQ ID NO: 199, and a light chain variable region as set forth in the amino acid sequence of SEQ ID NO: 200. In one embodiment, the anti-CD117 antibody, or antigen binding portion thereof, comprises a heavy chain variable region as set forth in the amino acid sequence of SEQ ID NO: 201, and a light chain variable region as set forth in the amino acid sequence of SEQ ID NO: 190. In one embodiment, the anti-CD117 antibody, or antigen binding portion thereof, comprises a heavy chain variable region as set forth in the amino acid sequence of SEQ ID NO: 202, and a light chain variable region as set forth in the amino acid sequence of SEQ ID NO: 203. In one embodiment, the anti-CD117 antibody, or antigen binding portion thereof, comprises a heavy chain variable region as set forth in the amino acid sequence of SEQ ID NO: 204, and a light chain variable region as set forth in the amino acid sequence of SEQ ID NO: 205. In one embodiment, the anti-CD117 antibody, or antigen binding portion thereof, comprises a heavy chain variable region as set forth in the amino acid sequence of SEQ ID NO: 206, and a light chain variable region as set forth in the amino acid sequence of SEQ ID NO: 207. In one embodiment, the anti-CD117 antibody, or antigen binding portion thereof, comprises a heavy chain variable region as set forth in the amino acid sequence of SEQ ID NO: 208, and a light chain variable region as set forth in the amino acid sequence of SEQ ID NO: 209. In one embodiment, the anti-CD117 antibody, or antigen binding portion thereof, comprises a heavy chain variable region as set forth in the amino acid sequence of SEQ ID NO: 210, and a light chain variable region as set forth in the amino acid sequence of SEQ ID NO: 211. In one embodiment, the anti-CD117 antibody, or antigen binding portion thereof, comprises a heavy chain variable region as set forth in the amino acid sequence of SEQ ID NO: 212, and a light chain variable region as set forth in the amino acid sequence of SEQ ID NO: 213. In one embodiment, the anti-CD117 antibody, or antigen binding portion thereof, comprises a heavy chain variable region as set forth in the amino acid sequence of SEQ ID NO: 214, and a light chain variable region as set forth in the amino acid sequence of SEQ ID NO: 215. In one embodiment, the anti-CD117 antibody, or antigen binding portion thereof, comprises a heavy chain variable region as set forth in the amino acid sequence of SEQ ID NO: 216, and a light chain variable region as set forth in the amino acid sequence of SEQ ID NO: 217. In one embodiment, the anti-CD117 antibody, or antigen binding portion thereof, comprises a heavy chain variable region as set forth in the amino acid sequence of SEQ ID NO: 218, and a light chain variable region as set forth in the amino acid sequence of SEQ ID NO: 219. In one embodiment, the anti-CD117 antibody, or antigen binding portion thereof, comprises a heavy chain variable region as set forth in the amino acid sequence of SEQ ID NO: 220, and a light chain variable region as set forth in the amino acid sequence of SEQ ID NO: 221. In one embodiment, the anti-CD117 antibody, or antigen binding portion thereof, comprises a heavy chain variable region as set forth in the amino acid sequence of SEQ ID NO: 222, and a light chain variable region as set forth in the amino acid sequence of SEQ ID NO: 223. In one embodiment, the anti-CD117 antibody, or antigen binding portion thereof, comprises a heavy chain variable region as set forth in the amino acid sequence of SEQ ID NO: 224, and a light chain variable region as set forth in the amino acid sequence of SEQ ID NO: 225. In one embodiment, the anti-CD117 antibody, or antigen binding portion thereof, comprises a heavy chain variable region as set forth in the amino acid sequence of SEQ ID NO: 226, and a light chain variable region as set forth in the amino acid sequence of SEQ ID NO: 227. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 228. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 229. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 230. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 231. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 232. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 233. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 234. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 235. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 236. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 237. In one embodiment, the anti-CD117 antibody, or antigen binding portion thereof, comprises a heavy chain variable region as set forth in the amino acid sequence of SEQ ID NO: 7, and a light chain variable region as set forth in the amino acid sequence of SEQ ID NO: 237. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 244. In one embodiment, the anti-CD117 antibody, or antigen binding portion thereof, comprises a heavy chain variable region as set forth in the amino acid sequence of SEQ ID NO: 251, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 243, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 259, and a light chain variable region as set forth in the amino acid sequence of SEQ ID NO: 256. In one embodiment, the anti-CD117 antibody, or antigen binding portion thereof, comprises a heavy chain variable region as set forth in the amino acid sequence of SEQ ID NO: 260, and a light chain variable region as set forth in the amino acid sequence of SEQ ID NO: 252. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 239. In one embodiment, the anti-CD117 antibody, or antigen binding portion thereof, comprises a heavy chain variable region as set forth in the amino acid sequence of SEQ ID NO: 147, and a light chain variable region as set forth in the amino acid sequence of SEQ ID NO: 240. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 241. In one embodiment, the anti-CD117 antibody, or antigen binding portion thereof, comprises a heavy chain variable region as set forth in the amino acid sequence of SEQ ID NO: 238, and a light chain variable region as set forth in the amino acid sequence of SEQ ID NO: 242.

As described below, an scFV phage display library screen of human antibodies was performed to identify novel anti-CD117 antibodies, and fragments thereof, having therapeutic use. Antibodies 85 (Ab85), 86 (Ab86), 87 (Ab87). 88 (Ab88), and 89 (Ab89), among others, were identified in this screen.

The heavy chain variable region (VH) amino acid sequence of Ab85 is provided below as SEQ ID NO: 243. The VH CDR amino acid sequences of Ab85 are underlined below and are as follows: NYWIG (VH CDR1; SEQ ID NO: 245); IINPRDSDTRYRPSFQG (VH CDR2; SEQ ID NO: 246); and HGRGYEGYEGAFDI (VH CDR3; SEQ ID NO: 247).

Ab85 VH sequence
(SEQ ID NO: 243)
EVQLVQSGAEVKKPGESLKISCKGSGYSFTNYWIGWVRQMPGKGLEWMAI
INPRDSDTRYRPSFQGQVTISADKSISTAYLQWSSLKASDTAMYYCARHG
RGYEGYEGAFDIWGQGTLVTVSS

The light chain variable region (VL) amino acid sequence of Ab85 is provided below as SEQ ID NO 244. The VL CDR amino acid sequences of Ab85 are underlined below and are as follows: RSSQGIRSDLG (VL CDR1; SEQ ID NO: 248); DASNLET (VL CDR2; SEQ ID NO: 249); and QQANGFPLT (VL CDR3; SEQ ID NO: 250).

Ab85 VL sequence
(SEQ ID NO: 244)
DIQMTOSPSSLSASVGDRVTITCRSSQGIRSDLGWYQQKPGKAPKLLIYD
ASNLETGVPSRFSGSGSGTDFTLTISSLQPEDFATYYCQQANGFPLTFGG
GTKVEIK

Antibody HC-86/LC-86 (Ab86)

The heavy chain variable region (VH) amino acid sequence of Ab8 is provided below as SEQ ID NO: 251. The VH CDR amino acid sequences Ab86 are underlined below and are as follows: NYWIG (VH CDR1; SEQ ID NO: 245); IIYPGDSDIRYSPSLQG (VH CDR2; SEQ ID NO: 253); and HGRGYNGYEGAFDI (VH CDR3; SEQ ID NO: 3).

Ab86 VH sequence
(SEQ ID NO: 251)
EVQLVQSGAEVKKPGESLKISCKGSGYSFTNYWIGWVRQMPGKGLEWMGI
IYPGDSDIRYSPSLQGQVTISVDTSTSTAYLQWNSLKPSDTAMYYCARHG
RGYNGYEGAFDIWGQGTLVTVSS

The light chain variable region (VL) amino acid sequence of Ab86 is provided below as SEQ ID NO 252. 5 The VL CDR amino acid sequences of Ab86 are underlined below and are as follows: RASQGIGDSLA (VL CDR1; SEQ ID NO: 254); DASNLET (VL CDR2; SEQ ID NO: 249); and QQLNGYPIT (VL CDR3; SEQ ID NO: 255).

Ab86 VL sequence
(SEQ ID NO: 252)
DIQMTQSPSSLSASVGDRVTITCRASQGIGDSLAWYQQKPGKAPKLLIYD
ASNLETGVPSRFSGSGSGTDFTLTISSLQPEDFATYYCQQLNGYPITFGQ
GTKVEIK

Antibody HC-87/LC-87 (Ab87)

The heavy chain variable region (VH) amino acid sequence of Ab87 is provided below as SEQ ID NO: 243. The VH CDR amino acid sequences of Ab87 are underlined below and are as follows: NYWIG (VH CDR1; SEQ ID NO: 245); IINPRDSDTRYRPSFQG (VH CDR2; SEQ ID NO: 246); and HGRGYEGYEGAFDI (VH CDR3; SEQ ID NO: 247).

Ab87 VH sequence
(SEQ ID NO: 243)
EVQLVQSGAEVKKPGESLKISCKGSGYSFTNYWIGWVRQMPGKGLEWMAI
INPRDSDTRYRPSFQGQVTISADKSISTAYLQWSSLKASDTAMYYCARHG
RGYEGYEGAFDIWGQGTLVTVSS

The light chain variable region (VL) amino acid sequence of Ab87 is provided below as SEQ ID NO 256. The VL CDR amino acid sequences of Ab87 are underlined below and are as follows: RASQGIRNDLG (VL CDR1; SEQ ID NO: 257); DASSLES (VL CDR2; SEQ ID NO: 5); and QQLNGYPIT (VL CDR3; SEQ ID NO: 255).

Ab87 VL sequence
(SEQ ID NO: 256)
DIQMTQSPSSLSASVGDRVTITCRASQGIRNDLGWYQQKPGKAPKLLIYD
ASSLESGVPSRFSGSGSGTDFTLTISSLQPEDFATYYCQQLNGYPITFGQ
GTKVEIK

Antibody HC-88/LC-88 (Ab88)

The heavy chain variable region (VH) amino acid sequence of Ab88 is provided below as SEQ ID NO: 258. The VH CDR amino acid sequences of Ab88 are underlined below and are as follows: NYWIG (VH CDR1; SEQ ID NO: 245); IIYPGDSLTRYSPSFQG (VH CDR2; SEQ ID NO: 259); and HGRGYNGYEGAFDI (VH CDR3; SEQ ID NO: 3).

Ab88 VH sequence
(SEQ ID NO: 258)
EVQLVQSGAEVKKPGESLKISCKGSGYSFTNYWIGWVRQMPGKGLEWMGI
IYPGDSLTRYSPSFQGQVTISADKSISTAYLQWSSLKASDTAMYYCARHG
RGYNGYEGAFDIWGQGTLVTVSS

The light chain variable region (VL) amino acid sequence of Ab88 is provided below as SEQ ID NO: 256. The VL CDR amino acid sequences of Ab88 are underlined below and are as follows: RASQGIRNDLG (VL CDR1; SEQ ID NO: 257); DASSLES (VL CDR2; SEQ ID NO: 5); and QQLNGYPIT (VL CDR3; SEQ ID NO: 255).

Ab88 VL sequence
(SEQ ID NO: 256)
DIQMTQSPSSLSASVGDRVTITCRASQGIRNDLGWYQQKPGKAPKLLIYD
ASSLESGVPSRFSGSGSGTDFTLTISSLQPEDFATYYCQQLNGYPITFGQ
GTKVEIK

Antibody HC-89/LC-89 (Ab89)

The heavy chain variable region (VH) amino acid sequence of Ab89 is provided below as SEQ ID NO: 260. The VH CDR amino acid sequences of Ab89 are underlined below and are as follows: NYWIG (VH CDR1; SEQ ID NO: 245); IIYPGDSDTRYSPSFQG (VH CDR2; SEQ ID NO: 2); and HGRGYNGYEGAFDI (VH CDR3; SEQ ID NO: 3).

Ab89 VH sequence
(SEQ ID NO: 260)
EVQLVQSGAEVKKPGESLKISCKGSGYSFTNYWIGWVRQMPGKGLEWMGI
IYPGDSDTRYSPSFQGQVTISADKSISTAYLQWSSLKASDTAMYYCARHG
RGYNGYEGAFDIWGQGTLVTVSS

The light chain variable region (VL) amino acid sequence of Ab89 is provided below as SEQ ID NO: 252. The VL CDR amino acid sequences of Ab89 are underlined below and are as follows: RASQGIGDSLA (VL CDR1; SEQ ID NO: 254); DASNLET (VL CDR2; SEQ ID NO: 249); and QQLNGYPIT (VL CDR3; SEQ ID NO: 255).

Ab89 VL sequence
(SEQ ID NO: 252)
DIQMTQSPSSLSASVGDRVTITCRASQGIGDSLAWYQQKPGKAPKLLIYD
ASNLETGVPSRFSGSGSGTDFTLTISSLQPEDFATYYCQQLNGYPITFGQ
GTKVEIK

Antibody HC-249/LC-249 (Ab249)

The heavy chain variable region (VH) amino acid sequence of Ab249 is provided below as SEQ ID NO: 238. The VH CDR amino acid sequences of Ab249 are underlined below and are as follows: TSWIG (VH CDR1; SEQ ID NO: 286); IIYPGDSDTRYSPSFQG (VH CDR2; SEQ ID NO: 2); and HGLGYNGYEGAFDI (VH CDR3; SEQ ID NO: 287).

Ab249 VH sequence
(SEQ ID NO: 238)
EVQLVQSGAEVKKPGESLKISCKGSGYRFTTSWIGWVRQMPGKGLEWMGI
IYPGDSDTRYSPSFQGQVTISADKSISTAYLQWSSLKASDTAMYYCARHG
LGYNGYEGAFDIWGQGTLVTVSS

The light chain variable region (VL) amino acid sequence of Ab249 is provided below as SEQ ID NO: 242. The VL CDR amino acid sequences of Ab249 are underlined below and are as follows: RASQGIGSALA (VL CDR1; SEQ ID NO: 288); DASNLET (VL CDR2; SEQ ID NO: 249); and QQLNGYPLT (VL CDR3; SEQ ID NO: 289).

Ab249 VL sequence
(SEQ ID NO: 242)
DIQMTQSPSSLSASVGDRVTITCRASQGIGSALAWYQQKPGKAPKLLIYD
ASNLETGVPSRFSGSGSGTDFTLTISSLQPEDFATYYCQQLNGYPLTFGQ
GTRLEIK

Human antibodies Ab85 and Ab249 were both derived from antibody CK6, which is an antagonist anti-CD117 antibody. Both antibodies have improved properties, e.g., improved binding characteristics, over CK6.

CK6 includes a potential deamidation site in the CDR3 domain of the heavy chain variable region. While advantageous to remove for future production of the antibody, the position of the asparagine presents a significant challenge. The potential deamidation site was successfully removed, however, in the Ab85 heavy chain CDR3 such that the antibody (having Ab85 heavy and light chain CDRs) was able to maintain a high affinity level specificity for human CD117 and the ability to internalize. Further, Ab85 has an improved off rate relative to its parent.

Thus, in certain embodiments, an anti-CD117 antibody comprises a heavy chain comprising a CDR set (CDR1, CDR2, and CDR3) as set forth in SEQ ID Nos: 245, 246, and 247, and a light chain comprising a CDR set as set forth in SEQ ID Nos: 248, 249, and 1250, internalizes in cells expressing CD117, and has a k_{off} rate of 5×10⁻⁴ s⁻¹ or less as measured by BLI.

Additional anti-CD117 antibodies that can be used in conjunction with the patient conditioning methods described herein include, for instance, antibodies produced and released from ATCC Accession No. 10716 (deposited as BA7.3C.9), such as the SR-1 antibody, which is described, for example, in U.S. Pat. No. 5,489,516, the disclosure of which is incorporated herein by reference as it pertains to anti-CD117 antibodies.

In one embodiment, an anti-CD117 antibody described herein comprises an Fc region comprising L235A, L235A, D265C, and H435A (EU index).

Additional anti-CD117 antibodies that can be used in conjunction with the patient conditioning methods described herein include those described in U.S. Pat. No. 7,915,391, which describes, e.g., humanized SR-1 antibodies; U.S. Pat. No. 5,808,002, which describes, e.g., the anti-CD117 A3C6E2 antibody, as well as those described in, for example, WO 2015/050959, which describes anti-CD117 antibodies that bind epitopes containing Pro317, Asn320, Glu329, Va1331, Asp332, Lus358, Glue360, Glue376, His378, and/or Thr380 of human CD117; and US 2012/0288506 (also published as U.S. Pat. No. 8,552,157), which describes, e.g., the anti-CD117 antibody CK6.

Additional anti-CD117 antibodies and antigen-binding fragments thereof that may be used in conjunction with the compositions and methods described herein include those described in US 2015/0320880, such as the clones 9P3, NEG024, NEG027, NEG085, NEG086, and 20376.

### Anti-CD45 Antibodies

Antibodies and antigen-binding fragments capable of binding human CD45 (mRNA NCBI Reference Sequence: NM_080921.3, Protein NCBI Reference Sequence: NP_563578.2), including those capable of binding the isoform CD45RO, can be used in conjunction with the compositions and methods disclosed herein, such as to promote engraftment of hematopoietic stem cell grafts in a patient in need of hematopoietic stem cell transplant therapy. In one embodiment, the compositions and methods disclosed herein include an anti-CD45 antibody or ADC that binds to human CD45RO as set forth in the amino acid sequence of SEQ ID NO: 290. Antibodies that bind to the various isoforms of CD45 disclosed herein are also contemplated for use in the methods and compositions disclosed herein. Multiple isoforms of CD45 arise from the alternative splicing of 34 exons in the primary transcript. Splicing of exons 4, 5, 6, and potentially 7 give rise to multiple CD45 variations.

Selective exon expression is observed in the CD45 isoforms described in Table 1, below.

TABLE 1
Exon expression in various CD45 isoforms

| CD45 isoform | Exon Expression Pattern |
|---|---|
| CD45RA (SEQ ID NO: 291) | Expresses exon 4 only |
| CD45RB (SEQ ID NO: 292) | Expresses exon 5 only |
| CD45RC (SEQ ID NO: 293) | Expresses exon 6 only |
| CD45RO (SEQ ID NO: 290) | Does not express exons 4-6 |

Alternative splicing can result in individual exons or combinations of exons expressed in various isoforms of the CD45 protein (for example, CD45RA, CD45RAB, CD45RABC). In contrast, CD45RO lacks expression of exons 4-6 and is generated from a combination of exons 1-3 and 7-34. There is evidence that exon 7 can also be excluded from the protein, resulting in splicing together of exons 1-3 and 8-34. This protein, designated E3-8, has been detected at the mRNA level but has not been currently identified by flow cytometry.

CD45RO is currently the only known CD45 isoform expressed on hematopoietic stem cells. CD45RA and CD45RABC have not been detected or are excluded from the phenotype of hematopoietic stem cells. There is evidence from studies conducted in mice that CD45RB is expressed on fetal hematopoietic stem cells, but it is not present on adult bone marrow hematopoietic stem cells. Notably, CD45RC has a high rate of polymorphism in exon 6 found within Asian populations (a polymorphism at exon 6 in CD45RC is found in approximately 25% of the Japanese population). This polymorphism leads to high expression of CD45RO and decreased levels of CD45RA, CD45RB, and CD45RC. Additionally, CD45RA variants (such as CD45RAB and CD45RAC) exhibit a polymorphism in exon 4 that has been associated with autoimmune disease.

The presence of CD45RO on hematopoietic stem cells and its comparatively limited expression on other immune cells (such as T and B lymphocyte subsets and various myeloid cells) renders CD45RO a particularly well-suited target for conditioning therapy for patients in need of a hematopoietic stem cell transplant. As CD45RO only lacks expression of exons 4, 5, and 6, its use as an immunogen enables the screening of pan CD45 Abs and CD45RO-specific antibodies.

Anti-CD45 antibodies that can be used in conjunction with the patient conditioning methods described herein include anti-CD45 antibodies, and antigen-binding portions thereof. Antigen-binding portions of antibodies are well known in the art, and can readily be constructed based on the antigen-binding region of the antibody. In exemplary embodiments, the anti-CD45 antibody used in conjunction with the conditioning methods described herein can be a monoclonal antibody or antigen-binding fragment thereof, a polyclonal antibody or antigen-binding fragment thereof, a humanized antibody or antigen-binding fragment thereof, a fully human antibody or antigen-binding fragment thereof, a chimeric antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab)₂ molecule, or a tandem di-scFv. Exemplary anti-CD45 antibodies which may be used in whole or in part in the ADCs or methods described herein are provided below.

In one embodiment, the anti-CD45 antibody is or is derived from clone H₁₃₀, which is commercially available from BIOLEGEND® (San Diego, Calif.), or a humanized variant thereof. Humanization of antibodies can be performed by replacing framework residues and constant region residues of a non-human antibody with those of a germline human antibody according to procedures known in the art (as described, for instance, in Example 7, below). Additional anti-CD45 antibodies that can be used in conjunction with the methods described herein include the anti-CD45 antibodies ab10558, EP322Y, MEM-28, ab10559, 0.N.125, F10-89-4, Hie-1, 2B11, YTH24.5, PD7/26/16, F10-89-4, 1B7, ab154885, B-A11, phosphor S1007, ab170444, EP350, Y321, GA90, D3/9, X1 6/99, and LT45, which are commercially available from ABCAM@ (Cambridge, Mass.), as well as humanized variants thereof. Further anti-CD45 antibodies that may be used in conjunction with the patient conditioning procedures described herein include anti-CD45 antibody HPA000440, which is commercially available from SIGMA-ALDRICH® (St. Louis, Mo.), and humanized variants thereof. Additional anti-CD45 antibodies that can be used in conjunction with the patient conditioning methods described herein include murine monoclonal antibody BC8, which is described, for instance, in Matthews et al., Blood 78:1864-1874, 1991, the disclosure of which is incorporated herein by reference as it pertains to anti-CD45 antibodies, as well as humanized variants thereof. Further anti-CD45 antibodies that can be used in conjunction with the methods described herein include monoclonal antibody YAML568, which is described, for instance, in Glatting et al., J. Nucl. Med. 8:1335-1341, 2006, the disclosure of which is incorporated herein by reference as it pertains to anti-CD45 antibodies, as well as humanized variants thereof. Additional anti-CD45 antibodies that can be used in conjunction with the patient conditioning procedures described herein include monoclonal antibodies YTH54.12 and YTH25.4, which are described, for instance, in Brenner et al., Ann. N.Y. Acad. Sci. 996:80-88, 2003, the disclosure of which is incorporated herein by reference as it pertains to anti-CD45 antibodies, as well as humanized variants thereof. Additional anti-CD45 antibodies for use with the patient conditioning methods described herein include UCHL1, 2H4, SN130, MD4.3, MBI, and MT2, which are described, for instance, in Brown et al., Immunology 64:331-336, 1998, the disclosure of which is incorporated herein by reference as it pertains to anti-CD45 antibodies, as well as humanized variants thereof. Additional anti-CD45 antibodies that can be used in conjunction with the methods described herein include those produced and released from American Type Culture Collection (ATCC) Accession Nos. RA3-6132, RA3-2C2, and TIB122, as well as monoclonal antibodies C363.16A, and 13/2, which are described, for instance, in Johnson et al., J. Exp. Med. 169:1179-1184, 1989, the disclosure of which is incorporated herein by reference as it pertains to anti-CD45 antibodies, as well as humanized variants thereof. Further anti-CD45 antibodies that can be used in conjunction with the patient conditioning methods described herein include the monoclonal antibodies AHN-12.1, AHN-12, AHN-12.2, AHN-12.3, AHN-12.4, HLe-1, and KC56(T200), which are described, for instance, in Harvath et al., J. Immunol. 146:949-957, 1991, the disclosure of which is incorporated herein by reference as it pertains to anti-CD45 antibodies, as well as humanized variants thereof.

Additional anti-CD45 antibodies that can be used in conjunction with the patient conditioning methods described herein include those described, for example, in U.S. Pat. No. 7,265,212 (which describes, e.g., anti-CD45 antibodies 39E11, 16C9, and 1G10, among other clones); U.S. Pat. No. 7,160,987 (which describe, e.g., anti-CD45 antibodies produced and released by ATCC Accession No. HB-11873, such as monoclonal antibody 6G3); and 6,099,838 (which describes, e.g., anti-CD45 antibody MT3, as well as antibodies produced and released by ATCC Accession Nos. HB220 (also designated MB23G2) and HB223), as well as US 2004/0096901 and US 2008/0003224 (which describes, e.g., anti-CD45 antibodies produced and released by ATCC Accession No. PTA-7339, such as monoclonal antibody 17.1), the disclosures of each of which are incorporated herein by reference as they pertain to anti-CD45 antibodies.

Further anti-CD45 antibodies that can be used in conjunction with the patient conditioning methods described herein include antibodies produced and released from ATCC Accession Nos. MB4B4, MB23G2, 14.8, GAP 8.3, 74-9-3, I/24.D6, 9.4, 4B2, M1/9.3.4.HL.2, as well as humanized and/or affinity-matured variants thereof. Affinity maturation can be performed, for instance, using in vitro display techniques described herein or known in the art, such as phage display.

Additional anti-CD45 antibodies that can be used in conjunction with the patient conditioning methods described herein include anti-CD45 antibody T29/33, which is described, for instance, in Morikawa et al., Int. J. Hematol. 54:495-504, 1991, the disclosure of which is incorporated herein by reference as it pertains to anti-CD45 antibodies.

In certain embodiments, the anti-CD45 antibody is selected from apamistamab (also known 90Y-BC8, lomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003/048327, WO2016/016442, US2017/0226209, US2016/0152733, U.S. Pat. No. 9,701,756; US2011/0076270, or U.S. Pat. No. 7,825,222, each of which is incorporated by reference in its entirety.

For example, in one embodiment, the anti-CD45 antibody, or antigen-binding fragment thereof, comprising binding regions, e.g., CDRs, variable regions, corresponding to those of apamistamab. The heavy chain variable region (VH) amino acid sequence of apamistamab is set forth in SEQ ID NO: 296 (see Table 3). The light chain variable region (VL) amino acid sequence of apamistamab is described in SEQ ID NO: 297 (see Table 3). In other embodiments, an anti-CD45 antibody, or antigen-binding portion thereof, comprises a variable heavy chain comprising the amino acid residues set forth in SEQ ID NO: 296, and a light chain variable region as set forth in SEQ ID NO: 297. In one embodiment, the anti-CD45 antibody comprises a heavy chain comprising a CDR1, CDR2 and CDR3 of apamistamab, and a light chain variable region comprising a CDR1. CDR2 and CDR3 of apamistamab.

In one embodiment, the anti-CD45 antibody comprises a heavy chain of an anti-CD45 antibody described herein, and a light chain variable region of anti-CD45 antibody described herein. In one embodiment, the anti-CD45 antibody comprises a heavy chain comprising a CDR1, CDR2 and CDR3 of an anti-CD45 antibody described herein, and a light chain variable region comprising a CDR1, CDR2 and CDR3 of an anti-CD45 antibody described herein.

In another embodiment, the antibody, or antigen-binding fragment thereof, comprises a heavy chain variable region that comprises an amino acid sequence having at least 95% identity to an anti-CD45 antibody herein, e.g., at least 95%, 96%, 97%, 98%, 99%, or 100% identity to an anti-CD45 antibody herein. In certain embodiments, an antibody comprises a modified heavy chain (HC) variable region comprising an HC variable domain of an anti-CD45 antibody herein, or a variant thereof, which variant (i) differs from the anti-CD45 antibody in 1, 2, 3, 4 or 5 amino acids substitutions, additions or deletions; (ii) differs from the anti-CD45 antibody in at most 5, 4, 3, 2, or 1 amino acids substitutions, additions or deletions; (iii) differs from the anti-CD45 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD45 antibody, wherein in any of (i)−(iv), an amino acid substitution may be a conservative amino acid substitution or a non-conservative amino acid substitution; and wherein the modified heavy chain variable region can have an enhanced biological activity relative to the heavy chain variable region of the anti-CD45 antibody, while retaining the CD45 binding specificity of the antibody.

Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD45 binding assay.

### Methods of Identifying Antibodies

Methods for high throughput screening of antibody, or antibody fragment libraries for molecules capable of binding an antigen (e.g., CD117 (e.g., GNNK+CD117), or CD45) expressed by hematopoietic stem be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate antibodies, or antigen-binding fragments, that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140. 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982. 1991; and Clackson et al., Nature 352:624-628, 1991, the disclosures of each of which are incorporated herein by reference as they pertain to in vitro display platforms for the discovery of antigen-binding molecules). Human anti-HC antibodies (e.g., anti-CD117 antibody or anti-CD45 antibody) can also be generated, for example, in the HuMAb-Mouse® or XenoMouse™. These techniques, among others, can be used to identify and improve the affinity of antibodies, antibody or fragments, capable of binding an antigen (e.g., CD117 (e.g., GNNK+CD117) or CD45) expressed by hematopoietic stem cells can in turn be used to deplete endogenous hematopoietic stem cells in a patient (e.g., a human patient) in need of hematopoietic stem cell transplant therapy.

In addition to in vitro display techniques, computational modeling techniques can be used to design and identify antibodies capable of binding an antigen (e.g., CD117 (e.g., GNNK+CD117), or CD45) expressed by hematopoietic stem cells, or antibody fragments in silico. For example, using computational modeling techniques, one of skill in the art can screen libraries of antibodies, or antibody fragments, in silico for molecules capable of binding specific epitopes on an antigen expressed by hematopoietic stem cells (e.g., CD117 (e.g., GNNK+CD117) or CD45), such as extracellular epitopes of the antigen.

Additional techniques can be used to identify antibodies, or antibody fragments, capable of binding an antigen expressed by hematopoietic stem cells (e.g., CD117 (e.g., GNNK+CD117) or CD45) and that are internalized by the cell, for instance, by receptor-mediated endocytosis. For example, the in vitro display techniques described above can be adapted to screen for antibodies, or antibody fragments, that bind an antigen expressed by hematopoietic stem cells (e.g., CD117 (e.g., GNNK+CD117) or CD45) and that are subsequently internalized. Phage display represents one such technique that can be used in conjunction with this screening paradigm. To identify an anti-HC antibody (e.g., anti-CD117 antibody or anti-CD45 antibody) or antibody fragment, and are subsequently internalized by hematopoietic stem cells, one of skill in the art can use the phage display techniques described in Williams et al., Leukemia 19:1432-1438, 2005, the disclosure of which is incorporated herein by reference in its entirety. For example, using mutagenesis methods known in the art, recombinant phage libraries can be produced that encode antibodies, antibody fragments, such as scFv fragments, Fab fragments, diabodies, triabodies, and ¹⁰Fn3 domains, among others, or ligands that contain randomized amino acid cassettes (e.g., in one or more, or all, of the CDRs or equivalent regions thereof or an antibody or antibody fragment). The framework regions, hinge, Fc domain, and other regions of the antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized antibodies, or antibody fragments, covalently bound to the phage particles can be incubated with an antigen (e.g., CD117 (e.g., GNNK+CD117) or CD45), for instance, by first incubating the phage library with blocking agents (such as, for instance, milk protein, bovine serum albumin, and/or IgG so as to remove phage encoding antibodies, or antibody fragments, that exhibit non-specific protein binding and phage that encode antibodies or fragments thereof that bind Fc domains, and then incubating the phage library with a population of hematopoietic stem cells or mature immune cells (e.g., T-cells), which express, e.g., CD117 (e.g., GNNK+CD117) or CD45. The phage library can be incubated with the hematopoietic stem cells for a time sufficient to allow anti-HC antibodies (e.g., anti-CD117 antibody or anti-CD45 antibody) or antibody fragments, to bind the cognate cell-surface antigen (e.g., CD117 (e.g., GNNK+CD117) or CD45) and to subsequently be internalized by the hematopoietic stem cells (e.g., from 30 minutes to 6 hours at 4° C., such as 1 hour at 4° C.). Phage containing antibodies, or antibody fragments, that do not exhibit sufficient affinity for the antigen (CD117 (e.g., GNNK+CD117) or CD45) so as to permit binding to, and internalization by, hematopoietic stem cells can subsequently be removed by washing the cells, for instance, with cold (4° C.) 0.1 M glycine buffer at pH 2.8. Phage bound to antibodies, or antibody fragments, that have been internalized by the hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the antibodies, or antibody fragments, inserted within the phage genome. The encoded antibodies, or antibody fragments, can subsequently be prepared de novo by chemical synthesis (for instance, of antibody fragments, such as scFv fragments) or by recombinant expression (for instance, of full-length antibodies).

The internalizing capacity of the prepared antibodies, or antibody fragments, can be assessed, for instance, using radionuclide internalization assays known in the art. For example, anti-HC antibodies (e.g., anti-CD117 antibody or anti-CD45 antibody) or antibody fragments, identified using in vitro display techniques described herein or known in the art can be functionalized by incorporation of a radioactive isotope, such as ¹⁸F, ⁷⁵Br, ⁷⁷Br, ¹²²I, ²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, or antibody fragments, using beads, such as polystyrene beads, containing electrophilic halogen reagents (e.g., Iodination Beads, Thermo Fisher Scientific, Inc., Cambridge, Mass.). Radiolabeled antibodies, fragments thereof, or ADCs, can be incubated with hematopoietic stem cells for a time sufficient to permit internalization (e.g., from 30 minutes to 6 hours at 4° C., such as 1 hour at 4° C.). The cells can then be washed to remove non-internalized antibodies or fragments thereof, (e.g., using cold (4° C.) 0.1 M glycine buffer at pH 2.8). Internalized antibodies, or antibody fragments, can be identified by detecting the emitted radiation (e.g., γ-radiation) of the resulting hematopoietic stem cells in comparison with the emitted radiation (e.g., γ-radiation) of the recovered wash buffer. The foregoing internalization assays can also be used to characterize ADCs.

Antibodies may be produced using recombinant methods and compositions, e.g., as described in U.S. Pat. No. 4,816,567. In one embodiment, isolated nucleic acid encoding an anti-HC antibody (e.g., anti-CD117 antibody or anti-CD45 antibody) described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-HC antibody (e.g., an anti-CD117 antibody or an anti-CD45 antibody) nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Nati. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Antibody Drug Conjugates

Antibodies and antigen-binding fragments thereof described herein can be conjugated (linked) to a cytotoxin via a linker. In some embodiments, the cytotoxic molecule is conjugated to a cell internalizing antibody, or antigen-binding fragment thereof as disclosed herein such that following the cellular uptake of the antibody, or fragment thereof, the cytotoxin may access its intracellular target and mediate hematopoietic cell death. Any number of cytotoxins can be conjugated to the anti-HC antibody, e.g., 1, 2, 3, 4, 5, 6, 7, or 8.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

### Cytotoxins

Various cytotoxins can be conjugated to an anti-HC antibody (e.g., an anti-CD117 antibody, an anti-CD45 antibody) via a linker for use in the therapies described herein. In particular, the anti-HC ADCs (e.g., anti-CD117 ADC or anti-CD45 ADC) include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

ADCs of the present disclosure therefore may be of the general formula Ab-(Z-L-D)ₙ, wherein an antibody or antigen-binding fragment thereof (Ab) is conjugated (covalently linked) to linker (L), through a chemical moiety (Z), to a cytotoxic moiety (“drug,” D), each as disclosed herein.

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-HC ADCs (e.g., anti-CD117 ADC or anti-CD45 ADC) may be limited by the number of attachment sites on the antibody. For example, where the attachment is a cysteine thiol, an antibody may have only one or several cysteine thiol groups, or may have only one or several sufficiently reactive thiol groups through which a linker may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as α-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, an indolinobenzodiazepine pseudodimer, or a variant thereof, or another cytotoxic compound described herein or known in the art.

In some embodiments, the cytotoxin of the antibody-drug conjugate is an RNA polymerase inhibitor. In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof.

Additional details regarding cytotoxins that can be used in the anti-HC ADCs (e.g., anti-CD117 ADC or anti-CD45 ADC) useful in the methods of the present disclosure are described below.

Amatoxins

The methods and compositions disclosed herein include ADCs comprising an RNA polymerase inhibitor, e.g., an amatoxin, as the cytotoxin conjugated to an anti-HC antibody (e.g., an anti-CD117 antibody). In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin or a derivative thereof. Structures of the various naturally occurring amatoxins are disclosed in, e.g., Zanotti et al., Int. J. Peptide Protein Res. 30, 1987, 450-459.

Amatoxins useful in conjunction with the compositions and methods described herein include compounds according to, but are not limited to, formula (III), including α-amanitin, γ-amanitin, γ-amanitin, ε-amanitin, amanin, amaninamide, amanullin, amanullinic acid, or proamanullin. Formula (III) is as follows:

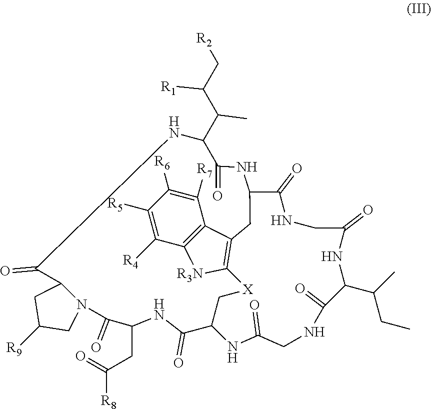

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₁-C₆ alkenyl), optionally substituted heteroalkenyl (e.g., C₂-C₆ heteroalkenyl), optionally substituted alkynyl (e.g., C₁-C₆ alkynyl), optionally substituted heteroalkynyl (e.g., C₁-C₆ heteroalkynyl), optionally substituted cycloalkyl, optionally substituted heterocycloalkyl, optionally substituted aryl, or optionally substituted heteroaryl.

For instance, in one embodiment, amatoxins useful in conjunction with the compositions and methods described herein include compounds according to formula (IIIA)

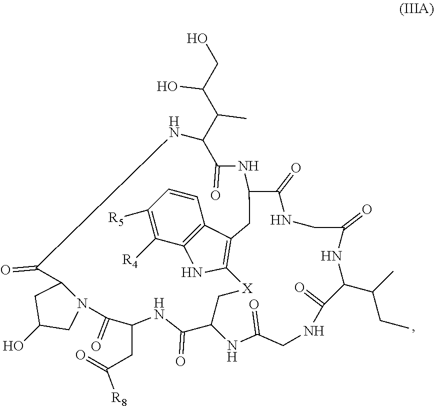

wherein R₄, R₅, X, and R₈ are each as defined above.

For instance, in one embodiment, amatoxins useful in conjunction with the compositions and methods described herein include compounds according to formula (IIIB), below:

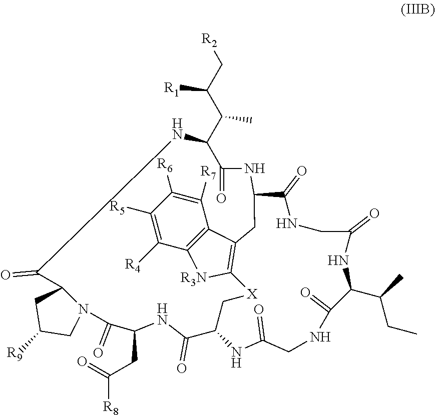

wherein R₁, is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₁-C₆ heteroalkenyl), optionally substituted alkynyl (e.g., C₁-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, amatoxins useful in conjunction with the compositions and methods described herein also include compounds according to formula (IIIC), below:

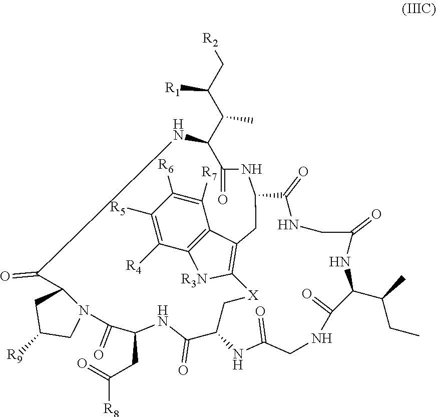

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, the cytotoxin is an amanitin.

For instance, the antibodies, and antigen-binding fragments, described herein may be bound to an amatoxin (e.g., of Formula III, IIIA, IIIB, or IIIC) so as to form a conjugate represented by the formula Ab-Z-L-Am, wherein Ab is the antibody, or antigen-binding fragment thereof, L is a linker, Z is a chemical moiety and Am is an amatoxin. Many positions on amatoxins or derivatives thereof can serve as the position to covalently bond the linking moiety L, and, hence the antibodies or antigen-binding fragments thereof. Exemplary methods of amatoxin conjugation and linkers useful for such processes are described below. Exemplary linker-containing amatoxins Am-L-Z useful for conjugation to an antibody, or antigen-binding fragment, in accordance with the compositions and methods described herein, are shown in structural formulas (I), (IA), (IB), (II), (IIA), and (IIB), recited herein.

In some embodiments, the amatoxin-linker conjugate Am-L-Z is represented by formula (I)

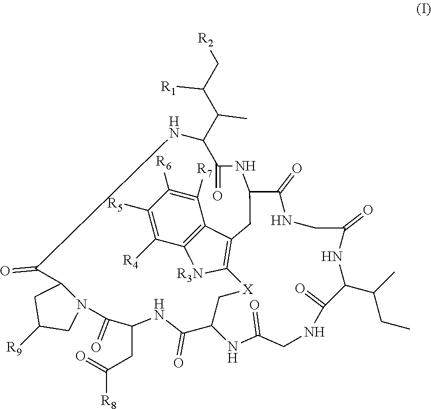

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₁-C₆ alkenyl), optionally substituted heteroalkenyl (e.g., C₁-C₆ heteroalkenyl), optionally substituted alkynyl (e.g., C₁-C₆ alkynyl), optionally substituted heteroalkynyl (e.g., C₁-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₁-C₆ alkenylene), optionally substituted heteroalkenylene (e.g., C₂-C₆ heteroalkenylene), optionally substituted alkynylene (e.g., C₁-C₆ alkynylene), optionally substituted heteroalkynylene (e.g., C₁-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof;

and

Z is a chemical moiety formed from a coupling reaction between a reactive substituent present on L and a reactive substituent present within an antibody, or antigen-binding fragment thereof, that binds a target antigen (e.g., CD117).

In some embodiments, Am contains exactly one R_{C} substituent.

In some embodiments, L-Z is

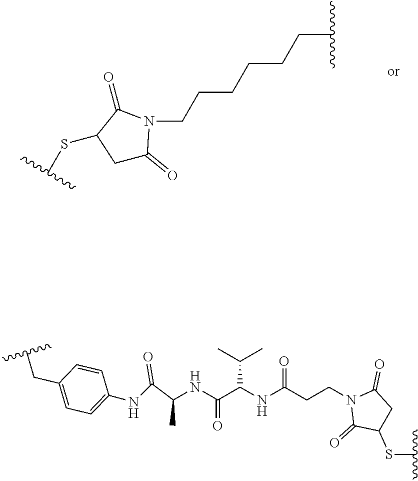

where S is a sulfur atom which represents the reactive substituent present within an antibody, or antigen-binding fragment thereof, that binds a target antigen (e.g., from the —SH group of a cysteine residue).
In some embodiments, L-Z is

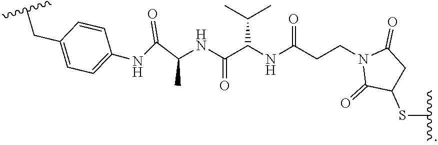

In some embodiments, the conjugate Am-L-Z-Ab is represented by one of formulas IV, IVA, or IVB:

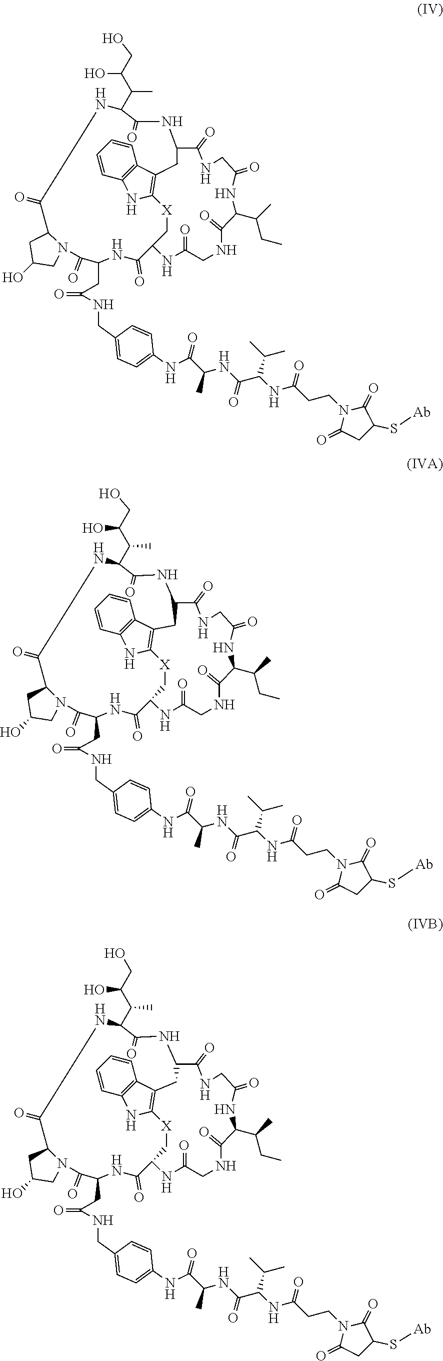

where X is S, SO or SO₂, and the Ab is shown to indicate the point of Ab attachment.

In some embodiments, Am-L-Z-Ab is

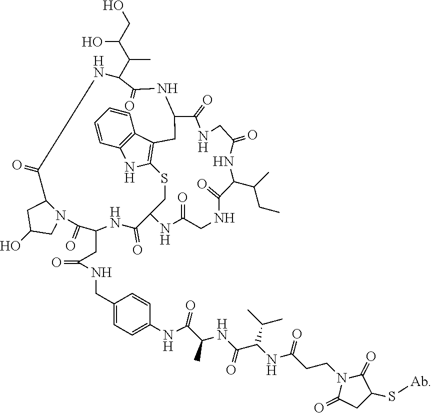

where Ab is shown to indicate the point of Ab attachment.

In some embodiments, Am-L-Z-Ab is

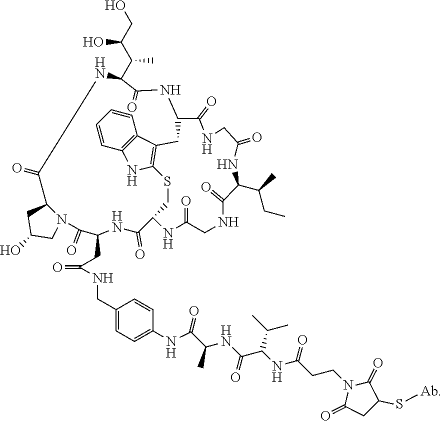

where Ab is shown to indicate the point of Ab attachment.

In some embodiments, Am-L-Z-Ab is

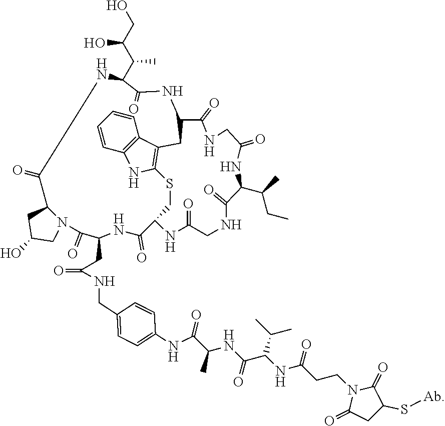

where Ab is shown to indicate the point of Ab attachment.

In some embodiments, the Am-L-Z-Ab precursor, Am-L-Z, is

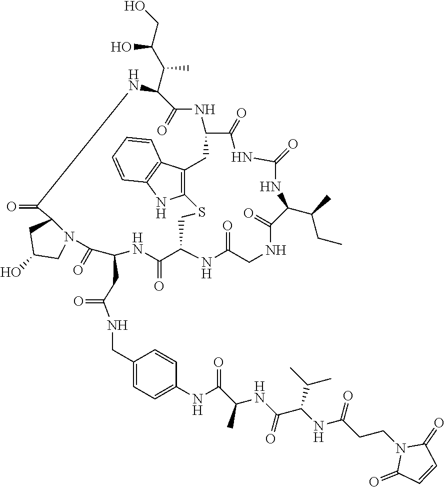 [SMILES: CC[C@H](C)[C@@H]1NC(=O)NC(=O)[C@@H]2Cc3c([nH]c4ccccc34)SC[C@H](NC(=O)CNC1=O)C(=O)N[C@@H](CC(=O)NCc1ccc(NC(=O)[C@H](C)NC(=O)[C@@H](NC(=O)CCN3C(=O)C=CC3=O)C(C)C)cc1)C(=O)N1C[C@H](O)C[C@@H]1C(=O)N[C@@H]([C@@H](C)[C@@H](O)CO)C(=O)N2]

wherein the maleimide reacts with a thiol group found on a cysteine in the antibody.

In some embodiments, Am-L-Z is represented by formula (IA)

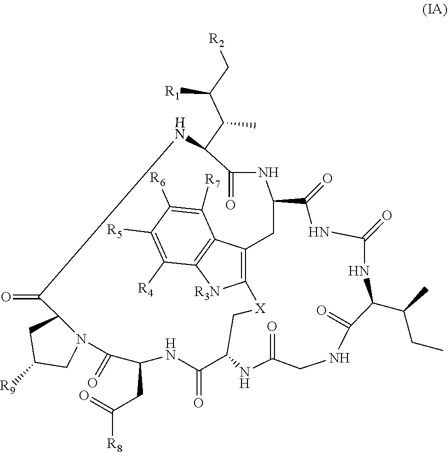

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₁-C₆ heteroalkenyl), optionally substituted alkynyl (e.g., C₁-C₆ alkynyl), optionally substituted heteroalkynyl (e.g., C₁-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₁-C₆ alkenylene), optionally substituted heteroalkenylene (e.g., C₁-C₆ heteroalkenylene), optionally substituted alkynylene (e.g., C₁-C₆ alkynylene), optionally substituted heteroalkynylene (e.g., C₁-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof;

Z is a chemical moiety formed from a coupling reaction between a reactive substituent present on L and a reactive substituent present within an antibody, or antigen-binding fragment thereof, that binds an HC antigen (i.e., an anti-HC antibody, e.g., anti-CD117 antibody or anti-CD45 antibody); and wherein Am contains exactly one R_{C} substituent.

In some embodiments, L-Z is

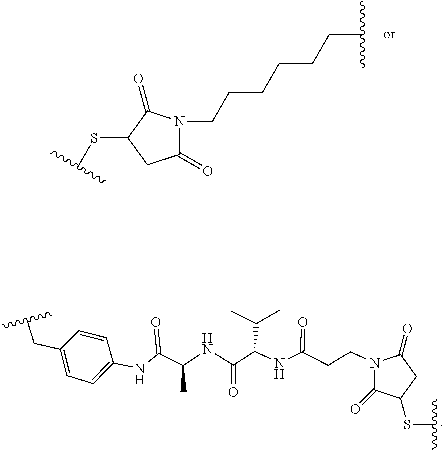 [SMILES: *CCCCCCN1C(=O)CC(S*)C1=O ; *CC1=CCC=C(NC(=O)[C@H](C)NC(=O)[C@@H](NC(=O)CCCN2C(=O)CC(S*)C2=O)C(C)C)C=C1]

In some embodiments, L-Z is

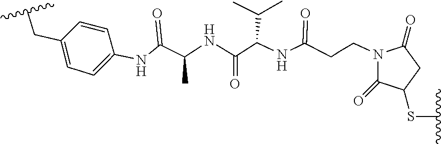 [SMILES: *CC1=CCC=C(NC(=O)[C@H](C)NC(=O)[C@@H](NC(=O)CCCN2C(=O)CC(S*)C2=O)C(C)C)C=C1]

In some embodiments, Am-L-Z is represented by formula (IB)

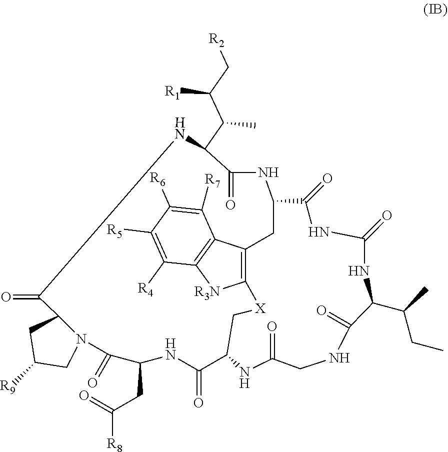

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or RO;

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₁-C₆ alkenyl), optionally substituted heteroalkenyl (e.g., C₁-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₁-C₆ alkynylene), optionally substituted heteroalkynylene (e.g., C₁-C₆ heteroalkynylene), optionally substituted cycloalkylene, optionally substituted heterocycloalkylene, optionally substituted arylene, optionally substituted heteroarylene, a peptide, a dipeptide, —(C═O)—, a disulfide, a hydrazone, or a combination thereof:

Z is a chemical moiety formed from a coupling reaction between a reactive substituent present on L and a reactive substituent present within an antibody, or antigen-binding fragment thereof, that binds an HC antigen (i.e., an anti-HC antibody, e.g., anti-CD117 antibody or anti-CD45 antibody); and wherein Am contains exactly one R_{C} substituent.

In some embodiments, L-Z is

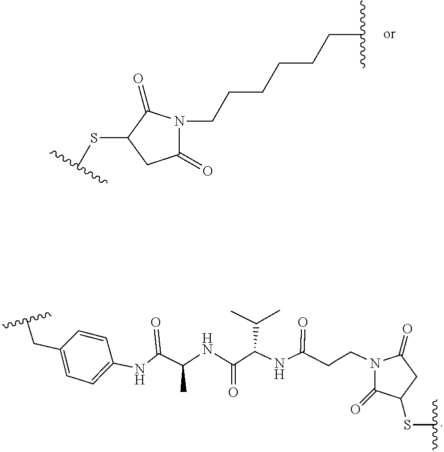 [SMILES: *CCCCCCN1C(=O)CC(S*)C1=O ; *CC1=CCC=C(NC(=O)[C@H](C)NC(=O)[C@@H](NC(=O)CCCN2C(=O)CC(S*)C2=O)C(C)C)C=C1]

In some embodiments, L-Z is

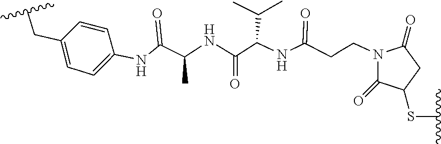 [SMILES: *CC1=CCC=C(NC(=O)[C@H](C)NC(=O)[C@@H](NC(=O)CCCN2C(=O)CC(S*)C2=O)C(C)C)C=C1]

In some embodiments, R_{A} and R_{B}, when present, together with the oxygen atoms to which they are bound, combine to form a 5-membered heterocycloalkyl group of formula:

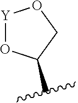

wherein Y is —(C═O)—, —(C═S)—, —(C═NR_{E})—, or —(CR_{E}R_{E′})—; and

R_{E} and R_{E′} are each independently optionally substituted C₁-C₆ alkylene-R_{C}, optionally substituted C₁-C₆ heteroalkylene-R_{C}, optionally substituted C₂-C₆ alkenylene-R_{C}, optionally substituted C₁-C₆ heteroalkenylene-R_{C}, optionally substituted C₂-C₆ alkynylene-R_{C}, optionally substituted C₂-C₆ heteroalkynylene-R_{C}, optionally substituted cycloalkylene-R_{C}, optionally substituted heterocycloalkylene-R_{C}, optionally substituted arylene-R_{C}, or optionally substituted heteroarylene-R_{C}.

In some embodiments, Am-L-Z is represented by formula (IA) or formula (IB), wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

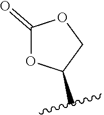 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, or NHR_{C};

R₉ is H or OH;

X is —S—, —S(O)—, or —SO₂; and

wherein R_{C} and R_{D} are each as defined above.

In some embodiments, Am-L-Z is represented by formula (IA) or formula (IB), wherein R₁ is H, OH, OR_{A}, or OR_{C};

R₂ is H, OH, OR_{B}, or OR_{C};

R_{A} and R_{B}, when present, together with the oxygen atoms to which they are bound, combine to form:

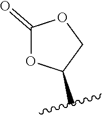 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH;

X is —S—, —S(O)—, or —SO₂—; and

wherein R_{C} is as defined above.

In some embodiments, Am-L-Z is represented by formula (IA) or formula (IB),

wherein R₁ is H, OH, or OR_{A};

R₂ is H, OH, or OR_{B};

R_{A} and R_{B}, when present, together with the oxygen atoms to which they are bound, combine to form:

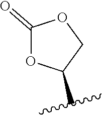 [SMILES: *[C@@H]1COC(=O)O1]

R₃, R₄, R₅, and R₇ are each H;

R₅ is OR_{C};

R₈ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2016/0002298, the disclosure of which is incorporated herein by reference in its entirety.

In some embodiments, Am-L-Z is represented by formula (IA) or formula (IB),

wherein R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2014/0294865, the disclosure of which is incorporated herein by reference in its entirety.

In some embodiments, Am-L-Z is represented by formula (IA) or formula (IB), wherein R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH;

X is —S—, —S(O)—, or —SO₂—; and

wherein R_{C} is as defined above. Such amatoxin conjugates are described, for example, in US Patent Application Publication No. 2015/0218220, the disclosure of which is incorporated herein by reference in its entirety.

In some embodiments, Am-L-Z is represented by formula (IA) or formula (IB),

wherein R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH;

X is —S—, —S(O)—, or —SO₂—; and

wherein R_{C} is as defined above. Such amatoxin conjugates are described, for example, in U.S. Pat. Nos. 9,233,173 and 9,399,681, as well as in US 2016/0089450, the disclosures of each of which are incorporated herein by reference in their entirety.

In some embodiments, Am-L-Z′ is

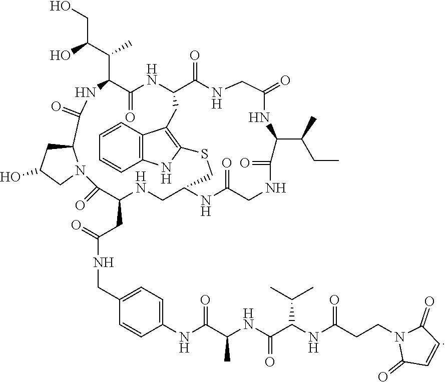

Additional amatoxins that may be used for conjugation to an antibody, or antigen-binding fragment thereof, in accordance with the compositions and methods described herein are described, for example, in WO 2016/142049; WO 2016/071856; WO 2017/149077; WO 2018/115468; and WO 2017/046858, the disclosures of each of which are incorporated herein by reference in their entirety.

In some embodiments, Am-L-Z is represented by formula (II), formula (IIA), or formula (IIB)

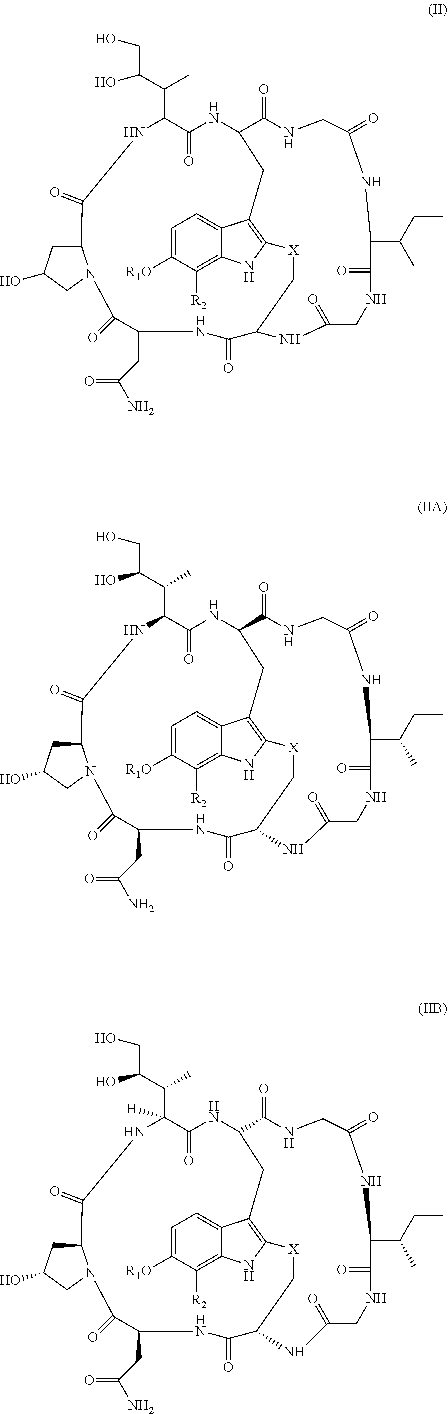

wherein X is S, SO, or SO₂; R₁ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; and R₂ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; wherein when R₁ is H, R₂ is the linker, and when R₂ is H, R₁ is the linker. In some embodiments, R₁ is the linker and R₂ is H, and the linker and chemical moiety, together as L-Z, is

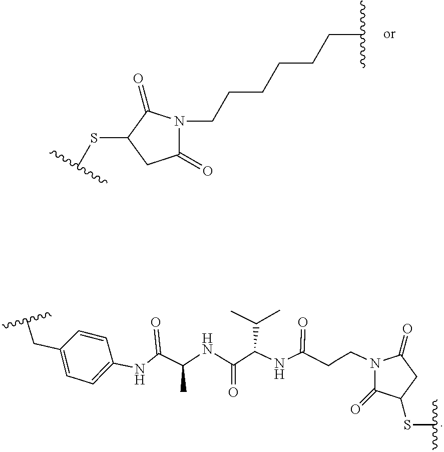 [SMILES: *CCCCCCN1C(=O)CC(S*)C1=O ; *CC1=CCC=C(NC(=O)[C@H](C)NC(=O)[C@@H](NC(=O)CCCN2C(=O)CC(S*)C2=O)C(C)C)C=C1]

In some embodiments, L-Z is

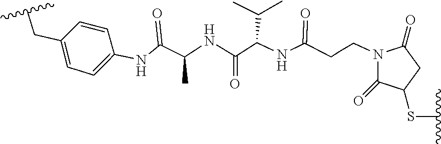 [SMILES: *CC1=CCC=C(NC(=O)[C@H](C)NC(=O)[C@@H](NC(=O)CCCN2C(=O)CC(S*)C2=O)C(C)C)C=C1]

In some embodiments, R₁ is the linker and R₂ is H, and the linker and chemical moiety, together as L-Z, is

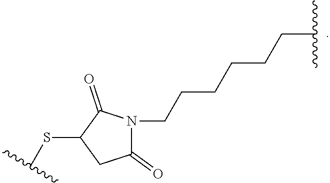 [SMILES: *CCCCCCN1C(=O)CC(S*)C1=O]

In one embodiment, Am-L-Z-Ab is:

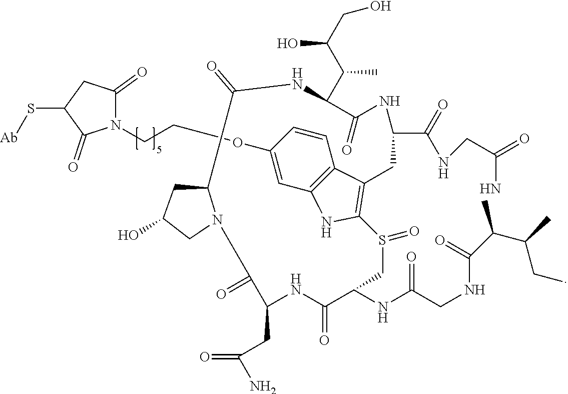

In one embodiment, Am-L-Z-Ab is:

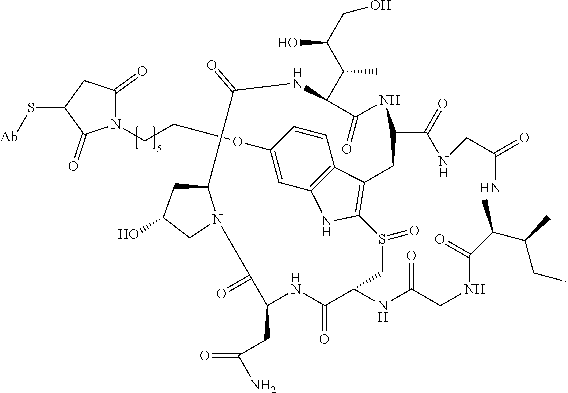

In some embodiments, the Am-L-Z-Ab precursor (i.e., Am-L-Z) is one of:

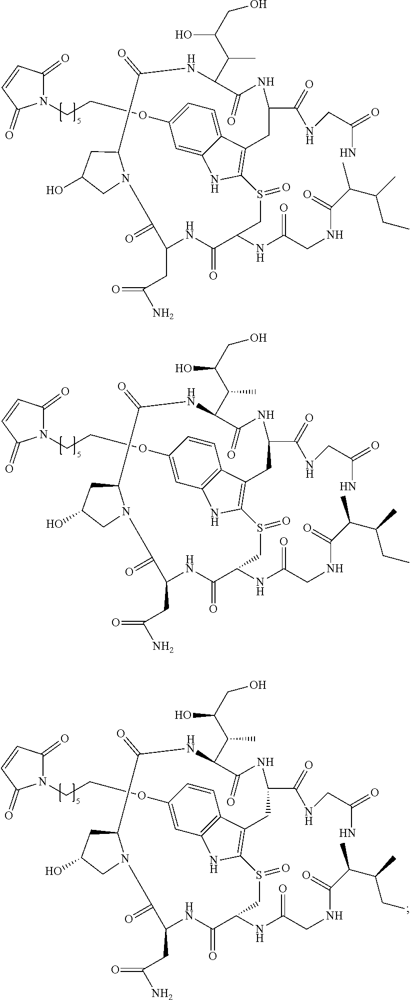

wherein the maleimide reacts with a thiol group found on a cysteine in the antibody.

In some embodiments, the cytotoxin is an α-amanitin. In some embodiments, the α-amanitin is attached to an anti-HC antibody (e.g., anti-CD117 antibody or anti-CD45 antibody) via a linker L. In some embodiments, the α-amanitin is a compound of formula III. The linker L may be attached to the α-amanitin of formula III at any one of several possible positions (e.g., any of R¹-R⁹) to provide an α-amanitin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

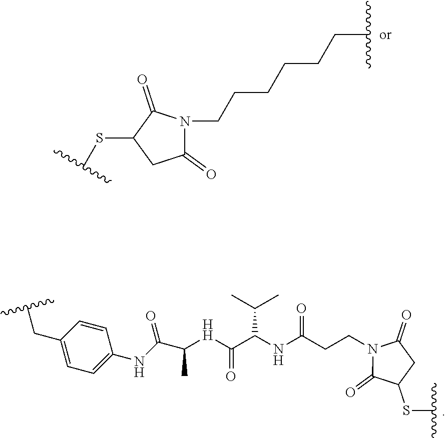

In some embodiments, the cytotoxin is a β-amanitin. In some embodiments, the β-amanitin is attached to an anti-HC antibody (e.g., anti-CD117 antibody or anti-CD45 antibody) via a linker L. In some embodiments, the β-amanitin is a compound of formula III. The linker L may be attached to the R-amanitin of formula III at any one of several possible positions (e.g., any of R¹-R⁹) to provide an β-amanitin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

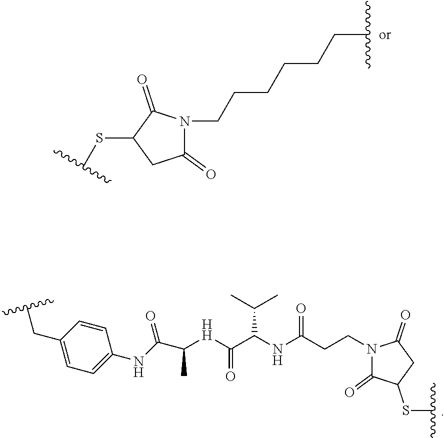

In some embodiments, the cytotoxin is a γ-amanitin. In some embodiments, the γ-amanitin is attached to an anti-HC antibody (e.g., anti-CD117 antibody or anti-CD45 antibody) via a linker L. In some embodiments, the γ-amanitin is a compound of formula III. The linker L may be attached to the γ-amanitin of formula III at any one of several possible positions (e.g., any of R¹-R⁹) to provide an γ-amanitin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

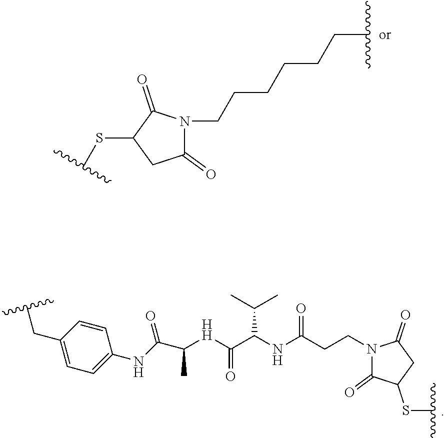

In some embodiments, the cytotoxin is a ε-amanitin. In some embodiments, the ε-amanitin is attached to an anti-HC antibody (e.g., anti-CD117 antibody or anti-CD45 antibody) via a linker L. In some embodiments, the ε-amanitin is a compound of formula III. The linker L may be attached to the ε-amanitin of formula III at any one of several possible positions (e.g., any of R¹-R⁹) to provide an ε-amanitin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

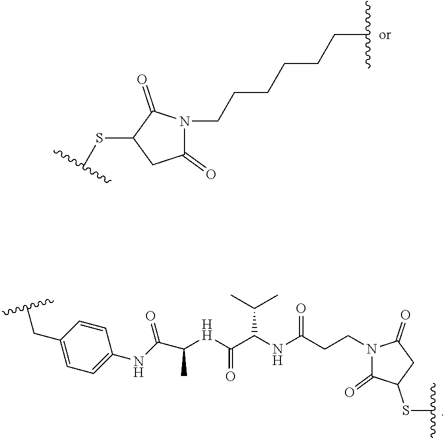

In some embodiments, the cytotoxin is an amanin. In some embodiments, the amanin is attached to an anti-HC antibody (e.g., anti-CD117 antibody or anti-CD45 antibody) via a linker L. In some embodiments, the amanin is a compound of formula III. The linker L may be attached to the amanin of formula III at any one of several possible positions (e.g., any of R¹-R⁹) to provide an amanin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

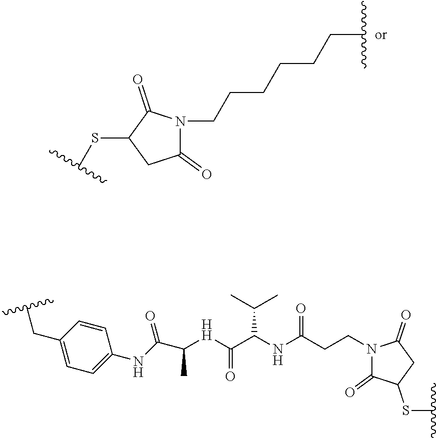

In some embodiments, the cytotoxin is an amaninamide. In some embodiments, the amaninamide is attached to an anti-HC antibody (e.g., anti-CD117 antibody or anti-CD45 antibody) via a linker L. In some embodiments, the amaninamide is a compound of formula III. The linker L may be attached to the amaninamide of formula III at any one of several possible positions (e.g., any of R¹-R⁹) to provide an amaninamide-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

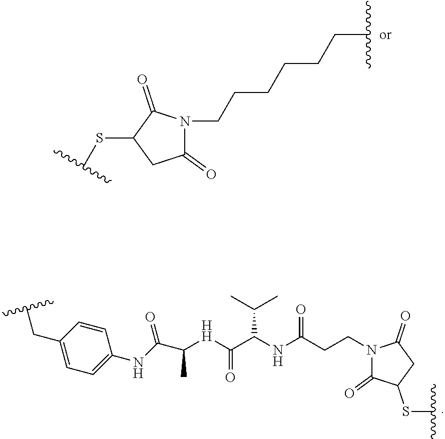

In some embodiments, the cytotoxin is an amanullin. In some embodiments, the amanullin is attached to an anti-HC antibody (e.g., anti-CD117 antibody or anti-CD45 antibody) via a linker L. In some embodiments, the amanullin is a compound of formula III. The linker L may be attached to the amanullin of formula III at any one of several possible positions (e.g., any of R¹-R⁹) to provide an amanullin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

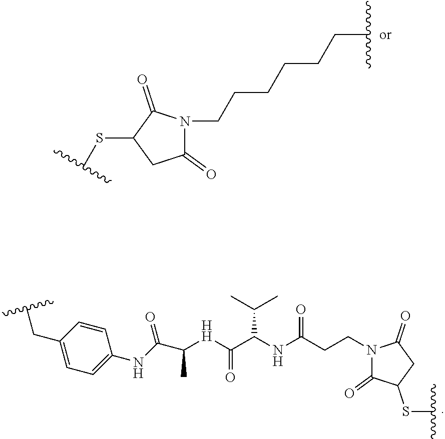

In some embodiments, the cytotoxin is an amanullinic acid. In some embodiments, the amanullinic acid is attached to an anti-HC antibody (e.g., ani-CD117 antibody or ant-CD45 antibody) via a linker L. In some embodiments, the amanullinic acid is a compound of formula III. The linker L may be attached to the amanullinic acid of formula III at any one of several possible positions (e.g., any of R¹-R⁹) to provide an amanullinic acid-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

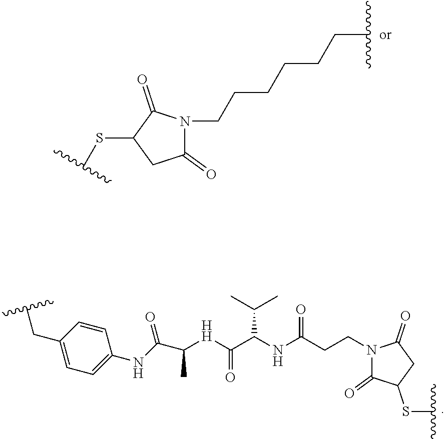

In some embodiments, the cytotoxin is a proamanullin. In some embodiments, the proamanullin is attached to an anti-HC antibody (e.g., anti-CD117 antibody or anti-CD45 antibody) via a linker L. In some embodiments, the proamanullin is a compound of formula III. The linker L may be attached to the proamanullin of formula III at any one of several possible positions (e.g., any of R¹-R⁹) to provide an proamanullin-linker conjugate of formula I, IA, IB, II, IIA, or IIB. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-8.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

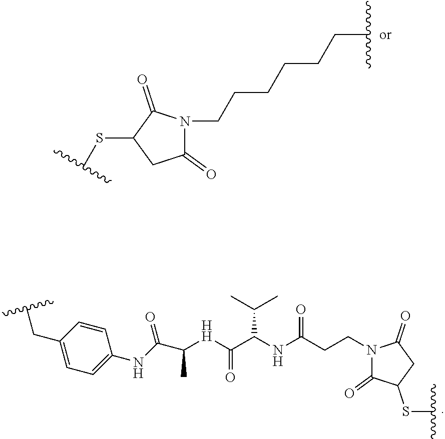

Synthetic methods of making amatoxin are described in U.S. Pat. No. 9,676,702, which is incorporated by reference herein.

Antibodies, and antigen-binding fragments, for use with the compositions and methods described herein can be conjugated to an amatoxin, such as α-amanitin or a variant thereof, using conjugation techniques known in the art or described herein. For instance, antibodies, and antigen-binding fragments thereof, that recognize and bind a target antigen (an anti-HC antibody, e.g., anti-CD117 antibody or anti-CD45 antibody) can be conjugated to an amatoxin, such as α-amanitin or a variant thereof, as described in US 2015/0218220, the disclosure of which is incorporated herein by reference as it pertains, for example, to amatoxins, such as α-amanitin and variants thereof, as well as covalent linkers that can be used for covalent conjugation.

Auristatins

Anti-HC antibodies (e.g., anti-CD117 antibody or anti-CD45 antibody) and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in Senter et al, Proceedings of the American Association for Cancer Research, Volume 45. Abstract Number 623, presented Mar. 28, 2004, the disclosure of which is expressly incorporated by reference in its entirety.

An exemplary auristatin embodiment is MMAE, wherein the wavy line indicates the point of covalent attachment to the linker of an antibody-linker conjugate (-L-Z-Ab or -L-Z′, as described herein).

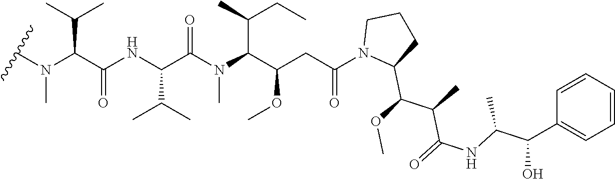 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Another exemplary auristatin embodiment is MMAF, wherein the wavy line indicates the point of covalent attachment to the linker of an antibody-linker conjugate (-L-Z-Ab or -L-Z′, as described herein), as disclosed in US 2005/0238649:

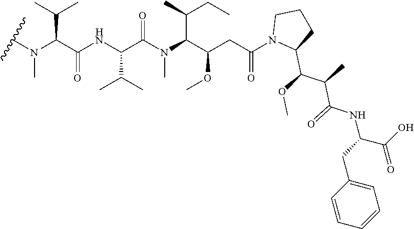 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis. 1996, 719-725; Pettit et al (1996) J. Chem. Soc. Perkin Trans. 15:859-863; and Doronina (2003) Nat. Biotechnol. 21(7):778-784.

Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317.821; 4,322.348; 4,331,598; 4,361,650; 4,364,866: 4,424,219: 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N²′-deacetyl-N²′-(3-mercapto-1-oxopropy)-maytansine, as the cytotoxic agent. DM1 is represented by the following structural formula V:

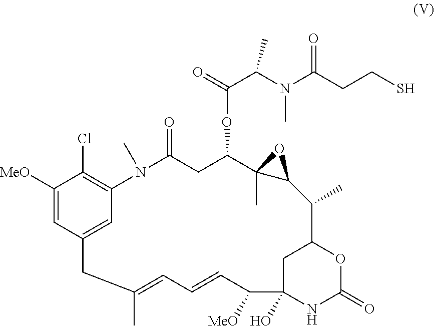 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present disclosure utilize the thiol-containing maytansinoid N²′-deacetyl-N²′(4-methyl-4-mercapto-1-oxopentyl)-maytansine (e.g., DM4) as the cytotoxic agent. DM4 is represented by the following structural formula VI:

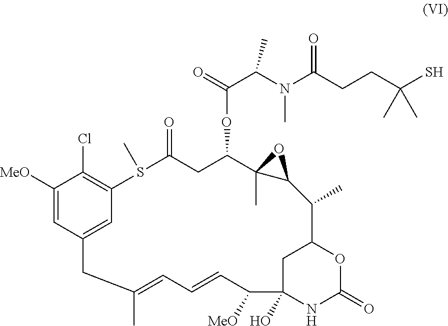

Another maytansinoid comprising a side chain that contains a sterically hindered thiol bond is N²′-deacetyl-N-²′(4-mercapto-1-oxopentyl)-maytansine (termed DM3), represented by the following structural formula VII:

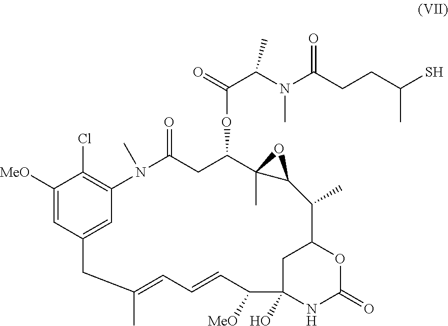 [SMILES: COc1cc2cc(c1Cl)N(C)C(=O)C[C@H](OC(=O)[C@H](C)N(C)C(=O)CCC(C)S)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugates of the present disclosure. In this regard, the entire disclosure of U.S. Pat. Nos. 5,208,020 and 7,276,697 is incorporated herein by reference.

Many positions on maytansinoids can serve as the position to covalently bond the linking moiety and, hence the antibodies or antigen-binding fragments thereof (-L-Z-Ab or -L-Z′, as described herein). For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to covalently bond the linker moiety, and in some particular embodiments, the C-3 position of maytansinol serves as the position to covalently bond the linking moiety. There are many linking groups known in the art for making antibody-maytansinoid conjugates, including, for example, those disclosed in U.S. Pat. Nos. 5,208,020, 6,441,163, and EP Patent No. 0425235 B1; Char et al., Cancer Research 52:127-131 (1992); and U.S. 2005/0169933 A1, the disclosures of which are hereby expressly incorporated by reference. Additional linking groups are described and exemplified herein.

The present disclosure also includes various isomers and mixtures of maytansinoids and conjugates. Certain compounds and conjugates of the present disclosure may exist in various stereoisomeric, enantiomeric, and diastereomeric forms. Several descriptions for producing such antibody-maytansinoid conjugates are provided in U.S. Pat. Nos. 5,208,020; 5,416,064; 6,333,410; 6,441,163; 6,716,821; and 7,368,565, each of which is incorporated herein in its entirety.

Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy; N.R. Bachur, “Free Radical Damage” id. at pp. 97-102]. Because of their cytotoxic potential anthracyclines have been used in the treatment of numerous cancers such as leukemia, breast carcinoma, lung carcinoma, ovarian adenocarcinoma and sarcomas [see e.g., P.H-Wiemik, in Anthracycline: Current Status and New Developments p 11]. Commonly used anthracyclines include doxorubicin, epirubicin, idarubicin and daunomycin.

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

Commonly used anthracyclines include doxorubicin, epirubicin, idarubicin and daunomycin. In some embodiments, the cytotoxin is an anthracycline selected from the group consisting of daunorubicin, doxorubicin, epirubicin, and idarubicin

Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.) The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by the structural formula:

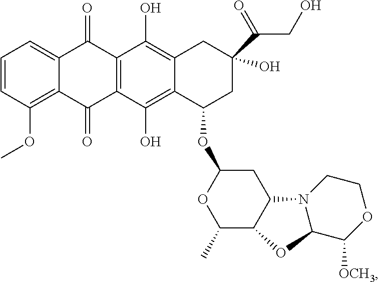

Multiple positions on anthracyclines such as PNU can serve as the position to covalently bond the linking moiety and, hence the anti-CD117 antibodies or antigen-binding fragments thereof as described herein. For example, linkers may be introduced through modifications to the hydroxymethyl ketone side chain.

In some embodiments, the cytotoxin is a PNU derivative represented by the structural formula:

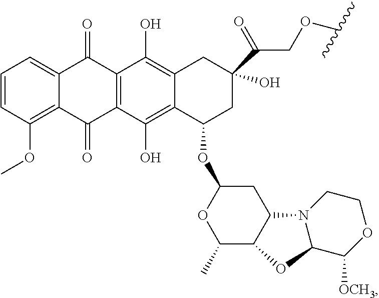

wherein the wavy line indicates the point of covalent attachment to the linker of the ADC as described herein.

In some embodiments, the cytotoxin is a PNU derivative represented by the structural formula:

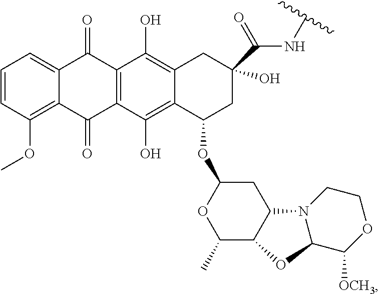

wherein the wavy line indicates the point of covalent attachment to the linker of the ADC as described herein.

Pyrrolobenzodiazepines (PBDs)

In other embodiments, the anti-HC antibodies (e.g., anti-CD117 antibody or anti-CD45 antibody,) or antigen-binding fragments thereof described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, J A (2011) The development of pyrrolobenzodiazepines as antitumour agents. Expert Opin Inv Drug, 20(6), 733-744 and Antonow D, Thurston D E (2011) Synthesis of DNA-interactive pyrrolo[2,1-c][1,4]benzodiazepines (PBDs). Chem Rev 111: 2815-2864.

In some embodiments, the cytotoxin is a pyrrolobenzodiazepine dimer represented by the structural formula:

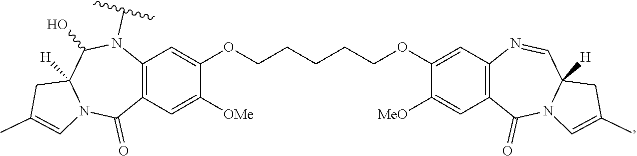

wherein the wavy line indicates the attachment point of the linker.

In some embodiments, the cytotoxin is conjugated to the antibody, or the antigen-binding fragment thereof, by way of a maleimidocaproyl linker.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula:

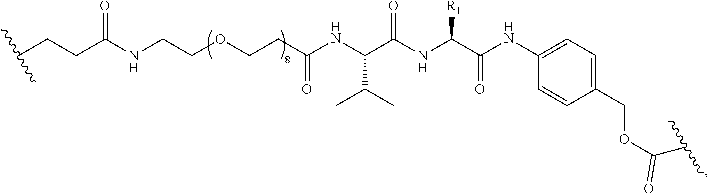

wherein R₁ is CH₃ (Ala) or (CH₂)₃NH(CO)NH₂ (Cit).

In some embodiments, the linker, prior to conjugation to the antibody and including the reactive substituent Z′, taken together as L-Z, has the structure:

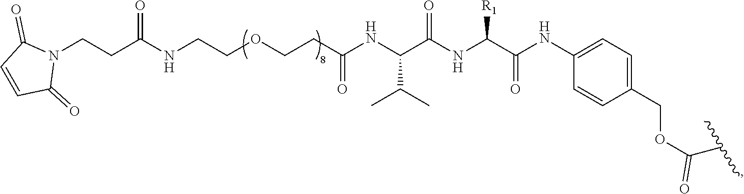

wherein the wavy line indicates the attachment point to the cytotoxin (e.g., a PBD). In certain embodiments, R₁ is CH₃.

In some embodiments, the cytotoxin-linker conjugate, prior to conjugation to the antibody and including the reactive substituent Z′, taken together as Cy-L-Z′, has the structural formula:

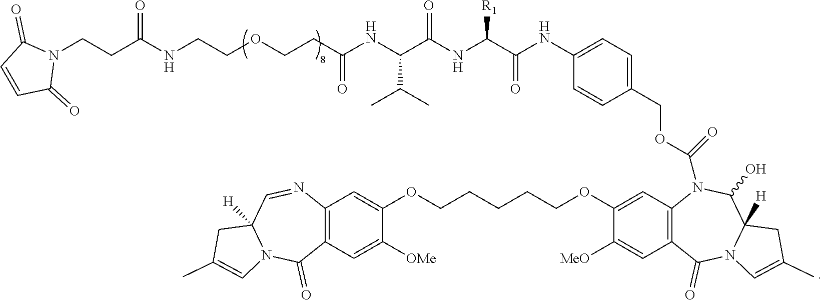

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In some embodiments, the cytotoxin is a pyrrolobenzodiazepine dimer represented by the structural formula:

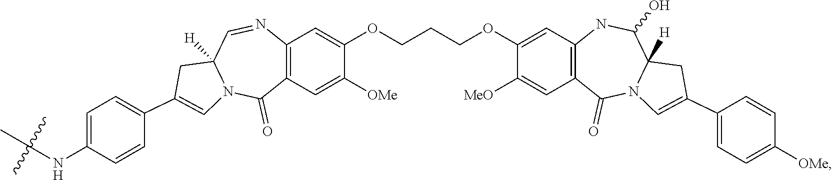 [SMILES: *Nc1ccc(C2=CN3C(=O)c4cc(OC)c(OCCCOc5cc6c(cc5OC)C(=O)N5C=C(c7ccc(OC)cc7)C[C@H]5C(O)N6)cc4N=C[C@@H]3C2)cc1]

wherein the wavy line indicates the attachment point of the linker.

In some embodiments, the cytotoxin-linker conjugate, prior to conjugation to the antibody and including the reactive substituent Z′, taken together as Cy-L-Z′, has the structural formula:

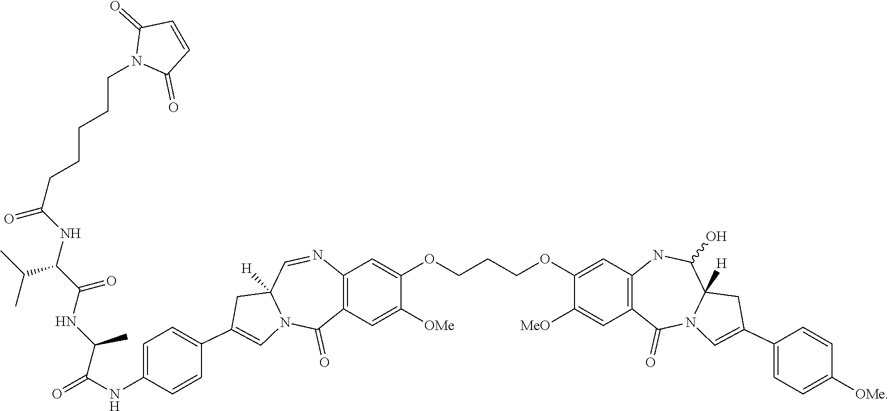 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4NC(O)[C@@H]3C2)cc1]

This particular cytotoxin-linker conjugate is known as talirine, and has been described, for example, in connection with the ADC Vadastuximab talirnne (SGN-CD33A), Mantaj et al., Angewandte Chemie International Edition English 2017,56, 462-488, the disclosure of which is incorporated by reference herein in its entirety.

In some embodiments, the cytotoxin is an indolinobenzodiazepine pseudodimer having the structural formula:

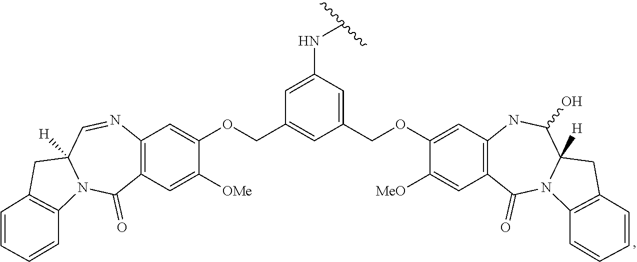 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2C(O)N3)c1]

wherein the wavy line indicates the attachment point of the linker.

In some embodiments, the cytotoxin-linker conjugate, prior to conjugation to the antibody and including the reactive substituent Z, taken together as Cy-L-Z′, has the structural formula:

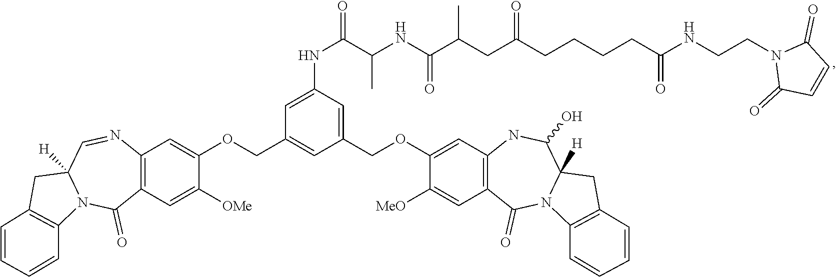 [SMILES: COc1cc2c(cc1OCc1cc(COc3cc4c(cc3OC)C(=O)N3c5ccccc5C[C@H]3C(O)N4)cc(NC(=O)C(C)NC(=O)C(C)CC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

Calicheamicin

In other embodiments, the antibodies and antigen-binding fragments thereof described herein can be conjugated to a cytotoxin that is an enediyne antitumor antibiotic (e.g., calicheamicins, ozogamicin). The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993). Lode et al., Cancer Research 58:2925-2928 (1998), and the aforementioned U.S. patents to American Cyanamid.

An exemplary calicheamicin is designated γ₁, which is herein referenced simply as gamma, and has the structural formula:

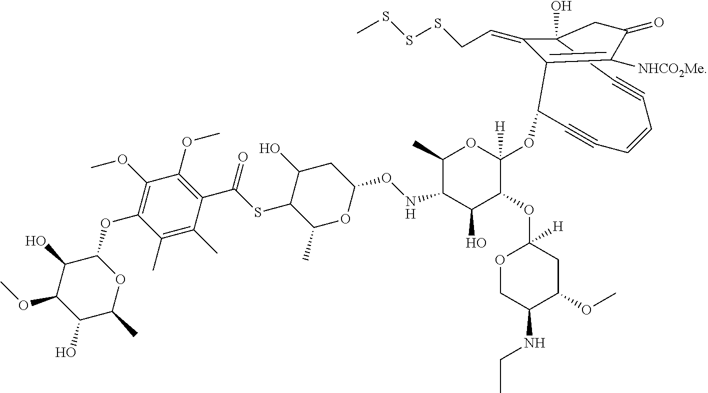

In some embodiments, the calicheamicin is a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD117 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein is a calicheamicin disulfide derivative represented by the structural formula:

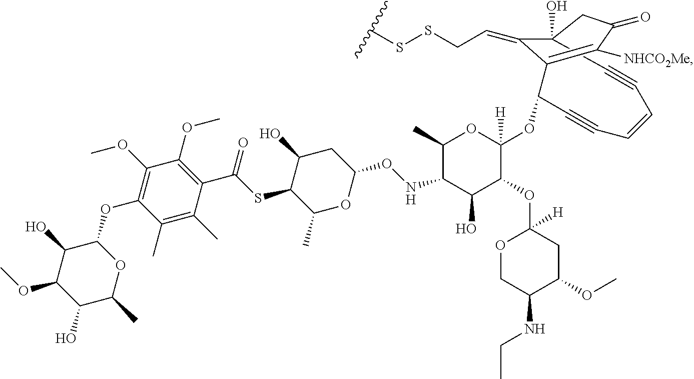

wherein the wavy line indicates the attachment point of the linker.

Additional Cytotoxins

In other embodiments, the antibodies and antigen-binding fragments thereof described herein can be conjugated to a cytotoxin other than or in addition to those cytotoxins disclosed herein above. Additional cytotoxins suitable for use with the compositions and methods described herein include, without limitation, 5-ethynyluracil, abiraterone, acylfulvene, adecypenol, adozelesin, aldesleukin, aftretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflomithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, merbarone, meterelin, methioninase, metoclopramide. MIF inhibitor, ifepristone, miltefosine, mirimostim, mithracin, mitoguazone, mitolactol, mitomycin and analogues thereof, mitonafide, mitoxantrone, mofarotene, moigramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaftine, vorozole, zeniplatin, and zilascorb, among others.

Linkers

A variety of linkers can be used to conjugate the antibodies, or antibody fragments thereof, described herein (e.g., an anti-CD117 antibody, or an anti-CD45 antibody) to a cytotoxic molecule.

The term “Linker” as used herein means a divalent chemical moiety comprising a covalent bond or a chain of atoms that covalently attaches an anti-HC antibody (e.g., an anti-CD117 antibody or an anti-CD45 antibody)-drug conjugates (ADC) of the present disclosure (ADCs; Ab-Z-L-D, where D is a cytotoxin). Suitable linkers have two reactive termini, one for conjugation to an antibody and the other for conjugation to a cytotoxin. The antibody conjugation reactive terminus of the linker (reactive moiety, Z′) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as reactive moiety Z′, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine. Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, or antibody fragments thereof described herein, to a cytotoxic molecule include those capable of releasing a cytotoxin by a 1,6-elimination process (a “self-immolative” group). Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0258030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C₁-C₆ alkylene, C₁-C₆ heteroalkylene, C₁-C₆ alkenylene, C₁-C₆ heteroalkenylene, C₁-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ-units, wherein p is an integer from 1-6, independently selected for each occasion.

Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

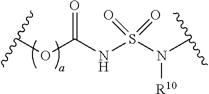

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₅ alkynyl, an optionally substituted C₂-C₅ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like.

In some embodiments, the linker L comprises the moiety *-L₁L₂-**, wherein:

L₁ is absent or is —(CH₂)ₘNR¹³C(═O)—, —(CH₂)ₘNR¹³—, —(CH₂)ₘX₃(CH₂)ₘ—,

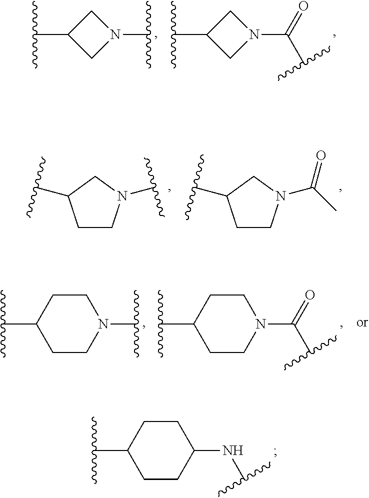

L₂ is absent or is —(CH₂)ₘ—, —NR¹³(CH₂)ₘ—, —(CH₂)ₘNR¹³C(═O)(CH₂)ₘ—, —X₄, —(CH₂)ₘNR¹³C(═O)X₄, —(CH₂)ₘNR¹³C(═O)—, —((CH₂)ₘO)ₙ(CH₂)ₘ—, —((CH₂)ₘO)ₙ(CH₂)ₘX₃(CH₂)ₘ—, —NR¹³((CH₂)ₘO)ₙX₃(CH₂)ₘ—, —NR¹³((CH₂)ₘO)ₙ(CH₂)ₘX₃(CH₂)ₘ—, —X₁X₂C(═O)(CH₂)ₘ—, —(CH₂)ₘ(O(CH₂)ₘ)ₙ—, —(CH₂)ₘNR¹³(CH₂)ₘ—, —(CH₂)ₘNR¹³C(═O)(CH₂)ₘX₃(CH₂)ₘ—, —(CH₂)ₘC(═O)NR¹³(CH₂)ₘNR¹³C(═O)(CH₂)ₘ—, —(CH₂)ₘC(═O)—, —(CH₂)ₘNR¹³(CH₂)ₘC(═O)X₂X₁C(═O)—, —(CH₂)ₘX₃(CH₂)ₘC(═O)X₂X₁C(═O)—, —(CH₂)ₘC(═O)NR¹³(CH₂)ₘ—, —(CH₂)ₘC(═O)NR³(CH₂)ₘX₃(CH₂)ₘ—, —(CH₂)ₘX₃(CH₂)ₘNR¹³C(═O)(CH₂)ₘ—, —(CH₂)ₘX₃(CH₂)ₘC(═O)NR¹³(CH₂)ₘ—, —(CH₂)ₘO)ₙ(CH₂)ₘNR¹³C(═O)(CH₂)ₘ—, —(CH₂)ₘC(═O)NR¹³(CH₂)ₘ(O(CH₂)ₘ)ₙ—, —(CH₂)ₘ(O(CH₂)ₘ)ₙC(═O)—, —(CH₂)ₘNR¹³(CH₂)ₘC(═O)—, —(CH₂)ₘC(═O)NR¹³(CH₂)ₘNR³C(═O)—, —(CH₂)ₘ(O(CH₂)ₘ)ₙX3(CH₂)ₘ—, —(CH₂)ₘX₃((CH₂)ₘO)ₙ(CH₂)ₘ—, —(CH₂)ₘX₃(CH₂)ₘC(═O)—, —(CH₂)ₘC(═O)NR¹³(CH₂)ₘO)ₙ(CH₂)ₘX₃(CH₂)ₘ—, —(CH₂)ₘX₃(CH₂)ₘ(O(CH₂)ₘ)ₙNR¹³C(═O)(CH₂)ₘ—, —(CH₂)ₘX₃(CH₂)ₘ(O(CH₂)ₘ)ₙC(═O)—, —(CH₂)ₘX₃(CH₂)ₘ(O(CH₂)ₘ)ₙ—, —(CH₂)ₘC(═O)NR¹³(CH₂)ₘC(═O)—, —(CH₂)ₘC(═O)NR¹3(CH₂)ₘ(O(CH₂)ₘ)ₙC(═O)—, —((CH₂)ₘO)ₙ(CH₂)ₘNR¹³C(═O)(CH₂)ₘ—, —(CH₂)ₘC(═O)NR¹3(CH₂)ₘC(═O)NR¹³(CH₂)ₘ—, —(CH₂)ₘNR¹³C(═O)(CH₂)ₘNR¹³C(═O)(CH₂)—(CH₂)ₘX₃(CH₂)ₘC(═O)NR¹³—, —(CH₂)ₘC(═O)NR¹3-, —(CH₂)ₘX₃—, —C(R¹³)₂(CH₂)ₘ—, —(CH₂)ₘC(R¹³)₂NR′3-, —(CH₂)ₘC(═O)NR¹³(CH₂)ₘNR¹³—, —(CH₂)ₘC(═O)NR^{t3}(CH₂)ₘNR^{t3}C(═O)NR¹³—, —(CH₂)ₘC(═O)X₂X₁C(═O)—, —C(R¹³)₂(CH₂)ₘNR¹³C(═O)(CH₂)ₘ—, —(CH₂)ₘC(═O)NR³(CH₂)ₘC(R¹³)₂NR¹³—, —C(R¹³)₂(CH₂)ₘX₃(CH₂)ₘ—, —(CH₂)ₘX₃(CH₂)ₘC(R¹³)₂NR′³—, —C(R¹³)₂(CH₂)ₘOC(═O)NR¹³(CH₂)ₘ—, —(CH₂)ₘNR¹³C(═O)O(CH₂)ₘC(R¹³)₂NR¹³—, —(CH₂)ₘX₃(CH₂)ₘNR¹³, —(CH₂)ₘX₃(CH₂)ₘ(O(CH₂)ₘ)ₙNR¹³—, —(CH₂)ₘNR¹³—, —(CH₂)ₘC(═O)NR¹³(CH₂)ₘ(O(CH₂)ₘ)ₙNR¹³—, —(CH₂)ₘ(O(CH₂)ₘ)ₙNR¹³—, —(CH₂CH₂O)ₙ(CH₂)ₘ—, —(CH₂)ₘ(OCH₂CH₂)ₙ, —(CH₂)ₘO(CH₂)ₘ—, —(CH₂)ₘS(═O)₂—, —(CH₂)ₘC(═O)NR¹³(CH₂)ₘS(═O)₂—, —(CH₂)ₘX₃(CH₂)ₘS(═O)₂—, —(CH₂)ₘX₂X₁C(═O)—, —(CH₂)ₘ(O(CH₂)ₘ)ₙC(═O)X₂X₁C(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X₁C(═O)—, —(CH₂)ₘX₃(CH₂)ₘC(═O)NR¹³(CH₂)ₘNR¹³C(═O)—, —(CH₂)ₘX₃(CH₂)ₘC(═O)NR¹³(CH₂)ₘC(═O)—, —(CH₂)ₘX₃(CH₂)ₘC(═O)NR¹³(CH₂)ₘ(O(CH₂)ₘ)ₙC(═O)—, —(CH₂)ₘC(═O)X₂X₁C(═O)NR¹³(CH₂)ₘ—, —(CH₂)ₘX₃(O(CH₂)ₘ)ₙC(═O)—, —(CH₂)ₘNR¹³C(═O)((CH₂)ₘO)ₙ(CH₂)ₘ—, —(CH₂)ₘ(O(CH₂))ₙC(═O)NR¹³(CH₂)ₘ—, —(CH₂)ₘNR¹³C(═O)NR¹³(CH₂)ₘ— or —(CH₂)ₘX₃(CH₂)ₘNR¹³C(═O)—; wherein

X₁ is

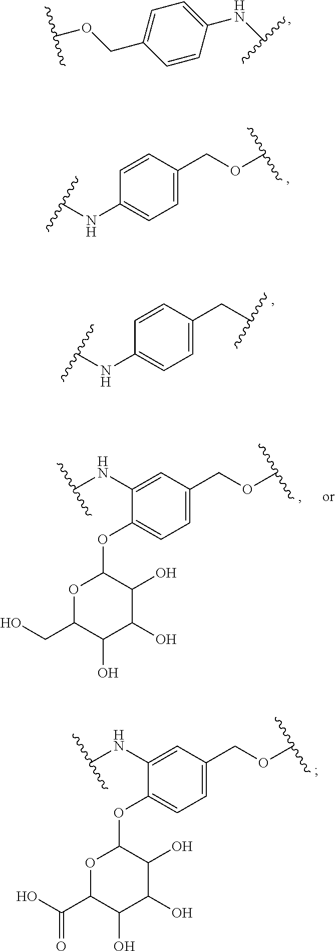 [SMILES: *Nc1ccc(CO*)cc1 ; *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is

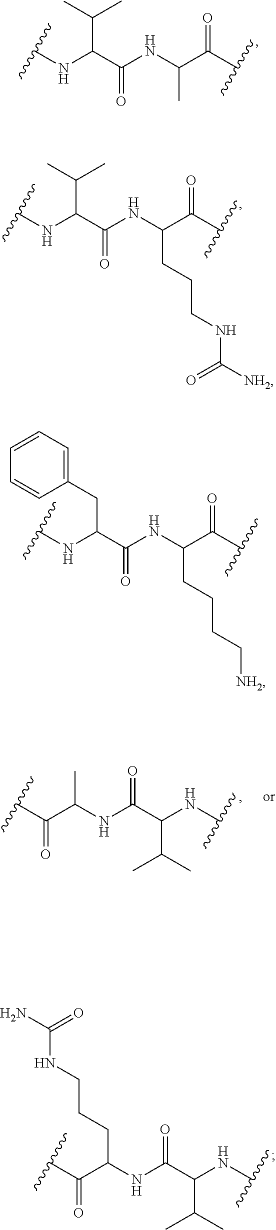 [SMILES: *NC(C(=O)NC(C)C(*)=O)C(C)C ; *NC(C(=O)NC(CCCNC(N)=O)C(*)=O)C(C)C ; *NC(Cc1ccccc1)C(=O)NC(CCCCN)C(*)=O ; *NC(C(=O)NC(C)C(*)=O)C(C)C ; *NC(C(=O)NC(CCCNC(N)=O)C(*)=O)C(C)C]

X₃ is

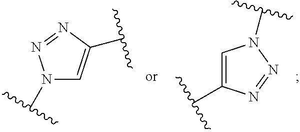 [SMILES: *c1cn(*)nn1 ; *c1cn(*)nn1]

and

X₄ is

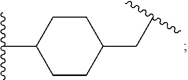

wherein

R¹³ is independently selected for each occasion from H and C₁-C₆ alkyl;

m is independently selected for each occasion from 1, 2. 3, 4, 5, 6. 7, 8, 9 and 10;

n is independently selected for each occasion from 1, 2, 3, 4, 5, 6, 7, 8, 9, 10, 11, 12, 13 and 14; and

wherein the single asterisk (*) indicates the attachment point to the cytotoxin (e.g., an amatoxin), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB. Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a —(C═O)(CH₂)ₚ— unit, wherein p is an integer from 1-6.

In some embodiments, the linker comprises a —(CH₂)ₙ— unit, wherein n is an integer from 2 to 6.

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises

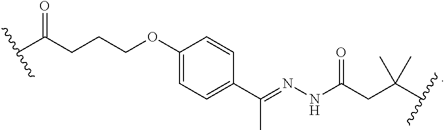 [SMILES: *C(=O)CCCOc1ccc(/C(C)=N/NC(=O)CC(*)(C)C)cc1]

In some embodiments, the linker comprises MCC (4-[N-maleimidomethyl]cyclohexane-1-carboxylate).

In one specific embodiment, the linker comprises the structure

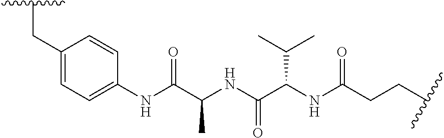 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z′. In another specific embodiment, the linker comprises the structure

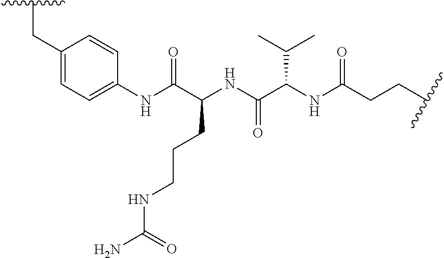 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein. Linkers that can be used to conjugate an antibody, or antigen-binding fragment thereof, to a cytotoxic agent include those that are covalently bound to the cytotoxic agent on one end of the linker and, on the other end of the linker, contain a chemical moiety formed from a coupling reaction between a reactive substituent present on the linker and a reactive substituent present within the antibody, or antigen-binding fragment thereof, that binds e.g. CD117, Reactive substituents that may be present within an antibody, or antigen-binding fragment thereof, that binds e.g. CD117 include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids.

Examples of linkers useful for the synthesis of drug-antibody conjugates include those that contain electrophiles, such as Michael acceptors (e.g., maleimides), activated esters, electron-deficient carbonyl compounds, and aldehydes, among others, suitable for reaction with nucleophilic substituents present within antibodies or antigen-binding fragments, such as amine and thiol moieties. For instance, linkers suitable for the synthesis of drug-antibody conjugates include, without limitation, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are particularly useful for the conjugation of microtubule-disrupting agents such as auristatins, are described by Doronina et al., Bioconjugate Chem. 17:14-24, 2006, the disclosure of which is incorporated herein by reference as it pertains to linkers for chemical conjugation.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 2, below. Curved lines designate points of attachment to the antibody or antigen-binding fragment, and the cytotoxic molecule, respectively.

TABLE 2
Exemplary chemical moieties Z formed by coupling reactions in
the formation of antibody-drug conjugates

| Exemplary | |
|---|---|
| Coupling Reactions | Chemical Moiety Z Formed by Coupling Reactions |
| [3 + 2] Cycloaddition | 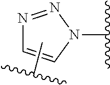 |
| [3 + 2] Cycloaddition | 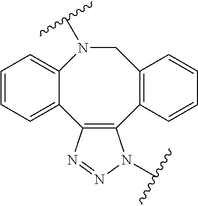 [SMILES: *N1Cc2ccccc2-c2c(nnn2*)-c2ccccc21] |
| [3 + 2] Cycloaddition, Esterification | 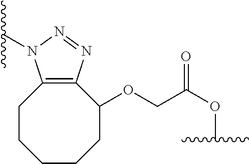 [SMILES: *OC(=O)COC1CCCCCc2c1nnn2*] |
| [3 + 2] Cycloaddition, Esterification | 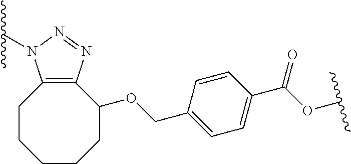 [SMILES: *OC(=O)c1ccc(COC2CCCCCc3c2nnn3*)cc1] |
| [3 + 2] Cycloaddition, Esterification | 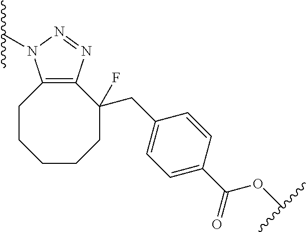 [SMILES: *OC(=O)c1ccc(CC2(F)CCCCCc3c2nnn3*)cc1] |
| [3 + 2] Cycloaddition, Esterification | 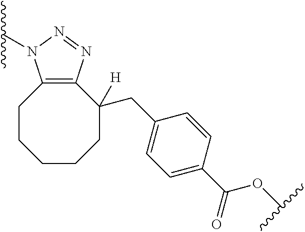 [SMILES: *OC(=O)c1ccc(CC2CCCCCc3c2nnn3*)cc1] |
| [3 + 2] Cycloaddition, Esterification | 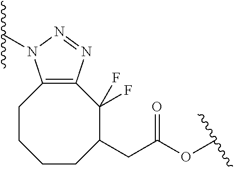 [SMILES: *OC(=O)CC1CCCCc2c(nnn2*)C1(F)F] |
| [3 + 2] Cycloaddition, Esterification | 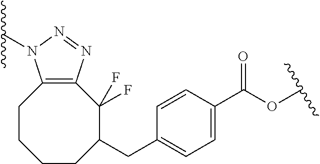 [SMILES: *OC(=O)c1ccc(CC2CCCCc3c(nnn3*)C2(F)F)cc1] |
| [3 + 2] Cycloaddition, Esterification | 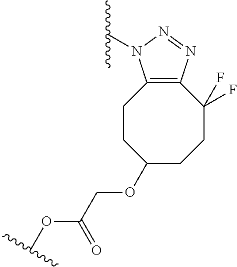 [SMILES: *OC(=O)COC1CCc2c(nnn2*)C(F)(F)CC1] |
| [3 + 2] Cycloaddition, Esterification | 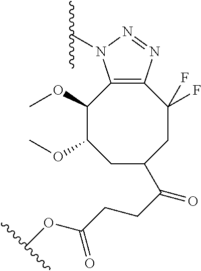 [SMILES: *OC(=O)CCC(=O)C1C[C@H](OC)[C@@H](OC)c2c(nnn2*)C(F)(F)C1] |
| [3 + 2] Cycloaddition, Esterification | 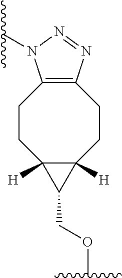 [SMILES: *OC[C@@H]1[C@@H]2CCc3c(nnn3*)CC[C@@H]21] |
| [3 + 2] Cycloaddition, Esterification | 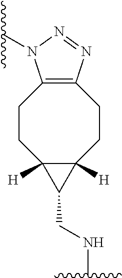 [SMILES: *NC[C@@H]1[C@@H]2CCc3c(nnn3*)CC[C@@H]21] |
| [3 + 2] Cycloaddition, Esterification | 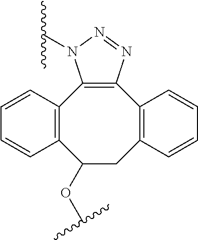 [SMILES: *OC1Cc2ccccc2-c2nnn(*)c2-c2ccccc21] |
| [3 + 2] Cycloaddition, Esterification | 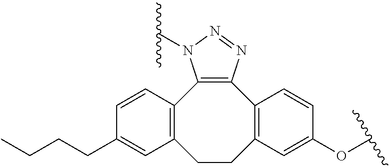 [SMILES: *Oc1ccc2c(c1)CCc1cc(CCCC)ccc1-c1c-2nnn1*] |
| [3 + 2] Cycloaddition | 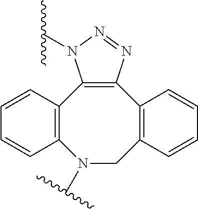 [SMILES: *N1Cc2ccccc2-c2nnn(*)c2-c2ccccc21] |
| Michael addition | 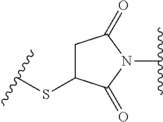 [SMILES: *SC1CC(=O)N(*)C1=O] |
| Michael addition | 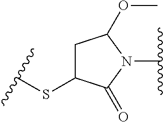 [SMILES: *SC1CC(OC)N(*)C1=O] |
| Imine condensation, Amidation | 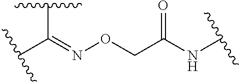 [SMILES: *NC(=O)CON=C(*)*] |
| Imine condensation | 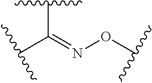 [SMILES: *ON=C(*)*] |
| Disulfide formation | 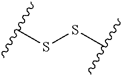 [SMILES: *SS*] |
| Thiol alkylation | 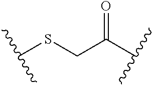 [SMILES: *SCC(*)=O] |
| Condensation, Michael addition | 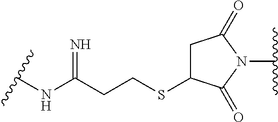 [SMILES: *NC(=N)CCSC1CC(=O)N(*)C1=O] |

One of skill in the art will recognize that a reactive substituent Z′ attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z, suitable for reaction with a reactive substituent on the antibody, or antigen-binding fragment thereof, to form the chemical moiety Z.

As depicted in Table 2, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substituents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition. [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

In some embodiments, Z′ is —NR¹³C(═O)CH═CH₂, —N₃, —SH, —S(═O)₂(CH═CH₂), —(CH₂)₂S(═O)₂(CH═CH₂), —NR¹³S(═O)₂(CH═CH₂), —NR¹³C(═O)CH₂R¹⁴, —NR¹³C(═O)CH₂Br, —NR¹³C(═O)CH₂₁, —NHC(═O)CH₂Br, —NHC(═O)CH₂1, —ONH₂, —C(O)NHNH₂, —CO₂H, —NH₂, —NH(C═O), —NC(═S),

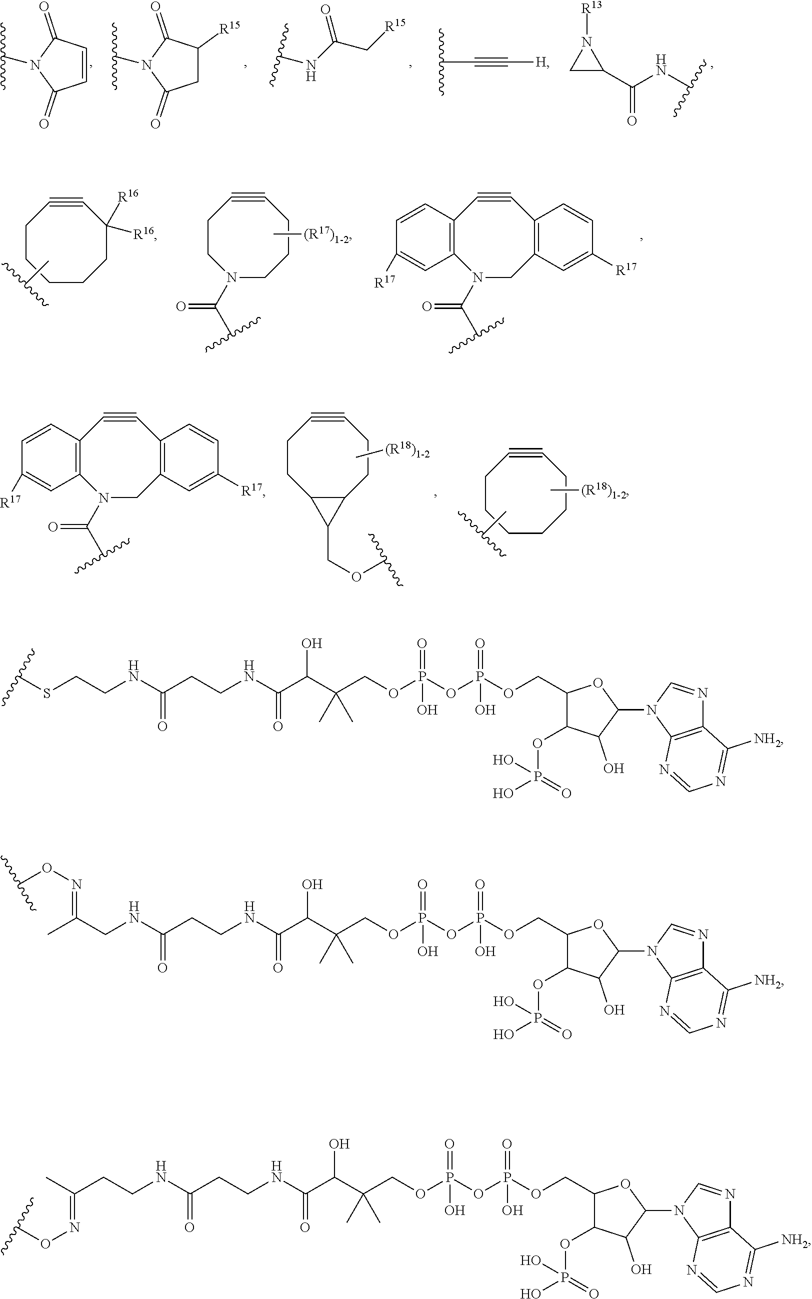

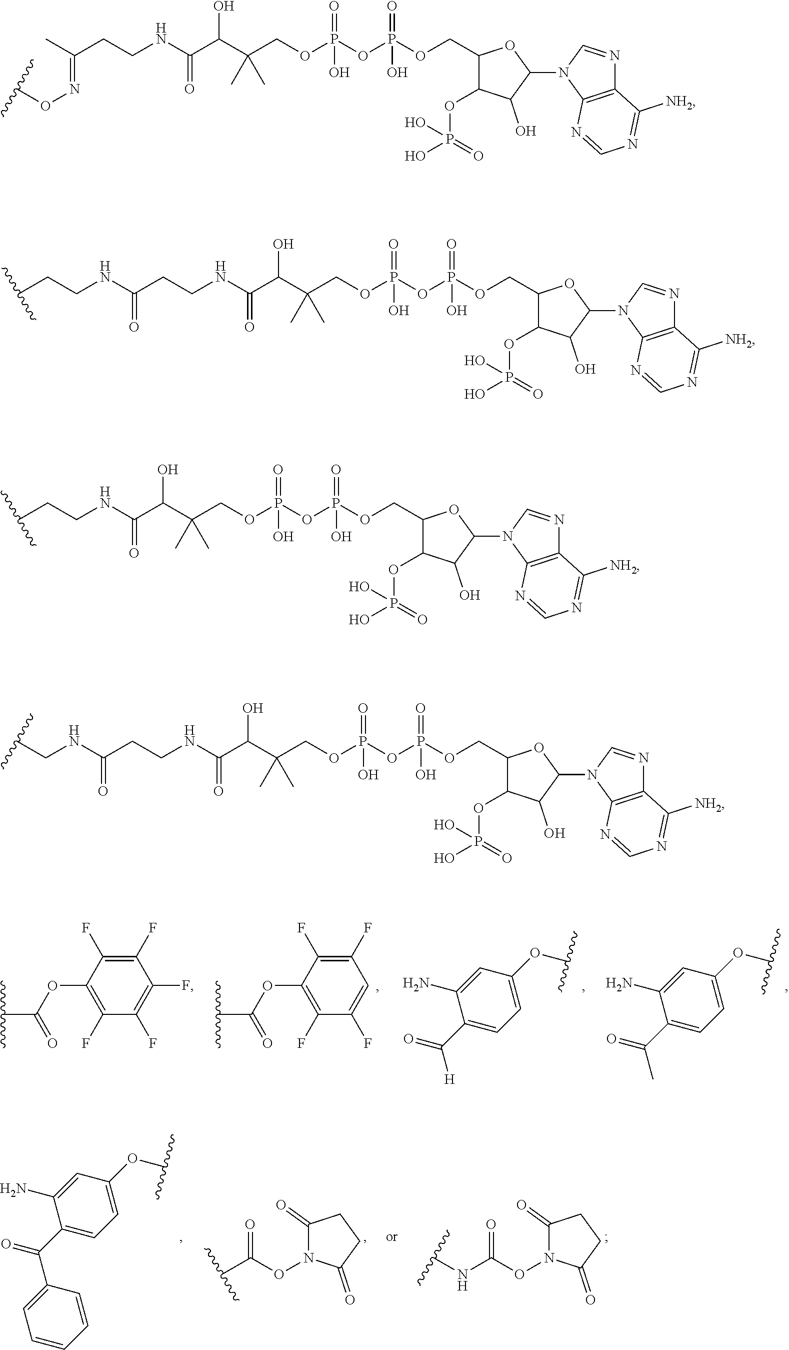

wherein

R¹³ is independently selected for each occasion from H and C₁-C₆ alkyl;

R¹⁴ is —S(CH₂)ₙCHR¹⁵NHC(═O)R¹³;

R¹⁵ is R¹³ or —C(═O)OR¹³;

R¹⁶ is independently selected for each occasion from H, C₁-C₆ alkyl, F, Cl, and —OH;

R¹⁷ is independently selected for each occasion from H, C₁-C₆ alkyl, F, Cl, —NH₂, —OCH₃, —OCH₂CH₃, —N(CH₃)₂, —CN, —NO₂ and —OH; and

R¹⁸ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH.

Reactive substituents that may be present within an antibody, or antigen-binding fragment thereof, as disclosed herein include, without limitation, nucleophilic groups such as (i)N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z. For instance, Z′ may be a Michael acceptor (e.g., maleimide), activated ester, electron-deficient carbonyl compound, and aldehyde, among others.

For instance, linkers suitable for the synthesis of ADCs include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substiuent comprises a cycloalkene group, a cycloalkyne group, or an optionally substituted (hetero)cycloalkynyl group.

Non-limiting examples of amatoxin-linker conjugates containing a reactive substituent Z′ suitable for reaction with a reactive residue on the antibody or antigen-binding fragment thereof include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl))cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methylcyclohexanecarboxamido)ethyl)piperidin-1-yl)-amatoxin; 7′C-(4-(2-(6-(4-((maleimido)methylcyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethypiperidin-1-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-ylmethyl-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-ylmethyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; TC-((3-(2-(6-(maleimido)hexanamido)ethyl)azetidin-1-yl)methyl)-amatoxin; 7′C-((3-((4-((maleimido)methylcyclohexanecarboxamido)methyl)azetidin-1-yl)methyl)-amatoxin; 7′C-((3-(2-(4-((maleimido)methylcyclohexanecarboxamido)ethyl)azetidin-1yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(8-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-1-yl)methyl)-amatoxin; 7′C-((2-(2-(6-(4-((maleimido)methyl)cyclohexanecarboxamido)hexanamido)ethyaziridin-1-yl)methyl)-amatoxin; 7′C-((4-(6-(6-(2-(aminooxy)acetamido)hexanamido)hexanoylpiperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acety)piperazin-1-yl)methyl)-amatoxin; 7′C-((4-(3-(2-(aminooxy)acetamido)propanoy)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosypiperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin.

One of skill in the art will recognize the linker-reactive substituent group structure, prior to conjugation with the antibody or antigen binding fragment thereof, includes a maleimide as the group Z. The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In some embodiments, the linker-reactive substituent group structure L-Z, prior to conjugation with the antibody or antigen binding fragment thereof, is:

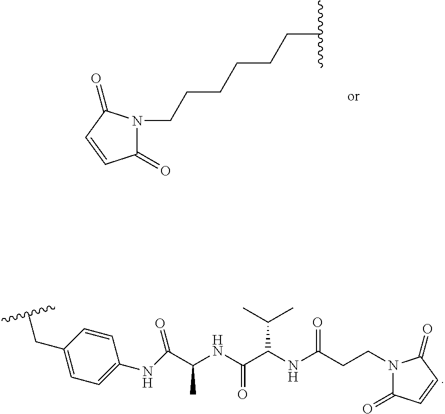

In some embodiments, an amatoxin as disclosed herein is conjugated to a linker-reactive moiety -L-Z having the following formula:

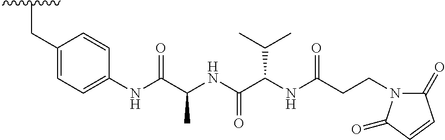 [SMILES: *Cc1ccc(NC(=O)[C@H](C)NC(=O)[C@@H](NC(=O)CCN2C(=O)C=CC2=O)C(C)C)cc1]

In some embodiments, an amatoxin as disclosed herein is conjugated to a linker-reactive moiety -L-Z having the following formula:

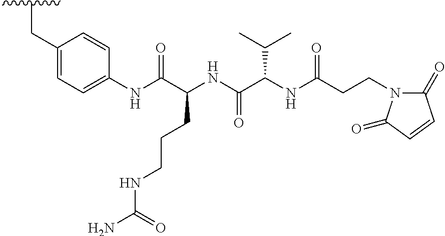 [SMILES: *Cc1ccc(NC(=O)[C@H](CCCNC(N)=O)NC(=O)[C@@H](NC(=O)CCN2C(=O)C=CC2=O)C(C)C)cc1]

In some embodiments, the ADC comprises an anti-CD117 antibody conjugated to an amatoxin of any of formulae III, IIIA, or IIIB as disclosed herein via a linker and a chemical moiety Z. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-8. In some embodiments, the linker is -PAB-Cit-Val-((C═O)(CH₂)ₙ—.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker is —(CH₂)ₙ—. In some embodiments, the linker is —((CH₂)ₙ—, wherein n is 6.

In some embodiments, the chemical moiety Z is selected from Table 2. In some embodiments, the chemical moiety Z is

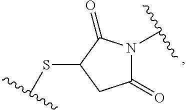

where S is a sulfur atom which represents the reactive substituent present within an antibody, or antigen-binding fragment thereof, that binds CD117 (e.g., from the —SH group of a cysteine residue).

In some embodiments, the linker L and the chemical moiety Z, taken together as L-Z, is

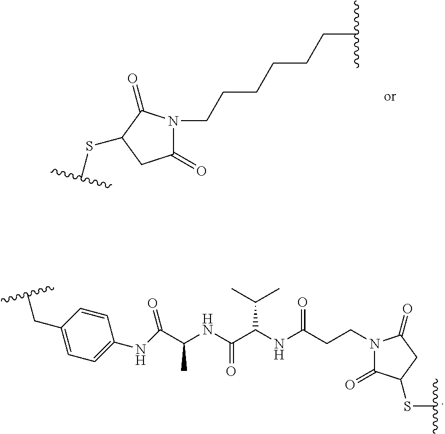

### Preparation of Antibody-Drug Conjugates

In the ADCs of formula I as disclosed herein, an anti-HC antibody (e.g., an anti-CD117 antibody or an anti-CD45 antibody) or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above to form an ADC of formula D-L-Z-Ab, such as Am-Z-L-Ab. Additional methods for preparing ADC are described herein.

In another aspect, the anti-HC antibody (e.g., anti-CD117 antibody or anti-CD45 antibody) or antigen binding fragment thereof has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-HC antibody (e.g., anti-CD117 antibody or anti-CD45 antibody) or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the anti-HC antibody (e.g., anti-CD117 antibody or anti-CD45 antibody) can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

ADCs described herein can be administered to a patient (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising anti-HC ADCs (e.g., anti-CD117 ADC or anti-CD45 ADC) as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Anti-CD46 and Anti-CD117-Antibody Drug Conjugates Enable Allogeneic Hematopoietic Stem Cell Transplantation

Antibody drug conjugates (ADCs) targeting mouse CD45 or mouse CD117 have recently been shown to effectively condition immunocompetent mice for whole bone marrow transplants (Palchaudhuri et al. Nature Biotech 2016 34:738-745: and Czechowicz et al. Blood 2016 128:493). This innovative targeted approach to conditioning using ADCs has the potential to be a therapeutic breakthrough if it can be successfully translated to humans. The anti-CD45 or anti-CD117 antibodies used previously were coupled to saporin (SAP), a ribosome-inhibiting protein, which once internalized elicits cytotoxicity in a cell cycle-independent manner. Both anti-CD45-saporin (CD45-SAP) and anti-CD117-saporin (CD117-SAP) ADCs have been shown to effectively deplete bone marrow hematopoietic stem cells (HSCs) as single entity agents, creating vacancies that enable efficient autologous HSC engraftment (>95% long-term donor chimerism).

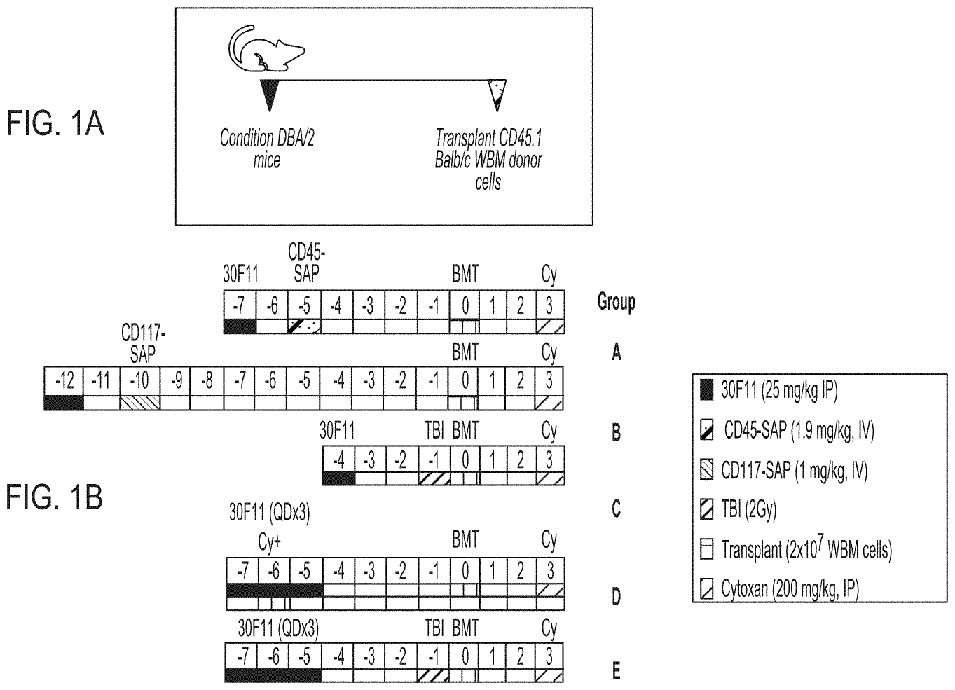

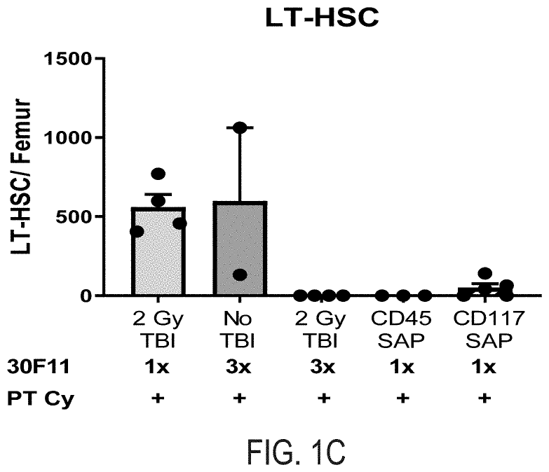

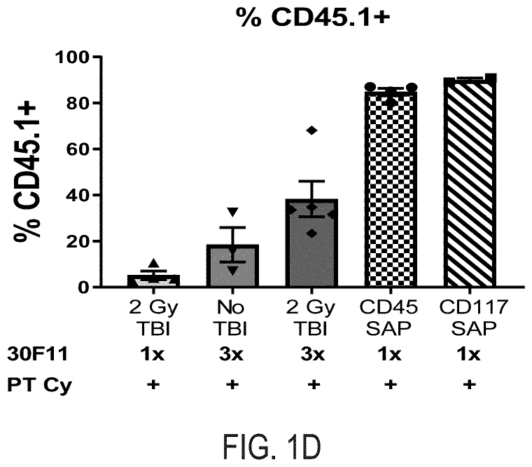

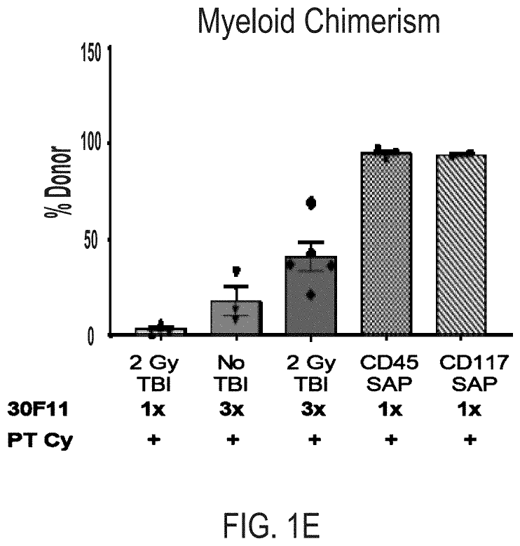

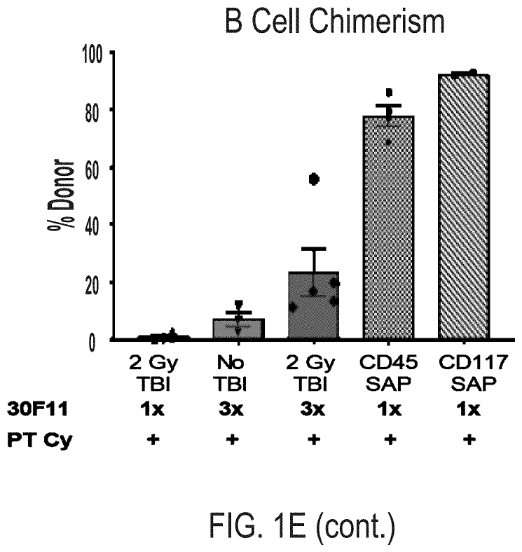

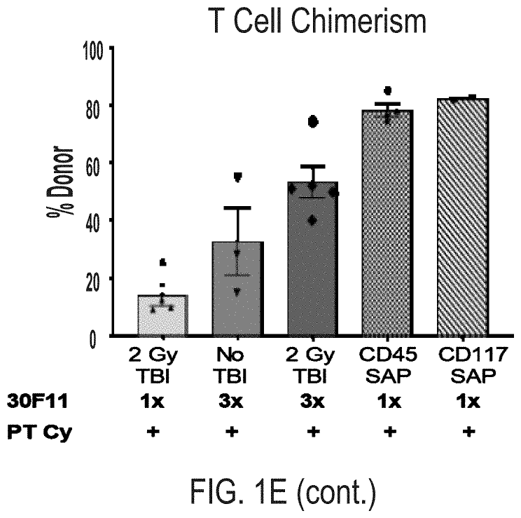

The anti-CD117 antibody used in the ADC in the following example is 2B8, and the anti-CD45 antibody used in the ADC is 104. To further investigate and expand the utility of these tool ADCs in murine transplant models, CD45-SAP (1.9 mg/kg, iv) and CD117-SAP (1 mg/kg, iv) were tested in an allogeneic minor mismatch transplant model (Balb/c donor into DBA/2 recipients). DBA/2 CD45.2 mice were transplanted with 2×10⁷ whole bone marrow cells harvested from pooled Balb/c CD45.1 congenic donors. As shown in the study design schematic in FIGS. 1A and 1B, DBA/2 mice received pre-transplant conditioning prior to transplant with CD45.1 Balb/c whole bone marrow donor cells. Conditioning treatments including CD45-SAP (1.9 mg/kg, i.v.) or CD117-SAP (1 mg/kg, i.v.) were evaluated in conjunction with additional immune modulating agents: clone 30F11 (25 mg/kg, IP), a naked anti-CD45 antibody that mimics ATG by relying on effector function to enable potent peripheral B- and T-cell depletion; pre-transplant Cytoxan (PreT-Cy, 200 mg/kg, IP); 2 Gy total body irradiation (TBI); or post-transplant Cytoxan (PTCy, 200 mg/kg, IP) to prevent graft versus host disease as well as block host versus graft rejection. 9 Gy TBI was used as the conventional conditioning positive control. Conditioned mice were transplanted with 2×10⁷ whole bone marrow cells, and the level of HSC depletion and donor cell chimerism were assessed over 12 weeks.

The results of the engraftment assay are shown in FIGS. 1C-1E, which show the long term-hematopoietic stem cell count (LT-HSC)/femur (FIG. 1C), the percent donor chimerism (FIG. 1D), and the percent myeloid chimerism, percent B cell chimerism, and percent T cell chimerism (FIG. 1E) following the conditioning with the indicated ADC and immunosuppressant.

CD45-SAP or CD117-SAP in combination with immunosuppressants (30F11 and post-transplant Cytoxan) enabled bone marrow depletion in C57Bl/6 mice (FIG. 1C; 7 days post-administration) and enabled complete donor chimerism (>85% donor chimerism (CD45.1+)) in the peripheral blood at 12 weeks post-transplantation (FIG. 1D). Multilineage reconstitution was observed in the T-, B- and myeloid cell compartments with >80%, >90% and >90% donor chimerism respectively in both CD45-SAP and CD117-SAP groups (FIG. 1E). In contrast, 2Gy TBI in combination with immunosuppressants (30F11 and post-transplant Cytoxan) resulted in only 5% donor engraftment. Multi-dosing with 30F11 (QDx3) plus 2Gy TBI and post-transplant Cytoxan increased the peripheral donor chimerism to 40%. Pre-transplant Cytoxan plus 30F11 (QDx3) and post-transplant Cytoxan yielded 20% donor chimerism. For all groups, stem cell chimerism in the bone marrow matched the peripheral chimerism.

These results indicate anti-CD45 and anti-CD117 ADCs may be used in combination with immunosuppression to enable highly efficient allogeneic transplants in a minor mismatch model (85% donor chimerism). CD45-SAP and CD117-SAP in combination with 30F11 and post-transplant Cytoxan were more effective at conditioning versus 2Gy TBI or pre-transplant Cytoxan.

### Example 2. CD45-Targeted Antibody Drug Conjugate Plus Post Transplant Cytoxan is Sufficient to Enable Allogeneic Bone Marrow Transplant in a Minor Mismatch Mouse Model

Bone Marrow Transplant (BMT) is a potentially curative treatment for malignant and non-malignant blood disorders. Current regimens for patient preparation, or conditioning, prior to BMT limit the use of this curative procedure due to regimen-related mortality and morbidities, including risks of organ toxicity, infertility and secondary malignancies. Targeted preparation using antibody drug conjugates (ADCs) to mouse CD45 has previously been shown to be sufficient to enable bone marrow transplant (BMT) in syngeneic immune competent mice (Palchaudhuri et al. Nature Biotech 2016 34:738-745), and this approach to preparation has the potential to expand the utility of BMT if it can be successfully translated to patients. To further investigate the utility of an anti-CD45 ADC (anti-CD45 antibody, 104, conjugated to saporin) in murine transplant models, we explored anti-CD45-saporin (CD45-SAP) in an allogeneic minor mismatch transplant model (Balb/c donor into DBA/2 recipients). The goal of the work was to identify the level of immune suppression, if any, that needs to be used in combination with CD45-SAP to enable high donor chimerism in the allogeneic setting. (CD45-SAP is alternatively referred to as CD45-SAB-SAP, indicating that saporin is conjugated to monoclonal antibody 104 using streptavidin/biotin (SAB) coupling).

CD45-SAP (1.9 mg/kg, iv) was evaluated alone or in combination with additional immune modulating agents: clone 30F11 (25 mg/kg, IP), a naked anti-CD45 antibody that mimics ATG by relying on effector function to enable potent peripheral B- and T-cell depletion; pre-transplant Cytoxan (PreTCy, 200 mg/kg, IP), 2 Gy total body irradiation (TBI), and post-transplant Cytoxan (PTCy, 200 mg/kg, IP) to prevent graft versus host disease as well as block host versus graft rejection. 9 Gy TBI was used as the conventional conditioning positive control. Conditioned mice were transplanted with 2×10⁷ whole bone marrow cells, and chimerism assessed over 12 weeks.

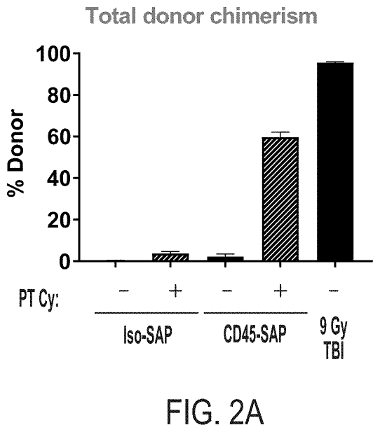

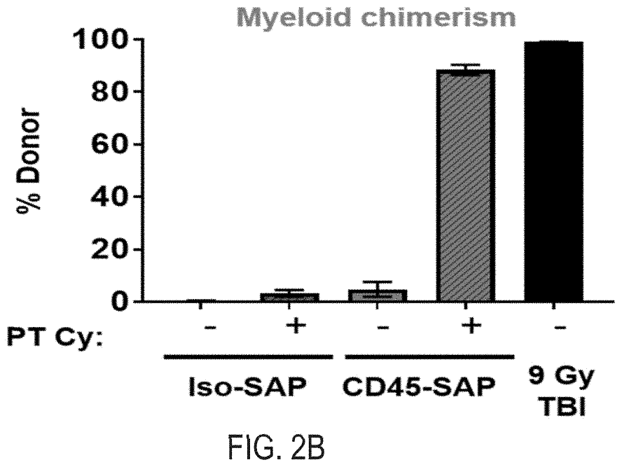

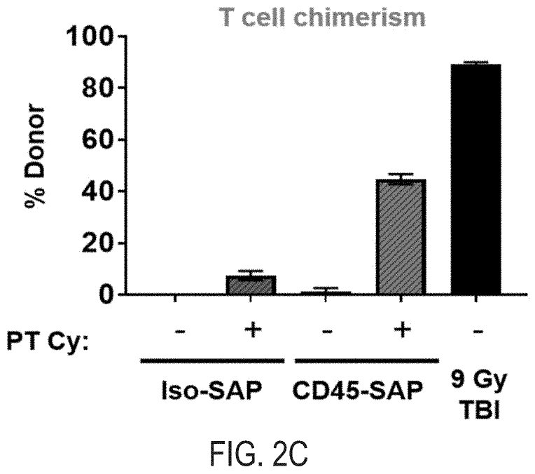

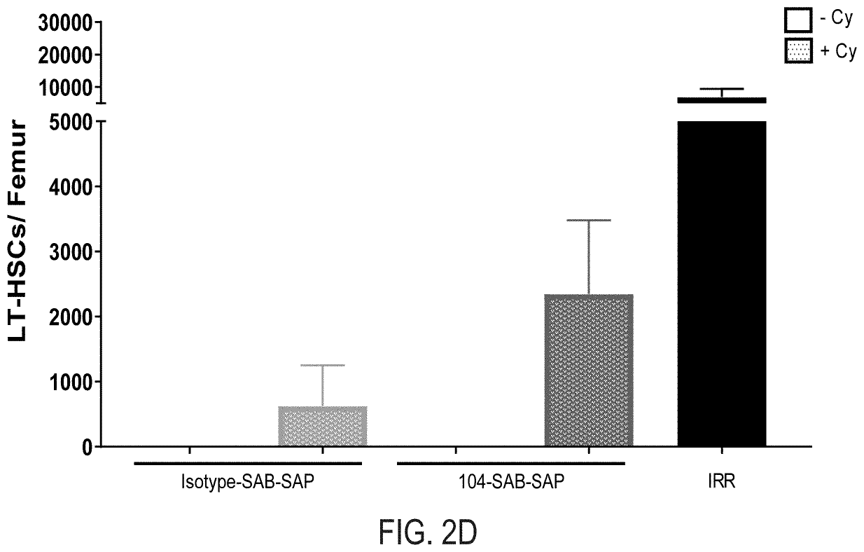

CD45-SAP in combination with PTCy achieved significant donor chimerism at 8 weeks post-transplantation (FIG. 2A), including a level of peripheral myeloid chimerism, a readout of stem cell engraftment, comparable to that achieved with 9 Gy TBI (>90%) (FIG. 21-2C). The addition of 30F11 to the CD45-SAP/PTCy protocol had no effect on peripheral donor chimerism (59% vs 61%), suggesting additional lymphodepletion is not required. In contrast, the single agents alone, 2 Gy TBI in combination with 30F11 and PTCy resulted in <5% donor engraftment. Other conditions tested that achieved low level donor myeloid chimerism were multi-dosing of 30F11 (QDx3) plus 2 Gy TBI with PTCy (40% donor chimerism) and PreTCy plus 30F11 (QDx3) with PTCy (20% donor chimerism). For all groups, stem cell chimerism in the bone marrow matched the peripheral chimerism.

Donor-derived long term HSCs were present in the bone marrow of recipient mice 12 weeks post-transplantation, in animals conditioned with CD45-SAP and Cytoxan (FIG. 2D). Results in FIG. 2D are presented from animals receiving Isotype control antibody coupled to saporin (Isotype-SAB-SAP), alone (left) or in combination with Cytoxan (right); animals receiving CD45 mAb 104 coupled to saporin (104-SAB-SAP), alone (left) or in combination with Cytoxan (right); and 9 Gy TBI (IRR), without Cytoxan.

These results indicate CD45-SAP in combination with PTCy is sufficient to enable high levels of donor chimerism in the minor mismatched setting without the need for additional immune suppression. CD45-SAP was more effective at conditioning than 2Gy TBI or PreTCy.

### Example 3. Anti-CD45 and Anti-CD117-Antibody Drug Conjugates Enable Allogeneic Hematopoietic Stem Cell Transplantation in Animal Models

Bone Marrow Transplant (BMT) is a potentially curative treatment for malignant and non-malignant blood disorders and has demonstrated impressive outcomes in autoimmune diseases. Prior to BMT, patients are prepared with high-dose chemotherapy alone or with total body irradiation, and both are associated with early and late morbidities, organ toxicities, infertility, secondary malignancies and substantial risk of mortality. This greatly limits the use of BMT in malignant and non-malignant conditions. To address these issues, we are developing antibody drug conjugates (ADCs) targeting hematopoietic stem cells (HSCs) and immune cells to safely condition patients for allogeneic BMT (35% of all transplants, CIBMTR) and autologous BMT (for autoimmune disease).

ADCs targeted to mouse CD45 or mouse CD117 have recently been shown to effectively condition immunocompetent mice for BMT (Palchaudhuri et al. Nature Biotech 2016 34:738-745; and Czechowicz et al. Blood 2016 128:493). These ADCs were created using saporin (SAP), a ribosome-inhibiting protein, which once internalized elicits cytotoxicity in a cell cycle-independent manner. Both anti-CD45-saporin (CD45-SAP) and anti-CD117-saporin (CD117-SAP) effectively depleted bone marrow HSCs as single dosed agents, and enabled efficient autologous HSC engraftment (>95% long-term donor chimerism). These ADCs have also enabled BMT in murine models of Fanconi Anemia.

To further investigate the utility of these murine ADCs, we explored CD45-SAP and CD117-SAP in the context of allogeneic minor mismatch transplant. Using the Balb/c donor into DBA/2 transplant model we sought to determine whether CD45-SAP or CD117-SAP could enable allogeneic transplant as single entity agents or needed to be combined with additional immunosuppressive agents (e.g. Cytoxan, ATG-mimic).

### Methods

Saporin (SAP)—Based Immunotoxins

Commercially available biotinylated anti-CD45.2 (clone 104) mAb was combined with streptavidin-saporin (ATS Bio, Catalog IT-27) in a 1:1 molar ratio just prior to injection. Similarly, to create CD117-SAP, biotinylated anti-CD117 (clone 2B8) mAb was combined with streptavidin saporin). Dosing was calculated based on the amount of antibody used to create the immunotoxin. The isotype-SAP was created by using a biotinylated mIgG2a isotype mAb.

Immunosuppressants

To mimic ATG, we used a naked anti-CD45 mAb (clone 30F11, 25 mg/kg IP) which relies on effector function to potently deplete peripheral lymphocytes without affecting bone marrow HSCs. Cytoxan was administered at 200 mg/kg IP 3 days post-transplant to prevent GvHD from the donor T cells, as shown in the schemes. Total body irradiation (2Gy or 9Gy) was performed using an X-ray irradiator.

Animal Studies

C57B16, DBA/2 and CD45.1 Balb/c mice were purchased from the Jackson Laboratories. DBA/2 mice were transplanted with 2×10⁷ whole bone marrow cells harvested from pooled Balb/c CD45.1 congenic donors. All in vivo research was conducted in accordance with the Guide for the Care and Use of Laboratory Animals published by the National Research Council of the National Academies and under the approval of the Institutional Animal Care and Use Committee.

### Murine HSC Depletion by CD45-SAP

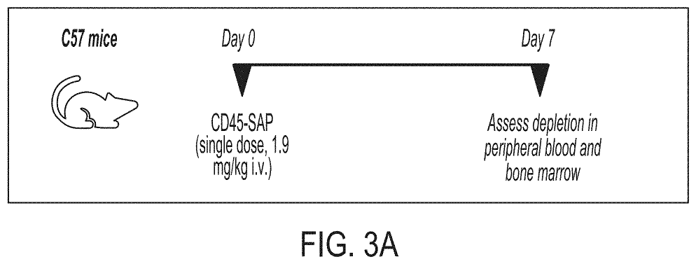

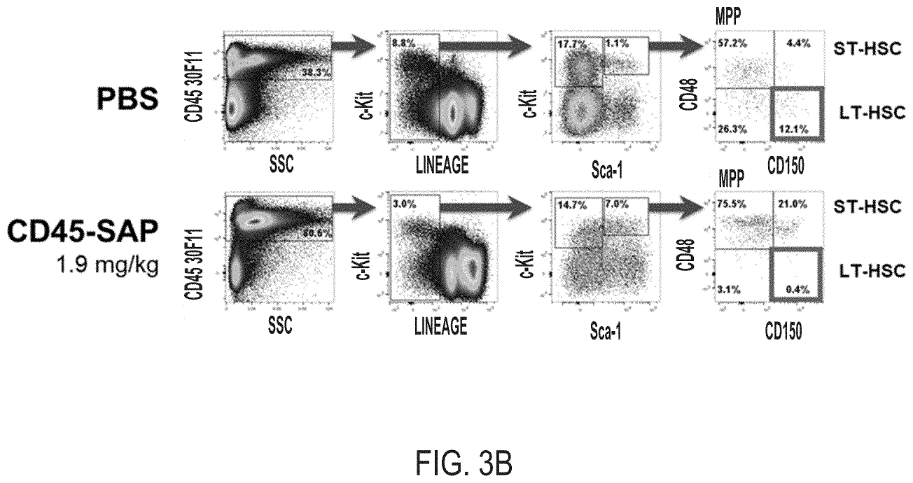

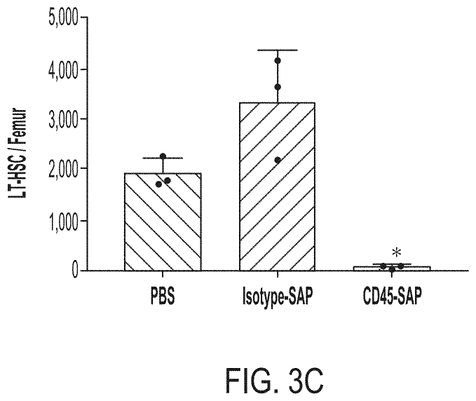

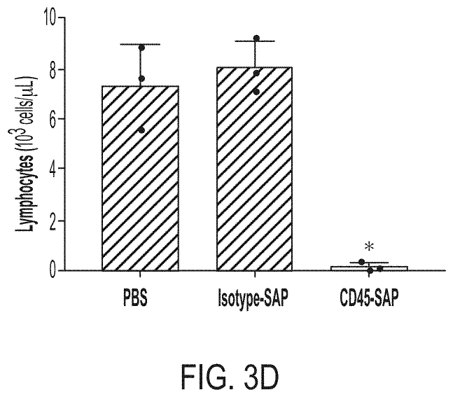

As outlined in the study design schematic in FIG. 3A, a single dose of CD45-SAP or controls (e.g., PBS or IgG1 isotype-SAP) was administered to C57 mice on day 0. Peripheral blood and bone marrow were collected on day 7 and examined by complete blood count (CBC) and flow cytometry. The bone marrow flow cytometry gating strategy and LT-HSC depletion by CD45-SAP are shown in FIG. 3B. Quantification of the level of LT-HSCs in the bone marrow of conditioned mice seven days post-dosing of PBS, isotype-SAP or CD45-SAP is shown in FIG. 3C. These results indicate that administration of CD45-SAP resulted in depletion of long-term HSCs (LT-HSCs) in bone marrow (FIGS. 3B and 3C). As shown in FIG. 3D, peripheral lymphocytes seven days post-dosing also showed effective depletion by CD45-SAP. Thus, CD45-SAP ADC effectively depletes murine HSCs and lymphocytes.

### Example 4. Conditioning with Antibody Drug Conjugate Targeted to CD45 Enables Allogeneic Hematopoietic Stem Cell Transplantation in Mice

The following study was conducted to examine whether an anti-mouse CD45 ADC (anti-CD45 antibody, 104, conjugated to PBD (“CD45-PBD”)) could be used to permit full-mismatch allogeneic-BMT in mice.

### Methods

An anti-mouse CD45 ADC containing mAb 104 coupled to PBD (CD45-PDB) was engineered to have rapid clearance (2-hour half-life) to enable bone marrow transplant. The optimal dose of the CD45-PBD was identified in a congenic autologous mouse transplant model. To determine if the ADC could successfully condition recipients for full allogeneic-BMT, CD45-PBD was evaluated alone or in combination with T cell depleting antibodies (anti-CD4 and anti-CD8, 0.25 mg/kg IP) in a full mismatch allogeneic-BMT model (Balb/c donor (H-2d, CD45.1+) into C57Bl/6 recipients (H-2b, CD45.2+). 9 Gy TBI was used as the conventional conditioning positive control. Conditioned mice were transplanted with 2×10⁷ whole bone marrow cells, and peripheral blood chimerism was assessed over 16 weeks. At 16 weeks, bone marrow stem cell chimerism was determined.

### Results

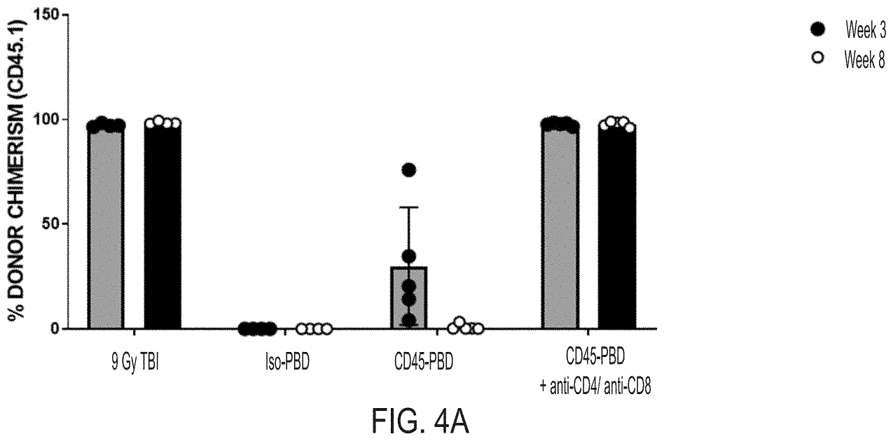

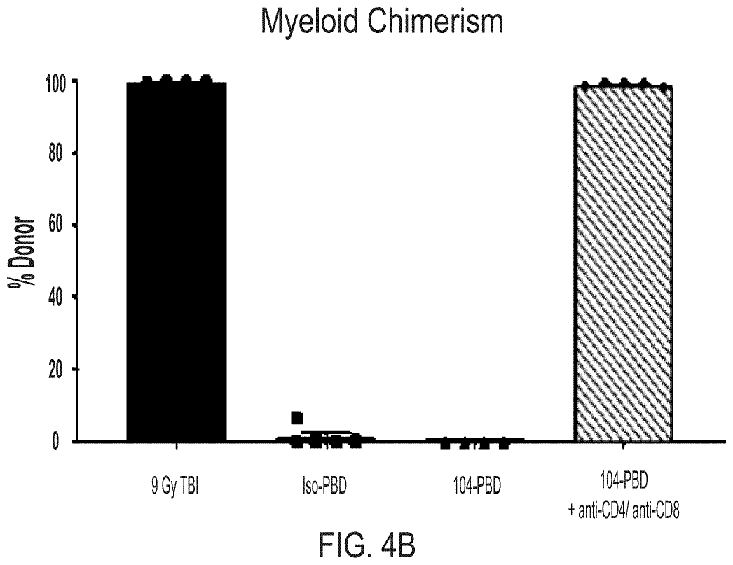

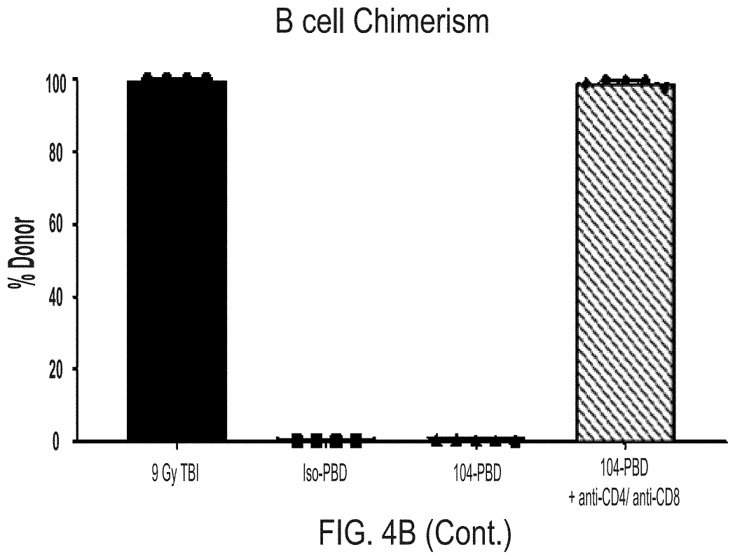

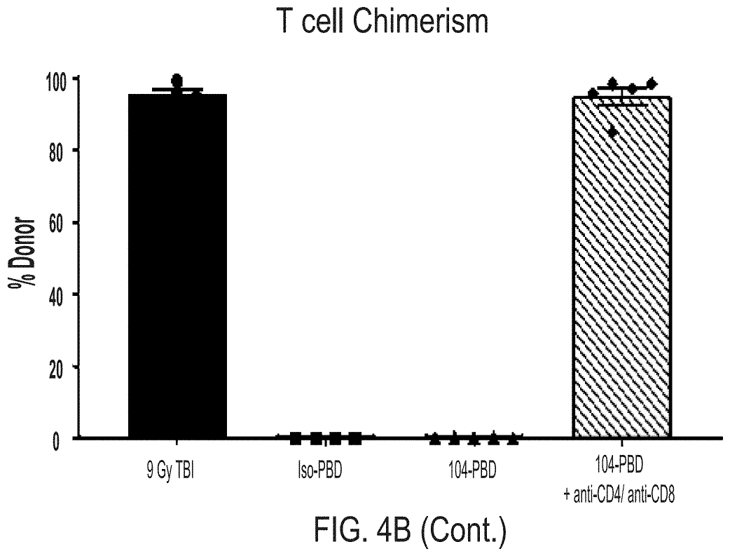

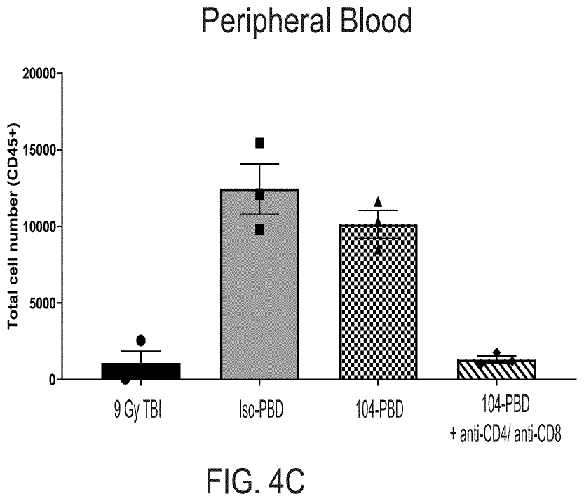

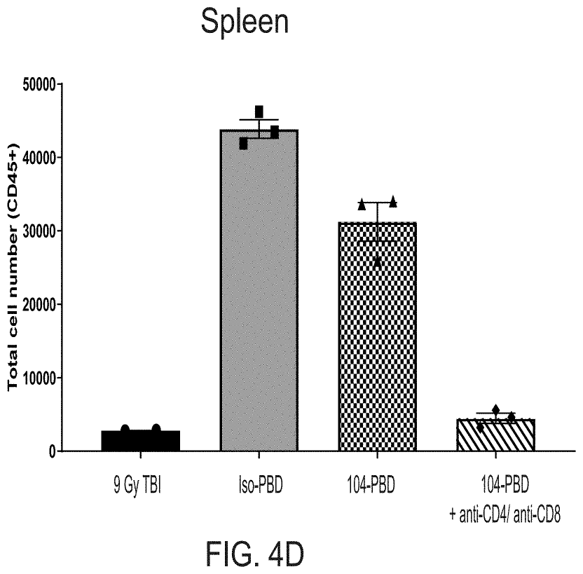

In a congenic autologous mouse model, a single dose of the CD45-PBD at 3 mg/kg was fully myeloablative, resulting in bone marrow failure at 11 days. Transplant of congenic bone marrow into CD45-PBD conditioned mice lead to full donor chimerism at a level that was comparable to animals that were conditioned with a myeloablative dose of irradiation (9Gy TBI). CD45-PBD was next evaluated in the fully mismatched Balb/c→C57Bl/6 allogeneic-BMT model, in which the donor and recipient mice have different MHC antigens. A single dose of the CD45-ADC at 3 mg/kg as a single agent enabled transient mixed myeloid chimerism out to 3 weeks (FIG. 4A). Supplementation of CD45-PBD with T cell depletion (using anti-CD4 and anti-CD8 antibodies) allows for durable, complete donor chimerism (>90% peripheral donor chimerism) at week 3 and week 8 post-transplantation (FIG. 4A), which was maintained through week 16. Multilineage reconstitution was observed in the T-, B-, and myeloid cell compartments with >90% donor chimerism at 8 weeks post-transplant seen in each compartment, indicative of hematopoietic stem cell engraftment (FIG. 4B). These results were comparable to chimerism seen in the 9 Gy TBI positive control for myeloablative conditioning (FIGS. 4A and 4B). Treatment with a non-targeting isotype matched ADC (Iso-PBD) was not effective. For all groups, stem cell chimerism in the bone marrow matched the peripheral chimerism. CD45-PBD in combination with T cell depletion (using anti-CD4 and anti-CD8 antibodies) enabled depletion of CD45+ cells from the peripheral blood and spleen two days post administration, as shown in FIGS. 4C and 4D.

These results demonstrate that a single dose of CD45-PBD is fully myeloablative and enables durable and complete donor chimerism in a full mismatch allogeneic-BMT model with supplemental T cell depletion. This targeted, readily translatable approach for safer conditioning could improve the risk benefit profile for allogenic and haploidentical BMT, and may extend the curative potential of this therapeutic modality to more patients suffering from blood cancers and other diseases that may benefit from BMT.

### Example 5. Bone Marrow Depletion and Allogenic Donor Chimerism Following Conditioning with Anti-CD45 ADC and Low Dose TBI

An CD45-ADC containing anti-CD45 mAb 104 and PBD (CD45-PBD, also referred to as 104-PBD) was evaluated alone or in combination with low dose (0.5-2 Gy) total body irradiation (TBI) in a full mismatch allogeneic-HSC transplant model (Balb/c donors (H-2d, CD45.1+) into C57Bl/6 recipients (H-2b, CD45.2+)). TBI doses below 9 Gy TBI (5 Gy, 4 Gy, 3Gy, 2 Gy, 1 Gy, 0.5 Gy, and 0 Gy) were assessed in combination with CD45-PBD. 9 Gy TBI served as the conventional conditioning positive control. Conditioned C57Bl/6 recipient mice were transplanted with 2×10⁷ whole bone marrow cells derived from Balb/c donors, and peripheral blood chimerism was assessed over 16 weeks.

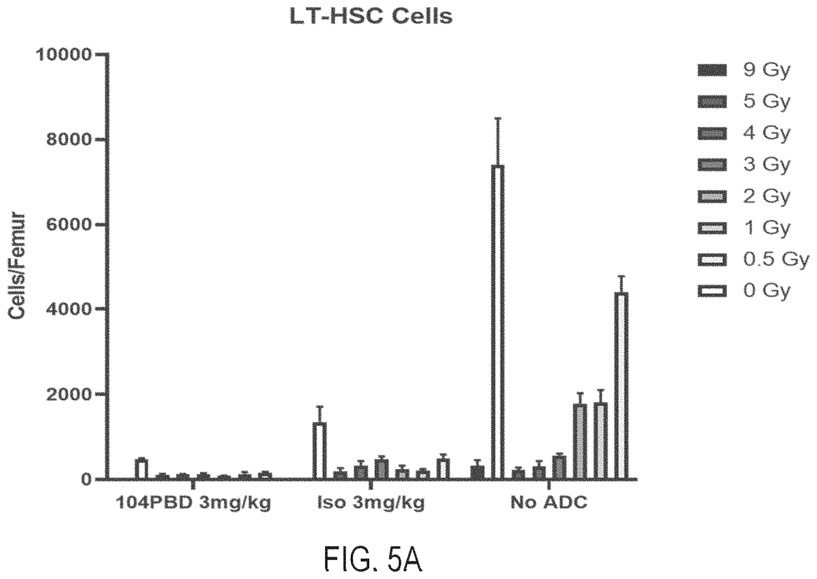

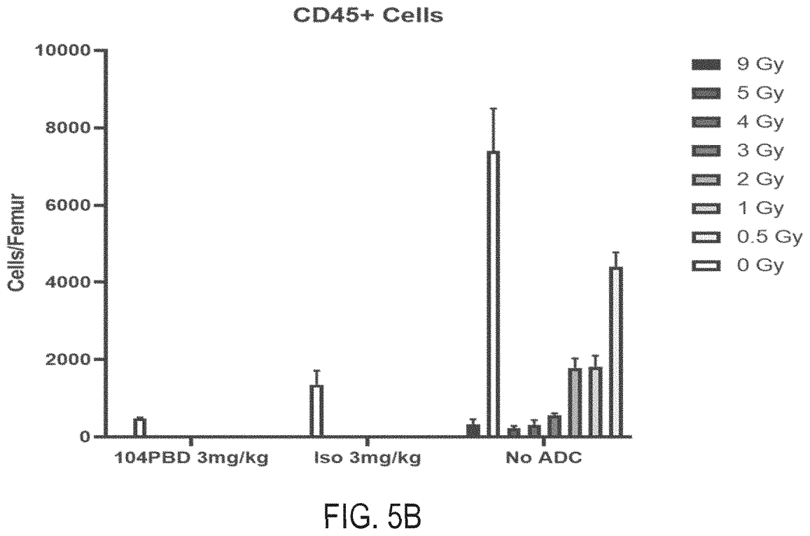

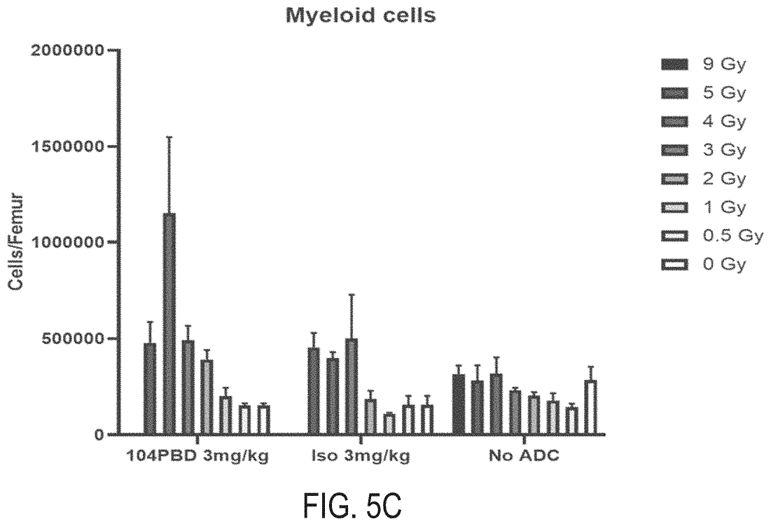

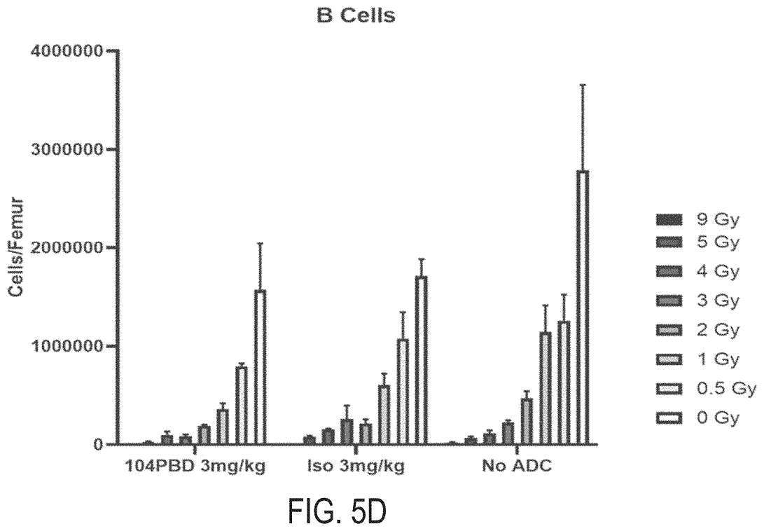

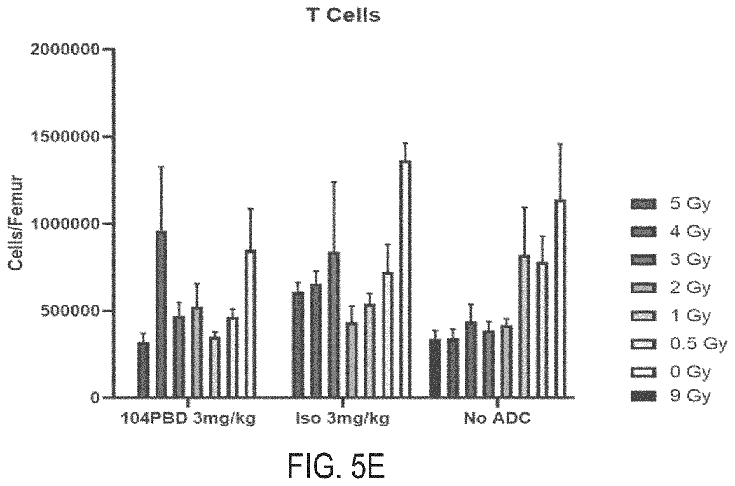

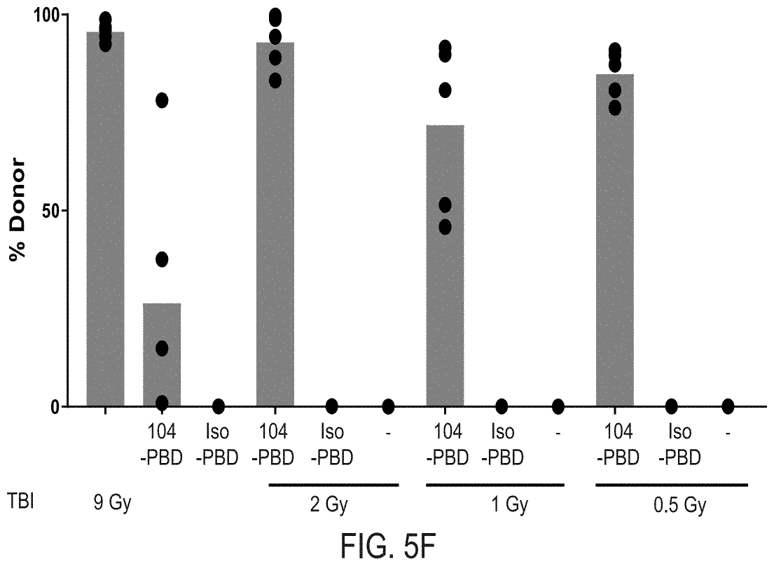

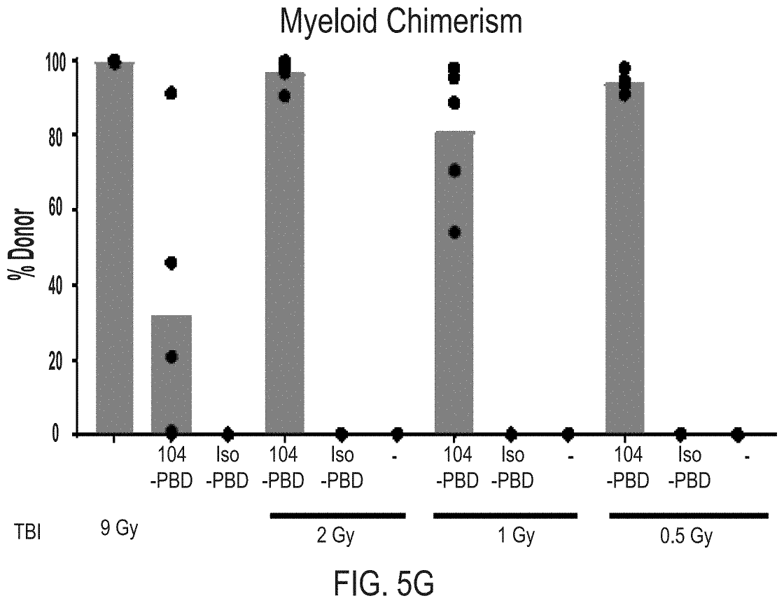

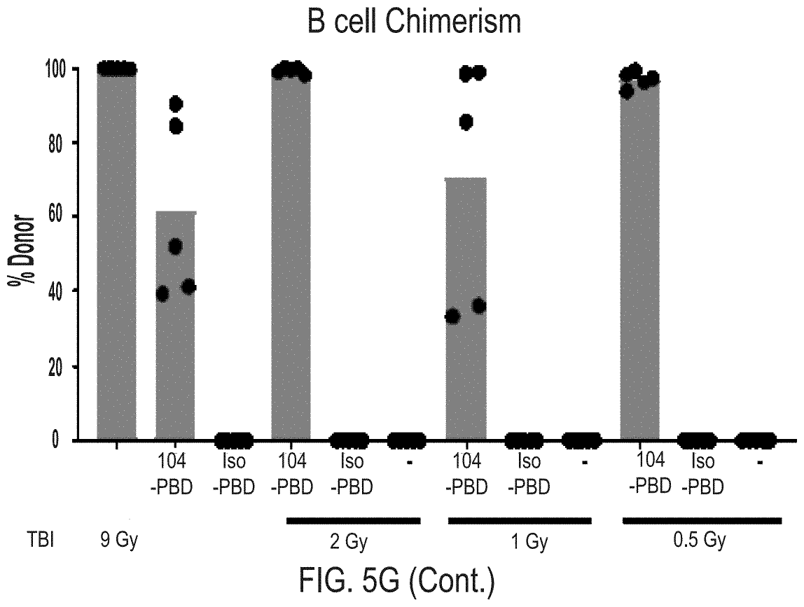

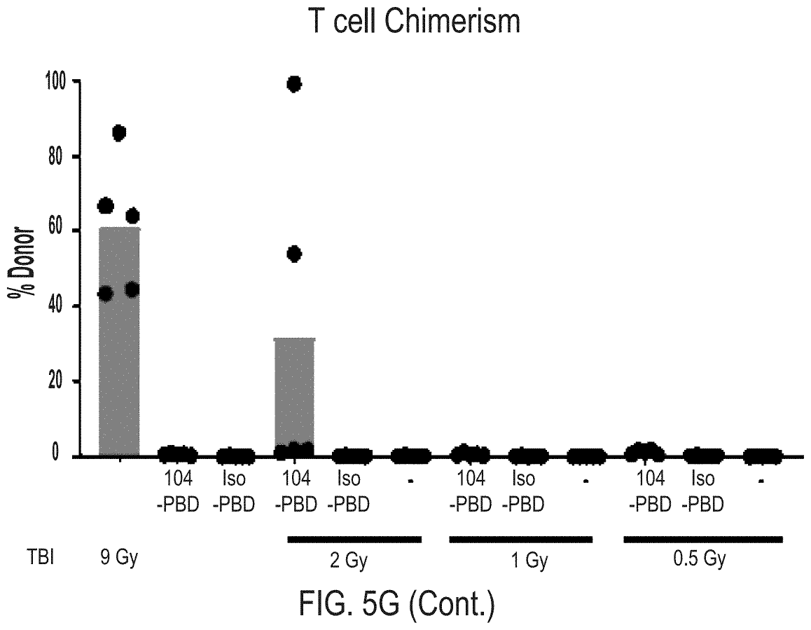

In combination with low dose TBI, CD45-PBD enabled depletion of LT-HSC cells (FIG. 5A) and depletion of CD45+ cells (FIG. 5B), myeloid cells (FIG. 5C). B cells (FIG. 50), and T cells (FIG. 5E) in the bone marrow two days post ADC administration. CD45-PBD in combination with low-dose TBI enabled full allogenic donor chimerism (>90% donor chimerism in the peripheral blood) by week 4 post-transplantation (FIG. 5F). Multilineage reconstitution of B- and myeloid cell compartments was observed (>90% donor chimerism; FIG. 5G) following conditioning with the CD45-PBD in combination with low-dose TBI (0.5 Gy) and was comparable to chimerism seen in the 9 Gy TBI positive control (FIGS. 5F and 5G). Treatment with a non-targeting isotype ADC was not effective (FIG. 5F, 5G).

TABLE 3
SEQUENCE SUMMARY

| Sequence | | |
|---|---|---|
| identifier | Description | Sequence |
| SEQ ID NO: 1 | CK6 CDR-H1 | SYWIG |
| SEQ ID NO: 2 | CK6 CDR-H2 | IIYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Consensus human Ab | EVQLVESGGGLVQPGGSLRLSCAASGFTFSDYAMSW |
| | Heavy chain variable | VRQAPGKGLEWVAVISENGSDTYYADSVKGRFTISRD |
| | domain | DSKNTLYLQMNSLRAEDTAVYYCARDRGGAVSYFDV |
| | | WGQGTLVTVSS |
| SEQ ID NO: 8 | Consensus human Ab | DIQMTQSPSSLSASVGDRVTITCRASQDVSSYLAWYQ |
| | Light chain variable | QKPGKAPKLLIYAASSLESGVPSRFSGSGSGTDFTLTI |
| | domain | SSLQPEDFATYYCQQYNSLPYTFGQGTKVEIKRT |
| SEQ ID NO: 9 | Ab67 Heavy chain | EVQLVESGGGLVQPGGSLRLSCAASGFTFSDADMDW |
| | variable region (e.g., | VRQAPGKGLEWVGRTRNKAGSYTTEYAASVKGRFTI |
| | as found in HC-67) | SRDDSKNSLYLQMNSLKTEDTAVYYCAREPKYWIDFD |
| | higG1 backbone | LWGRGTLVTVSS |
| | (CDRs in bold) | |
| SEQ ID NO: 10 | Ab67 Light chain | DIQMTQSPSSLSASVGDRVTITCRASQSISSYLNWYQQ |
| | variable region (e.g., | KPGKAPKLLIYAASSLQSGVPSRFSGSGSGTDFTLTIS |
| | as found in LC-67) | SLQPEDFATYYCQQSYIAPYTFGGGTKVEIK |
| | hKappa backbone | |
| | (CDRs in bold) | |
| SEQ ID NO: 11 | Ab67 CDR-H1 | FTFSDADMD |
| SEQ ID NO: 12 | Ab67 CDR-H2 | RTRNKAGSYTTEYAASVKG |
| SEQ ID NO: 13 | Ab67 CDR-H3 | AREPKYWIDFDL |
| SEQ ID NO: 14 | Ab67 CDR-L1 | RASQSISSYLN |
| SEQ ID NO: 15 | Ab67 CDR-L2 | AASSLQS |
| SEQ ID NO: 16 | Ab67 CDR-L3 | QQSYIAPYT |
| SEQ ID NO: 17 | Ab67 Heavy chain | GAGGTGCAGCTGGTGGAGTCTGGGGGAGGCTTGGT |
| | variable region (nucl) | CCAGCCTGGAGGGTCCCTGAGACTCTCCTGTGCAG |
| | | CCTCTGGATTCACCTTCAGTGACGCCGACATGGACT |
| | | GGGTCCGCCAGGCTCCAGGGAAGGGGCTGGAGTG |
| | | GGTTGGCCGTACTAGAAACAAAGCAGGAAGTTACAC |
| | | CACAGAATACGCCGCGTCTGTGAAAGGCAGATTCAC |
| | | CATCTCAAGAGATGATTCAAAGAACTCACTGTATCTG |
| | | CAAATGAACAGCCTGAAAACCGAGGACACGGCGGT |
| | | GTACTACTGCGCCAGAGAGCCTAAATACTGGATCGA |
| | | CTTCGACCTATGGGGGAGAGGTACCTTGGTCACCGT |
| | | CTCCTCA |
| SEQ ID NO: 18 | Ab67 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAGCAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAAGCTACATCGCCCCTTACA |
| | | CTTTTGGCGGAGGGACCAAGGTTGAGATCAAA |
| SEQ ID NO: 19 | Ab55 Heavy chain | QVQLVQSGAEVKKPGSSVKVSCKASGGTFRIYAISWV |
| | variable region (e.g., | RQAPGQGLEWMGGIIPDFGVANYAQKFQGRVTITADE |
| | as found in HC-55) | STSTAYMELSSLRSEDTAVYYCARGGLDTDEFDLWGR |
| | hlgG1 backbone | GTLVTVSS |
| | CDRs in bold) | |
| SEQ ID NO: 20 | Ab55 Light chain | DIQMTQSPSSLSASVGDRVTITCRASQSINSYLNWYQ |
| | variable region (e.g., | QKPGKAPKLLIYAASSLQSGVPSRFSGSGSGTDFTLTI |
| | aS fond in LC-55) | SSLQPEDFATYYCQQGVSDITFGGGTKVEIK |
| | hKappa backbone | |
| | (CDRs in bold) | |
| SEQ ID NO: 21 | Ab55 CDR-H1 | GTFRIYAIS |
| SEQ ID NO: 22 | Ab55 CDR-H2 | GIIPDFGVANYAQKFQG |
| SEQ ID NO: 23 | Ab55 CDR-H3 | ARGGLDTDEFDL |
| SEQ ID NO: 24 | Ab55 CDR-L1 | RASQSINSYLN |
| SEQ ID NO: 25 | Ab55 CDR-L2 | AASSLQS |
| SEQ ID NO: 26 | Ab55 CDR-L3 | QQGVSDIT |
| SEQ ID NO: 27 | Ab55 Heavy chain | CAGGTGCAGCTGGTGCAGTCTGGGGCTGAGGTGAA |
| | variable region (nucl) | GAAGCCTGGGTCCTCGGTGAAGGTCTCCTGCAAGG |
| | | CTTCTGGAGGCACCTTCCGAATCTATGCTATCAGCT |
| | | GGGTGCGACAGGCCCCTGGACAAGGGCTTGAGTGG |
| | | ATGGGAGGGATCATCCCTGACTTCGGTGTAGCAAAC |
| | | TACGCACAGAAGTTCCAGGGCAGAGTCACGATTACC |
| | | GCGGACGAATCCACGAGCACAGCCTACATGGAGCT |
| | | GAGCAGCCTGAGATCTGAGGACACGGCGGTGTACT |
| | | ACTGCGCCAGAGGTGGATTGGACACAGACGAGTTC |
| | | GACCTATGGGGGAGAGGTACCTTGGTCACCGTCTC |
| | | CTCA |
| SEQ ID NO: 28 | Ab55 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAACAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAGGAGTCAGTGACATCACTTT |
| | | TGGCGGAGGGACCAAGGTTGAGATCAAA |
| SEQ ID NO: 29 | Ab54 Heavy chain | QVQLVQSGAEVKKPGSSVKVSCKASGGTFSSYAISWV |
| | variable region (e.g., | RQAPGQGLEWMGGIIPIFGTANYAQKFQGRVTITADES |
| | as found in HC-54) | TSTAYMELSSLRSEDTAVYYCARGGLDTDEFDLWGR |
| | hlgG1 backbone | GTLVTVSS |
| | (CDRs in bold) | |
| SEQ ID NO: 30 | Ab54 Light chain | DIQMTQSPSSLSASVGDRVTITCRASQSINSYLNWYQ |
| | variable region (e.g., | QKPGKAPKLLIYAASSLQSGVPSRFSGSGSGTDFTLTI |
| | as found in LC-54) | SSLQPEDFATYYCQQGVSDITFGGGTKVEIK |
| | hKappa backbone | |
| | CDRs in bold) | |
| SEQ ID NO: 31 | Ab54 CDR-H1 | GTFSSYAIS |
| SEQ ID NO: 32 | Ab54 CDR-H2 | GIIPIFGTANYAQKFQG |
| SEQ ID NO: 33 | Ab54 CDR-H3 | ARGGLDTDEFDL |
| SEQ ID NO: 34 | Ab54 CDR-L1 | RASQSINSYLN |
| SEQ ID NO: 35 | Ab54 CDR-L2 | AASSLQS |
| SEQ ID NO: 36 | Ab54 CDR-L3 | QQGVSDIT |
| SEQ ID NO: 37 | Ab54 Heavy chain | CAGGTGCAGCTGGTGCAGTCTGGGGCTGAGGTGAA |
| | variable region (nucl) | GAAGCCTGGGTCCTCGGTGAAGGTCTCCTGCAAGG |
| | | CTTCTGGAGGCACCTTCAGCAGCTATGCTATCAGCT |
| | | GGGTGCGACAGGCCCCTGGACAAGGGCTTGAGTGG |
| | | ATGGGAGGGATCATCCCTATCTTTGGTACAGCAAAC |
| | | TACGCACAGAAGTTCCAGGGCAGAGTCACGATTACC |
| | | GCGGACGAATCCACGAGCACAGCCTACATGGAGCT |
| | | GAGCAGCCTGAGATCTGAGGACACGGCGGTGTACT |
| | | ACTGCGCCAGAGGTGGATTGGACACAGACGAGTTC |
| | | GACCTATGGGGGAGAGGTACCTTGGTCACCGTCTC |
| | | CTCA |
| SEQ ID NO: 38 | Ab54 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAACAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAGGAGTCAGTGACATCACTTT |
| | | TGGCGGAGGGACCAAGGTTGAGATCAAA |
| SEQ ID NO: 39 | Ab56 Heavy chain | QVQLVQSGAEVKKPGSSVKVSCKASGGTFSLYAISWV |
| | variable region | RQAPGQGLEWMGGIIPAFGTANYAQKFQGRVTITADE |
| | (e.g., as found in HC- | STSTAYMELSSLRSEDTAVYYCARGGLDTDEFDLWGR |
| | 56) | GTLVTVSS |
| | hlgG1 backbone | |
| | (CDRs in bold) | |
| SEQ ID NO: 40 | Ab56 Light chain | DIQMTQSPSSLSASVGDRVTITCRASQSINSYLNWYQ |
| | variable region (e.g., | QKPGKAPKLLIYAASSLQSGVPSRFSGSGSGTDFTLTI |
| | as found in LC-56) | SSLQPEDFATYYCQQGVSDITFGGGTKVEIK |
| | hKappa backbone | |
| | (CDRs in bold) | |
| SEQ ID NO: 41 | Ab56 CDR-H1 | GTFSLYAIS |
| SEQ ID NO: 42 | Ab56 CDR-H2 | GIIPAFGTANYAQKFQG |
| SEQ ID NO: 43 | Ab56 CDR-H3 | ARGGLDTDEFDL |
| SEQ ID NO: 44 | Ab56 CDR-L1 | RASQSINSYLN |
| SEQ ID NO: 45 | Ab56 CDR-L2 | AASSLQS |
| SEQ ID NO: 46 | Ab56 CDR-L3 | QQGVSDIT |
| SEQ ID NO: 47 | Ab56 Heavy chain | CAGGTGCAGCTGGTGCAGTCTGGGGCTGAGGTGAA |
| | variable region (nucl) | GAAGCCTGGGTCCTCGGTGAAGGTCTCCTGCAAGG |
| | | CTTCTGGAGGCACCTTCAGCCTCTATGCTATCTCCT |
| | | GGGTGCGACAGGCCCCTGGACAAGGGCTTGAGTGG |
| | | ATGGGAGGGATCATCCCTGCCTTCGGTACCGCAAAC |
| | | TACGCACAGAAGTTCCAGGGCAGAGTCACGATTACC |
| | | GCGGACGAATCCACGAGCACAGCCTACATGGAGCT |
| | | GAGCAGCCTGAGATCTGAGGACACGGCGGTGTACT |
| | | ACTGCGCCAGAGGTGGATTGGACACAGACGAGTTC |
| | | GACCTATGGGGGAGAGGTACCTTGGTCACCGTCTC |
| | | CTCA |
| SEQ ID NO: 48 | Ab56 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAACAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAGGAGTCAGTGACATCACTTT |
| | | TGGCGGAGGGACCAAGGTTGAGATCAAA |
| SEQ ID NO: 49 | Ab57 Heavy chain | QVQLVQSGAEVKKPGSSVKVSCKASGGTFSLYAISWV |
| | variable region (e.g., | RQAPGQGLEWMGGIIPHFGLANYAQKFQGRVTITADE |
| | as found in HC-57) | STSTAYMELSSLRSEDTAVYYCARGGLDTDEFDLWGR |
| | hlgG1 backbone | GTLVTVSS |
| | (CDRs in bold) | |
| SEQ ID NO: 50 | Ab57 Light chain | DIQMTQSPSSLSASVGDRVTITCRASQSINSYLNWYQ |
| | variable region (e.g., | QKPGKAPKLLIYAASSLQSGVPSRFSGSGSGTDFTLTI |
| | as found in LC-57) | SSLQPEDFATYYCQQGVSDITFGGGTKVEIK |
| | hKappa backbone | |
| | (CDRs in bold) | |
| SEQ ID NO: 51 | Ab57 CDR-H1 | GTFSLYAIS |
| SEQ ID NO: 52 | Ab57 CDR-H2 | GIIPHFGLANYAQKFQG |
| SEQ ID NO: 53 | Ab57 CDR-H3 | ARGGLDTDEFDL |
| SEQ ID NO: 54 | Ab57 CDR-L1 | RASQSINSYLN |
| SEQ ID NO: 55 | Ab57 CDR-L2 | AASSLQS |
| SEQ ID NO: 56 | Ab57 CDR-L3 | QQGVSDIT |
| SEQ ID NO: 57 | Ab57 Heavy chain | CAGGTGCAGCTGGTGCAGTCTGGGGCTGAGGTGAA |
| | variable region (nucl) | GAAGCCTGGGTCCTCGGTGAAGGTCTCCTGCAAGG |
| | | CTTCTGGAGGCACCTTCTCCCTCTATGCTATCAGCT |
| | | GGGTGCGACAGGCCCCTGGACAAGGGCTTGAGTGG |
| | | ATGGGAGGGATCATCCCTCACTTCGGTCTCGCAAAC |
| | | TACGCACAGAAGTTCCAGGGCAGAGTCACGATTACC |
| | | GCGGACGAATCCACGAGCACAGCCTACATGGAGCT |
| | | GAGCAGCCTGAGATCTGAGGACACGGCGGTGTACT |
| | | ACTGCGCCAGAGGTGGATTGGACACAGACGAGTTC |
| | | GACCTATGGGGGAGAGGTACCTTGGTCACCGTCTC |
| | | CTCA |
| SEQ ID NO: 58 | Ab57 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAACAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGICAGCAAGGAGICAGTGACATCACTTT |
| | | TGGCGGAGGGACCAAGGTTGAGATCAAA |
| SEQ ID NO: 59 | Ab58 Heavy chain | EVQLLESGGGLVQPGGSLRLSCAASGFTFSNYAMSW |
| | variable region (e.g., | VRQAPGKGLEWVSAISGSGGSTYYADSVKGRFTISRD |
| | as found in HC-58) | NSKNTLYLQMNSLRAEDTAVYYCAKGPPTYHTNYYYM |
| | hlgG1 backbone | DVWGKGTTVTVSS |
| | (CDRs in bold) | |
| SEQ ID NO: 60 | Ab58 Light chain | DIQMTQSPSSVSASVGDRVTITCRASQGISSWLAWYQ |
| | variable region (e.g., | QKPGKAPKLLIYAASSLQSGVPSRFSGSGSGTDFTLTI |
| | as found in LC-58) | SSLQPEDFATYYCQQTNSFPYTFGGGTKVEIK |
| | hKappa backbone | |
| | (CDRs in bold) | |
| SEQ ID NO: 61 | Ab58 CDR-H1 | FTFSNYAMS |
| SEQ ID NO: 62 | Ab58 CDR-H2 | AISGSGGSTYYADSVKG |
| SEQ ID NO: 63 | Ab58 CDR-H3 | AKGPPTYHTNYYYMDV |
| SEQ ID NO: 64 | Ab58 CDR-L1 | RASQGISSWLA |
| SEQ ID NO: 65 | Ab58 CDR-L2 | AASSLQS |
| SEQ ID NO: 66 | Ab58 CDR-L3 | QQTNSFPYT |
| SEQ ID NO: 67 | Ab58 Heavy chain | GAGGTGCAGCTGTTGGAGTCTGGGGGAGGCTTGGT |
| | variable region (nucl) | ACAGCCTGGGGGGTCCCTGAGACTCTCCTGTGCAG |
| | | CCTCTGGATTCACCTTTAGCAATTATGCCATGAGCTG |
| | | GGTCCGCCAGGCTCCAGGGAAGGGGCTGGAGTGG |
| | | GTCTCAGCTATTAGTGGTAGTGGTGGTAGCACATAC |
| | | TACGCAGACTCCGTGAAGGGCCGGTTCACCATCTCC |
| | | AGAGACAATTCCAAGAACACGCTGTATCTGCAAATG |
| | | AACAGCCTGAGAGCCGAGGACACGGCGGTGTACTA |
| | | CTGCGCCAAGGGCCCTCCTACATACCACACAAACTA |
| | | CTACTACATGGACGTATGGGGCAAGGGTACAACTGT |
| | | CACCGTCTCCTCA |
| SEQ ID NO: 68 | Ab58 Light chain | GACATCCAGATGACCCAGTCTCCATCTTCCGTGTCT |
| | variable region (nucl) | GCATCTGTAGGAGACAGAGTCACCATCACTTGTCGG |
| | | GCGAGTCAGGGTATTAGCAGCTGGTTAGCCTGGTAT |
| | | CAGCAGAAACCAGGGAAAGCCCCTAAGCTCCTGATC |
| | | TATGCTGCATCCAGTTTGCAAAGTGGGGTCCCATCA |
| | | AGGTTCAGCGGCAGTGGATCTGGGACAGATTTCACT |
| | | CTCACCATCAGCAGCCTGCAGCCTGAAGATTTTGCA |
| | | ACTTATTACTGTCAGCAAACAAATAGTTTCCCTTACA |
| | | CTTTTGGCGGAGGGACCAAGGTTGAGATCAAA |
| SEQ ID NO: 69 | Ab61 Heavy chain | EVQLLESGGGLVQPGGSLRLSCAASGFTFSSYVMIWV |
| | variable region (e.g., | RQAPGKGLEWVSSISGDSVTTYYADSVKGRFTISRDN |
| | as found in HC-61) | SKNTLYLQMNSLRAEDTAVYYCAKGPPTYHTNYYYMD |
| | hlgG1 backbone | VWGKGTTVTVSS |
| | CDRs in bold) | |
| SEQ ID NO: 70 | Ab61 Light chain | DIQMTQSPSSVSASVGDRVTITCRASQGISSWLAWYQ |
| | variable region (e.g.. | QKPGKAPKLLIYAASSLQSGVPSRFSGSGSGTDFTLTI |
| | as found in LC-61) | SSLQPEDFATYYCQQTNSFPYTFGGGTKVEIK |
| | hKapba backbone | |
| | (CDRs in bold) | |
| SEQ ID NO: 71 | Ab61 CDR-H1 | FTFSSYVMI |
| SEQ ID NO: 72 | Ab61 CDR-H2 | SISGDSVTTYYADSVKG |
| SEQ ID NO: 73 | Ab61 CDR-H3 | AKGPPTYHTNYYYMDV |
| SEQ ID NO: 74 | Ab61 CDR-L1 | RASQGISSWLA |
| SEQ ID NO: 75 | Ab61 CDR-L2 | AASSLQS |
| SEQ ID NO: 76 | Ab61 CDR-L3 | QQTNSFPYT |
| SEQ ID NO: 77 | Ab61 Heavy chain | GAGGTGCAGCTGTTGGAGTCTGGGGGAGGCTTGGT |
| | variable region (nucl) | ACAGCCTGGGGGGTCCCTGAGACTCTCCTGTGCAG |
| | | CCTCTGGATTCACCTTTAGCAGCTATGTCATGATCTG |
| | | GGTCCGCCAGGCTCCAGGGAAGGGGCTGGAGTGG |
| | | GTCTCAAGCATTAGTGGTGACAGCGTAACAACATAC |
| | | TACGCAGACTCCGTGAAGGGCCGGTTCACCATCTCC |
| | | AGAGACAATTCCAAGAACACGCTGTATCTGCAAATG |
| | | AACAGCCTGAGAGCCGAGGACACGGCGGTGTACTA |
| | | CTGCGCCAAGGGCCCTCCTACATACCACACAAACTA |
| | | CTACTACATGGACGTATGGGGCAAGGGTACAACTGT |
| | | CACCGTCTCCTCA |
| SEQ ID NO: 78 | Ab61 Light chain | GACATCCAGATGACCCAGTCTCCATCTTCCGTGTCT |
| | variable region (nucl) | GCATCTGTAGGAGACAGAGTCACCATCACTTGTCGG |
| | | GCGAGTCAGGGTATTAGCAGCTGGTTAGCCTGGTAT |
| | | CAGCAGAAACCAGGGAAAGCCCCTAAGCTCCTGATC |
| | | TATGCTGCATCCAGTTTGCAAAGTGGGGTCCCATCA |
| | | AGGTTCAGCGGCAGTGGATCTGGGACAGATTTCACT |
| | | CTCACCATCAGCAGCCTGCAGCCTGAAGATTTTGCA |
| | | ACTTATTACTGTCAGCAAACAAATAGTTTCCCTTACA |
| | | CTTTTGGCGGAGGGACCAAGGTTGAGATCAAA |
| SEQ ID NO: 79 | Ab66 Heavy chain | EVQLVESGGGLVQPGGSLRLSCAASGFTFSDHYMDW |
| | variable region (e.g., | VRQAPGKGLEWVGRTRNKASSYTTEYAASVKGRFTIS |
| | as found in HC-66) | RDDSKNSLYLQMNSLKTEDTAVYYCAREPKYWIDFDL |
| | hlgG1 backbone | WGRGTLVTVSS |
| | (CDRs in bold) | |
| SEQ ID NO: 80 | Ab66 Light chain | DIQMTQSPSSLSASVGDRVTITCRASQSISSYLNWYQQ |
| | variable region (e.g., | KPGKAPKLLIYAASSLQSGVPSRFSGSGSGTDFTLTIS |
| | as found in LC-66) | SLQPEDFATYYCQQSYIAPYTFGGGTKVEIK |
| | hKappa backbone | |
| | (CDRs in bold) | |
| SEQ ID NO: 81 | Ab66 CDR-H1 | FTFSDHYMD |
| SEQ ID NO: 82 | Ab66 CDR-H2 | RTRNKASSYTTEYAASVKG |
| SEQ ID NO: 83 | Ab66 CDR-H3 | AREPKYWIDFDL |
| SEQ ID NO: 84 | Ab66 CDR-L1 | RASQSISSYLN |
| SEQ ID NO: 85 | Ab66 CDR-L2 | AASSLQS |
| SEQ ID NO: 86 | Ab66 CDR-L3 | QQSYIAPYT |
| SEQ ID NO: 87 | Ab66 Heavy chain | GAGGTGCAGCTGGTGGAGTCTGGGGGAGGCTTGGT |
| | variable region (nucl) | CCAGCCTGGAGGGTCCCTGAGACTCTCCTGTGCAG |
| | | CCTCTGGATTCACCTTCAGTGACCACTACATGGACT |
| | | GGGTCCGCCAGGCTCCAGGGAAGGGGCTGGAGTG |
| | | GGTTGGCCGTACTAGAAACAAAGCTAGTAGTTACAC |
| | | CACAGAATACGCCGCGTCTGTGAAAGGCAGATTCAC |
| | | CATCTCAAGAGATGATTCAAAGAACTCACTGTATCTG |
| | | CAAATGAACAGCCTGAAAACCGAGGACACGGCGGT |
| | | GTACTACTGCGCCAGAGAGCCTAAATACTGGATCGA |
| | | CTTCGACCTATGGGGGAGAGGTACCTTGGTCACCGT |
| | | CTCCTCA |
| SEQ ID NO: 88 | Ab66 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAGCAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAAGCTACATCGCCCCTTACA |
| | | CTTTTGGCGGAGGGACCAAGGTTGAGATCAAA |
| SEQ ID NO: 89 | Ab68 Heavy chain | EVQLVESGGGLVQPGRSLRLSCTASGFTFSDHDMNW |
| | variable region (e.g., | VRQAPGKGLEWVGRTRNAAGSYTTEYAASVKGRFTI |
| | as found in HC-68) | SRDDSKNSLYLQMNSLKTEDTAVYYCAREPKYWIDFD |
| | hlgG1 backbone | LWGRGTLVTVSS |
| | (CDRs in bold) | |
| SEQ ID NO: 90 | Ab68 Light chain | DIQMTQSPSSLSASVGDRVTITCRASQSISSYLNWYQQ |
| | variable region (e.g., | KPGKAPKLLIYAASSLQSGVPSRFSGSGSGTDFTLTIS |
| | as found in LC-68) | SLQPEDFATYYCQQSYIAPYTFGGGTKVEIK |
| | hKappa backbone | |
| | (CDRs in bold) | |
| SEQ ID NO: 91 | Ab68 CDR-H1 | FTFSDHDMN |
| SEQ ID NO: 92 | Ab68 CDR-H2 | RTRNAAGSYTTEYAASVKG |
| SEQ ID NO: 93 | Ab68 CDR-H3 | AREPKYWIDFDL |
| SEQ ID NO: 94 | Ab68 CDR-L1 | RASQSISSYLN |
| SEQ ID NO: 95 | Ab68 CDR-L2 | AASSLQS |
| SEQ ID NO: 96 | Ab68 CDR-L3 | QQSYIAPYT |
| SEQ ID NO: 97 | Ab68 Heavy chain | GAGGTGCAGCTGGTGGAGTCTGGGGGAGGCTTGGT |
| | variable region (nucl) | ACAGCCAGGGCGGTCCCTGAGACTCTCCTGTACAG |
| | | CTTCTGGATTCACCTTCAGTGACCACGACATGAACT |
| | | GGGTCCGCCAGGCTCCAGGGAAGGGGCTGGAGTG |
| | | GGTTGGCCGTACTAGAAACGCCGCTGGAAGTTACAC |
| | | CACAGAATACGCCGCGTCTGTGAAAGGCAGATTCAC |
| | | CATCTCAAGAGATGATTCAAAGAACTCACTGTATCTG |
| | | CAAATGAACAGCCTGAAAACCGAGGACACGGCGGT |
| | | GTACTACTGCGCCAGAGAGCCTAAATACTGGATCGA |
| | | CTTCGACCTATGGGGGAGAGGTACCTTGGTCACCGT |
| | | CTCCTCA |
| SEQ ID NO: 98 | Ab68 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAGCAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAAGCTACATCGCCCCTTACA |
| | | CTTTTGGCGGAGGGACCAAGGTTGAGATCAAA |
| SEQ ID NO: 99 | Ab69 Heavy chain | EVQLVESGGGLVQPGGSLRLSCAASGFTFVDHDMDW |
| | variable region (e.g., | VRQAPGKGLEWVGRTRNKLGSYTTEYAASVKGRFTIS |
| | as found in HC-69) | RDDSKNSLYLQMNSLKTEDTAVYYCAREPKYWIDFDL |
| | hlgG1 backbone | WGRGTLVTVSS |
| | (CDRs in bold) | |
| SEQ ID NO: 100 | Ab69 Light chain | DIQMTQSPSSLSASVGDRVTITCRASQSISSYLNWYQQ |
| | variable region (e.g., | KPGKAPKLLIYAASSLQSGVPSRFSGSGSGTDFTLTIS |
| | as found in LC-69) | SLQFEDFATYYCQQSYIAPYTFGGGTKVEIK |
| | hKappa backbone | |
| | (CDRs in bold) | |
| SEQ ID NO: 101 | Ab69 CDR-H1 | FTFVDHDMD |
| SEQ ID NO: 102 | Ab69 CDR-H2 | RTRNKLGSYTTEYAASVKG |
| SEQ ID NO: 103 | Ab69 CDR-H3 | AREPKYWIDFDL |
| SEQ ID NO: 104 | Ab69 CDR-L1 | RASQSISSYLN |
| SEQ ID NO: 105 | Ab69 CDR-L2 | AASSLQS |
| SEQ ID NO: 106 | Ab69 CDR-L3 | QQSYIAPYT |
| SEQ ID NO: 107 | Ab69 Heavy chain | GAGGTGCAGCTGGTGGAGTCTGGGGGAGGCTTGGT |
| | variable region (nucl) | CCAGCCTGGAGGGTCCCTGAGACTCTCCTGTGCAG |
| | | CCTCTGGATTCACCTTCGTAGACCACGACATGGACT |
| | | GGGTCCGCCAGGCTCCAGGGAAGGGGCTGGAGTG |
| | | GGTTGGCCGTACTAGAAACAAACTAGGAAGTTACAC |
| | | CACAGAATACGCCGCGTCTGTGAAAGGCAGATTCAC |
| | | CATCTCAAGAGATGATTCAAAGAACTCACTGTATCTG |
| | | CAAATGAACAGCCTGAAAACCGAGGACACGGCGGT |
| | | GTACTACTGCGCCAGAGAGCCTAAATACTGGATCGA |
| | | CTTCGACCTATGGGGGAGAGGTACCTTGGTCACCGT |
| | | CTCCTCA |
| SEQ ID NO: 108 | Ab69 Light chain | GACATCCAGATGACCCAGTCTCCATCCTCCCTGTCT |
| | variable region (nucl) | GCATCTGTAGGAGACAGAGTCACCATCACTTGCCGG |
| | | GCAAGTCAGAGCATTAGCAGCTATTTAAATTGGTATC |
| | | AGCAGAAACCAGGGAAAGCCCCTAAGCTCCTGATCT |
| | | ATGCTGCATCCAGTTTGCAAAGTGGGGTCCCATCAA |
| | | GGTTCAGTGGCAGTGGATCTGGGACAGATTTCACTC |
| | | TCACCATCAGCAGTCTGCAACCTGAAGATTTTGCAA |
| | | CTTACTACTGTCAGCAAAGCTACATCGCCCCTTACA |
| | | CTTTTGGCGGAGGGACCAAGGTTGAGATCAAA |
| SEQ ID NO: 109 | Ab67 Light chain | DIQMTQSPSSLSASVGDRVTITCRASQSISSYLN |
| | LC constant region | WYQQKPGKAPKLLIYAASSLQSGVPSRFSGSGS |
| | underlined | GTDFTLTISSLQPEDFATYYCQQSYIAPYTFGGG |
| | | TKVEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGLS |
| | | SPVTKSFNRGEC |
| SEQ ID NO: 110 | Ab67 Heavy chain | EVQLVESGGGLVQPGGSLRLSCAASGFTFSDAD |
| | HC constant region | MDWVRQAPGKGLEWVGRTRNKAGSYTTEYAAS |
| | underlined | VKGRFTISRDDSKNSLYLQMNSLKTEDTAVYYCA |
| | | REPKYWIDFDLWGRGTLVTVSSASTKGPSVFPLA |
| | | PSSKSTSGGTAALGCLVKDYFPEPVTVSWNSGA |
| | | LTSGVHTFPAVLQSSGLYSLSSVVIVPSSSLGTQ |
| | | TYICNVNHKPSNTKVDKKVEPKSCDKTHTCPPCP |
| | | APELLGGPSVFLFPPKPKDTLMISRTPEVTCVVV |
| | | DVSHEDPEVKFNWYVDGVEVHNAKTKPREEQYN |
| | | STYRVVSVLTVLHQDWLNGKEYKCKVSNKALPA |
| | | PIEKTISKAKGQPREPQVYTLPPSRDELTKNQVSL |
| | | TCLVKGFYPSDIAVEWESNGQPENNYKTTPPVLD |
| | | SDGSFFLYSKLTVDKSRWQQGNVFSCSVMHEAL |
| | | HNHYTQKSLSLSPGK |
| SEQ ID NO: 111 | Ab67 Heavy chain | EVQLVESGGGLVQPGGSLRLSCAASGFTFSDAD |
| | (D265C)* | MDWVRQAPGKGLEWVGRTRNKAGSYTTEYAAS |
| | HC constant region | VKGRFTISRDDSKNSLYLQMNSLKTEDTAVYYCA |
| | underlined | REPKYWIDFDLWGRGTLVTVSSASTKGPSVFPLA |
| | | PSSKSTSGGTAALGCLVKDYFPEPVTVSWNSGA |
| | | LTSGVHTFPAVLQSSGLYSLSSVVIVPSSSLGTQ |
| | | TYICNVNHKPSNTKVDKKVEPKSCDKTHTCPPCP |
| | | APELLGGPSVFLFPPKPKDTLMISRTPEVTCVVV |
| | | CVSHEDPEVKFNWYVDGVEVHNAKTKPREEQYN |
| | | STYRVVSVLTVLHQWLNGKEYKCKVSNKALPA |
| | | PIEKTISKAKGQPREPQVYTLPPSRDELTKNQVSL |
| | | TCLVKGFYPSDIAVEWESNGQPENNYKTTPPVLD |
| | | SDGSFFLYSKLTVDKSRWQQGNVFSCSVMHEAL |
| | | HNHYTQKSLSLSPGK |
| SEQ ID NO: 112 | Ab67 Heavy chain | EVQLVESGGGLVQPGGSLRLSCAASGFTFSDAD |
| | (L234A/L235A/ | MDWVRQAPGKGLEWVGRTRNKAGSYTTEYAAS |
| | D265C)* | VKGRFTISRDDSKNSLYLQMNSLKTEDTAVYYCA |
| | HC constant region | REPKYWIDFDLWGRGTLVTVSSASTKGPSVFPLA |
| | underlined | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | CVSHEDPEVKFNWYVDGVEVHNAKTKPREEQYN |
| | | STYRVVSVLTVLHQDWLNGKEYKCKVSNKALPA |
| | | PIEKTISKAKGQPREPQVYTLPPSRDELTKNQVSL |
| | | TCLVKGFYPSDIAVEWESNGQPENNYKTTPPVLD |
| | | SDGSFFLYSKLTVDKSRWQQGNVFSCSVMHEAL |
| | | HNHYTQKSLSLSPGK |
| SEQ ID NO: 113 | Ab67 Heavy chain | EVQLVESGGGLVQPGGSLRLSOAASGFTFSDAD |
| | (D265C/H435A)* | MDWVRQAPGKGLEWVGRTRNKAGSYTTEYAAS |
| | HC constant region | VKGRFTISRDDSKNSLYLQMNSLKTEDTAVYYCA |
| | underlined | REPKYWIDFDLWGRGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHTCPPCP |
| | | APELLGGPSVFLFPPKPKDTLMISRTPEVTCVVV |
| | | CVSHEDPEVKFNWYVDGVEVHNAKTKPREEQYN |
| | | STYRVVSVLTVLHQDWLNGKEYKCKVSNKALPA |
| | | PIEKTISKAKGQPREPQVYTLPPSRDELTKNQVSL |
| | | TCLVKGFYPSDIAVEWESNGQPENNYKTTPPVLD |
| | | SDGSFFLYSKLTVDKSRWQQGNVFSCSVMHEAL |
| | | HNAYTQKSLSLSPGK |
| SEQ ID NO: 114 | Ab67 Heavy chain | EVQLVESGGGLVQPGGSLRLSCAASGFTFSDAD |
| | (L234A/L235A/ | MDWVRQAPGKGLEWVGRTRNKAGSYTTEYAAS |
| | D265C/H435A)* | VKGRFTISRDDSKNSLYLQMNSLKTEDTAVYYCA |
| | HC constant region | REPKYWIDFDLWGRGTLVTVSSASTKGPSVFPLA |
| | underlined | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHTCPPCP |
| | | APEAAGGPSVFLFPPKPKDTLMISRTPEVTCVVV |
| | | CVSHEDPEVKFNWYVDGVEVHNAKTKPREEQYN |
| | | STYRVVSVLTVLHQDWLNGKEYKCKVSNKALPA |
| | | PIEKTISKAKGQPREPQVYTLPPSRDELTKNQVSL |
| | | TCLVKGFYPSDIAVEEESNGQPENNYKTTPPVLD |
| | | SDGSFFLYSKLTVDKSRWQQGNVFSCSVMHEAL |
| | | HNAYTQKSLSLSPGK |
| SEQ ID NO: 115 | Ab55 Light chain | DIQMTQSPSSLSASVGDRVTITCRASQSINSYLNWYQQ |
| | LC constant region | KPGKAPKLLIYAASSLQSGVPSRFSGSGSGTDFTLTISS |
| | underlined | LQPEDFATYYCQQGVSDITFGGGTKVEIKRTVAAPSV |
| | | FIFPPSDEQLKSGTASVVCLLNNFYPREAKVQWK |
| | | VDNALQSGNSQESVTEQDSKDSTYSLSSTLTLSK |
| | | ADYEKHKVYACEVTHQGLSSPVTKSFNRGEC |
| SEQ ID NO: 116 | Ab55 Heavy chain | QVQLVQSGAEVKKPGSSVKVSCKASGGTFRIYAISWV |
| | HC constant region | RQAPGQGLEWMGGIIPDFGVANYAQKFQGRVTITADE |
| | underlined | STSTAYMELSSLRSEDTAVYYCARGGLDTDEFDLWGR |
| | | GTLVTVSSASTKGPSVFPLAPSSKSTSGGTAALGC |
| | | LVKDYFPEPVTVSWNSGALTSGVHTFPAVLQSS |
| | | GLYSLSSVVTVPSSSLGTQTYICNVNHKPSNTKV |
| | | DKKVEPKSCDKTHTCPPCPAPELLGGPSVFLFPP |
| | | KPKDTLMISRTPEVTCVVVDVSHEDPEVKFNWYV |
| | | DGVEVHNAKTKPREEQYNSTYRVVSVLTVLHQD |
| | | WLNGKEYKCKVSNKALPAPIEKTISKAKGQPREP |
| | | QVYTLPPSRDELTKNQVSLTCLVKGFYPSDIAVE |
| | | WESNGQPENNYKTTPPVLDSDGSFFLYSKLTVD |
| | | KSRWQQGNVFSCSVMHEALHNHYTQKSLSLSP |
| | | GK |
| SEQ ID NO: 117 | Ab55 Heavy chain | QVQLVQSGAEVKKPGSSVKVSCKASGGTFRIYAISWV |
| | (D265C)* | RQAPGQGLEWMGGIIPDFGVANYAQKFQGRVTITADE |
| | HC constant region | STSTAYMELSSLRSEDTAVYYCARGGLDTDEFDLWGR |
| | underlined | GTLVTVSSASTKGPSVFPLAPSSKSTSGGTAALGC |
| | | LVKDYFPEPVTVSWNSGALTSGVHTFPAVLQSS |
| | | GLYSLSSVVTVPSSSLGTQTYICNVNHKPSNTKV |
| | | DKKVEPKSCDKTHTCPPCPAPELLGGPSVFLFPP |
| | | KPKDTLMISRTPEVTCVVVCVSHEDPEVKFNWYV |
| | | DGVEVHNAKTKPREEQYNSTYRVVSVLTVLHQD |
| | | WLNGKEYKCKVSNKALPAPIEKTISKAKGQPREP |
| | | QVYTLPPSRDELTKNQVSLTCLVKGFYPSDIAVE |
| | | WESNGQPENNYKTTPPVLDSDGSFFLYSKLTVD |
| | | KSRWQQGNVFSCSVMHEALHNHYTQKSLSLSP |
| | | GK |
| SEQ ID NO: 118 | Ab55 Heavy chain | QVQLVQSGAEVKKPGSSVKVSCKASGGTFRIYAISWV |
| | (L234A/L235A/ | RQAPGQGLEWMGGIIPDFGVANYAQKFQGRVTITADE |
| | D285C)* | STSTAYMELSSLRSEDTAVYYCARGGLDTDEFDLWGR |
| | HC constant region | GTLVTVSSASTKGPSVFPLAPSSKSTSGGTAALGC |
| | underlined | LVKDYFPEPVTVSWNSGALTSGVHTFPAVLQSS |
| | | GLYSLSSVVTVPSSSLGTQTYICNVNHKPSNTKV |
| | | DKKVEPKSCDKTHTCPPCPAPEAAGGPSVFLFP |
| | | PKPKDTLMISRTPEVTCVVVCVSHEDPEVKFNWY |
| | | VDGVEVHNAKTKPREEQYNSTYRVVSVLTVLHQ |
| | | DWLNGKEYKCKVSNKALPAPIEKTISKAKGQPRE |
| | | PQVYTIPPSRDELTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSLSP |
| | | GK |
| SEQ ID NO: 119 | Ab55 Heavy chain | QVQLVQSGAEVKKPGSSVKVSCKASGGTFRIYAISWV |
| | (D265C/H435A)* | RQAPGQGLEWMGGIIPDFGVANYAQKFQGRVTITADE |
| | HC constant region | STSTAYMELSSLRSEDTAVYYCARGGLDTDEFDLWGR |
| | underlined | GTLVTVSSASTKGPSVFPLAPSSKSTSGGTAALGC |
| | | LVKDYFPEPVTVSWNSGALTSGVHTFPAVLQSS |
| | | GLYSLSSVVTVPSSSLGTQTYICNVNHKPSNTKV |
| | | DKKVEPKSCDKTHTCPPCPAPELLGGPSVFLFPP |
| | | KPKDTLMISRTPEVTCVVVCVSHEDPEVKFNWYV |
| | | DGVEVHNAKTKPREEQYNSTYRVVSVLTVLHQD |
| | | WLNGKEYKCKVSNKALPAPIEKTISKAKGQPREP |
| | | QVYTLPPSRDELTKNQVSLTCLVKGFYPSDIAVE |
| | | WESNGQPENNYKTTPPVLDSDGSFFLYSKLTVD |
| | | KSRWQQGNVFSCSVMHEALHNAYTQKSLSLSP |
| | | GK |
| SEQ ID NO: 120 | Ab55 Heavy chain | QVQLVQSGAEVKKPGSSVKVSCKASGGTFRIYAISWV |
| | (L234A/L235A/ | RQAPGQGLEWMGGIIPDFGVANYAQKFQGRVTITADE |
| | D265C/H435A)* | STSTAYMELSSLRSEDTAVYYCARGGLDTDEFDLWGR |
| | HC constant region | GTLVTVSSASTKGPSVFPLAPSSKSTSGGTAALGC |
| | underlined | LVKDYFPEPVTVSWNSGALTSGVHTFPAVLQSS |
| | | GLYSLSSVVTVPSSSLGTQTYICNVNHKPSNTKV |
| | | DKKVEPKSCDKTHTCPPCPAPEAAGGPSVFLFP |
| | | PKPKDTLMISRTPEVTCVVVCVSHEDPEVKFNWY |
| | | VDGVEVHNAKTKPREEQYNSTYRVVSVLTVLHQ |
| | | DWLNGKEYKCKVSNKALPAPIEKTISKAKGQPRE |
| | | PQVYTLPPSRDELTKNQVSLTCLVKGFYPSDIAV |
| | | EWESNGQPENNYKTTPPVLDSDGSFFLYSKLTV |
| | | DKSRWQQGNVFSCSVMHEALHNAYTQKSLSLSP |
| | | GK |
| SEQ ID NO: 121 | Light chain constant | RTVAAPSVFIFPPSDEQLKSGTASVVCLLNNFYP |
| | region of LC-54, LC- | REAKVQWKVDNALQSGNSQESVTEQDSKDSTY |
| | 55, LC-56, LC-57, LC- | SLSSTLTLSKADYEKHKVYACEVTHQGLSSPVTK |
| | 58, LC-61, LC-66, LC- | SFNRGEC |
| | 67, LC-68, LC-69 | |
| SEQ ID NO: 122 | Heavy chain constant | ASTKGPSVFPLAPSSKSTSGGTAALGCLVKDYFP |
| | region of WT | EPVTVSWNSGALTSGVHTFPAVLQSSGLYSLSS |
| | | VVTVPSSSLGTQTYICNVNHKPSNTKVDKKVEPK |
| | | SCDKTHTCPPCPAPELLGGPSVFLFPFKRKDTLM |
| | | ISRTPEVTCVVVDVSHEDPEVKFNWYVDGVEVH |
| | | NAKTKPREEQYNSTYRVVSVLTVLHQDWLNGKE |
| | | YKCKVSNKALPAPIEKTISKAKGQPREPQVYTLPP |
| | | SRDELTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| SEQ ID NO: 123 | Heavy chain constant | ASTKGPSVFPLAPSSKSTSGGTAALGCLVKDYFP |
| | region (D265C)* | EPVTVSWNSGALTSGVHTFPAVLQSSGLYSLSS |
| | | VVTVPSSSLGTQTYICNVNHKPSNTKVDKKVEPK |
| | | SCDKTHTCPPCPAPELLGGPSVFLFPPKPKDTLM |
| | | ISRTPEVTCVVVCVSHEDPEVKFNWYVDGVEVH |
| | | NAKTKPREEQYNSTYRVVSVLTVLHQDWLNGKE |
| | | YKCKVSNKALPAPIEKTISKAKGQPREPQVYTLPP |
| | | SRDELTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| SEQ ID NO: 124 | Heavy chain constant | ASTKGPSVFPLAPSSKSTSGGTAALGCLVKDYFP |
| | region (L234A/L235A/ | EPVTVSWNSGALTSGVHTFPAVLQSSGLYSLSS |
| | D265C)* | VVTVPSSSLGTQTYICNVNHKPSNTKVDKKVEPK |
| | | SCDKTHTCPPCPAPEAAGGPSVFLFPPKPKDTL |
| | | MISRTPEVTCVVVCVSHEDPEVKFNWYVDGVEV |
| | | HNAKTKPREEQYNSTYRVVSVLTVLHQDWLNGK |
| | | EYKCKVSNKALPAPIEKTISKAKGQPREPQVYTLP |
| | | PSRDELTKNQVSLTCLVKGFYPSDIAVEWESNG |
| | | QPENNYKTTPPVLDSDGSFFLYSKLTVDKSRWQ |
| | | QGNVFSCSVMHEALHNHYTQKSLSLSPGK |
| SEQ ID NO: 125 | Heavy chain constant | ASTKGPSVFPLAPSSKSTSGGTAALGCLVKDYFP |
| | region (H435A/ | EPVTVSWNSGALTSGVHTFPAVLQSSGLYSLSS |
| | D265C)* | VVTVPSSSLGTQTYICNVNHKPSNTKVDKKVEPK |
| | | SCDKTHTCPPCPAPELLGGPSVFLFPPKPKDTLM |
| | | ISRTPEVICVVVCVSHEDPEVKFNWYVDGVEVH |
| | | NAKTKPREEQYNSTYRVVSVLTVLHQDWLNGKE |
| | | YKCKVSNKALPAPIEKTISKAKGQPREPQVYTLPP |
| | | SRDELTKNQVSLTCLVKGFYPSDIAVEWESNGQ |
| | | PENNYKTTPPVLDSDGSFFLYSKLTVDKSRWQQ |
| | | GNVFSCSVMHEALHNAYTQKSLSLSPGK |
| SEQ ID NO: 126 | Heavy chain constant | ASTKGPSVFPLAPSSKSTSGGTAALGCLVKDYFP |
| | region (L234A/ | EPVTVSWNSGALTSGVHTFPAVLQSSGLYSLSS |
| | L235A/H435A/ | VVTVPSSSLGTQTYICNVNHKPSNTKVDKKVEPK |
| | D265C)* | SCDKTHTCPPCPAPEAAGGPSVFLFPPKPKDTL |
| | | MISRTPEVTCVVVCVSHEDPEVKFNWYVDGVEV |
| | | HNAKTKPREEQYNSTYRVVSVLTVLHQDWLNGK |
| | | EYKCKVSNKALPAPIEKTISKAKGQPREPQVYTLP |
| | | PSRDELTKNQVSLTCLVKGFYPSDIAVEWESNG |
| | | QPENNYKTTPPVLDSDGSFFLYSKLTVDKSRWQ |
| | | QGNVFSCSVMHEALHNAYTQKSLSLSPGK |
| SEQ ID NO: 127 | Consensus sequence | GTF(S/R)(S/I/L)YAIS |
| | of variable heavy chain | |
| | CDR1 (Abs 54-57) | |
| SEQ ID NO: 128 | Consensus sequence | GIIP(I/D/A/H)FG(T/V/L)ANYAQKFQG |
| | of variable heavy chain | |
| | CDR2 (Abs 54-57) | |
| SEQ ID NO: 129 | Variable heavy chain | ARGGLDTDEFDL |
| | CDR3 (Abs 54-57) | |
| SEQ ID NO: 130 | Variable light chain | RASQSINSYLN |
| | CDR1 (Abs 54-57) | |
| SEQ ID NO: 131 | Variable light chain | AASSLQS |
| | CDR2 (Abs 54-57) | |
| SEQ ID NO: 132 | Variable light chain | QQGVSDIT |
| | CDR3 (Abs 54-57) | |
| SEQ ID NO: 133 | Consensus sequence | FTFS(N/S)Y(A/V)M(S/I) |
| | of variable heavy chain | |
| | CDR1 (Abs 58, 61) | |
| SEQ ID NO: 134 | Consensus sequence | (A/S)ISG(S/D)(G/S)(G/V)(S/T)TYYADSVKG |
| | of variable heavy chain | |
| | CDR2 (Abs 58, 61) | |
| SEQ ID NO: 135 | Variable heavy chain | AKGPPTYHTNYYYMDV |
| | CDR3 (Abs 58, 61) | |
| SEQ ID NO: 136 | Variable light chain | RASQGISSWLA |
| | CDR1 (Abs 58, 61) | |
| SEQ ID NO: 137 | Variable light chain | AASSLQS |
| | CDR2 (Abs 58, 61) | |
| SEQ ID NO: 138 | Variable light chain | QQTNSFPYT |
| | CDR3 (Abs 58, 61) | |
| SEQ ID NO: 139 | Consensus sequence | FTF(S/V)D(H/A)(Y/D)M(D/N) |
| | of variable heavy chain | |
| | CDR1 (Abs 66-69) | |
| SEQ ID NO: 140 | Consensus sequence | RTRN(K/A)(A/L)(S/G)SYTTEYAASVKG |
| | of variable heavy chain | |
| | CDR2 (Abs 66-69) | |
| SEQ ID NO: 141 | Variable heavy chain | AREPKYWIDFDL |
| | CDR3 (Abs 66-69) | |
| SEQ ID NO: 142 | Variable light chain | RASQSISSYLN |
| | CDR1 (Abs 66-69) | |
| SEQ ID NO: 143 | Variable light chain | AASSLQS |
| | CDR2 (Abs 66-69) | |
| SEQ ID NO: 144 | Variable light chain | QQSYIAPYT |
| | CDR3 (Abs 66-69) | |
| SEQ ID NO: 145 | Human CD117 | MRGARGAWDFLCVLLLLLRVQTGSSQPSVSPGE |
| | (mast/stem cell growth | PSPPSIHPGKSDLIVRVGDEIRLLCTDPGFVKWTF |
| | factor receptor Kit | EILDETNENKQNEWITEKAEATNTGKYTCTNKHG |
| | isoform 1 precursor) | LSNSIYVFVRDPAKLFLVDRSLYGKEDNDTLVRC |
| | Protein NCBI | PLTDPEVTNYSLKGCQGKPLPKDLRFIPDPKAGI |
| | Reference Sequence: | MIKSVKRAYHRLCLHCSVDQEGKSVLSEKFILKV |
| | NP_000213.1 | RPAFKAVPVVSVSKASYLLREGEEFTVTCTIKDV |
| | | SSSVYSTWKRENSQTKLQEKYNSWHHGDFNYE |
| | | RQATLTISSARVNDSGVFMCYANNTFGSANVTTT |
| | | LEVVDKGFINIFPMINTTVFVNDGENVDLIVEYEAF |
| | | PKPEHQQWIYMNRTFTDKWEDYPKSENESNIRY |
| | | VSELHLTRLKGTEGGTYTFLVSNSDVNAAIAFNV |
| | | YVNTKPEILTYDRLVNGMLQCVAAGFPEPTIDWY |
| | | FCPGTEQRCSASVLPVDVQIINSSGPPFGKLVV |
| | | QSSIDSSAFKHNGTVECKAYNDVGKTSAYFNFAF |
| | | KGNNKEQIHPHTLFTPLLIGFVIVAGMMCIIVMILT |
| | | YKYLQKPMYEVQWKVVEEINGNNYVYIDPTQLPY |
| | | DHKWEFPRNRLSFGKTLGAGAFGKVVEATAYGLI |
| | | KSDAAMTVAVKMLKPSAHLTEREALMSELKVLSY |
| | | LGNHMNIVNLLGACTIGGPTLVITEYCCYGDLLNF |
| | | LRRKRDSFICSKQEDHAEAALYKNLLHSKESSCS |
| | | DSTNEYMDMKPGVSYVVPTKADKRRSVRIGSYIE |
| | | RDVTPAIMEDDELALDLEDLLSFSYQVAKGMAFL |
| | | ASKNCIHRDLAARNILLTHGRITKICDFGLARDIKN |
| | | DSNYVVKGNARLPVKWMAPESIFNCVYTFESDV |
| | | WSYGIFLWELFSLGSSPYPGMPVDSKFYKMIKEG |
| | | FRMLSPEHAPAEMYDIMKTCWDADPLKRPTFKQI |
| | | VQLIEKQISESTNHIYSNLANCSPNRQKPVVDHSV |
| | | RINSVGSTASSSQPLLVHDDV |
| SEQ ID NO: 146 | Human CD117 | MRGARGAWDFLCVLLLLLRVQTGSSQPSVSPGE |
| | (mast/stem cell growth | PSPPSIHPGKSDLIVRVGDEIRLICTDPGFVKWTF |
| | factor receptor Kit | EILDETNENKQNEWITEKAEATNTGKYTCTNKHG |
| | isoform 2 precursor) | LSNSIYVFVRDPAKLFLVDRSLYGKEDNDTLVRC |
| | Protein NCBI | PLTDPEVTNYSLKGCQGKPLPKDLRFIPDPKAGI |
| | Reference Sequence: | MIKSVKRAYHRLCLHCSVDQEGKSVLSEKFILKV |
| | NP_001087241.1 | RPAFKAVPVVSVSKASYLLREGEEFTVTCTIKDV |
| | | SSSVYSTWKRENSQTKLQEKYNSWHHGDFNYE |
| | | RQATLTISSARVNDSGVFMCYANNTFGSANVTTT |
| | | LEVVDKGFINIFPMINTTVFVNDGENVDLIVEYEAF |
| | | PKPEHQQWIYMNRTFTDKWEDYPKSENESNIRY |
| | | VSELHLTRLKGTEGGTYTFLVSNSDVNAAIAFNV |
| | | YVNTKPEILTYDRLVNGMLQCVAAGFPEPTIDWY |
| | | FCPGTEQRCSASVLPVDVQTLNSSGPPFGKLVV |
| | | QSSIDSSAFKHNGTVECKAYNDVGKTSAYFNFAF |
| | | KEQIHPHTLFTPLLIGFVIVAGMMCIIVMILTYKYL |
| | | QKPMYEVQWKVVEEINGNNYVYIDPTQLPYDHK |
| | | WEFPRNRLSFGKTLGAGAFGKVVEATAYGLIKSD |
| | | AAMTVAVKMLKPSAHLTEREALMSELKVLSYLGN |
| | | HMNIVNLLGACTIGGPTLVITEYCCYGDLLNFLRR |
| | | KRDSFICSKQEDHAEAALYKNLLHSKESSCSDST |
| | | NEYMDMKPGVSYVVPTKADKRRSVRIGSYIERD |
| | | VTPAIMEDDELALDLEDLLSFSYQVAKGMAFLAS |
| | | KNCIHRDLAARNILLTHGRITKICDFGLARDIKNDS |
| | | NYVVKGNARLPVKWMAPESIFNCVYTFESDVWS |
| | | YGIFLWELFSLGSSPYPGMPVDSKFYKMIKEGFR |
| | | MLSPEHAPAEMYDIMKTCWDADPLKRPTFKQIV |
| | | QLIEKQISESTNHIYSNLANCSPNRQKPVVDHSV |
| | | RINSVGSTASSSQPLLVHDDV |
| SEQ ID NO: 147 | Heavy chain variable | QVQLVQSGAAVKKPGESLKISCKGSGYRFTTYWIGWV |
| | region of HC-1 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 148 | Light chain variable | AIQLTOSPSSLSASVGDRVTITCRASQGVSSALAWYQQ |
| | region of LC-1 | KPGKAPKLLIYDASSLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-2 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 149 | Light chain variable | DIQLTQSPSSLSASVGDRVTITCRASQGIRTDLGWYQQ |
| | region of LC-2 | KPGKAPKLLIYDASSLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-3 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 150 | Light chain variable | AIRMTQSPSSLSASVGDRVTITCRASQGIRNDLAWYQQ |
| | region of LC-3 | KPGKTPKLLIYDASSLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-4 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 151 | Light chain variable | AIQMTQSPSSLSASVGDRVTITCRASQGIRNDLGWYQ |
| | region of LC-4 | QKPGKAPKLLIYDASSLESGVPSRFSGSGSGTDFTLTIS |
| | | SLQPEDFATYYCQQFNSYPLTFGGGTKVDIK |
| SEQ ID NO: 147 | Heavy chain variable | QVQLVQSGAAVKKPGESLKISCKGSGYRFTTYWIGWV |
| | region of HC-5 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 152 | Light chain variable | NIQMTQSPSSLSASVGDRVTITCRASQAISDYLAWFQQ |
| | region of LC-5 | KPGKAPKLLIYDASNLETGVPSRFSGSGSGTDFTLTISS |
| | | LQPEDFATYYCQQLNSYPLTFGGGTKVEIK |
| SEQ ID NO: 147 | Heavy chain variable | QVQLVQSGAAVKKPGESLKISCKGSGYRFTTYWIGWV |
| | region of HC-6 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 153 | Light chain variable | AIRMTQSPSSLSASVGDRVIIACRASQGIGGALAWYQQ |
| | region of LC-6 | KPGNAPKVLVYDASTLESGVPSRFSGGGSGTDFTLTIS |
| | | SLQPEDFATYYCQQFNSYPLTFGGGTKLEIK |
| SEQ ID NO: 147 | Heavy chain variable | QVQLVQSGAAVKKPGESLKISCKGSGYRFTTYWIGWV |
| | region of HC-7 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 154 | Light chain variable | DIAMTQSPPSLSAFVGDRVTITCRASQGIISSLAWYQQ |
| | region of LC-7 | KPGKAPKLLIYDASSLESGVPSRFSGSGSGTDFTLTIRS |
| | | LQPEDFATYYCQQFNSYPLTFGGGTKLEIK |
| SEQ ID NO: 147 | Heavy chain variable | QVQLVQSGAAVKKPGESLKISCKGSGYRFTTYWIGWV |
| | region of HC-8 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 155 | Light chain variable | DIQMTQSPSSLSASVGDRVTITCRASQGISSALAWYQQ |
| | region of LC-8 | KAGKAPKVLISDASSLESGVPSRFSGSGSGTDFTLSIS |
| | | SLQPEDFATYYCQQFNGYPLTFGGGTKVDIK |
| SEQ ID NO: 147 | Heavy chain variable | QVQLVQSGAAVKKPGESLKISCKGSGYRFTTYWIGWV |
| | region of HC-9 amino | RQMPGKGLEWMGIIYPGDSDTRYSPSFQGQVTISAGK |
| | acid sequence | SISTAYLQWSSLKASDTAMYYCARHGRGYNGYEGAFD |
| | | IWGQGTMVTVSS |
| SEQ ID NO: 156 | Light chain variable | AIRMTQSPSSLSASVGDRVTITCQASQGIRNDLGWYQ |
| | region of LC-9 | QKPGKAPKLLIYDASNLETGVPSRFSGSGSGTDFTFTI |
| | | SSLQPEDIATYYCQQFNSYPLTFGGGTKLEIK |
| SEQ ID NO: 147 | Heavy chain variable | QVQLVQSGAAVKKPGESLKISCKGSGYRFTTYWIGWV |
| | region of HC-10 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 157 | Light chain variable | NIQMTQSPSSLSTSVGDRVTITCRASQGIGTSLAWYQQ |
| | region of LC-10 | KPGKPPKLLIYDASSLESGVPSRLSGSGSGTDFTLTISS |
| | | LQPEDFATYYCQQSNSYPITFGQGTRLEIK |
| SEQ ID NO: 147 | Heavy chain variable | QVQLVQSGAAVKKPGESLKISCKGSGYRFTTYWIGWV |
| | region of HC-11 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 158 | Light chain variable | AIQLTQSPSSLSASVGDRVTITCRASQSIGDYLTWYQQ |
| | region of LC-11 | KPGKAPKVLIYGASSLQSGVPPRFSGSGSGTDFTLTVS |
| | | SLQPEDFATYYCQQLNSYPLTFGGGTKLEIK |
| SEQ ID NO: 147 | Heavy chain variable | QVQLVQSGAAVKKPGESLKISCKGSGYRFTTYWIGWV |
| | region of HC-12 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 159 | Light chain variable | DIQLTQSPSSLSASVGDRVTITCRASQGVRSTLAWYQ |
| | region of LC-12 | QKPGKAPKLLIYDASILESGVPSRFSGSGSGTDFTLTIS |
| | | SLQPEDFATYYCQQFNGYPLTFGQGTRLEIK |
| SEQ ID NO: 147 | Heavy chain variable | QVQLVQSGAAVKKPGESLKISCKGSGYRFTTYWIGWV |
| | region of HC-13 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 160 | Light chain variable | DIVMTQSPSSLSASVGDRVTITCRASQGIRNDLGWYQ |
| | region of LC-13 | QKPGKAPKLLIYDASSLESGVPSRFSGSGSGTDFTLTIS |
| | | SLQPEDFATYYCQQFNSYPLTFGGGTKLEIK |
| SEQ ID NO: 147 | Heavy chain variable | QVQLVQSGAAVKKPGESLKISCKGSGYRFTTYWIGWV |
| | region of HC-14 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 161 | Light chain variable | DIQLTQSPSSLSASVGDRVTITCRASQGISSFLAWYQQ |
| | region of LC-14 | KPGKAPKLLIYDASTLQSGVPSRFSGSASGTDFTLTISS |
| | | LQPEDFATYYCQQLNGYPLTFGGGTKVEIK |
| SEQ ID NO: 147 | Heavy chain variable | QVQLVQSGAAVKKPGESLKISCKGSGYRFTTYWIGWV |
| | region of HC-15 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 162 | Light chain variable | AIQLTQSPSSLSASVGDRVTITCRASQGIGSALAWYQQ |
| | region of LC-15 | KPGIGPKLLIYDASTLESGVPARFSGSGSRIDFTLTITSL |
| | | QPEDFATYYCQQFNGYPLTFGGGTKLEIK |
| SEQ ID NO: 147 | Heavy chain variable | QVQLVQSGAAVKKPGESLKISCKGSGYRFTTYWIGWV |
| | region of HC-16 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 163 | Light chain varabe | AIQLTQSPSSLSASVGDRVTITCRASQGITSALAWYQE |
| | region of LC-16 | KPGKAPNLLIYDASSLESGVPSRFSGSGYGTDFTLTISS |
| | | LQPEDFATYYCQQLNSYPLTFGGGTKVDIK |
| SEQ ID NO: 164 | Heavy chain variable | QIQLVQSGPELRKPGESVKISCKASGYTFTDYAMYWV |
| | region of HC-17 | KQAPGKGLKWMGWINTYTGKPTYADDFKGRFVFSLEA |
| | | SANTANLQISNLKNEDTATYFCARARGLVDDYVMDAW |
| | | GQGTSVTVSS |
| SEQ ID NO: 165 | Light chain variable | SYELIQPPSASVTLGNTVSLTCVGDELSKRYAQWYQQ |
| | region of LC-17 | KPDKTIVSVIYKDSERPSGISDRFSGSSSGTTATLTIHG |
| | | TLAEDEADYYCLSTYSDDNLPVFGGGTKLTVL |
| SEQ ID NO: 166 | Heavy chain variable | EVQLQQYGAELGKPGTSVRLSCKVSGYNIRNTYIHWV |
| | region of HC-18 | NQRPGEGLEWIGRIDPINGNTISAEKFKTKATLTADTS |
| | | SHTAYLQFSQLKSDDTAIYFCALNYEGYADYWGQGVM |
| | | VTGSS |
| SEQ ID NO: 167 | Light chain variable | DIQMTQSPSFLSASVGDRVTINCKASQNINKYLNWYQQ |
| | region of LC-18 | KVGEAPKRLIFKTNSLQTGIPSRFSGSGSGTDYTLTISS |
| | | LQTEDVATYFCFQYNIGYTFGAGTKVELK |
| SEQ ID NO: 168 | Heavy chain variable | EVQLQESGPGLVKPSQSLSLTCSVTGYSISSNYRWNW |
| | region of HC-19 | IRKFPGNKVEWMGYINSAGSTNYNPSLKSRISMTRDTS |
| | | KNQFFLQVNSVTTEDTATYYCARSLRGYITDYSGFFDY |
| | | WGQGVMVTVSS |
| SEQ ID NO: 169 | Light chain variable | DIRMTQSPASLSASLGETVNIECLASEDIFSDLAWYQQ |
| | region of LC-19 | KPGKSPQLLIYNANSLQNGVPSRFSGSGSGTRYSLKIN |
| | | SLQSEDVATYFCQQYKNYPLTFGSGTKLEIK |
| SEQ ID NO: 170 | Heavy chain variable | EVQLQQYGAELGKPGTSVRLSCKLSGYKIRNTYIHWV |
| | region of HC-20 | NQRPGKGLEWIGRIDPANGNTIYAEKFKSKVTLTADTS |
| | | SNTAYMQLSQLKSDDTALYFCAMNYEGYEDYWGQGV |
| | | MVTVSS |
| SEQ ID NO: 171 | Light chain variable | DIQMTQSPSFLSASVGDSVTINCKASQNINKYLNWYQQ |
| | region of LC-20 | KLGEAPKRLIHKTDSLQTGIPSRFSGSGSGTDYTLTISS |
| | | LQPEDVATYFCFQYKSGFMFGAGTKLELK |
| SEQ ID NO: 172 | Heavy chain variable | QIQLVQSGPELKKPGESVKISCKASGYTFTDYAVYWVI |
| | region of HC-21 | QAPGKGLKWMGWINTYTGKPTYADDFKGRFVFSLETS |
| | | ASTANLQISNLKNEDTATYFCARGAGMTKDYVMDAWG |
| | | RGVLVTVS |
| SEQ ID NO: 173 | Light chain variable | SYELIQPPSASVTLGNTVSLTCVGDELSKRYAQWYQQ |
| | region of LC-21 | KPDKTIVSVIYKDSERPSDISDRFSGSSSGTTATLTIHGT |
| | | LAEDEADYYCLSTYSDDNLPVFGGGTKLTVL |
| SEQ ID NO: 174 | Heavy chain variable | QVQLKESGPGLVQPSQTLSLTCTVSGFSLTSYLVHWV |
| | region of HC-22 | RQPPGKTLEWVGLMWNDGDTSYNSALKSRLSISRDTS |
| | | KSQVFLKMHSLQAEDTATYYCARESNLGFTYWGHGTL |
| | | VTVSS |
| SEQ ID NO: 175 | Light chain variable | DIQMTQSPASLSASLEEIVTITCKASQGIDDDLSWYQQK |
| | region of LC-22 | PGKSPQLLIYDVTRLADGVPSRFSGSRSGTQYSLKISR |
| | | PQVADSGIYYCLQSYSTPYTFGAGTKLELK |
| SEQ ID NO: 176 | Heavy chain variable | EVQLQQYGAELGKPGTSVRLSCKVSGYNIRNTYIHWV |
| | region of HC-23 | HQRPGEGLEWIGRIDPTNGNTISAEKFKSKATLTADTS |
| | | SNTAYMQFSQLKSDDTAIYFCAMNYEGYADYWGQGV |
| | | MVTVSS |
| SEQ ID NO: 177 | Light chain variable | DIQMTQSPSFLSASVGDRLTINCKASQNINKYLNWYQQ |
| | region of LC-23 | KLGEAPKRLIFKTNSLQTGIPSRFSGSGSGTDYILTISS |
| | | LQPEDVATYFCFQYNIGFTFGAGTKLELK |
| SEQ ID NO: 178 | Heavy chain variable | EVQLVESGGGLVQSGRSLKLSCAASGFTVSDYYMAW |
| | region of HC-24 | VRQAPTKGLEWVATINYDGSTTYHRDSVKGRFTISRD |
| | | NAKSTLYLQMDSLRSEDTATYYCARHGDYGYHYGAYY |
| | | FDYWGQGVMVTVSS |
| SEQ ID NO: 179 | Light chain variable | DIVLTQSPALAVSLGQRATISCRASQTVSLSGYNLIHWY |
| | region of LC-24 | QQRTGQQPKLLIYRASNLAPGIPARFSGSGSGTDFTLTI |
| | | SPVQSDDIATYYCQQSRESWTFGGGTNLEMK |
| SEQ ID NO: 180 | Heavy chain variable | QIQLVQSGPELKKPGESVKISCKASGYTFTDYAIHWVK |
| | region of HC-25 | QAPGQGLRWMAWINTETGKPTYADDFKGRFVFSLEA |
| | | SASTAHLQISNLKNEDTATFFCAGGSHWFAYWGQGTL |
| | | VTVSS |
| SEQ ID NO: 181 | Light chain variable | SYELIQPPSASVTLENTVSITCSGDELSNKYAHWYQQK |
| | region of LC-25 | PDKTILEVIYNDSERPSGISDRFSGSSSGTTAILTIRDAQ |
| | | AEDEADYYCLSTFSDDDLPIFGGGTKLTVL |
| SEQ ID NO: 172 | Heavy chain variable | QIQLVQSGPELKKPGESVKISCKASGYTFTDYAVYWVI |
| | region of HC-28 | QAPGKGLKWMGWINTYTGKPTYADDFKGRFVFSLETS |
| | | ASTANLQISNLKNEDTATYFCARGAGMTKDYVMDAWG |
| | | RGVLVTVS |
| SEQ ID NO: 182 | Light chain variable | SYELIQPPSTSVTLGNTVSLTCVGNELPKRYAYWFQQK |
| | region of LC-26 | PDQSIVRLIYDDDRRPSGISDRFSGSSSGTTATLTIRDA |
| | | QAEDEAYYYCHSTYTDDKVPIFGGGTKLTVL |
| SEQ ID NO: 183 | Heavy chain variable | EVQLVESGGGLVQPGRSMKLSCKASGFTFSNYDMAW |
| | region of HC-27 | VRQAPTRGLEWVASISYDGITAYYRDSVKGRFTISREN |
| | | AKSTLYLQLVSLRSEDTATYYCTTEGGYVYSGPHYFDY |
| | | WGQGVMVTVSS |
| SEQ ID NO: 184 | Light chain variable | DIQMTQSPSSMSVSLGDTVTITCRASQDVGIFVNWFQ |
| | region of LC-27 | QKPGRSPRRMIYRATNLADGVPSRFSGSRSGSDYSLT |
| | | ISSLESEDVADYHCLQYDEFPRTFGGGTKLELK |
| SEQ ID NO: 185 | Heavy chain variable | EVQLQQYGAELGKPGTSVRLSCKVSGYKIRNTYIHWV |
| | region of HC-28 | NQRPGKGLEWIGRIDPANGNTIYAEKFKSKVTLTADTS |
| | | SNTAYMQLSQLKSDDTALYFCAMNYEGYEDYWGQGV |
| | | MVTVSS |
| SEQ ID NO: 186 | Light chain variable | DIQMTQSPSFLSASVGDSVTINCKASQNINKYLNWYQQ |
| | region of LC-28 | KLGEAPKRLIHKTNSLQPGFPSRFSGSGSGTDYTLTIS |
| | | SLQPEDVAAYFCFQYNSGFTFGAGTKLELK |
| SEQ ID NO: 187 | Heavy chain variable | QVQLVQSGAEVKKPGASVKVSCKASGYTFTDYYIHWV |
| | region of HC-29 | RQAPGQGLEWMGWMNPHSGDTGYAQKFQGRVTMT |
| | | RDTSTSTVYMELSSLRSEDTAVYYCARHGRGYNGYEG |
| | | AFDIWGQGTLVTVSSAS |
| SEQ ID NO: 188 | Light chain variable | DIQMTQSPSSLSASVGDRVTITCRASQGIGNELGWYQ |
| | region of LC-29 | QKPGKAPKLLIYAASNLQSGVPSRFSGSGSGTDFTLTI |
| | | SSLQPEDFATYYCQQYDNLPLTFGQGTKVEIK |
| SEQ ID NO: 189 | Heavy chain variable | QVQLVQSGAEVKKPGASVKVSCKASGYTFTGYYLHW |
| | region of HC-30 | VRQAPGQGLEWMGWINPNSGDTNYAQNFQGRVTMT |
| | | RDTSTSTVYMELSSLRSEDTAVYYCARHGRGYNGYEG |
| | | AFDIWGQGTLVTVSSAS |
| SEQ ID NO: 190 | Light chain variable | DIQMTQSPSSLSASVGDRVTITCRASQGIRNDLGWYQ |
| | region of LC-30 | QKPGKAPKLLIYDASSLESGVPSRFSGSGSGTDFTLTIS |
| | | SLQPEDFATYYCQQLNGYPLTFGGGTKVEIK |
| SEQ ID NO: 191 | Heavy chain variable | QVQLVQSGAEVKKPGASVKVSCKASGYTFTGYYLHW |
| | region of HC-31 | VRQAPGQGLEWMGWINPNSGGTNYAQKFQGRVTMT |
| | | RDTSTSTVYMELSSLRSEDTAVYYCARHGRGYEGYEG |
| | | AFDIWGQGTLVTVSSAS |
| SEQ ID NO: 192 | Light chain variable | DIQMTQSPSSLSASVGDRVTITCRASQGIRNDLGWYQ |
| | region of LC-31 | QKPGKAPKLLIYDASELETGVPSRFSGSGSGTDFTLTIS |
| | | SLQPEDFATYYCQQLNGYPITFGQGTKVEIK |
| SEQ ID NO: 193 | Heavy chain variable | QVQLVQSGAEVKKPGASVKVSCKASGYTFTSYYIHWV |
| | region of HC-32 | RQAPGQGLEWMGWLNPSGGGTSYAQKFQGRVTMTR |
| | | DTSTSTVYMELSSLRSEDTAVYYCARHGRGYDGYEGA |
| | | FDIWGQGTLVTVSSAS |
| SEQ ID NO: 194 | Light chain variable | DIQMTQSPSSLSASVGDRVTITCRASQGIRNDLGWYQ |
| | region of LC-32 | QKPGKAPKLLIYDASNLETGVPSRFSGSGSGTDFTLTIS |
| | | SLQPEDFATYYCQQLNGYPLTFGGGTKVEIK |
| SEQ ID NO: 195 | Heavy chain variable | QVQLVQSGAEVKKPGASVKVSCKASGYTFSTYYMHW |
| | region of HC-33 | VRQAPGQGLEWMGIINPSGGSTSYAQKFQGRVTMTR |
| | | DTSTSTVYMKLSSLRSEDTAVYYCARHGRGYEGYEGA |
| | | FDIWGQGTLVIVSSAS |
| SEQ ID NO: 196 | Light chain variable | DIQMTQSPSSLSASVGDRVTITCRASQGIRDDLGWYQ |
| | region of LC-33 | QKPGKAPKLLIYDASNLETGVPSRFSGSGSGTDFTLTIS |
| | | SLQPEDFATYYCQQANGFPLTFGGGTKVEIK |
| SEQ ID NO: 197 | Heavy chain variable | QVQLVQSGAEVKKPGASVKVSCKASGYTFTGYYIHWV |
| | region of HC-34 | RQAPGQGLEWMGIINPSGGNTNYAQNFQGRVTMTRD |
| | | TSTSTVYMELSSLRSEDTAVYYCARHGRGYNAYEGAF |
| | | DIWGQGTLVTVSSAS |
| SEQ ID NO: 198 | Light chain variable | DIQMTQSPSSLSASVGDRVTITCRASQGIRNDLGWYQ |
| | region of LC-34 | QKPGKAPKLLIYDASNLETGVPSRFSGSGSGTDFTLTIS |
| | | SLQPEDFATYYCQQVNGYPLTFGGGTKVEIK |
| SEQ ID NO: 199 | Heavy chain variable | QVQLVQSGAEVKKPGASVKVSCKASGGTFSSYAISWV |
| | region of HC-35 | RQAPGQGLEWMGVINPTVGGANYAQKFQGRVTMTRD |
| | | TSTSTVYMELSSLRSEDTAVYYCARHGRGYNEYEGAF |
| | | DIWGQGTLVTVSSAS |
| SEQ ID NO: 200 | Light chain variable | DIQMTQSPSSLSASVGDRVTITCQASQDISDYLNWYQ |
| | region of LC-35 | QKPGKAPKLLIYDASNLETGVPSRFSGSGSGTDFTLTIS |
| | | SLQPEDFATYYCQQGNSFPLTFGGGTKLEIK |
| SEQ ID NO: 201 | Heavy chain variable | QVQLVQSGAEVKKLGASVKVSCKASGYTFSSYYMHW |
| | region of HC-36 | VRQAPGQGLEWMGVINPNGAGTNFAQKFQGRVTMTR |
| | | DTSTSTVYMELSSLRSEDTAVYYCARHGRGYEGYEGA |
| | | FDIWGQGTLVIVSSAS |
| SEQ ID NO: 190 | Light chain variable | DIQMTQSPSSLSASVGDRVTITCRASQGIRNDLGWYQ |
| | region of LC-36 | QKPGKAPKLLIYDASSLESGVPSRFSGSGSGTDFTLTIS |
| | | SLQPEDFATYYCQQLNGYPLTFGGGTKVEIK |
| SEQ ID NO: 202 | Heavy chain variable | QVQLVQSGAEVKKPGASVKVSCKASGYTFTTYYMHW |
| | region of HC-37 | VRQAPGQGLEWMGWINPTGGGTNYAQNFQGRVTMT |
| | | RDTSTSTVYMELSSLRSEDTAVYYCARHGRGYEGYEG |
| | | AFDIWGQGTLVTVSSAS |
| SEQ ID NO: 203 | Light chain variable | DIQMTQSPSSLSASVGDRVTITCRASQGIRNDVSWYQ |
| | region of LC-37 | QKPGKAPKLLIYDASNLETGVPSRFSGSGSGTDFTLTIS |
| | | SLQPEDFATYYCQQLSGYPITFGQGTKLEIK |
| SEQ ID NO: 204 | Heavy chain variable | QVQLVQSGAEVKKPGASVKVSCKASGYTFTSYYIHWV |
| | region of HC-38 | RQAPGQGLEWMGMINPSGGSTNYAQKFQGRVTMTR |
| | | DTSTSTVYMELSSLRSEDTAVYYCARHGRGYNDYEGA |
| | | FDIWGQGTLVTVSSAS |
| SEQ ID NO: 205 | Light chain variable | DIQMTQSPSSLSASVGDRVTITCRASQSISDWLAWYQ |
| | region of LC-38 | QKPGKAPKLLIYEASNLEGGVPSRFSGSGSGTDFTLTI |
| | | SSLQPEDFATYYCQQANSFPYTFGQGTKVEIK |
| SEQ ID NO: 206 | Heavy chain variable | QVQLVQSGAEVKKPGASVKVSCKASGYIFSAYYIHWV |
| | region of HC-39 | RQAPGQGLEWMGIINPSGGSTRYAQKFQGRVTMTRD |
| | | TSTSTVYMELSSLRSEDTAVYYCARHGRGYGGYEGAF |
| | | DIWDQGTLVTVSSAS |
| SEQ ID NO: 207 | Light chain variable | DIQMTQSPSSLSASVGDRVTITCRASQGIGDYVAWYQ |
| | region of LC-39 | QKPGKAPKLLIYDASNLETGVPSRFSGSGSGTDFTLTIS |
| | | SLQPEDFATYYCQQLNGYPITFGQGTRLEIK |
| SEQ ID NO: 208 | Heavy chain variable | EVQLVQSGAEVKKPGESLKISCKGSGYRFTSYWIGWV |
| | region of HC-40 | RQMPGKGLEWMGIIYPDDSDTRYSPSFQGQVTISVDK |
| | | SNSTAYLQWSSLKASDTAMYYCARHGRGYNGYEGAF |
| | | DIWGQGTLVTVSSAS |
| SEQ ID NO: 209 | Light chain variable | DIQMTQSPSSLSASVGDRVTITCRASQGISSYLAWYQQ |
| | region of LC-40 | KPGKAPKLLIYDASNLETGVPSRFSGSGSGTYFTLTISS |
| | | LQPEDFATYYCQQGASFPITFGQGTKVEIK |
| SEQ ID NO: 210 | Heavy chain variable | EVQLVQSGAEVKKPGESLKISCKGSGSSFPNSWIAWV |
| | region of HC-41 | RQMPGKGLEWMGIIYPSDSDTRYSPSFQGQVTISADK |
| | | SISTAYLQWSSLEASDTAMYYCARHGRGYNGYEGAFD |
| | | IWGQGTLVTVSSAS |
| SEQ ID NO: 211 | Light chain variable | DIQMTQSPSSLSASVGDRVTITCRASQGIRNYLAWYQ |
| | region of LC-41 | QKPGKAPKLLIYDASSLQSGVPSRFSGSGSGTDFTLTI |
| | | SSLQPEDFATYYCQQLNSYPLTFGGGTKVEIK |
| SEQ ID NO: 212 | Heavy chain variable | EVQLVQSGAEVKKPGESLKISCKGSGYSFDSYWIGWV |
| | region of HC-42 | RQMPGKGLEWMGIMYPGDSDTRYSPSFQGQVTISAD |
| | | KSISTAYLQWSSLKASDTAMYYCARHGRGYNAYEGAF |
| | | DIWGQGTLVTVSSAS |
| SEQ ID NO: 213 | Light chain variable | DIQMTQSPSSLSASVGDRVTITCRASQSINNWLAWYQ |
| | region of LC-42 | QKPGKAPKLLIYDAFILQSGVPSRFSGSGSGTDFTLTIS |
| | | SLQPEDFATYYCLQLNSYPLTFGPGTKVDIK |
| SEQ ID NO: 214 | Heavy chain variable | EVQLVQSGAEVKKPGESLKISCKGSGYSFTNWIAWVR |
| | region of HC-43 | QMPGKGLEWMGIIYPGDSETRYSPSFQGQVTISADKSI |
| | | STAYLQWSSLKASDTAMYYCARHGRGYYGYEGAFDI |
| | | WGQGTLVTVSSAS |
| SEQ ID NO: 215 | Light chain variable | DIQMTQSPSSLSASVGDRVTITCRASQGISDNLNWYQ |
| | region of LC-43 | QKPGKAPKLLIYDASNLETGVPSRFSGSGSGTDFTLTIS |
| | | SLQPEDFATYYCQQAISFPLTFGQGTKVEIK |
| SEQ ID NO: 216 | Heavy chain variable | EVQLVQSGAEVKKPGESLKISCKGSGYNFTSYWIGWV |
| | region of HC-44 | RQMPGKGLEWMGVIYPDDSETRYSPSFQGQVTISADK |
| | | SISTAYLQWSSLKASDTAMYYCARHGRGYNGYEGAFD |
| | | IWGQGTLVTVSSAS |
| SEQ ID NO: 217 | Light chain variable | DIQMTQSPSSLSASVGDRVTITCRASRDIRDDLGWYQ |
| | region of LC-44 | QKPGKAPKLLIYDASNLETGVPSRFSGSGSGTDFTLTIS |
| | | SLQPEDFATYYCQQANSFPLTFGGGTKVEIK |
| SEQ ID NO: 218 | Heavy chain variable | EVQLVQSGAEVKKPGESLKISCKGSGYTFNTYIGWVR |
| | region of HC-45 | QMPGKGLEWMGIIYPGDSGTRYSPSFQGQVTISADKAI |
| | | STAYLQWSSLKASDTAMYYCARHSRGYNGYEGAFDI |
| | | WGQGTLVTVSSAS |
| SEQ ID NO: 219 | Light chain variable | DIQMTQSPSSLSASVGDRVTITCRASQGISNYLAWYQQ |
| | region of LC-45 | KPGKAPKLLIYDASNLETGVPSRFSGSGSGTDFTLTISS |
| | | LQPEDFATYYCQQANSFPVTFGQGTKVEIK |
| SEQ ID NO: 220 | Heavy chain variable | EVQLVQSGAEVKKPGESLKISCKGSGYNFTTYWIGWV |
| | region of HC-46 | RQMPGKGLEWMGIIHPADSDTRYNPSFQGQVTISADK |
| | | SISTAYLQWSSLKASDTAMYYCARHGRGYNGYEGAFD |
| | | IWGQGTLVTVSSAS |
| SEQ ID NO: 221 | Light chain variable | DIQMTQSPSSLSASVGDRVTITCRVSQGISSYLAWYQQ |
| | region of LC-46 | KPGKAPKLLIYDASNLETGVPSRFSGSGSGTDFTLTISS |
| | | LQPEDFATYYCQQANSFPLTFGGGTKVEIK |
| SEQ ID NO: 222 | Heavy chain variable | EVQLVQSGAEVKKPGESLKISCKGSGYRFSNYWIAWV |
| | region of HC-47 | RQMPGKGLEWMGIIYPDNSDTRYSPSFQGQVTISADK |
| | | SISTAYLQWSSLKASDTAMYYCARHGRGYDGYEGAFD |
| | | IWGQGTLVTVSSAS |
| SEQ ID NO: 223 | Light chain variable | DIQMTQSPSSLSASVGDRVTITCRASQGIRSDLAWYQ |
| | region of LC-47 | QKPGKAPKLLIYGASSLQSGVPSRFSGSGSGTDFTLTI |
| | | SSLQPEDFATYYCQQANSFPLSFGQGTKVEIK |
| SEQ ID NO: 224 | Heavy chain variable | EVQLVQSGAEVKKPGESLKISCKGSGYRFASYWIGWV |
| | region of HC-48 | RQMPGKGLEWMGITYPGDSETRYNPSQGQVTISADK |
| | | SISTAYLQWSSLKASDTAMYYCARHGRGYGGYEGAFD |
| | | IWGQGTLVTVSSAS |
| SEQ ID NO: 225 | Light chain variable | DIQMTQSPSSLSASVGDRVTITCRASQGIRNDLGWYQ |
| | region of LC-48 | QKPGKAPKLLIYDASNLETGVPSRFSGSGSGTDFTLTIS |
| | | SLQPEDFATYYCQQANSFPLTFGGGTKVEIK |
| SEQ ID NO: 226 | Heavy chain variable | EVQLVQSGAEVKKPGESLKISCKGSGYSFTSYWIGWV |
| | region of HC-49 | RQMPGKGLEWMGIIYPGDSDTRYSPSFQGQVTISADK |
| | | SISTAYLQWSSLKASDTAMYYCARHGRGYNGYEGAFD |
| | | IWGQGTLVTVSSAS |
| SEQ ID NO: 227 | Light chain variable | DIQMTQSPSSLSASVGDRVTITCRASQSISNWLAWYQ |
| | region of LC-49 | QKPGKAPKLLIYDASNLETGVPSRFSGSGSGTDFTLTIS |
| | | SLQPEDFATYYCQQTNSFPLTFGQGTRLEIK |
| SEQ ID NO: 147 | Heavy chain variable | QVQLVQSGAAVKKPGESLKISCKGSGYRFTTYWIGWV |
| | region of HC-74 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 228 | Light chain variable | DIQLTQSPSSLSASVGDRVTITCRASQGVISALAWYQQ |
| | region of LC-74 | KPGKAPKLLIYDASSLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-75 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 229 | Light chain variable | DIQLTQSPSSLSASVGDRVTITCRASQGIRSALAWYQQ |
| | region of LC-75 | KPGKAPKLLIYDASSLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-76 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 230 | Light chain variable | DIQLTQSPSSLSASVGDRVTITCRASQGVGSALAWYQ |
| | region of LC-76 | QKPGKAPKLLIYDASSLESGVPSRFSGSGSGTDFTLTIS |
| | | SLQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-77 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 231 | Light chain variable | DIQLTQSPSSLSASVGDRVTITCRASQGVISALAWYQQ |
| | region of LC-77 | KPGKAPKLLIYDASILESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-78 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 232 | Light chain variable | DIQLTQSPSSLSASVGDRVTITCRASQGIRSALAWYQQ |
| | region of LC-78 | KPGKAPKLLIYDASILESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-79 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 233 | Light chain variable | DIQLTQSPSSLSASVGDRVTITCRASQGVGSALAWYQ |
| | region of LC-79 | QKPGKAPKLLIYDASILESGVPSRFSGSGSGTDFTLTIS |
| | | SLQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-80 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 234 | Light chain variable | DIQLTQSPSSLSASVGDRVTITCRASQGISSALAWYQQ |
| | region of LC-80 | KPGKAPKLLIYDASILESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-81 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 235 | Light chain variable | DIQLTQSPSSLSASVGDRVTITCRASQGVISALAWYQQ |
| | region of LC-81 | KPGKAPKLLIYDASTLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-82 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 236 | Light chain variable | DIQLTQSPSSLSASVGDRVTITCRASQGIRSALAWYQQ |
| | region of LC-82 | KPGKAPKLLIYDASTLESGVPSRFSGSGSGTDFTLTISS |
| | | LQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-83 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 237 | Light chain variable | DIQLTQSPSSLSASVGDRVTITCRASQGVGSALAWYQ |
| | region of LC-83 | QKPGKAPKLLIYDASTLESGVPSRFSGSGSGTDFTLTIS |
| | | SLQPEDFATYYCQQFNSYPLTFGGGTKVEIK |
| SEQ ID NO: 147 | Heavy chain variable | QVQLVQSGAAVKKPGESLKISCKGSGYRFTTYWIGWV |
| | region of HC-84 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 237 | Light chain variable | DIQLTQSPSSLSASVGDRVTITCRASQGVGSALAWYQ |
| | region of LC-84 | QKPGKAPKLLIYDASTLESGVPSRFSGSGSGTDFTLTIS |
| | | SLQPEDFATYYCQQFNSYPLTFGGGTKVEIK |
| SEQ ID NO: 238 | Heavy chain variable | EVQLVQSGAEVKKPGESLKISCKGSGYRFTTSWIGWV |
| | region of HC-245 | RQMPGKGLEWMGIIYPGDSDTRYSPSFQGQVTISADK |
| | | SISTAYLQWSSLKASDTAMYYCARHGLGYNGYEGAFD |
| | | IWGQGTLVTVSS |
| SEQ ID NO: 239 | Light chain variable | DIQMTQSPSSLSASVGDRVTITCRASQGIGSALAWYQ |
| | region of LC-245 | QKPGKAPKLLIYDASTLESGVPSRFSGSGSGTDFTLTIS |
| | | SLQPEDFATYYCQQFNGYPLTFGQGTRLEIK |
| SEQ ID NO: 147 | Heavy chain variable | QVQLVQSGAAVKKPGESLKISCKGSGYRFTTYWIGWV |
| | region of HC-246 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 239 | Light chain variable | DIQMTQSPSSLSASVGDRVTITCRASQGIGSALAWYQ |
| | region of LC-246 | QKPGKAPKLLIYDASTLESGVPSRFSGSGSGTDFTLTIS |
| | | SLQPEDFATYYCQQFNGYPLTFGQGTRLEIK |
| SEQ ID NO: 147 | Heavy chain variable | QVQLVQSGAAVKKPGESLKISCKGSGYRFTTYWIGWV |
| | region of HC-247 | RQMPGKGLEWMGIIYPGDSDTRYSPSFQGQVTISAGK |
| | | SISTAYLQWSSLKASDTAMYYCARHGRGYNGYEGAFD |
| | | IWGQGTMVTVSS |
| SEQ ID NO: 240 | Light chain variable | DIQMTQSPSSLSASVGDRVTITCRASRGISDYLAWYQQ |
| | region of LC-247 | KPGKAPKLLIYDASNLETGVPSRFSGSGSGTDFTLTISS |
| | | LQPEDFATYYCQQANSFPITFGQGTRLEIK |
| SEQ ID NO: 238 | Heavy chain variable | EVQLVQSGAEVKKPGESLKISCKGSGYRFTTSWIGWV |
| | region of HC-248 | RQMPGKGLEWMGIIYPGDSDTRYSPSFQGQVTISADK |
| | | SISTAYLQWSSLKASDTAMYYCARHGLGYNGYEGAFD |
| | | IWGQGTLVTVSS |
| SEQ ID NO: 241 | Light chain variable | DIQMTQSPSSLSASVGDRVTITCRASQGIGSALAWYQ |
| | region of LC-248 | QKPGKAPKLLIYDASTLESGVPSRFSGSGSGTDFTLTIS |
| | | SLQPEDFATYYCQQLNGYPLTFGQGTRLEIK |
| SEQ ID NO: 238 | Heavy chain variable | EVQLVQSGAEVKKPGESLKISCKGSGYRFTTSWIGWV |
| | region of HC-249 | RQMPGKGLEWMGIIYPGDSDTRYSPSFQGQVTISADK |
| | | SISTAYLQWSSLKASDTAMYYCARHGLGYNGYEGAFD |
| | | IWGQGTLVTVSS |
| SEQ ID NO: 242 | Light chain variable | DIQMTQSPSSLSASVGDRVTITCRASQGIGSALAWYQ |
| | region of LC-249 | QKPGKAPKLLIYDASNLETGVPSRFSGSGSGTDFTLTIS |
| | | SLQPEDFATYYCQQLNGYPLTFGQGTRLEIK |
| SEQ ID NO: 243 | Heavy chain variable | EVQLVQSGAEVKKPGESLKISCKGSGYSFTNYWIGWV |
| | region of Ab 85 | RQMPGKGLEWMAIINPRDSDTRYRPSFQGQVTISADK |
| | | SISTAYLQWSSLKASDTAMYYCARHGRGYEGYEGAFD |
| | | IWGQGTLVTVSS |
| SEQ ID NO: 244 | Light chain variable | DIQMTQSPSSLSASVGDRVTITCRSSQGIRSDLGWYQ |
| | region of Ab 85 | QKPGKAPKLLIYDASNLETGVPSRFSGSGSGTDFTLTIS |
| | | SLQPEDFATYYCQQANGFPLTFGGGTKVEIK |
| SEQ ID NO: 245 | Ab85 CDR-H1 | NYWIG |
| SEQ ID NO: 246 | Ab85 CDR-H2 | IINPRDSDTRYRPSFQG |
| SEQ ID NO: 247 | Ab85 CDR-H3 | HGRGYEGYEGAFDI |
| SEQ ID NO: 248 | Ab85 CDR-L1 | RSSQGIRSDLG |
| SEQ ID NO: 249 | Ab85 CDR-L2 | DASNLET |
| | Ab249 CDR-L2 | |
| SEQ ID NO: 250 | Ab85 CDR-L3 | QQANGFPLT |
| SEQ ID NO: 251 | Heavy chain variable | EVQLVQSGAEVKKPGESLKISCKGSGYSFTNYWIGWV |
| | region of Ab 86 | RQMPGKGLEWMGIIYPGDSDIRYSPSLQGQVTISVDTS |
| | | TSTAYLQWNSLKPSDTAMYYCARHGRGYNGYEGAFDI |
| | | WGQGTLVTVSS |
| SEQ ID NO: 252 | Light chain variable | DIQMTQSPSSLSASVGDRVTITCRASQGIGDSLAWYQ |
| | region of Ab 86 | QKPGKAPKLLIYDASNLETGVPSRFSGSGSGTDFTLTIS |
| | | SLQPEDFATYYCQQLNGYPITFGQGTKVEIK |
| SEQ ID NO: 245 | Ab86 CDR-H1 | NYWIG |
| SEQ ID NO: 253 | Ab86 CDR-H2 | IIYPGDSDIRYSPSLQG |
| SEQ ID NO: 3 | Ab86 CDR-H3 | HGRGYNGYEGAFDI |
| SEQ ID NO: 254 | Ab86 CDR-L1 | RASQGIGDSLA |
| SEQ ID NO: 249 | Ab86 CDR-L2 | DASNLET |
| SEQ ID NO: 255 | Ab86 CDR-L3 | QQLNGYPIT |
| SEQ ID NO: 243 | Heavy chain variable | EVQLVQSGAEVKKPGESLKISCKGSGYSFTNYWIGWV |
| | region of Ab 87 | RQMPGKGLEWMAIINPRDSDTRYRPSFQGQVTISADK |
| | | SISTAYLQWSSLKASDTAMYYCARHGRGYEGYEGAFD |
| | | IWGQGTLVTVSS |
| SEQ ID NO: 256 | Light chain variable | DIQMTQSPSSLSASVGDRVTITCRASQGIRNDLGWYQ |
| | region of Ab 87 | QKPGKAPKLLIYDASSLESGVPSRFSGSGSGTDFTLTIS |
| | | SLQPEDFATYYCQQLNGYPITFGQGTKVEIK |
| SEQ ID NO: 245 | Ab87 CDR-H1 | NYWIG |
| SEQ ID NO: 246 | Ab87 CDR-H2 | IINPRDSDTRYRPSFQG |
| SEQ ID NO: 247 | Ab87 CDR-H3 | HGRGYEGYEGAFDI |
| SEQ ID NO: 257 | Ab87 CDR-L1 | RASQGIRNDLG |
| SEQ ID NO: 5 | Ab87 CDR-L2 | DASSLES |
| SEQ ID NO: 255 | Ab87 CDR-L3 | QQLNGYPIT |
| SEQ ID NO: 258 | Heavy chain variable | EVQLVQSGAEVKKPGESLKISCKGSGYSFTNYWIGWV |
| | region of Ab 88 | RQMPGKGLEWMGIIYPGDSLTRYSPSFQGQVTISADK |
| | | SISTAYLQWSSLKASDTAMYYCARHGRGYNGYEGAFD |
| | | IWGQGTLVTVSS |
| SEQ ID NO: 256 | Light chain variable | DIQMTQSPSSLSASVGDRVTITCRASQGIRNDLGWYQ |
| | region of Ab 88 | QKPGKAPKLLIYDASSLESGVPSRFSGSGSGTDFTLTIS |
| | | SLQPEDFATYYCQQLNGYPITFGQGTKVEIK |
| SEQ ID NO: 245 | Ab88 CDR-H1 | NYWIG |
| SEQ ID NO: 259 | Ab88 CDR-H2 | IIYPGDSLTRYSPSFQG |
| SEQ ID NO: 3 | Ab88 CDR-H3 | HGRGYNGYEGAFDI |
| SEQ ID NO: 257 | Ab88 CDR-L1 | RASQGIRNDLG |
| SEQ ID NO: 5 | Ab88 CDR-L2 | DASSLES |
| SEQ ID NO: 255 | Ab88 CDR-L3 | QQLNGYPIT |
| SEQ ID NO: 260 | Heavy chain variable | EVQLVQSGAEVKKPGESLKISCKGSGYSFTNYWIGWV |
| | region of Ab89 | RQMPGKGLEWMGIIYPGDSDTRYSPSFQGQVTISADK |
| | | SISTAYLQWSSLKASDTAMYYCARHGRGYNGYEGAFD |
| | | IWGQGTLVTVSS |
| SEQ ID NO: 252 | Light chain variable | DIQMTQSPSSLSASVGDRVTITCRASQGIGDSLAWYQ |
| | region of Ab89 | QKPGKAPKLLIYDASNLETGVPSRFSGSGSGTDFTLTIS |
| | | SLQPEDFATYYCQQLNGYPITFGQGTKVEIK |
| SEQ ID NO: 245 | Ab89 CDR-H1 | NYWIG |
| SEQ ID NO: 2 | Ab89 CDR-H2 | IIYPGDSDTRYSPSFQG |
| SEQ ID NO: 3 | Ab89 CDR-H3 | HGRGYNGYEGAFDI |
| SEQ ID NO: 254 | Ab89 CDR-L1 | RASQGIGDSLA |
| SEQ ID NO: 249 | Ab89 CDR-L2 | DASNLET |
| SEQ ID NO: 255 | Ab89 CDR-L3 | QQLNGYPIT |
| SEQ ID NO: 261 | Heavy chain variable | QVQLVQSGAAVKKPGESLKISCKGSGYRFTSYWIGWV |
| | region amino acid | RQMPGKGLEWMGIIYPGDSDTRYSPSFQGQVTISAGK |
| | sequence of CK6 | SISTAYLQWSSLKASDTAMYYCARHGRGYNGYEGAFD |
| | | IWGQGTMVTVSS |
| SEQ ID NO: 262 | Light chain variable | AIQLTQSPSSLSASVGDRVTITCRASQGISSALAWYQQ |
| | region amino acid | KPGKAPKLLIYDASSLESGVPSRFSGSGSGTDFTLTISS |
| | sequence of CK6 | LQPEDFATYYCQQFNSYPLTFGGGTKVEIK |
| SEQ ID NO: 263 | Ab77 CDR-H1 | TYWIG |
| SEQ ID NO: 2 | Ab77 CDR-H2 | IIYPGDSDTRYSPSFQG |
| SEQ ID NO: 3 | Ab77 CDR-H3 | HGRGYNGYEGAFDI |
| SEQ ID NO: 267 | Ab77 CDR-L1 | RASQGVISALA |
| SEQ ID NO: 265 | Ab77 CDR-L2 | DASILES |
| SEQ ID NO: 266 | Ab77 CDR-L3 | QQFNSYPLT |
| SEQ ID NO: 263 | Ab79 CDR-H1 | TYWIG |
| SEQ ID NO. 2 | Ab79 CDR-H2 | IIYPGDSDTRYSPSFQG |
| SEQ ID NO: 3 | Ab79 CDR-H3 | HGRGYNGYEGAFDI |
| SEQ ID NO. 267 | Ab79 CDR-L1 | RASQGVGSALA |
| SEQ ID NO. 265 | Ab79 CDR-L2 | DASILES |
| SEQ ID NO: 266 | Ab79 CDR-L3 | QQFNSYPLT |
| SEQ ID NO: 263 | Ab81 CDR-H1 | TYWIG |
| SEQ ID NO: 2 | Ab81 CDR-H2 | IIYPGDSDTRYSPSFQG |
| SEQ ID NO: 3 | Ab81 CDR-H3 | HGRGYNGYEGAFDI |
| SEQ ID NO: 264 | Ab81 CDR-L1 | RASQGVISALA |
| SEQ ID NO: 268 | Ab81 CDR-L2 | DASTLES |
| SEQ ID NO: 266 | Ab81 CDR-L3 | QQFNSYPLT |
| SEQ ID NO: 269 | Heavy chain constant | ASTKGPSVFPLAPSSKSTSGGTAALGCLVKDYFPEPVT |
| | region (Wild type (WT)) | VSWNSGALTSGVHTFPAVLQSSGLYSLSSVVTVPSSS |
| | | LGTQTYICNVNHKPSNTKVDKKVEPKSCDKTHTCPPC |
| | | PAPELLGGPSVFLFPPKPKDTLMISRTPEVTCVVVDVS |
| | | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| SEQ ID NO: 270 | Heavy chain constant | ASTKGPSVFPLAPSSKSTSGGTAALGCLVKDYFPEPVT |
| | region with L234A, | VSWNSGALTSGVHTFPAVLQSSGLYSLSSVVTVPSSS |
| | L235A (LALA) | LGTQTYICNVNHKPSNIKVDKKVEPKSCDKTHTCPPC |
| | mutations (mutations in | PAPEAAGGPSVFLFPPKPKDTLMISRTPEVTCVVVDVS |
| | bold)* | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| SEQ ID NO: 271 | Heavy chain constant | ASTKGPSVFPLAPSSKSTSGGTAALGCLVKDYFPEPVT |
| | region with D265C | VSWNSGALTSGVHTFPAVLQSSGLYSLSSVVTVPSSS |
| | mutation | LGTQTYICNVNHKPSNTKVDKKVEPKSCDKTHTCPPC |
| | (mutation in bold)* | PAPELLGGPSVFLFPPKPKDTLMISRTPEVTCVVVCVS |
| | | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| SEQ ID NO: 272 | Heavy chain constant | ASTKGPSVFPLAPSSKSTSGGTAALGCLVKDYFPEPVT |
| | region with H435A | VSWNSGALTSGVHTFPAVLQSSGLYSLSSVVTVPSSS |
| | mutation | LGTQTYICNVNHKPSNTKVDKKVEPKSCDKTHTCPPC |
| | mutation in bold)* | PAPELLGGPSVFLFPPKPKDTLMISRTPEVTCVVVDVS |
| | | HEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNAYTQKSLSLSPGK |
| SEQ ID NO: 273 | Heavy chain constant | ASTKGPSVFPLAPSSKSTSGGTAALGCLVKDYFPEPVT |
| | region: modified Fc | VSWNSGALTSGVHTFPAVLQSSGLYSLSSVVTVPSSS |
| | region with L234A, | LGTQTYICNVNHKPSNTKVDKKVEPKSCDKTHTCPPC |
| | L235A, D265C | PAPEAAGGPSVFLFPPKPKDTLMISRTPEVTCVVVCVS |
| | mutations (mutations in | HEDPEVKFNWYVDGVEVHNAKTKPREEQYNSTYRVV |
| | bold)* | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| SEQ ID NO: 274 | Heavy chain constant | ASTKGPSVFPLAPSSKSTSGGTAALGCLVKDYFPEPVT |
| | region: modified Fc | VSWNSGALTSGVHTFPAVLQSSGLYSLSSVVTVPSSS |
| | region with L234A, | LGTQTYICNVNHKPSNTKVDKKVEPKSCDKTHTCPPC |
| | L235A, D265C, H435A | PAPEAAGGPSVFLFPPKPKDTLMISRTPEVTCVVVCVS |
| | mutations (mutations in | HEDPEVKFNWYVDGVEVHNAKTKPREEQYNSTYRVV |
| | bold)* | SVLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNAYTQKSLSLSPGK |
| SEQ ID NO: 275 | Ab85 full length heavy | EVQLVQSGAEVKKPGESLKISCKGSGYSFTNYWIGWV |
| | chain sequence; | RQMPGKGLEWMAIINPRDSDTRYRPSFQGQVTISADK |
| | constant region | SISTAYLQWSSLKASDTAMYYCARHGRGYEGYEGAFD |
| | underlined | IWGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 276 | Ab85 full length heavy | EVQLVQSGAEVKKPGESLKISCKGSGYSFTNYWIGWV |
| | chain sequence; | RQMPGKGLEWMAIINPRDSDTRYRPSFQGQVTISADK |
| | constant region | SISTAYLQWSSLKASDTAMYYCARHGRGYEGYEGAFD |
| | underlined; modified | IWGQGTLVTVSSASTKGPSVFPLAPSSKSTSGGTAALG |
| | Fc region with L234A, | CLVKDYFPEPVTVSWNSGALTSGVHTFPAVLQSSGLY |
| | L235A mutations | SLSSVVTVPSSSLGTQTYICNVNHKPSNTKVDKKVEPK |
| | mutations in bold)* | SCDKTHTCPPCPAPEAAGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 277 | Ab85 full length heavy | EVQLVQSGAEVKKPGESLKISCKGSGYSFTNYWIGWV |
| | chain sequence: | RQMPGKGLEWMAIINPRDSDTRYRPSFQGQVTISADK |
| | constant region | SISTAYLQWSSLKASDTAMYYCARHGRGYEGYEGAFD |
| | underlined; modified | IWGQGTLVTVSSASTKGPSVFPLAPSSKSTSGGTAALG |
| | Fc region with L234A, | CLVKDYFPEPVTVSWNSGALTSGVHTFPAVLQSSGLY |
| | L235A, D265C | SLSSVVTVPSSSLGTQTYICNVNHKPSNTKVDKKVEPK |
| | mutations (mutations | SCDKTHTCPPCPAPEAAGGPSVFLFPPKPKDTLMISRT |
| | in bold)* | PEVTCVVVCVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 278 | Ab85 full length heavy | EVQLVQSGAEVKKPGESLKISCKGSGYSFTNYWIGWV |
| | chain sequence (LALA- | RQMPGKGLEWMAIINPRDSDTRYRPSFQGQVTISADK |
| | D265C-H435A | SISTAYLQWSSLKASDTAMYYCARHGRGYEGYEGAFD |
| | mutant); constant | IWGQGTLVTVSSASTKGPSVFPLAPSSKSTSGGTAALG |
| | region underlined | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPEAAGGPSVFLFPPKPKDTLMISRT |
| | | PEVTCVVVCVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTIPPSRDELTKNQVSLTCL |
| | | VKGFYPSDIAVEWESNGQPENNYKTTPPVLDSDGSFF |
| | | LYSKLTVDKSRWQQGNVFSCSVMHEALHNAYTQKSL |
| | | SLSPGK |
| SEQ ID NO: 279 | Ab249 full length heavy | EVQLVQSGAEVKKPGESLKISCKGSGYRFTTSWIGWV |
| | chain sequence; | RQMPGKGLEWMGIIYPGDSDTRYSPSFQGQVTISADK |
| | constant region | SISTAYLQWSSLKASDTAMYYCARHGLGYNGYEGAFD |
| | underlined | IWGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 280 | Ab249 full length heavy | EVQLVQSGAEVKKPGESLKISCKGSGYRFTTSWIGWV |
| | chain sequence; | RQMPGKGLEWMGIIYPGDSDTRYSPSFQGQVTISADK |
| | constant region | SISTAYLQWSSLKASDTAMYYCARHGLGYNGYEGAFD |
| | underlined (LALA | IWGQGTLVTVSSASTKGPSVFPLAPSSKSTSGGTAALG |
| | mutations)* | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPEAAGGPSVFLFPFKPKDTLMISRT |
| | | PEVTCVVVDVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 281 | Ab249 full length heavy | EVQLVQSGAEVKKPGESLKISCKGSGYRFTTSWIGWV |
| | chain sequence; | RQMPGKGLEWMGIIYPGDSDTRYSPSFQGQVTISADK |
| | constant region | SISTAYLQWSSLKASDTAMYYCARHGLGYNGYEGAFD |
| | underlined (LALA- | IWGQGTLVTVSSASTKGPSVFPLAPSSKSTSGGTAALG |
| | D265C mutations)* | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPEAAGGPSVFLFPPKPKDTLMISRT |
| | | PEVTCVVVCVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNHYTQKSL |
| | | SLSPGK |
| SEQ ID NO: 282 | Ab249 full length heavy | EVQLVQSGAEVKKPGESLKISCKGSGYRFTTSWIGWV |
| | chain sequence; | RQMPGKGLEWMGIIYPGDSDTRYSPSFQGQVTISADK |
| | constant region | SISTAYLQWSSLKASDTAMYYCARHGLGYNGYEGAFD |
| | underlined; (LALA- | IWGQGTLVTVSSASTKGPSVFPLAPSSKSTSGGTAALG |
| | D265C-H435A | CLVKDYFPEPVTVSWNSGALTSGVHTFPAVLQSSGLY |
| | mutations)* | SLSSVVTVPSSSLGTQTYICNVNHKPSNTKVDKKVEPK |
| | | SCDKTHTCPPCPAPEAAGGPSVFLFPPKPKDTLMISRT |
| | | PEVTCVVVCVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFF |
| | | LYSKLTVDKSRWQQGNVFSCSVMHEALHNAYTQKSL |
| | | SLSPGK |
| SEQ ID NO: 283 | Light chain constant | RTVAAPSVFIFPPSDEQLKSGTASVVCLLNNFYPREAK |
| | region | VQWKVDNALQSGNSQESVTEQDSKDSTYSLSSTLTLS |
| | | KADYEKHKVYACEVTHQGLSSPVTKSFNRGEC |
| SEQ ID NO: 284 | Ab85 full length light | DIQMTQSPSSLSASVGDRVTITCRSSQGIRSDLGWYQ |
| | chain; constant region | QKPGKAPKLLIYDASNLETGVPSRFSGSGSGTDFTLTIS |
| | underlined | SLQPEDFATYYCQQANGFPLTFGGGTKVEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVIKSFNRGEC |
| SEQ ID NO: 285 | Ab249 light chain; | DIQMTQSPSSLSASVGDRVTITCRASQGIGSALAWYQ |
| | constant region | QKPGKAPKLLIYDASNLETGVPSRFSGSGSGTDFTLTIS |
| | underlined | SLQPEDFATYYCQQLNGYPLTFGQGTRLEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| SEQ ID NO: 286 | Ab249 HC-CDR1 | TSWIG |
| SEQ ID NO: 287 | Ab249 HC-CDR3 | HGLGYNGYEGAFDI |
| SEQ ID NO: 288 | Ab249 LC-CDR1 | RASQGIGSALA |
| SEQ ID NO: 289 | Ab249 LC-CDR3 | QQLNGYPLT |
| SEQ ID NO: 290 | CD45RO (Human | MTMYLWLKLLAFGFAFLDTEVFVTGQSPTPSPTDAYLN |
| | CD45 Isoform) | ASETTTLSPSGSAVISTTTIATTPSKPTCDEKYANITVDY |
| | | LYNKETKLFTAKLNVNENVECGNNTCTNNEVHNLTEC |
| | | KNASVSISHNSCTAPDKTLILDVPPGVEKFQLHDCTQV |
| | | EKADTTICLKWKNIETFTCDTQNITYRFQCGNMIFDNKE |
| | | IKLENLEPEHEYKCDSEILYNNHKFTNASKIIKTDFGSPG |
| | | EPQIIFCRSEAAHQGVITWNPPQRSFHNFTLCYIKETEK |
| | | DCLNLDKNLIKYDLQNLKPYTKYVLSLHAYIIAKVQRNG |
| | | SAAMCHFITKSAPPSQVWNMTVSMTSDNSMHVKCRP |
| | | PRDRNGPHERYHLEVEAGNTLVRNESHKNCDFRVKDL |
| | | QYSTDYTFKAYFHNGDYPGEPFILHHSTSYNSKALIAFL |
| | | AFLIIVTSIALLVVLYKIYDLHKKRSCNLDEQQELVERDD |
| | | EKQLMNVEPIHADILLETYKRKIADEGRLFLAEFQSIPRV |
| | | FSKFPIKEARKPFNQNKNRYVDILPYDYNRVELSEINGD |
| | | AGSNYINASYIDGFKEPRKYIAAQGPRDETVDDFWRMI |
| | | WEQKATVIVMVTRCEEGNRNKCAEYWPSMEEGTRAF |
| | | GDVVVKINQHKRCPDYIIQKLNIVNKKEKATGREVTHIQ |
| | | FTSWPDHGVPEDPHLLLKLRRRVNAFSNFFSGPIVVHC |
| | | SAGVGRTGTYIGIDAMLEGLEAENKVDVYGYVVKLRR |
| | | QRCLMVQVEAQYILIHQALVEYNQFGETEVNLSELHPY |
| | | LHNMKKRDPPSEPSPLEAEFQRLPSYRSWRTQHIGNQ |
| | | EENKSKNRNSNVIPYDYNRVPLKHELEMSKESEHDSD |
| | | ESSDDDSDSEEPSKYINASFIMSYWKPEVMIAAQGPLK |
| | | ETIGDFWQMIFQRKVKVIVMLTELKHGDQEICAQYWGE |
| | | GKQTYGDIEVDLKDTDKSSTYTLRVFELRHSKRKDSRT |
| | | VYQYQYTNWSVEQLPAEPKELISMIQVVKQKLPQKNS |
| | | SEGNKHHKSTPLLIHCRDGSQQTGIFCALLNLLESAET |
| | | EEVVDIFQVVKALRKARPGMVSTFEQYQFLYDVIASTY |
| | | PAQNGQVKKNNHQEDKIEFDNEVDKVKQDANCVNPL |
| | | GAPEKLPEAKEQAEGSEPTSGTEGPEHSVNGPASPAL |
| | | NQGS |
| SEQ ID NO: 291 | CD45RA (Human CD45 | MTMYLWLKLLAFGFAFLDTEVFVTGQSPTPSPTGLTTA |
| | Isoform) | KMPSVPLSSDPLPTHTTAFSPASTFERENDFSETTTSL |
| | | SPDNTSTQVSPDSLDNASAFNTTDAYLNASETTTLSPS |
| | | GSAVISTTTIATTPSKPTCDEKYANITVDYLYNKETKLFT |
| | | AKLNVNENVECGNNTCTNNEVHNLTECKNASVSISHN |
| | | SCTAPDKTLILDVPPGVEKFQLHDCTQVEKADTTICLK |
| | | WKNIETFTCDTQNITYRFQCGNMIFDNKEIKLENLEPEH |
| | | EYKCDSEILYNNHKFTNASKIIKTDFGSPGEPQIIFCRSE |
| | | AAHQGVITWNPPQRSFHNFTLCYIKETEKDCLNLDKNLI |
| | | KYDLONLKPYTKYVLSLHAYIIAKVQRNGSAAMCHFTT |
| | | KSAPPSQVWNMTVSMTSDNSMHVKCRPPRDRNGPH |
| | | ERYHLEVEAGNTLVRNESHKNCDFRVKDLQYSTDYTF |
| | | KAYFHNGDYPGEPFILHHSTSYNSKALIAFLAFLIIVTSIA |
| | | LLVVLYKIYDLHKKRSCNLDEQQELVERDDEKQLMNVE |
| | | PIHADILLETYKRKIADEGRLFLAEFQSIPRVFSKFPIKEA |
| | | RKPFNQNKNRYVDILPYDYNRVELSEINGDAGSNYINA |
| | | SYIDGFKEPRKYIAAQGPRDETVDDFWRMIWEQKATVI |
| | | VMVTRCEEGNRNKCAEYWPSMEEGTRAFGDVVVKIN |
| | | QHKRCPDYIIQKLNIVNKKEKATGREVTHIQFTSWPDH |
| | | GVPEDPHLLLKLRRRVNAFSNFFSGPIVVHCSAGVGRT |
| | | GTYIGIDAMLEGLEAENKVDVYGYVVKLRRQRCLMVQ |
| | | VEAQYILIHQALVEYNQFGETEVNLSELHPYLHNMKKR |
| | | DPPSEPSPLEAEFQRLPSYRSWRTQHIGNQEENKSKN |
| | | RNSNVIPYDYNRVPLKHELEMSKESEHDSDESSDDDS |
| | | DSEEPSKYINASFIMSYWKPEVMIAAQGPLKETIGDFW |
| | | QMIFQRKVKVIVMLTELKHGDQEICAQYWGEGKQTYG |
| | | DIEVDLKDTDKSSTYTLRVFELRHSKRKDSRTVYQYQY |
| | | TNWSVEQLPAEPKELISMIQVVKQKLPQKNSSEGNKH |
| | | HKSTPLLIHCRDGSQQTGIFCALLNLLESAETEEVVDIF |
| | | QVVKALRKARPGMVSTFEQYQFLYDVIASTYPAQNGQ |
| | | VKKNNHQEDKIEFDNEVDKVKQDANCVNPLGAPEKLP |
| | | EAKEQAEGSEPTSGTEGPEHSVNGPASPALNQGS |
| SEQ ID NO: 292 | CD45RB (Human CD45 | MTMYLWLKLLAFGFAFLDTEVFVTGQSPTPSPTGVSS |
| | Isoform) | VQTPHLPTHADSQTPSAGTDTQTFSGSAANAKLNPTP |
| | | GSNAISDAYLNASETTTLSPSGSAVISTTTIATTPSKPTC |
| | | DEKYANITVDYLYNKETKLFTAKLNVNENVECGNNTCT |
| | | NNEVHNLTECKNASVSISHNSCTAPDKTLILDVPPGVE |
| | | KFQLHDCTQVEKADTTICLKWKNIETFTCDTQNITYRFQ |
| | | CGNMIEDNKEIKLENLEPEHEYKCDSEILYNNHKFTNAS |
| | | KIIKTDFGSPGEPQIIFCRSEAAHQGVITWNPPQRSFHN |
| | | FTLCYIKETEKDCLNLDKNLIKYDLQNLKPYTKYVLSLH |
| | | AYIIAKVQRNGSAAMCHFTTKSAPPSQVWNMTVSMTS |
| | | DNSMHVKCRPPRDRNGPHERYHLEVEAGNTLVRNES |
| | | HKNCDFRVKDLQYSTDYTFKAYFHNGDYPGEPFILHH |
| | | STSYNSKALIAFLAFLIIVTSIALLVVLYKIYDLHKKRSCNL |
| | | DEQQELVERDDEKQLMNVEPIHADILLETYKRKIADEG |
| | | RLFLAEFQSIPRVFSKFPIKEARKPFNQNKNRYVDILPY |
| | | DYNRVELSEINGDAGSNYINASYIDGFKEPRKYIAAQGP |
| | | RDETVDDFWRMIWEQKATVIVMVTRCEEGNRNKCAE |
| | | YWPSMEEGTRAFGDVVVKINQHKRCPDYIIQKLNIVNK |
| | | KEKATGREVTHIQFTSWPDHGVPEDPHLLLKLRRRVN |
| | | AFSNFFSGPIVVHCSAGVGRTGTYIGIDAMLEGLEAEN |
| | | KVDVYGYVVKLRRQRCLMVQVEAQYILIHQALVEYNQF |
| | | GETEVNLSELHPYLHNMKKRDPFSEPSPLEAEFQRLP |
| | | SYRSWRTQHIGNQEENKSKNRNSNVIPYDYNRVPLKH |
| | | ELEMSKESEHDSDESSDDDSDSEEPSKYINASFIMSY |
| | | WKPEVMIAAQGPLKETIGDFWQMIFQRKVKVIVMLTEL |
| | | KHGDQEICAQYWGEGKQTYGDIEVDLKDTDKSSTYTL |
| | | RVFELRHSKRKDSRTVYQYQYTNWSVEQLPAEPKELI |
| | | SMIQVVKQKLPQKNSSEGNKHHKSTPLLIHCRDGSQQ |
| | | TGIFCALLNLLESAETEEVVDIFQVVKALRKARPGMVST |
| | | FEQYQFLYDVIASTYPAQNGQVKKNNHQEDKIEFDNEV |
| | | DKVKQDANCVNPLGAPEKLPEAKEQAEGSEPTSGTEG |
| | | PEHSVNGPASPALNQGS |
| SEQ ID NO: 293 | CD45RC (Human | MTMYLWLKLLAFGFAELDTEVEVTGQSPTPSPTDVPG |
| | VD45 Isoform) | ERSTASTEPTDPVSPLTTTLSLAHHSSAALPARTSNTTI |
| | | TANTSDAYLNASETTTLSPSGSAVISTTTIATTPSKPTC |
| | | DEKYANITVDYLYNKETKLFTAKLNVNENVECGNNTCT |
| | | NNEVHNLTECKNASVSISHNSCTAPDKTLILDVPPGVE |
| | | KFQLHDCTQVEKADTTICLKWKNIETFTCDTQNITYRFQ |
| | | CGNMIFDNKEIKLENLEPEHEYKCDSEILYNNHKFTNAS |
| | | KIIKTDFGSPGEPQIIFCRSEAAHQGVITWNPPQRSFHN |
| | | FTLCYIKETEKDCLNLDKNLIKYDLQNLKPYTKYVLSLH |
| | | AYIIAKVQRNGSAAMCHFTTKSAPPSQVWNMTVSMTS |
| | | DNSMHVKCRPPRDRNGPHERYHLEVEAGNTLVRNES |
| | | HKNCDFRVKDLQYSTDYTFKAYFHNGDYPGEPFILHH |
| | | STSYNSKALIAFLAFLIIVTSIALLVVLYKIYDLHKKRSCNL |
| | | DEQQELVERDDEKQLMNVEPIHADILLETYKRKIADEG |
| | | RLFLAEFQSIPRVFSKFPIKEARKPFNQNKNRYVDILPY |
| | | DYNRVELSEINGDAGSNYINASYIDGFKEPRKYIAAQGP |
| | | RDETVDDFWRMIWEQKATVIVMVTRCEEGNRNKCAE |
| | | YWPSMEEGTRAFGDVVVKINQHKRCPDYIIQKLNIVNK |
| | | KEKATGREVTHIQFTSWPDHGVPEDPHLLLKLRRRVN |
| | | AFSNFFSGPIVVHCSAGVGRIGTYIGIDAMLEGLEAEN |
| | | KVDVYGYVVKLRRQRCLMVQVEAQYILIHQALVEYNQF |
| | | GETEVNLSELHPYLHNMKKRDPPSEPSPLEAEFQRLP |
| | | SYRSWRTQHIGNQEENKSKNRNSNVIPYDYNRVPLKH |
| | | ELEMSKESEHDSDESSDDDSDSEEPSKYINASFIMSY |
| | | WKPEVMIAAQGPLKETIGDFWQMIFQRKVKVIVMLTEL |
| | | KHGDQEICAQYWGEGKQTYGDIEVDLKDTDKSSTYTL |
| | | RVFELRHSKRKDSRTVYQYQYTNWSVEQLPAEPKELI |
| | | SMIQVVKQKLPQKNSSEGNKHHKSTPLLIHCRDGSQQ |
| | | TGIFCALLNLLESAETEEVVDIFQVVKALRKARPGMVST |
| | | FEQYQFLYDVIASTYPAQNGQVKKNNHQEDKIEFDNEV |
| | | DKVKQDANCVNPLGAPEKLPEAKEQAEGSEPTSGTEG |
| | | PEHSVNGPASPALNQGS |
| SEQ ID NO: 294 | Apamistamab Heavy | EVKLLESGGGLVQPGGSLKLSCAASGFDFSRYWMSW |
| | Chain | VRQAPGKGLEWIGEINPTSSTINFTPSLKDKVFISRDNA |
| | | KNTLYLQMSKVRSEDTALYYCARGNYYRYGDAMDYW |
| | | GQGTSVIVSSAKTTPPSVYPLAPGSAAQTNSMVTLGC |
| | | LVKGYFPEPVTVTWNSGSLSSGVHTFPAVLOSDLYTLS |
| | | SSVTVPSSTWPSETVTCNVAHPASSTKVDKKIVPRDC |
| | | GCKPCICTVPEVSSVFIFPPKPKDVLTITLTPKVTCVVVD |
| | | ISKDDPEVQFSWFVDDVEVHTAQTQPREEQFNSTERS |
| | | VSELPIMHQDWLNGKEFKCRVNSAAFPAPIEKTISKTK |
| | | GRPKAPQVYTIPPPKEQMAKDKVSLTCMITDFFPEDITV |
| | | EWQWNGQPAENYKNTQPIMDTDGSYFVYSKLNVQKS |
| | | NWEAGNTFTCSVLHEGLHNHHTEKSLSHSPGK |
| SEQ iD NO: 295 | Apamistamab Light | DIALTQSPASLAVSLGQRATISCRASKSVSTSGYSYLH |
| | Chain | WYQQKPGQPPKLLIYLASNLESGVPARFSGSGSGTDF |
| | | TLNIHPVEEEDAATYYCQHSRELPFTFGSGTKLEIKRAD |
| | | AAPTVSIFPPSSEQLTSGGASVVCFLNNEYPKDINVKW |
| | | KIDGSERQNGVLNSWTDQDSKDSTYSMSSTLTLTKDE |
| | | YERHNSYTCEATHKTSTSPIVKSFNRNEC |
| SEQ ID NO: 296 | Apamistamab Heavy | EVKLLESGGGLVQPGGSLKLSCAASGFDFSRYWMSW |
| | Chain Variable Region | VRQAPGKGLEWIGEINPTSSTINFTPSLKDKVFISRDNA |
| | | KNTLYLQMSKVRSEDTALYYCARGNYYRYGDAMDYW |
| | | GQGTSVTVSSA |
| SEQ ID NO: 297 | Apamistamab Light | DIALTQSPASLAVSLGQRATISCRASKSVSTSGYSYLH |
| | Chain Variable Region | WYQQKPGQPPKLLIYLASNLESGVPARFSGSGSGTDF |
| | | TLNIHPVEEEDAATYYCQHSRELPFTFGSGTKLEIKR |
| SEQ ID NO: 298 | mAb 104 Heavy Chain | EVQLVESGGDLVQPGGSLKLSCTASGFTFSNYGMSWI |
| | Variable Region | RQTPDKRLEWVATIVGNDYTYFPDSMKGRFTVSRDNA |
| | | KSILYLQMNSLASADTAMYYCTRHDWVFDYWGQGTPL |
| | | TVSSAKTTAPSVYPLAPVCGGTTGSSVTLGCLVKGYFP |
| | | EPVTLTWNSGSLSSGVHTFPALLQSGLYTLSSSVTVTS |
| | | NTWPSQTITCNVAHPASSTKVDKKIEPRVPITQNPCPP |
| | | LKECPPCAAPDLLGGPSVFIFPPKIKDVLMISLSPMVTC |
| | | VVVDVSEDDPDVQISWFVNNVEVHTAQTQTHREDYNS |
| | | TLRVVSALPIQHQDWMSGKEFKCKVNNRALPSPIEKTI |
| | | SKPRGPVRAPCNYVLPPPAEEMTKKEFSLTCMITGFLP |
| | | AEIAVDWTSNGRTEQNYKNTATVLDSDGSYFMYSKLR |
| | | VQKSTWERGSLFACSVVHEGLHNHLTTKTISRSLGK |
| SEQ ID NO: 299 | mAb 104 Light Chain | DIVLTQSPASLAVSLGQRAILSCKASQSVSFAGSSLMH |
| | Variable Region | WYQQKPGQQPKLLIYRASDLETGIPTRFSGGGSGTDF |
| | | TLNIHPVEEDDAATYYCQQSREYPYTFGGGTRLEIKRA |
| | | DAAPTVSIFPFSSEQLTSGGASVVCFLNNFYPRDINVK |
| | | WKIDGSERQNGVLNSWTDQDSKDSTYSMSSTLTLTKD |
| | | EYERHNSYTCEATHKTSTSPIVKSFNRNEC |
| SEQ ID NO: 300 | mAb 2B8 Heavy Chain | EVKLVESGGGLLKPGGSLKLSCAASGFTFSKYWMHW |
| | Variable Region | VRQAPGKGLEWIGEIEYDGTETNYAPSMKDRFTISRDN |
| | | AKNTLYLQMSSVRSEDTATYFCTTLQIYNNYLFDYWG |
| | | QGVMVTVSSAQTTAPSVYPLAPGCGDTTSSTVTLGCL |
| | | VKGYFPEPVTVTWNSGALSSDVHTFPAVLQSGLYTLT |
| | | SSVTSSTWPSQTVTCNVAHPASSTKVDKKVERRNGGI |
| | | GHKCPTCPTCHKCPVPELLGGPSVFIFPPKPKDILLISQ |
| | | NAKVTCVVVDVSEEEPDVQFSWFVNNVEVHTAQTQP |
| | | REEQYNSTERVVSALPIQHQDWMSGKEFKCKVNNKAL |
| | | PSPIEKTISKPKGLVRKPQVYVMGPPTEQLTEQTVSLT |
| | | CLTSGELPNDIGVEWTSNGHIEKNYKNTEPVMDSDGS |
| | | FFMYSKLNVERSRWDSRAPFVCSVVHEGLHNHHVEK |
| | | SISRPPGK |
| SEQ ID NO: 301 | mAb 288 Light Chain | DIQMTQSPSFLSASVGDRVTINCKPSQNINKYLNWYQQ |
| | Variable Region | KLGEAPKRLIYNTNSLQTGIPSRFSGSGSGTDYTLTITS |
| | | LQPEDVATYFCLQHNRGVTFGSGTKLEIKRADAAPTVS |
| | | IFPPSMEQLTSGGATVVCFVNNFYPRDISVKWKIDGSE |
| | | QRDGVLDSVTDQDSKDSTYSMSSTLSLTKVEYERHNL |
| | | YTCEVVHKTSSSPVVKSFNRNEC |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of depleting a population of CD117+ cells in a human patient in need of a hematopoietic stem cell transplant, the method comprising administering to the patient an effective amount of an anti-CD117 antibody drug conjugate and an immunosuppressant prior to the patient receiving a transplant comprising allogeneic hematopoietic stem cells.

2. The method of claim 1, further comprising subsequently administering to the patient a transplant comprising allogeneic hematopoietic stem cells.

3. A method comprising administering to a human patient a transplant comprising allogeneic hematopoietic stem cells, wherein the patient has been previously administered either an anti-CD117 or an anti-CD45 antibody drug conjugate and an immunosuppressant in an amount sufficient to deplete a population of hematopoietic stem cells in the patient.

4. (canceled)

5. A method of depleting a population of CD45+ cells in a human patient in need of a hematopoietic stem cell transplant, the method comprising administering to the patient an effective amount of the conjugate of an anti-CD45 antibody drug conjugate and an immunosuppressant prior to the patient receiving a transplant comprising allogeneic hematopoietic stem cells.

6. The method of claim 5, further comprising subsequently administering to the patient a transplant comprising allogeneic hematopoietic stem cells.

7-8. (canceled)

9. The method of claim 1, further comprising administering the immunosuppressant to the patient after the patient has received the transplant.

10. A method of depleting a population of CD117+ or CD45+ cells in a human patient in need of a hematopoietic stem cell transplant, the method comprising
a. administering to the human patient an anti-CD117 antibody drug conjugate in an amount sufficient to deplete a population of CD117+ cells in the patient or administering to the human patient an anti-CD45 antibody drug conjugate in an amount sufficient to deplete a population of CD45+ cells in the patient;
b. administering to the human patient a transplant comprising allogeneic hematopoietic stem cells; and
c. subsequently administering an immunosuppressant to the patient.

11. (canceled)

12. The method of claim 1, wherein the transplant comprises allogeneic hematopoietic stem cells in which all of the HLA antigens match the HLA antigens in the human patient.

13. The method of claim 1, wherein the transplant comprises allogeneic hematopoietic stem cells that comprise at least one HLA-mismatch, at least two HLA-mismatches, or at least five HLA-mismatches relative to the HLA antigens in the patient.

14-15. (canceled)

16. The method of claim 13, wherein the allogeneic hematopoietic stem cells comprise a full HLA-mismatch relative to the HLA antigens in the patient.

17. The method of claim 1, wherein the transplant comprises allogeneic hematopoietic stem cells that comprise at least one minor histocompatibility antigen (miHA)-mismatch relative to the minor histocompatibility antigens in the patient.

18. The method of claim 1, wherein the method is effective to establish at least 80% donor chimerism, at least 85% donor chimerism, at least 90% donor chimerism, or at least 95% donor chimerism.

19-21. (canceled)

22. The method of claim 18, wherein the donor chimerism is assessed at least 6 weeks, 7 weeks, 8 weeks, 9 weeks, or 10 weeks post-transplantation.

23-24. (canceled)

25. The method of claim 1, wherein the immunosuppressant is cyclophosphamide or total body irradiation (TBI).

26. (canceled)

27. The method of claim 1, wherein the immunosuppressant is low-dose TBI.

28. The method of claim 1, wherein the immunosuppressant is an anti-CD8 antibody, an anti-CD4 antibody, or both an anti-CD8 antibody and an anti-CD4 antibody.

29. The method of claim 1, wherein the immunosuppressant is administered post-transplant.

30. The method of claim 1, wherein the immunosuppressant is administered pre-transplant.

31. (canceled)

32. The method of claim 1, wherein the transplant comprising hematopoietic stem cells is administered to the patient after the concentration of the conjugate has substantially cleared from the blood of the patient.

33. The method of claim 1, wherein the hematopoietic stem cells or progeny thereof maintain hematopoietic stem cell functional potential after two or more days following transplantation of the hematopoietic stem cells into the patient.

34-35. (canceled)

36. The method of claim 1, wherein the patient is suffering from a stem cell disorder, a hemoglobinopathy disorder, an autoimmune disorder, myelodysplastic disorder, immunodeficiency disorder, a metabolic disorder, or a cancer.

37-38. (canceled)

39. The method of claim 1, wherein ADC comprises an anti-CD117 antibody comprising a heavy chain/light chain (HC/LC) CDR set (CDR1, CDR2, or CDR3) or a HC/LC variable region set as described in Table 3.

40-41. (canceled)

42. The method of claim 1, wherein the antibody of the conjugate is a human antibody, an intact antibody, or an IgG antibody.

43-45. (canceled)

46. The method of claim 1, wherein the antibody is conjugated to a cytotoxin via a linker.

47. The method of claim 46, wherein the cytotoxin is an RNA polymerase inhibitor.

48. The method of claim 47, wherein the RNA polymerase inhibitor is an amatoxin.

49. The method of claim 47, wherein the RNA polymerase inhibitor is an amanitin.

50. The method of claim 49, wherein the amanitin is selected from the group consisting of α-amanitin, β-amanitin, γ-amanitin, ε-amanitin, amanin, amaninamide, amanullin, amanullinic acid, and proamanullin.

51. The method of claim 46, wherein the cytotoxin selected from the group consisting of an pseudomonas exotoxin A, deBouganin, diphtheria toxin, saporin, maytansine, a maytansinoid, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, and an indolinobenzodiazepine pseudodimer.

52. The method of claim 51, wherein the auristatin is MMAE or MMAF.

53. The method of claim 46, wherein the antibody is conjugated to the toxin by way of a cysteine residue in the Fc domain of the antibody.

54. (canceled)

55. The method of claim 53, wherein the cysteine residue is D265C.


SEQUENCE LISTING (301 sequences)

SEQ ID NO: 1 - protein, 5 aa, Artificial Sequence
  Description of Artificial Sequence Synthetic peptide
SYWIG

SEQ ID NO: 2 - protein, 17 aa, Artificial Sequence
  Description of Artificial Sequence Synthetic peptide
IIYPGDSDTRYSPSFQG

SEQ ID NO: 3 - protein, 14 aa, Artificial Sequence
  Description of Artificial Sequence Synthetic peptide
HGRGYNGYEGAFDI

SEQ ID NO: 4 - protein, 11 aa, Artificial Sequence
  Description of Artificial Sequence Synthetic peptide
RASQGISSALA

SEQ ID NO: 5 - protein, 7 aa, Artificial Sequence
  Description of Artificial Sequence Synthetic peptide
DASSLES

SEQ ID NO: 6 - protein, 10 aa, Artificial Sequence
  Description of Artificial Sequence Synthetic peptide
CQQFNSYPLT

SEQ ID NO: 7 - protein, 120 aa, Artificial Sequence
  Description of Artificial Sequence Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 8 - protein, 109 aa, Artificial Sequence
  Description of Artificial Sequence Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 9 - protein, 121 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 10 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 11 - protein, 9 aa, Artificial Sequence
  Description of Artificial Sequence Synthetic peptide
FTFSDADMD

SEQ ID NO: 12 - protein, 19 aa, Artificial Sequence
  Description of Artificial Sequence Synthetic peptide
RTRNKAGSYTTEYAASVKG

SEQ ID NO: 13 - protein, 12 aa, Artificial Sequence
  Description of Artificial Sequence Synthetic peptide
AREPKYWIDFDL

SEQ ID NO: 14 - protein, 11 aa, Artificial Sequence
  Description of Artificial Sequence Synthetic peptide
RASQSISSYLN

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQSYIAPYT

SEQ ID NO: 17 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgacgccgacatggactgggtccgccaggctccagggaaggggctggagtgggttggccgtactagaaacaaagcaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 18 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaagctacatcgccccttacacttttggcggagggaccaaggttgagatcaaa

SEQ ID NO: 19 - protein, 119 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSS

SEQ ID NO: 20 - protein, 106 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIK

SEQ ID NO: 21 - protein, 9 aa, Artificial Sequence
  Description of Artificial Sequence Synthetic peptide
GTFRIYAIS

SEQ ID NO: 22 - protein, 17 aa, Artificial Sequence
  Description of Artificial Sequence Synthetic peptide
GIIPDFGVANYAQKFQG

SEQ ID NO: 23 - protein, 12 aa, Artificial Sequence
  Description of Artificial Sequence Synthetic peptide
ARGGLDTDEFDL

SEQ ID NO: 24 - protein, 11 aa, Artificial Sequence
  Description of Artificial Sequence Synthetic peptide
RASQSINSYLN

SEQ ID NO: 25 - protein, 7 aa, Artificial Sequence
  Description of Artificial Sequence Synthetic peptide
AASSLQS

SEQ ID NO: 26 - protein, 8 aa, Artificial Sequence
  Description of Artificial Sequence Synthetic peptide
QQGVSDIT

SEQ ID NO: 27 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttccgaatctatgctatcagctgggtgcgacaggcccctggacaagggcttgagtggatgggagggatcatccctgacttcggtgtagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 28 - DNA, 318 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattaacagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaggagtcagtgacatcacttttggcggagggaccaaggttgagatcaaa

SEQ ID NO: 29 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGLDTDEFDLWGRGTLVTVSS

SEQ ID NO: 30 - protein, 106 aa, Artificial Sequence; identical to SEQ ID NO: 20
  Description of Artificial Sequence Synthetic polypeptide

SEQ ID NO: 31 - protein, 9 aa, Artificial Sequence
  Description of Artificial Sequence Synthetic peptide
GTFSSYAIS

SEQ ID NO: 32 - protein, 17 aa, Artificial Sequence
  Description of Artificial Sequence Synthetic peptide
GIIPIFGTANYAQKFQG

SEQ ID NO: 33 - protein, 12 aa, Artificial Sequence
  Description of Artificial Sequence Synthetic peptide
ARGGLDTDEFDL

SEQ ID NO: 34 - protein, 11 aa, Artificial Sequence
  Description of Artificial Sequence Synthetic peptide
RASQSINSYLN

SEQ ID NO: 35 - protein, 7 aa, Artificial Sequence
  Description of Artificial Sequence Synthetic peptide
AASSLQS

SEQ ID NO: 36 - protein, 8 aa, Artificial Sequence
  Description of Artificial Sequence Synthetic peptide
QQGVSDIT

SEQ ID NO: 37 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 38 - DNA, 318 bp, Artificial Sequence; identical to SEQ ID NO: 28
  Description of Artificial Sequence Synthetic polynucleotide

SEQ ID NO: 39 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPAFGTANYAQKFQGRVTITADESTSTAYMELSSLRSEDTAVYYCARGGLDTDEFDLWGRGTLVTVSS

SEQ ID NO: 40 - protein, 106 aa, Artificial Sequence; identical to SEQ ID NO: 20
  Description of Artificial Sequence Synthetic polypeptide

SEQ ID NO: 41 - protein, 9 aa, Artificial Sequence
  Description of Artificial Sequence Synthetic peptide
GTFSLYAIS

SEQ ID NO: 42 - protein, 17 aa, Artificial Sequence
  Description of Artificial Sequence Synthetic peptide
GIIPAFGTANYAQKFQG

SEQ ID NO: 43 - protein, 12 aa, Artificial Sequence
  Description of Artificial Sequence Synthetic peptide
ARGGLDTDEFDL

SEQ ID NO: 44 - protein, 11 aa, Artificial Sequence
  Description of Artificial Sequence Synthetic peptide
RASQSINSYLN

SEQ ID NO: 45 - protein, 7 aa, Artificial Sequence
  Description of Artificial Sequence Synthetic peptide
AASSLQS

SEQ ID NO: 46 - protein, 8 aa, Artificial Sequence
  Description of Artificial Sequence Synthetic peptide
QQGVSDIT

SEQ ID NO: 47 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcctctatgctatctcctgggtgcgacaggcccctggacaagggcttgagtggatgggagggatcatccctgccttcggtaccgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 48 - DNA, 318 bp, Artificial Sequence; identical to SEQ ID NO: 28
  Description of Artificial Sequence Synthetic polynucleotide

SEQ ID NO: 49 - protein, 119 aa, Artificial Sequence
  Description of Artificial Sequence Synthetic polypeptide
QVQLVQSGAEVKKPGSSVKVSCKASGGTFSLYAISWVRQAPGQGLEWMGGIIPHFGLANYAQKFQGRVTITADESTSTAYMELSSLRSEDTAVYYCARGGLDTDEFDLWGRGTLVTVSS

SEQ ID NO: 50 - protein, 106 aa, Artificial Sequence; identical to SEQ ID NO: 20
  Description of Artificial Sequence Synthetic polypeptide

SEQ ID NO: 51 - protein, 9 aa, Artificial Sequence
  Description of Artificial Sequence Synthetic peptide
GTFSLYAIS

SEQ ID NO: 52 - protein, 17 aa, Artificial Sequence
  Description of Artificial Sequence Synthetic peptide
GIIPHFGLANYAQKFQG

SEQ ID NO: 53 - protein, 12 aa, Artificial Sequence
  Description of Artificial Sequence Synthetic peptide
ARGGLDTDEFDL

SEQ ID NO: 54 - protein, 11 aa, Artificial Sequence
  Description of Artificial Sequence Synthetic peptide
RASQSINSYLN

SEQ ID NO: 55 - protein, 7 aa, Artificial Sequence
  Description of Artificial Sequence Synthetic peptide
AASSLQS

SEQ ID NO: 56 - protein, 8 aa, Artificial Sequence
  Description of Artificial Sequence Synthetic peptide
QQGVSDIT

SEQ ID NO: 57 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttctccctctatgctatcagctgggtgcgacaggcccctggacaagggcttgagtggatgggagggatcatccctcacttcggtctcgcaaactacgcacagaagttccagggcagagtcacgattaccgcggacgaatccacgagcacagcctacatggagctgagcagcctgagatctgaggacacggcggtgtactactgcgccagaggtggattggacacagacgagttcgacctatgggggagaggtaccttggtcaccgtctcctca

SEQ ID NO: 58 - DNA, 318 bp, Artificial Sequence; identical to SEQ ID NO: 28
  Description of Artificial Sequence Synthetic polynucleotide

SEQ ID NO: 59 - protein, 123 aa, Artificial Sequence
  Description of Artificial Sequence Synthetic polypeptide
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 60 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 61 - protein, 9 aa, Artificial Sequence
  Description of Artificial Sequence Synthetic peptide
FTFSNYAMS

SEQ ID NO: 62 - protein, 17 aa, Artificial Sequence
  Description of Artificial Sequence Synthetic peptide
AISGSGGSTYYADSVKG

SEQ ID NO: 63 - protein, 16 aa, Artificial Sequence
  Description of Artificial Sequence Synthetic peptide
AKGPPTYHTNYYYMDV

SEQ ID NO: 64 - protein, 11 aa, Artificial Sequence
  Description of Artificial Sequence Synthetic peptide
RASQGISSWLA

SEQ ID NO: 65 - protein, 7 aa, Artificial Sequence
  Description of Artificial Sequence Synthetic peptide
AASSLQS

SEQ ID NO: 66 - protein, 9 aa, Artificial Sequence
  Description of Artificial Sequence Synthetic peptide
QQTNSFPYT

SEQ ID NO: 67 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcaattatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 68 - DNA, 321 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaaacaaatagtttcccttacacttttggcggagggaccaaggttgagatcaaa

SEQ ID NO: 69 - protein, 123 aa, Artificial Sequence
  Description of Artificial Sequence Synthetic polypeptide
EVQLLESGGGLVQPGGSLRLSCAASGFTFSSYVMIWVRQAPGKGLEWVSSISGDSVTTYYADSVKGRFTISRDNSKNTLYLQMNSLRAEDTAVYYCAKGPPTYHTNYYYMDVWGKGTTVTVSS

SEQ ID NO: 70 - protein, 107 aa, Artificial Sequence; identical to SEQ ID NO: 60
  Description of Artificial Sequence Synthetic polypeptide

SEQ ID NO: 71 - protein, 9 aa, Artificial Sequence
  Description of Artificial Sequence Synthetic peptide
FTFSSYVMI

SEQ ID NO: 72 - protein, 17 aa, Artificial Sequence
  Description of Artificial Sequence Synthetic peptide
SISGDSVTTYYADSVKG

SEQ ID NO: 73 - protein, 16 aa, Artificial Sequence
  Description of Artificial Sequence Synthetic peptide
AKGPPTYHTNYYYMDV

SEQ ID NO: 74 - protein, 11 aa, Artificial Sequence
  Description of Artificial Sequence Synthetic peptide
RASQGISSWLA

SEQ ID NO: 75 - protein, 7 aa, Artificial Sequence
  Description of Artificial Sequence Synthetic peptide
AASSLQS

SEQ ID NO: 76 - protein, 9 aa, Artificial Sequence
  Description of Artificial Sequence Synthetic peptide
QQTNSFPYT

SEQ ID NO: 77 - DNA, 369 bp, Artificial Sequence
  Description of Artificial Sequence Synthetic polynucleotide
gaggtgcagctgttggagtctgggggaggcttggtacagcctggggggtccctgagactctcctgtgcagcctctggattcacctttagcagctatgtcatgatctgggtccgccaggctccagggaaggggctggagtgggtctcaagcattagtggtgacagcgtaacaacatactacgcagactccgtgaagggccggttcaccatctccagagacaattccaagaacacgctgtatctgcaaatgaacagcctgagagccgaggacacggcggtgtactactgcgccaagggccctcctacataccacacaaactactactacatggacgtatggggcaagggtacaactgtcaccgtctcctca

SEQ ID NO: 78 - DNA, 321 bp, Artificial Sequence; identical to SEQ ID NO: 68
  Description of Artificial Sequence Synthetic polynucleotide

SEQ ID NO: 79 - protein, 121 aa, Artificial Sequence
  Description of Artificial Sequence Synthetic polypeptide
EVQLVESGGGLVQPGGSLRLSCAASGFTFSDHYMDWVRQAPGKGLEWVGRTRNKASSYTTEYAASVKGRFTISRDDSKNSLYLQMNSLKTEDTAVYYCAREPKYWIDFDLWGRGTLVTVSS

SEQ ID NO: 80 - protein, 107 aa, Artificial Sequence; identical to SEQ ID NO: 10
  Description of Artificial Sequence Synthetic polypeptide

SEQ ID NO: 81 - protein, 9 aa, Artificial Sequence
  Description of Artificial Sequence Synthetic peptide
FTFSDHYMD

SEQ ID NO: 82 - protein, 19 aa, Artificial Sequence
  Description of Artificial Sequence Synthetic peptide
RTRNKASSYTTEYAASVKG

SEQ ID NO: 83 - protein, 12 aa, Artificial Sequence
  Description of Artificial Sequence Synthetic peptide
AREPKYWIDFDL

SEQ ID NO: 84 - protein, 11 aa, Artificial Sequence
  Description of Artificial Sequence Synthetic peptide
RASQSISSYLN

SEQ ID NO: 85 - protein, 7 aa, Artificial Sequence
  Description of Artificial Sequence Synthetic peptide
AASSLQS

SEQ ID NO: 86 - protein, 9 aa, Artificial Sequence
  Description of Artificial Sequence Synthetic peptide
QQSYIAPYT

SEQ ID NO: 87 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctagtagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 88 - DNA, 321 bp, Artificial Sequence; identical to SEQ ID NO: 18
  Description of Artificial Sequence Synthetic polynucleotide

SEQ ID NO: 89 - protein, 121 aa, Artificial Sequence
  Description of Artificial Sequence Synthetic polypeptide
EVQLVESGGGLVQPGRSLRLSCTASGFTFSDHDMNWVRQAPGKGLEWVGRTRNAAGSYTTEYAASVKGRFTISRDDSKNSLYLQMNSLKTEDTAVYYCAREPKYWIDFDLWGRGTLVTVSS

SEQ ID NO: 90 - protein, 107 aa, Artificial Sequence; identical to SEQ ID NO: 10
  Description of Artificial Sequence Synthetic polypeptide

SEQ ID NO: 91 - protein, 9 aa, Artificial Sequence
  Description of Artificial Sequence Synthetic peptide
FTFSDHDMN

SEQ ID NO: 92 - protein, 19 aa, Artificial Sequence
  Description of Artificial Sequence Synthetic peptide
RTRNAAGSYTTEYAASVKG

SEQ ID NO: 93 - protein, 12 aa, Artificial Sequence
  Description of Artificial Sequence Synthetic peptide
AREPKYWIDFDL

SEQ ID NO: 94 - protein, 11 aa, Artificial Sequence
  Description of Artificial Sequence Synthetic peptide
RASQSISSYLN

SEQ ID NO: 95 - protein, 7 aa, Artificial Sequence
  Description of Artificial Sequence Synthetic peptide
AASSLQS

SEQ ID NO: 96 - protein, 9 aa, Artificial Sequence
  Description of Artificial Sequence Synthetic peptide
QQSYIAPYT

SEQ ID NO: 97 - DNA, 363 bp, Artificial Sequence
  Description of Artificial Sequence Synthetic polynucleotide
gaggtgcagctggtggagtctgggggaggcttggtacagccagggcggtccctgagactctcctgtacagcttctggattcaccttcagtgaccacgacatgaactgggtccgccaggctccagggaaggggctggagtgggttggccgtactagaaacgccgctggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 98 - DNA, 321 bp, Artificial Sequence; identical to SEQ ID NO: 18
  Description of Artificial Sequence Synthetic polynucleotide

SEQ ID NO: 99 - protein, 121 aa, Artificial Sequence
  Description of Artificial Sequence Synthetic polypeptide
EVQLVESGGGLVQPGGSLRLSCAASGFTFVDHDMDWVRQAPGKGLEWVGRTRNKLGSYTTEYAASVKGRFTISRDDSKNSLYLQMNSLKTEDTAVYYCAREPKYWIDFDLWGRGTLVTVSS

SEQ ID NO: 100 - protein, 107 aa, Artificial Sequence; identical to SEQ ID NO: 10
  Description of Artificial Sequence Synthetic polypeptide

SEQ ID NO: 101 - protein, 9 aa, Artificial Sequence
  Description of Artificial Sequence Synthetic peptide
FTFVDHDMD

SEQ ID NO: 102 - protein, 19 aa, Artificial Sequence
  Description of Artificial Sequence Synthetic peptide
RTRNKLGSYTTEYAASVKG

SEQ ID NO: 103 - protein, 12 aa, Artificial Sequence
  Description of Artificial Sequence Synthetic peptide
AREPKYWIDFDL

SEQ ID NO: 104 - protein, 11 aa, Artificial Sequence
  Description of Artificial Sequence Synthetic peptide
RASQSISSYLN

SEQ ID NO: 105 - protein, 7 aa, Artificial Sequence
  Description of Artificial Sequence Synthetic peptide
AASSLQS

SEQ ID NO: 106 - protein, 9 aa, Artificial Sequence
  Description of Artificial Sequence Synthetic peptide
QQSYIAPYT

SEQ ID NO: 107 - DNA, 363 bp, Artificial Sequence
  Description of Artificial Sequence Synthetic polynucleotide
gaggtgcagctggtggagtctgggggaggcttggtccagcctggagggtccctgagactctcctgtgcagcctctggattcaccttcgtagaccacgacatggactgggtccgccaggctccagggaaggggctggagtgggttggccgtactagaaacaaactaggaagttacaccacagaatacgccgcgtctgtgaaaggcagattcaccatctcaagagatgattcaaagaactcactgtatctgcaaatgaacagcctgaaaaccgaggacacggcggtgtactactgcgccagagagcctaaatactggatcgacttcgacctatgggggagaggtaccttggtcaccgtctcctca

SEQ ID NO: 108 - DNA, 321 bp, Artificial Sequence; identical to SEQ ID NO: 18
  Description of Artificial Sequence Synthetic polynucleotide

SEQ ID NO: 109 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 114 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 115 - protein, 213 aa, Artificial Sequence
  Description of Artificial Sequence Synthetic polypeptide
DIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 120 - protein, 449 aa, Artificial Sequence
  Description of Artificial Sequence Synthetic polypeptide
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 121 - protein, 107 aa, Artificial Sequence [sequence omitted]

SEQ ID NO: 122 - protein, 330 aa, Homo sapiens [sequence omitted]

SEQ ID NO: 123 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 126 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 127 - protein, 9 aa, Artificial Sequence
  Description of Artificial Sequence Synthetic peptide
  MOD_RES (4)..(4): Ser or Arg
  MOD_RES (5)..(5): Ser, Ile, or Leu
GTFXXYAIS

SEQ ID NO: 128 - protein, 17 aa, Artificial Sequence
  Description of Artificial Sequence Synthetic peptide
  MOD_RES (5)..(5): Ile, Asp, Ala, or His
  MOD_RES (8)..(8): Thr, Val, or Leu
GIIPXFGXANYAQKFQG

SEQ ID NO: 129 - protein, 12 aa, Artificial Sequence
  Description of Artificial Sequence Synthetic peptide
ARGGLDTDEFDL

SEQ ID NO: 130 - protein, 11 aa, Artificial Sequence
  Description of Artificial Sequence Synthetic peptide
RASQSINSYLN

SEQ ID NO: 131 - protein, 7 aa, Artificial Sequence
  Description of Artificial Sequence Synthetic peptide
AASSLQS

SEQ ID NO: 132 - protein, 8 aa, Artificial Sequence
  Description of Artificial Sequence Synthetic peptide
QQGVSDIT

SEQ ID NO: 133 - protein, 9 aa, Artificial Sequence
  Description of Artificial Sequence Synthetic peptide
  MOD_RES (5)..(5): Asn or Ser
  MOD_RES (7)..(7): Ala or Val
  MOD_RES (9)..(9): Ser or Ile
FTFSXYXMX

SEQ ID NO: 134 - protein, 17 aa, Artificial Sequence
  Description of Artificial Sequence Synthetic peptide
  MOD_RES (1)..(1): Ala or Ser
  MOD_RES (5)..(5): Ser or Asp
  MOD_RES (6)..(6): Gly or Ser
  MOD_RES (7)..(7): Gly or Val
  MOD_RES (8)..(8): Ser or Thr
XISGXXXXTYYADSVKG

SEQ ID NO: 135 - protein, 16 aa, Artificial Sequence
  Description of Artificial Sequence Synthetic peptide
AKGPPTYHTNYYYMDV

SEQ ID NO: 136 - protein, 11 aa, Artificial Sequence
  Description of Artificial Sequence Synthetic peptide
RASQGISSWLA

SEQ ID NO: 137 - protein, 7 aa, Artificial Sequence
  Description of Artificial Sequence Synthetic peptide
AASSLQS

SEQ ID NO: 138 - protein, 9 aa, Artificial Sequence
  Description of Artificial Sequence Synthetic peptide
QQTNSFPYT

SEQ ID NO: 139 - protein, 9 aa, Artificial Sequence
  Description of Artificial Sequence Synthetic peptide
  MOD_RES (4)..(4): Ser or Val
  MOD_RES (6)..(6): His or Ala
  MOD_RES (7)..(7): Tyr or Asp
  MOD_RES (9)..(9): Asp or Asn
FTFXDXXMX

SEQ ID NO: 140 - protein, 19 aa, Artificial Sequence
  Description of Artificial Sequence Synthetic peptide
  MOD_RES (5)..(5): Lys or Ala
  MOD_RES (6)..(6): Ala or Leu
  MOD_RES (7)..(7): Ser or Gly
RTRNXXXSYTTEYAASVKG

SEQ ID NO: 141 - protein, 12 aa, Artificial Sequence
  Description of Artificial Sequence Synthetic peptide
AREPKYWIDFDL

SEQ ID NO: 142 - protein, 11 aa, Artificial Sequence
  Description of Artificial Sequence Synthetic peptide
RASQSISSYLN

SEQ ID NO: 143 - protein, 7 aa, Artificial Sequence
  Description of Artificial Sequence Synthetic peptide
AASSLQS

SEQ ID NO: 144 - protein, 9 aa, Artificial Sequence
  Description of Artificial Sequence Synthetic peptide
QQSYIAPYT

SEQ ID NO: 145 - protein, 976 aa, Homo sapiens [sequence omitted]

SEQ ID NO: 146 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 147 - protein, 123 aa, Artificial Sequence
  Description of Artificial Sequence Synthetic polypeptide
QVQLVQSGAAVKKPGESLKISCKGSGYRFTTYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 148 - protein, 107 aa, Artificial Sequence
  Description of Artificial Sequence Synthetic polypeptide
AIQLTQSPSSLSASVGDRVTITCRASQGVSSALAWYQQKPGKAPKLLIYDASSLESGVPSRFSGSGSGTDFTLTISSLQPEDFATYYCQQFNSYPLTFGGGTKVEIK

SEQ ID NO: 149 - protein, 107 aa, Artificial Sequence
  Description of Artificial Sequence Synthetic polypeptide
DIQLTQSPSSLSASVGDRVTITCRASQGIRTDLGWYQQKPGKAPKLLIYDASSLESGVPSRFSGSGSGTDFTLTISSLQPEDFATYYCQQFNSYPLTFGGGTKVEIK

SEQ ID NO: 150 - protein, 107 aa, Artificial Sequence
  Description of Artificial Sequence Synthetic polypeptide
AIRMTQSPSSLSASVGDRVTITCRASQGIRNDLAWYQQKPGKTPKLLIYDASSLESGVPSRFSGSGSGTDFTLTISSLQPEDFATYYCQQFNSYPLTFGGGTKVEIK

SEQ ID NO: 151 - protein, 107 aa, Artificial Sequence
  Description of Artificial Sequence Synthetic polypeptide
AIQMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKVDIK

SEQ ID NO: 152 - protein, 107 aa, Artificial Sequence
  Description of Artificial Sequence Synthetic polypeptide
NIQMTQSPSSLSASVGDRVTITCRASQAISDYLAWFQQKPGKAPKLLIYDASNLETGVPSRFSGSGSGTDFTLTISSLQPEDFATYYCQQLNSYPLTFGGGTKVEIK

SEQ ID NO: 153 - protein, 107 aa, Artificial Sequence
  Description of Artificial Sequence Synthetic polypeptide
AIRMTQSPSSLSASVGDRVIIACRASQGIGGALAWYQQKPGNAPKVLVYDASTLESGVPSRFSGGGSGTDFTLTISSLQPEDFATYYCQQFNSYPLTFGGGTKLEIK

SEQ ID NO: 154 - protein, 107 aa, Artificial Sequence
  Description of Artificial Sequence Synthetic polypeptide
DIAMTQSPPSLSAFVGDRVTITCRASQGIISSLAWYQQKPGKAPKLLIYDASSLESGVPSRFSGSGSGTDFTLTIRSLQPEDFATYYCQQFNSYPLTFGGGTKLEIK

SEQ ID NO: 155 - protein, 107 aa, Artificial Sequence
  Description of Artificial Sequence Synthetic polypeptide
DIQMTQSPSSLSASVGDRVTITCRASQGISSALAWYQQKAGKAPKVLISDASSLESGVPSRFSGSGSGTDFTLSISSLQPEDFATYYCQQFNGYPLTFGGGTKVDIK

SEQ ID NO: 156 - protein, 107 aa, Artificial Sequence
  Description of Artificial Sequence Synthetic polypeptide
AIRMTQSPSSLSASVGDRVTITCQASQGIRNDLGWYQQKPGKAPKLLIYDASNLETGVPSRFSGSGSGTDFTFTISSLQPEDIATYYCQQFNSYPLTFGGGTKLEIK

SEQ ID NO: 157 - protein, 107 aa, Artificial Sequence
  Description of Artificial Sequence Synthetic polypeptide
NIQMTQSPSSLSTSVGDRVTITCRASQGIGTSLAWYQQKPGKPPKLLIYDASSLESGVPSRLSGSGSGTDFTLTISSLQPEDFATYYCQQSNSYPITFGQGTRLEIK

SEQ ID NO: 158 - protein, 107 aa, Artificial Sequence
  Description of Artificial Sequence Synthetic polypeptide
AIQLTQSPSSLSASVGDRVTITCRASQSIGDYLTWYQQKPGKAPKVLIYGASSLQSGVPPRFSGSGSGTDFTLTVSSLQPEDFATYYCQQLNSYPLTFGGGTKLEIK

SEQ ID NO: 159 - protein, 107 aa, Artificial Sequence
  Description of Artificial Sequence Synthetic polypeptide
DIQLTQSPSSLSASVGDRVTITCRASQGVRSTLAWYQQKPGKAPKLLIYDASILESGVPSRFSGSGSGTDFTLTISSLQPEDFATYYCQQFNGYPLTFGQGTRLEIK

SEQ ID NO: 160 - protein, 107 aa, Artificial Sequence
  Description of Artificial Sequence Synthetic polypeptide
DIVMTQSPSSLSASVGDRVTITCRASQGIRNDLGWYQQKPGKAPKLLIYDASSLESGVPSRFSGSGSGTDFTLTISSLQPEDFATYYCQQFNSYPLTFGGGTKLEIK

SEQ ID NO: 161 - protein, 107 aa, Artificial Sequence
  Description of Artificial Sequence Synthetic polypeptide
DIQLTQSPSSLSASVGDRVTITCRASQGISSFLAWYQQKPGKAPKLLIYDASTLQSGVPSRFSGSASGTDFTLTISSLQPEDFATYYCQQLNGYPLTFGGGTKVEIK

SEQ ID NO: 162 - protein, 107 aa, Artificial Sequence
  Description of Artificial Sequence Synthetic polypeptide
AIQLTQSPSSLSASVGDRVTITCRASQGIGSALAWYQQKPGIGPKLLIYDASTLESGVPARFSGSGSRTDFTLTITSLQPEDFATYYCQQFNGYPLTFGGGTKLEIK

SEQ ID NO: 163 - protein, 107 aa, Artificial Sequence
  Description of Artificial Sequence Synthetic polypeptide
AIQLTQSPSSLSASVGDRVTITCRASQGITSALAWYQEKPGKAPNLLIYDASSLESGVPSRFSGSGYGTDFTLTISSLQPEDFATYYCQQLNSYPLTFGGGTKVDIK

SEQ ID NO: 164 - protein, 121 aa, Artificial Sequence
  Description of Artificial Sequence Synthetic polypeptide
QIQLVQSGPELRKPGESVKISCKASGYTFTDYAMYWVKQAPGKGLKWMGWINTYTGKPTYADDFKGRFVFSLEASANTANLQISNLKNEDTATYFCARARGLVDDYVMDAWGQGTSVTVSS

SEQ ID NO: 165 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSGISDRFSGSSSGTTATLTIHGTLAEDEADYYCLSTYSDDNLPVFGGGTKLTVL

SEQ ID NO: 166 - protein, 117 aa, Artificial Sequence
  Description of Artificial Sequence Synthetic polypeptide
EVQLQQYGAELGKPGTSVRLSCKVSGYNIRNTYIHWVNQRPGEGLEWIGRIDPTNGNTISAEKFKTKATLTADTSSHTAYLQFSQLKSDDTAIYFCALNYEGYADYWGQGVMVTGSS

SEQ ID NO: 167 - protein, 106 aa, Artificial Sequence
  Description of Artificial Sequence Synthetic polypeptide
DIQMTQSPSFLSASVGDRVTINCKASQNINKYLNWYQQKVGEAPKRLIFKTNSLQTGIPSRFSGSGSGTDYTLTISSLQTEDVATYFCFQYNIGYTFGAGTKVELK

SEQ ID NO: 168 - protein, 124 aa, Artificial Sequence
  Description of Artificial Sequence Synthetic polypeptide
EVQLQESGPGLVKPSQSLSLTCSVTGYSISSNYRWNWIRKFPGNKVEWMGYINSAGSTNYNPSLKSRISMTRDTSKNQFFLQVNSVTTEDTATYYCARSLRGYITDYSGFFDYWGQGVMVTVSS

SEQ ID NO: 169 - protein, 107 aa, Artificial Sequence
  Description of Artificial Sequence Synthetic polypeptide
DIRMTQSPASLSASLGETVNIECLASEDIFSDLAWYQQKPGKSPQLLIYNANSLQNGVPSRFSGSGSGTRYSLKINSLQSEDVATYFCQQYKNYPLTFGSGTKLEIK

SEQ ID NO: 170 - protein, 117 aa, Artificial Sequence
  Description of Artificial Sequence Synthetic polypeptide
EVQLQQYGAELGKPGTSVRLSCKLSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 171 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTDSLQTGIPSRFSGSGSGTDYTLTISSLQPEDVATYFCFQYKSGFMFGAGTKLELK

SEQ ID NO: 172 - protein, 120 aa, Artificial Sequence
  Description of Artificial Sequence Synthetic polypeptide
QIQLVQSGPELKKPGESVKISCKASGYTFTDYAVYWVIQAPGKGLKWMGWINTYTGKPTYADDFKGRFVFSLETSASTANLQISNLKNEDTATYFCARGAGMTKDYVMDAWGRGVLVTVS

SEQ ID NO: 173 - protein, 108 aa, Artificial Sequence
  Description of Artificial Sequence Synthetic polypeptide
SYELIQPPSASVTLGNTVSLTCVGDELSKRYAQWYQQKPDKTIVSVIYKDSERPSDISDRFSGSSSGTTATLTIHGTLAEDEADYYCLSTYSDDNLPVFGGGTKLTVL

SEQ ID NO: 174 - protein, 116 aa, Artificial Sequence
  Description of Artificial Sequence Synthetic polypeptide
QVQLKESGPGLVQPSQTLSLTCTVSGFSLTSYLVHWVRQPPGKTLEWVGLMWNDGDTSYNSALKSRLSISRDTSKSQVFLKMHSLQAEDTATYYCARESNLGFTYWGHGTLVTVSS

SEQ ID NO: 175 - protein, 107 aa, Artificial Sequence
  Description of Artificial Sequence Synthetic polypeptide
DIQMTQSPASLSASLEEIVTITCKASQGIDDDLSWYQQKPGKSPQLLIYDVTRLADGVPSRFSGSRSGTQYSLKISRPQVADSGIYYCLQSYSTPYTFGAGTKLELK

SEQ ID NO: 176 - protein, 117 aa, Artificial Sequence
  Description of Artificial Sequence Synthetic polypeptide
EVQLQQYGAELGKPGTSVRLSCKVSGYNIRNTYIHWVHQRPGEGLEWIGRIDPTNGNTISAEKFKSKATLTADTSSNTAYMQFSQLKSDDTAIYFCAMNYEGYADYWGQGVMVTVSS

SEQ ID NO: 177 - protein, 106 aa, Artificial Sequence
  Description of Artificial Sequence Synthetic polypeptide
DIQMTQSPSFLSASVGDRLTINCKASQNINKYLNWYQQKLGEAPKRLIFKTNSLQTGIPSRFSGSGSGTDYTLTISSLQPEDVATYFCFQYNIGFTFGAGTKLELK

SEQ ID NO: 178 - protein, 124 aa, Artificial Sequence
  Description of Artificial Sequence Synthetic polypeptide
EVQLVESGGGLVQSGRSLKLSCAASGFTVSDYYMAWVRQAPTKGLEWVATINYDGSTTYHRDSVKGRFTISRDNAKSTLYLQMDSLRSEDTATYYCARHGDYGYHYGAYYFDYWGQGVMVTVSS

SEQ ID NO: 179 - protein, 109 aa, Artificial Sequence
  Description of Artificial Sequence Synthetic polypeptide
DIVLTQSPALAVSLGQRATISCRASQTVSLSGYNLIHWYQQRTGQQPKLLIYRASNLAPGIPARFSGSGSGTDFTLTISPVQSDDIATYYCQQSRESWTFGGGTNLEMK

SEQ ID NO: 180 - protein, 116 aa, Artificial Sequence
  Description of Artificial Sequence Synthetic polypeptide
QIQLVQSGPELKKPGESVKISCKASGYTFTDYAIHWVKQAPGQGLRWMAWINTETGKPTYADDFKGRFVFSLEASASTAHLQISNLKNEDTATFFCAGGSHWFAYWGQGTLVTVSS

SEQ ID NO: 181 - protein, 108 aa, Artificial Sequence
  Description of Artificial Sequence Synthetic polypeptide
SYELIQPPSASVTLENTVSITCSGDELSNKYAHWYQQKPDKTILEVIYNDSERPSGISDRFSGSSSGTTAILTIRDAQAEDEADYYCLSTFSDDDLPIFGGGTKLTVL

SEQ ID NO: 182 - protein, 108 aa, Artificial Sequence
  Description of Artificial Sequence Synthetic polypeptide
SYELIQPPSTSVTLGNTVSLTCVGNELPKRYAYWFQQKPDQSIVRLIYDDDRRPSGISDRFSGSSSGTTATLTIRDAQAEDEAYYYCHSTYTDDKVPIFGGGTKLTVL

SEQ ID NO: 183 - protein, 123 aa, Artificial Sequence
  Description of Artificial Sequence Synthetic polypeptide
EVQLVESGGGLVQPGRSMKLSCKASGFTFSNYDMAWVRQAPTRGLEWVASISYDGITAYYRDSVKGRFTISRENAKSTLYLQLVSLRSEDTATYYCTTEGGYVYSGPHYFDYWGQGVMVTVSS

SEQ ID NO: 184 - protein, 107 aa, Artificial Sequence
  Description of Artificial Sequence Synthetic polypeptide
DIQMTQSPSSMSVSLGDTVTITCRASQDVGIFVNWFQQKPGRSPRRMIYRATNLADGVPSRFSGSRSGSDYSLTISSLESEDVADYHCLQYDEFPRTFGGGTKLELK

SEQ ID NO: 185 - protein, 117 aa, Artificial Sequence
  Description of Artificial Sequence Synthetic polypeptide
EVQLQQYGAELGKPGTSVRLSCKVSGYKIRNTYIHWVNQRPGKGLEWIGRIDPANGNTIYAEKFKSKVTLTADTSSNTAYMQLSQLKSDDTALYFCAMNYEGYEDYWGQGVMVTVSS

SEQ ID NO: 186 - protein, 106 aa, Artificial Sequence
  Description of Artificial Sequence Synthetic polypeptide
DIQMTQSPSFLSASVGDSVTINCKASQNINKYLNWYQQKLGEAPKRLIHKTNSLQPGFPSRFSGSGSGTDYTLTISSLQPEDVAAYFCFQYNSGFTFGAGTKLELK

SEQ ID NO: 187 - protein, 125 aa, Artificial Sequence
  Description of Artificial Sequence Synthetic polypeptide
QVQLVQSGAEVKKPGASVKVSCKASGYTFTDYYIHWVRQAPGQGLEWMGWMNPHSGDTGYAQKFQGRVTMTRDTSTSTVYMELSSLRSEDTAVYYCARHGRGYNGYEGAFDIWGQGTLVTVSSAS

SEQ ID NO: 188 - protein, 107 aa, Artificial Sequence
  Description of Artificial Sequence Synthetic polypeptide
DIQMTQSPSSLSASVGDRVTITCRASQGIGNELGWYQQKPGKAPKLLIYAASNLQSGVPSRFSGSGSGTDFTLTISSLQPEDFATYYCQQYDNLPLTFGQGTKVEIK

SEQ ID NO: 189 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGDTNYAQNFQGRVTMTRDTSTSTVYMELSSLRSEDTAVYYCARHGRGYNGYEGAFDIWGQGTLVTVSSAS

SEQ ID NO: 190 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPLTFGGGTKVEIK

SEQ ID NO: 191 - protein, 125 aa, Artificial Sequence
  Description of Artificial Sequence Synthetic polypeptide
QVQLVQSGAEVKKPGASVKVSCKASGYTFTGYYLHWVRQAPGQGLEWMGWINPNSGGTNYAQKFQGRVTMTRDTSTSTVYMELSSLRSEDTAVYYCARHGRGYEGYEGAFDIWGQGTLVTVSSAS

SEQ ID NO: 192 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASELETGVPSRFSGSGSGTDFTLTISSLQPEDFATYYCQQLNGYPITFGQGTKVEIK

SEQ ID NO: 193 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGWLNPSGGGTSYAQKFQGRVTMTRDTSTSTVYMELSSLRSEDTAVYYCARHGRGYDGYEGAFDIWGQGTLVTVSSAS

SEQ ID NO: 194 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQLNGYPLTFGGGTKVEIK

SEQ ID NO: 195 - protein, 125 aa, Artificial Sequence
  Description of Artificial Sequence Synthetic polypeptide
QVQLVQSGAEVKKPGASVKVSCKASGYTFSTYYMHWVRQAPGQGLEWMGIINPSGGSTSYAQKFQGRVTMTRDTSTSTVYMKLSSLRSEDTAVYYCARHGRGYEGYEGAFDIWGQGTLVTVSSAS

SEQ ID NO: 196 - protein, 107 aa, Artificial Sequence
  Description of Artificial Sequence Synthetic polypeptide
DIQMTQSPSSLSASVGDRVTITCRASQGIRDDLGWYQQKPGKAPKLLIYDASNLETGVPSRFSGSGSGTDFTLTISSLQPEDFATYYCQQANGFPLTFGGGTKVEIK

SEQ ID NO: 197 - protein, 125 aa, Artificial Sequence
  Description of Artificial Sequence Synthetic polypeptide
QVQLVQSGAEVKKPGASVKVSCKASGYTFTGYYIHWVRQAPGQGLEWMGIINPSGGNTNYAQNFQGRVTMTRDTSTSTVYMELSSLRSEDTAVYYCARHGRGYNAYEGAFDIWGQGTLVTVSSAS

SEQ ID NO: 198 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQVNGYPLTFGGGTKVEIK

SEQ ID NO: 199 - protein, 125 aa, Artificial Sequence
  Description of Artificial Sequence Synthetic polypeptide
QVQLVQSGAEVKKPGASVKVSCKASGGTFSSYAISWVRQAPGQGLEWMGVINPTVGGANYAQKFQGRVTMTRDTSTSTVYMELSSLRSEDTAVYYCARHGRGYNEYEGAFDIWGQGTLVTVSSAS

SEQ ID NO: 200 - protein, 107 aa, Artificial Sequence
  Description of Artificial Sequence Synthetic polypeptide
DIQMTQSPSSLSASVGDRVTITCQASQDISDYLNWYQQKPGKAPKLLIYDASNLETGVPSRFSGSGSGTDFTLTISSLQPEDFATYYCQQGNSFPLTFGGGTKLEIK

SEQ ID NO: 201 - protein, 125 aa, Artificial Sequence
  Description of Artificial Sequence Synthetic polypeptide
QVQLVQSGAEVKKLGASVKVSCKASGYTFSSYYMHWVRQAPGQGLEWMGVINPNGAGTNFAQKFQGRVTMTRDTSTSTVYMELSSLRSEDTAVYYCARHGRGYEGYEGAFDIWGQGTLVTVSSAS

SEQ ID NO: 202 - protein, 125 aa, Artificial Sequence
  Description of Artificial Sequence Synthetic polypeptide
QVQLVQSGAEVKKPGASVKVSCKASGYTFTTYYMHWVRQAPGQGLEWMGWINPTGGGTNYAQNFQGRVTMTRDTSTSTVYMELSSLRSEDTAVYYCARHGRGYEGYEGAFDIWGQGTLVTVSSAS

SEQ ID NO: 203 - protein, 107 aa, Artificial Sequence
  Description of Artificial Sequence Synthetic polypeptide
DIQMTQSPSSLSASVGDRVTITCRASQGIRNDVSWYQQKPGKAPKLLIYDASNLETGVPSRFSGSGSGTDFTLTISSLQPEDFATYYCQQLSGYPITFGQGTKLEIK

SEQ ID NO: 204 - protein, 125 aa, Artificial Sequence
  Description of Artificial Sequence Synthetic polypeptide
QVQLVQSGAEVKKPGASVKVSCKASGYTFTSYYIHWVRQAPGQGLEWMGMINPSGGSTNYAQKFQGRVTMTRDTSTSTVYMELSSLRSEDTAVYYCARHGRGYNDYEGAFDIWGQGTLVTVSSAS

SEQ ID NO: 205 - protein, 107 aa, Artificial Sequence
  Description of Artificial Sequence Synthetic polypeptide
DIQMTQSPSSLSASVGDRVTITCRASQSISDWLAWYQQKPGKAPKLLIYEASNLEGGVPSRFSGSGSGTDFTLTISSLQPEDFATYYCQQANSFPYTFGQGTKVEIK

SEQ ID NO: 206 - protein, 125 aa, Artificial Sequence
  Description of Artificial Sequence Synthetic polypeptide
QVQLVQSGAEVKKPGASVKVSCKASGYIFSAYYIHWVRQAPGQGLEWMGIINPSGGSTRYAQKFQGRVTMTRDTSTSTVYMELSSLRSEDTAVYYCARHGRGYGGYEGAFDIWDQGTLVTVSSAS

SEQ ID NO: 207 - protein, 107 aa, Artificial Sequence
  Description of Artificial Sequence Synthetic polypeptide
DIQMTQSPSSLSASVGDRVTITCRASQGIGDYVAWYQQKPGKAPKLLIYDASNLETGVPSRFSGSGSGTDFTLTISSLQPEDFATYYCQQLNGYPITFGQGTRLEIK

SEQ ID NO: 208 - protein, 125 aa, Artificial Sequence
  Description of Artificial Sequence Synthetic polypeptide
EVQLVQSGAEVKKPGESLKISCKGSGYRFTSYWIGWVRQMPGKGLEWMGIIYPDDSDTRYSPSFQGQVTISVDKSNSTAYLQWSSLKASDTAMYYCARHGRGYNGYEGAFDIWGQGTLVTVSSAS

SEQ ID NO: 209 - protein, 107 aa, Artificial Sequence
  Description of Artificial Sequence Synthetic polypeptide
DIQMTQSPSSLSASVGDRVTITCRASQGISSYLAWYQQKPGKAPKLLIYDASNLETGVPSRFSGSGSGTYFTLTISSLQPEDFATYYCQQGASFPITFGQGTKVEIK

SEQ ID NO: 210 - protein, 125 aa, Artificial Sequence
  Description of Artificial Sequence Synthetic polypeptide
EVQLVQSGAEVKKPGESLKISCKGSGSSFPNSWIAWVRQMPGKGLEWMGIIYPSDSDTRYSPSFQGQVTISADKSISTAYLQWSSLEASDTAMYYCARHGRGYNGYEGAFDIWGQGTLVTVSSAS

SEQ ID NO: 211 - protein, 107 aa, Artificial Sequence
  Description of Artificial Sequence Synthetic polypeptide
DIQMTQSPSSLSASVGDRVTITCRASQGIRNYLAWYQQKPGKAPKLLIYDASSLQSGVPSRFSGSGSGTDFTLTISSLQPEDFATYYCQQLNSYPLTFGGGTKVEIK

SEQ ID NO: 212 - protein, 125 aa, Artificial Sequence
  Description of Artificial Sequence Synthetic polypeptide
EVQLVQSGAEVKKPGESLKISCKGSGYSFDSYWIGWVRQMPGKGLEWMGIMYPGDSDTRYSPSFQGQVTISADKSISTAYLQWSSLKASDTAMYYCARHGRGYNAYEGAFDIWGQGTLVTVSSAS

SEQ ID NO: 213 - protein, 107 aa, Artificial Sequence
  Description of Artificial Sequence Synthetic polypeptide
DIQMTQSPSSLSASVGDRVTITCRASQSINNWLAWYQQKPGKAPKLLIYDAFILQSGVPSRFSGSGSGTDFTLTISSLQPEDFATYYCLQLNSYPLTFGPGTKVDIK

SEQ ID NO: 214 - protein, 124 aa, Artificial Sequence
  Description of Artificial Sequence Synthetic polypeptide
EVQLVQSGAEVKKPGESLKISCKGSGYSFTNWIAWVRQMPGKGLEWMGIIYPGDSETRYSPSFQGQVTISADKSISTAYLQWSSLKASDTAMYYCARHGRGYYGYEGAFDIWGQGTLVTVSSAS

SEQ ID NO: 215 - protein, 107 aa, Artificial Sequence
  Description of Artificial Sequence Synthetic polypeptide
DIQMTQSPSSLSASVGDRVTITCRASQGISDNLNWYQQKPGKAPKLLIYDASNLETGVPSRFSGSGSGTDFTLTISSLQPEDFATYYCQQAISFPLTFGQGTKVEIK

SEQ ID NO: 216 - protein, 125 aa, Artificial Sequence
  Description of Artificial Sequence Synthetic polypeptide
EVQLVQSGAEVKKPGESLKISCKGSGYNFTSYWIGWVRQMPGKGLEWMGVIYPDDSETRYSPSFQGQVTISADKSISTAYLQWSSLKASDTAMYYCARHGRGYNGYEGAFDIWGQGTLVTVSSAS

SEQ ID NO: 217 - protein, 107 aa, Artificial Sequence
  Description of Artificial Sequence Synthetic polypeptide
DIQMTQSPSSLSASVGDRVTITCRASRDIRDDLGWYQQKPGKAPKLLIYDASNLETGVPSRFSGSGSGTDFTLTISSLQPEDFATYYCQQANSFPLTFGGGTKVEIK

SEQ ID NO: 218 - protein, 124 aa, Artificial Sequence
  Description of Artificial Sequence Synthetic polypeptide
EVQLVQSGAEVKKPGESLKISCKGSGYTFNTYIGWVRQMPGKGLEWMGIIYPGDSGTRYSPSFQGQVTISADKAISTAYLQWSSLKASDTAMYYCARHSRGYNGYEGAFDIWGQGTLVTVSSAS

SEQ ID NO: 219 - protein, 107 aa, Artificial Sequence
  Description of Artificial Sequence Synthetic polypeptide
DIQMTQSPSSLSASVGDRVTITCRASQGISNYLAWYQQKPGKAPKLLIYDASNLETGVPSRFSGSGSGTDFTLTISSLQPEDFATYYCQQANSFPVTFGQGTKVEIK

SEQ ID NO: 220 - protein, 125 aa, Artificial Sequence
  Description of Artificial Sequence Synthetic polypeptide
EVQLVQSGAEVKKPGESLKISCKGSGYNFTTYWIGWVRQMPGKGLEWMGIIHPADSDTRYNPSFQGQVTISADKSISTAYLQWSSLKASDTAMYYCARHGRGYNGYEGAFDIWGQGTLVTVSSAS

SEQ ID NO: 221 - protein, 107 aa, Artificial Sequence
  Description of Artificial Sequence Synthetic polypeptide
DIQMTQSPSSLSASVGDRVTITCRVSQGISSYLAWYQQKPGKAPKLLIYDASNLETGVPSRFSGSGSGTDFTLTISSLQPEDFATYYCQQANSFPLTFGGGTKVEIK

SEQ ID NO: 222 - protein, 125 aa, Artificial Sequence
  Description of Artificial Sequence Synthetic polypeptide
EVQLVQSGAEVKKPGESLKISCKGSGYRFSNYWIAWVRQMPGKGLEWMGIIYPDNSDTRYSPSFQGQVTISADKSISTAYLQWSSLKASDTAMYYCARHGRGYDGYEGAFDIWGQGTLVTVSSAS

SEQ ID NO: 223 - protein, 107 aa, Artificial Sequence
  Description of Artificial Sequence Synthetic polypeptide
DIQMTQSPSSLSASVGDRVTITCRASQGIRSDLAWYQQKPGKAPKLLIYGASSLQSGVPSRFSGSGSGTDFTLTISSLQPEDFATYYCQQANSFPLSFGQGTKVEIK

SEQ ID NO: 224 - protein, 124 aa, Artificial Sequence
  Description of Artificial Sequence Synthetic polypeptide
EVQLVQSGAEVKKPGESLKISCKGSGYRFASYWIGWVRQMPGKGLEWMGITYPGDSETRYNPSQGQVTISADKSISTAYLQWSSLKASDTAMYYCARHGRGYGGYEGAFDIWGQGTLVTVSSAS

SEQ ID NO: 225 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASNLETGVPSRFSGSGSGTDFTLTISSLQPEDFATYYCQQANSFPLTFGGGTKVEIK

SEQ ID NO: 226 - protein, 125 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHGRGYNGYEGAFDIWGQGTLVTVSSAS

SEQ ID NO: 227 - protein, 107 aa, Artificial Sequence
  Description of Artificial Sequence Synthetic polypeptide
DIQMTQSPSSLSASVGDRVTITCRASQSISNWLAWYQQKPGKAPKLLIYDASNLETGVPSRFSGSGSGTDFTLTISSLQPEDFATYYCQQTNSFPLTFGQGTRLEIK

SEQ ID NO: 228 - protein, 107 aa, Artificial Sequence
  Description of Artificial Sequence Synthetic polypeptide
DIQLTQSPSSLSASVGDRVTITCRASQGVISALAWYQQKPGKAPKLLIYDASSLESGVPSRFSGSGSGTDFTLTISSLQPEDFATYYCQQFNSYPLTFGGGTKVEIK

SEQ ID NO: 229 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASSLESGVPSRFSGSGSGTDFTLTISSLQPEDFATYYCQQFNSYPLTFGGGTKVEIK

SEQ ID NO: 230 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASSLESGVPSRFSGSGSGTDFTLTISSLQPEDFATYYCQQFNSYPLTFGGGTKVEIK

SEQ ID NO: 231 - protein, 107 aa, Artificial Sequence
  Description of Artificial Sequence Synthetic polypeptide
DIQLTQSPSSLSASVGDRVTITCRASQGVISALAWYQQKPGKAPKLLIYDASILESGVPSRFSGSGSGTDFTLTISSLQPEDFATYYCQQFNSYPLTFGGGTKVEIK

SEQ ID NO: 232 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASILESGVPSRFSGSGSGTDFTLTISSLQPEDFATYYCQQFNSYPLTFGGGTKVEIK

SEQ ID NO: 233 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASILESGVPSRFSGSGSGTDFTLTISSLQPEDFATYYCQQFNSYPLTFGGGTKVEIK

SEQ ID NO: 234 - protein, 107 aa, Artificial Sequence
  Description of Artificial Sequence Synthetic polypeptide
DIQLTQSPSSLSASVGDRVTITCRASQGISSALAWYQQKPGKAPKLLIYDASILESGVPSRFSGSGSGTDFTLTISSLQPEDFATYYCQQFNSYPLTFGGGTKVEIK

SEQ ID NO: 235 - protein, 107 aa, Artificial Sequence
  Description of Artificial Sequence Synthetic polypeptide
DIQLTQSPSSLSASVGDRVTITCRASQGVISALAWYQQKPGKAPKLLIYDASTLESGVPSRFSGSGSGTDFTLTISSLQPEDFATYYCQQFNSYPLTFGGGTKVEIK

SEQ ID NO: 236 - protein, 107 aa, Artificial Sequence
  Description of Artificial Sequence Synthetic polypeptide
DIQLTQSPSSLSASVGDRVTITCRASQGIRSALAWYQQKPGKAPKLLIYDASTLESGVPSRFSGSGSGTDFTLTISSLQPEDFATYYCQQFNSYPLTFGGGTKVEIK

SEQ ID NO: 237 - protein, 107 aa, Artificial Sequence
  Description of Artificial Sequence Synthetic polypeptide
DIQLTQSPSSLSASVGDRVTITCRASQGVGSALAWYQQKPGKAPKLLIYDASTLESGVPSRFSGSGSGTDFTLTISSLQPEDFATYYCQQFNSYPLTFGGGTKVEIK

SEQ ID NO: 238 - protein, 123 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSS

SEQ ID NO: 239 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQFNGYPLTFGQGTRLEIK

SEQ ID NO: 240 - protein, 107 aa, Artificial Sequence
  Description of Artificial Sequence Synthetic polypeptide
DIQMTQSPSSLSASVGDRVTITCRASRGISDYLAWYQQKPGKAPKLLIYDASNLETGVPSRFSGSGSGTDFTLTISSLQPEDFATYYCQQANSFPITFGQGTRLEIK

SEQ ID NO: 241 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASTLESGVPSRFSGSGSGTDFTLTISSLQPEDFATYYCQQLNGYPLTFGQGTRLEIK

SEQ ID NO: 242 - protein, 107 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIK

SEQ ID NO: 243 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 244 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 245 - protein, 5 aa, Artificial Sequence
  Description of Artificial Sequence Synthetic peptide
NYWIG

SEQ ID NO: 246 - protein, 17 aa, Artificial Sequence
  Description of Artificial Sequence Synthetic peptide
IINPRDSDTRYRPSFQG

SEQ ID NO: 247 - protein, 14 aa, Artificial Sequence
  Description of Artificial Sequence Synthetic peptide
HGRGYEGYEGAFDI

SEQ ID NO: 248 - protein, 11 aa, Artificial Sequence
  Description of Artificial Sequence Synthetic peptide
RSSQGIRSDLG

SEQ ID NO: 249 - protein, 7 aa, Artificial Sequence
  Description of Artificial Sequence Synthetic peptide
DASNLET

SEQ ID NO: 250 - protein, 9 aa, Artificial Sequence
  Description of Artificial Sequence Synthetic peptide
QQANGFPLT

SEQ ID NO: 251 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDIRYSPSLQGQVTISVDTSTSTAYLQWNSLKPSDTAMYYCARHGRGYNGYEGAFDIWGQGTLVTVSS

SEQ ID NO: 252 - protein, 107 aa, Artificial Sequence
  Description of Artificial Sequence Synthetic polypeptide
DIQMTQSPSSLSASVGDRVTITCRASQGIGDSLAWYQQKPGKAPKLLIYDASNLETGVPSRFSGSGSGTDFTLTISSLQPEDFATYYCQQLNGYPITFGQGTKVEIK

SEQ ID NO: 253 - protein, 17 aa, Artificial Sequence
  Description of Artificial Sequence Synthetic peptide
IIYPGDSDIRYSPSLQG

SEQ ID NO: 254 - protein, 11 aa, Artificial Sequence
  Description of Artificial Sequence Synthetic peptide
RASQGIGDSLA

SEQ ID NO: 255 - protein, 9 aa, Artificial Sequence
  Description of Artificial Sequence Synthetic peptide
QQLNGYPIT

SEQ ID NO: 256 - protein, 107 aa, Artificial Sequence
  Description of Artificial Sequence Synthetic polypeptide
DIQMTQSPSSLSASVGDRVTITCRASQGIRNDLGWYQQKPGKAPKLLIYDASSLESGVPSRFSGSGSGTDFTLTISSLQPEDFATYYCQQLNGYPITFGQGTKVEIK

SEQ ID NO: 257 - protein, 11 aa, Artificial Sequence
  Description of Artificial Sequence Synthetic peptide
RASQGIRNDLG

SEQ ID NO: 258 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSLTRYSPSFQGQVTISADKSISTAYLQWSSLKASDTAMYYCARHGRGYNGYEGAFDIWGQGTLVTVSS

SEQ ID NO: 259 - protein, 17 aa, Artificial Sequence
  Description of Artificial Sequence Synthetic peptide
IIYPGDSLTRYSPSFQG

SEQ ID NO: 260 - protein, 123 aa, Artificial Sequence
  Description of Artificial Sequence Synthetic polypeptide
EVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGRGYNGYEGAFDIWGQGTLVTVSS

SEQ ID NO: 261 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 262 - protein, 107 aa, Artificial Sequence [sequence omitted]

SEQ ID NO: 263 - protein, 5 aa, Artificial Sequence
  Description of Artificial Sequence Synthetic peptide
TYWIG

SEQ ID NO: 264 - protein, 11 aa, Artificial Sequence
  Description of Artificial Sequence Synthetic peptide
RASQGVISALA

SEQ ID NO: 265 - protein, 7 aa, Artificial Sequence
  Description of Artificial Sequence Synthetic peptide
DASILES

SEQ ID NO: 266 - protein, 9 aa, Artificial Sequence
  Description of Artificial Sequence Synthetic peptide
QQFNSYPLT

SEQ ID NO: 267 - protein, 11 aa, Artificial Sequence
  Description of Artificial Sequence Synthetic peptide
RASQGVGSALA

SEQ ID NO: 268 - protein, 7 aa, Artificial Sequence
  Description of Artificial Sequence Synthetic peptide
DASTLES

SEQ ID NO: 269 - protein, 330 aa, Homo sapiens [sequence omitted]

SEQ ID NO: 270 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 330 aa, Artificial Sequence; identical to SEQ ID NO: 123
  Description of Artificial Sequence Synthetic polypeptide

SEQ ID NO: 272 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 273 - protein, 330 aa, Artificial Sequence; identical to SEQ ID NO: 124
  Description of Artificial Sequence Synthetic polypeptide

SEQ ID NO: 274 - protein, 330 aa, Artificial Sequence; identical to SEQ ID NO: 126
  Description of Artificial Sequence Synthetic polypeptide

SEQ ID NO: 275 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 279 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 453 aa, Artificial Sequence
  Description of Artificial Sequence Synthetic polypeptide
EVQLVQSGAEVKKPGESLKISCKGSGYRFTTSWIGWVRQMPGKGLEWMGIIYPGDSDTRYSPSFQGQVTISADKSISTAYLQWSSLKASDTAMYYCARHGLGYNGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 283 - protein, 107 aa, Artificial Sequence [sequence omitted]

SEQ ID NO: 284 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 214 aa, Artificial Sequence
  Description of Artificial Sequence Synthetic polypeptide
DIQMTQSPSSLSASVGDRVTITCRASQGIGSALAWYQQKPGKAPKLLIYDASNLETGVPSRFSGSGSGTDFTLTISSLQPEDFATYYCQQLNGYPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 5 aa, Artificial Sequence
  Description of Artificial Sequence Synthetic peptide
TSWIG

SEQ ID NO: 287 - protein, 14 aa, Artificial Sequence
  Description of Artificial Sequence Synthetic peptide
HGLGYNGYEGAFDI

SEQ ID NO: 288 - protein, 11 aa, Artificial Sequence
  Description of Artificial Sequence Synthetic peptide
RASQGIGSALA

SEQ ID NO: 289 - protein, 9 aa, Artificial Sequence
  Description of Artificial Sequence Synthetic peptide
QQLNGYPLT

SEQ ID NO: 290 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 291 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 292 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 293 - protein, 1193 aa, Homo sapiens
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 294 - protein, 445 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTERSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 295 - protein, 218 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNEYPKDINVKWKIDGSERQNGVLNSWTDQDSKDSTYSMSSTLTLTKDEYERHNSYTCEATHKTSTSPIVKSFNRNEC

SEQ ID NO: 296 - protein, 122 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSA

SEQ ID NO: 297 - protein, 112 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKR

SEQ ID NO: 298 - protein, 450 aa, Artificial Sequence
  Description of Artificial Sequence Synthetic peptide
EVQLVESGGDLVQPGGSLKLSCTASGFTFSNYGMSWIRQTPDKRLEWVATIVGNDYTYFPDSMKGRFTVSRDNAKSILYLQMNSLASADTAMYYCTRHDWVFDYWGQGTPLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 299 - protein, 218 aa, Artificial Sequence
  Description of Artificial Sequence Synthetic peptide
DIVLTQSPASLAVSLGQRAILSCKASQSVSFAGSSLMHWYQQKPGQQPKLLIYRASDLETGIPTRFSGGGSGTDFTLNIHPVEEDDAATYYCQQSREYPYTFGGGTRLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 300 - protein, 453 aa, Artificial Sequence
  Description of Artificial Sequence Synthetic peptide
EVKLVESGGGLLKPGGSLKLSCAASGFTFSKYWMHWVRQAPGKGLEWIGEIEYDGTETNYAPSMKDRFTISRDNAKNTLYLQMSSVRSEDTATYFCTTLQIYNNYLFDYWGQGVMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKVERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 301 - protein, 213 aa, Artificial Sequence
  Description of Artificial Sequence Synthetic peptide
DIQMTQSPSFLSASVGDRVTINCKPSQNINKYLNWYQQKLGEAPKRLIYNTNSLQTGIPSRFSGSGSGTDYTLTITSLQPEDVATYFCLQHNRGVTFGSGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC